(19) United States
(12) Patent Application Publication
(10) Pub. No.: US 2019/0247517 A1
(43) Pub. Date: Aug. 15, 2019

(54) COMPOSITIONS AND METHODS FOR GENE EDITING FOR HEMOPHILIA A

(71) Applicant: Casebia Therapeutics Limited Liability Partnership, San Francisco, CA (US)

(72) Inventor: Alan Richard BROOKS, Cambridge, MA (US)

(21) Appl. No.: 16/163,536

(22) Filed: Oct. 17, 2018

Related U.S. Application Data
(60) Provisional application No. 62/573,633, filed on Oct. 17, 2017.

(51) Int. Cl. A61K 48/00; C12N 9/22; A61P 7/04; A61K 9/127; A61K 9/51; A61K 9/00

(52) U.S. Cl. CPC A61K 48/0058; C12N 9/22; A61P 7/04; A61K 48/0066; A61K 9/51; A61K 9/0019; A61K 48/0075; A61K 9/127

(57) ABSTRACT

Provided include materials and methods for treating Hemophilia A in a subject ex vivo or in vivo. Also provided include materials and methods for knocking in a FVIII-encoding gene in a genome, in particular the locus of albumin gene.

### CROSS-REFERENCE TO RELATED APPLICATIONS

This application claims the benefit of priority to U.S. Provisional Patent Application No. 62/573,633, filed Oct. 17, 2017, the disclosures of which is incorporated herein by reference in its entirety.

### FIELD

The disclosures provided herewith relate to materials and methods for treating a patient with Hemophilia A, both ex vivo and in vivo. In addition, the present disclosures provide materials and methods for editing to modulate the expression, function or activity of a blood-clotting protein such as Factor VIII (FVIII) in a cell by genome editing.

### BACKGROUND

Hemophilia A (HemA) is caused by a genetic defect in the Factor VIII (FVIII) gene that results in low or undetectable levels of FVIII protein in the blood. This results in ineffective clot formation at sites of tissue injury leading to uncontrolled bleeding which can be fatal if not treated. Replacement of the missing FVIII protein is an effective treatment for HemA patients and is the current standard of care. However, protein replacement therapy requires frequent intravenous injection of FVIII protein which is inconvenient in adults, problematic in children, cost prohibitive (>200,000/year), and can result in break through bleeding events if the treatment regimen is not closely followed.

The FVIII gene is expressed primarily in sinusoidal endothelial cells that are present in the liver as well as other sites in the body. Exogenous FVIII can be expressed in and secreted from the hepatocytes of the liver generating FVIII in the circulation and thus affecting a cure of the disease. Gene delivery methods have been developed that target the hepatocytes of the liver and these have thus been used to deliver a FVIII gene as a treatment for HemA both in animal models and in patients in clinical trials

A permanent cure for Hemophilia A is highly desirable. While traditional virus based gene therapy using Adeno Associated Virus (AAV) might show promise in pre-clinical animal models and in patients, it has a number of dis-advantages. AAV based gene therapy uses a FVIII gene driven by a liver specific promoter that is encapsulated inside a AAV virus capsid (typically using the serotypes AAV5, AAV8 or AAV9 or AAVrh10, among others). All AAV viruses used for gene therapy deliver the packaged gene cassette into the nucleus of the transduced cells where the gene cassette remains almost exclusively episomal and it is the episomal copies of the therapeutic gene that give rise to the therapeutic protein. AAV does not have a mechanism to integrate its encapsulated DNA into the genome of the host cells but instead is maintained as an episome that is therefore not replicated when the host cell divides. Episomal DNA can also be subject to degradation over time. It has been demonstrated that when liver cells containing AAV episomes are induced to divide, the AAV genome is not replicated but is instead diluted. As a result, AAV based gene therapy is not expected to be effective when given to children whose livers have not yet achieved adult size. In addition, it is currently unknown how long a AAV based gene therapy will persist when given to adult humans, although animal data have demonstrated only small losses in therapeutic effect over periods as long as 10 years. Therefore, there is a critical need for developing new effective and permeant treatments for HemA.

### SUMMARY

In one aspect, provided herein is a guide RNA (gRNA) sequence having a sequence that is complementary to a genomic sequence within or near an endogenous albumin locus.

In some embodiments, the gRNA comprises a spacer sequence selected from those listed in Table 3 and variants thereof having at least 85% homology to any of those listed in Table 3.

In another aspect, provided herein is a composition having any of the above-mentioned gRNAs.

In some embodiments, the gRNA of the composition comprises a spacer sequence selected from those listed in Table 3 and variants thereof having at least 85% homology to any of those listed in Table 3.

In some embodiments, the composition further comprises one or more of the following: a deoxyribonucleic acid (DNA) endonuclease or a nucleic acid encoding the DNA endonuclease; and a donor template having a nucleic acid sequence encoding a Factor VIII (FVIII) protein or functional derivative thereof.

In some embodiments, the DNA endonuclease is selected from the group consisting of a Cas1, Cas1B, Cas2, Cas3, Cas4, Cas5, Cas6, Cas7, Cas8, Cas9 (also known as Csn1 and Csx12), Cas100, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, or Cpf1 endonuclease, or a functional derivative thereof.

In some embodiments, the DNA endonuclease is Cas9. In some embodiments, the Cas9 is from Streptococcus pyogenes (spCas9). In some embodiments, the Cas9 is from Staphylococcus lugdunensis (SluCas9).

In some embodiments, the nucleic acid encoding the DNA endonuclease is codon optimized.

In some embodiments, the nucleic acid sequence encoding a Factor VIII (FVIII) protein or functional derivative thereof is codon optimized.

In some embodiments, the nucleic acid encoding the DNA endonuclease is a deoxyribonucleic acid (DNA).

In some embodiments, the nucleic acid encoding the DNA endonuclease is a ribonucleic acid (RNA).

In some embodiments, the RNA encoding the DNA endonuclease is linked to the gRNA via a covalent bond.

In some embodiments, the composition further comprises a liposome or lipid nanoparticle.

In some embodiments, the donor template is encoded in an Adeno Associated Virus (AAV) vector.

In some embodiments, the DNA endonuclease is formulated in a liposome or lipid nanoparticle.

In some embodiments, the liposome or lipid nanoparticle also comprises the gRNA.

In some embodiments, the DNA endonuclease is precomplexed with the gRNA, forming a Ribonucleoprotein (RNP) complex.

In another aspect, provided herein is a kit having any of the compositions described above and further having instructions for use.

In another aspect, provided herein is a system comprising a deoxyribonucleic acid (DNA) endonuclease or nucleic acid encoding said DNA endonuclease; a guide RNA (gRNA) comprising a spacer sequence from any one of SEQ ID NOs: 22, 21, 28, 30, 18-20, 23-27, 29, 31-44, and 104; and a donor template comprising a nucleic acid sequence encoding a Factor VIII (FVIII) protein or functional derivative thereof.

In some embodiments, the gRNA comprises a spacer sequence from any one of SEQ ID NOs: 22, 21, 28, and 30. In some embodiments, the gRNA comprises a spacer sequence from SEQ ID NO: 22. In some embodiments, the gRNA comprises a spacer sequence from SEQ ID NO: 21. In some embodiments, the gRNA comprises a spacer sequence from SEQ ID NO: 28. In some embodiments, the gRNA comprises a spacer sequence from SEQ ID NO: 30.

In some embodiments, the DNA endonuclease is selected from the group consisting of a Cas1, Cas1B, Cas2, Cas3, Cas4, Cas5, Cas6, Cas7, Cas8, Cas9 (also known as Csn1 and Csx12), Cas100, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, or Cpf1 endonuclease, or a functional derivative thereof. In some embodiments, the DNA endonuclease is Cas9. In some embodiments, the Cas9 is from Streptococcus pyogenes (spCas9). In some embodiments, the Cas9 is from Staphylococcus lugdunensis (SluCas9).

In some embodiments, the nucleic acid encoding said DNA endonuclease is codon optimized for expression is a host cell. In some embodiments, the host cell is a human cell.

In some embodiments, the nucleic acid encoding a Factor VIII (FVIII) protein or functional derivative thereof is codon optimized for expression in a host cell. In some embodiments, the host cell is a human cell.

In some embodiments, the nucleic acid encoding said DNA endonuclease is a deoxyribonucleic acid (DNA).

In some embodiments, the nucleic acid encoding said DNA endonuclease is a ribonucleic acid (RNA). In some embodiments, the RNA encoding said DNA endonuclease is an mRNA.

In some embodiments, the donor template is encoded in an Adeno Associated Virus (AAV) vector.

In some embodiments, the donor template comprises a donor cassette comprising the nucleic acid sequence encoding a Factor VIII (FVIII) protein or functional derivative, and the donor cassette is flanked on one or both sides by a gRNA target site. In some embodiments, the donor cassette is flanked on both sides by a gRNA target site. In some embodiments, the gRNA target site is a target site for a gRNA in the system. In some embodiments, the gRNA target site of the donor template is the reverse complement of a genomic gRNA target site for a gRNA in the system.

In some embodiments, the DNA endonuclease or nucleic acid encoding the DNA endonuclease is formulated in a liposome or lipid nanoparticle. In some embodiments, the liposome or lipid nanoparticle also comprises the gRNA.

In some embodiments, the system comprises the DNA endonuclease precomplexed with the gRNA, forming a Ribonucleoprotein (RNP) complex.

In another aspect, provided herein is a method of editing a genome in a cell, the method comprising providing the following to the cell: (a) any of the gRNAs described above; (b) a deoxyribonucleic acid (DNA) endonuclease or a nucleic acid encoding the DNA endonuclease; and (c) a donor template having a nucleic acid sequence encoding a Factor VIII (FVIII) protein or functional derivative.

In some embodiments, the gRNA comprises a spacer sequence from any one of SEQ ID NOs: 22, 21, 28, 30, 18-20, 23-27, 29, 31-44, and 104. In some embodiments, the gRNA comprises a spacer sequence from any one of SEQ ID NOs: 22, 21, 28, and 30. In some embodiments, the gRNA comprises a spacer sequence from SEQ ID NO: 22. In some embodiments, the gRNA comprises a spacer sequence from SEQ ID NO: 21. In some embodiments, the gRNA comprises a spacer sequence from SEQ ID NO: 28. In some embodiments, the gRNA comprises a spacer sequence from SEQ ID NO: 30.

In some embodiments, the gRNA has a spacer sequence selected from those listed in Table 3 and variants thereof having at least 85% homology to any of those listed in Table 3.

In some embodiments, the DNA endonuclease is selected from the group consisting of a Cas1, Cas1B, Cas2, Cas3, Cas4, Cas5, Cas6, Cas7, Cas8, Cas9 (also known as Csn1 and Csx12), Cas100, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, or Cpf1 endonuclease; or a functional derivative thereof.

In some embodiments, the DNA endonuclease is Cas9. In some embodiments, the Cas9 is from Streptococcus pyogenes (spCas9). In some embodiments, the Cas9 is from Staphylococcus lugdunensis (SluCas9).

In some embodiments, the nucleic acid encoding the DNA endonuclease is codon optimized for expression in the cell.

In some embodiments, the nucleic acid sequence encoding a Factor VIII (FVIII) protein or functional derivative thereof is codon optimized for expression in the cell.

In some embodiments, the nucleic acid encoding the DNA endonuclease is a deoxyribonucleic acid (DNA).

In some embodiments, the nucleic acid encoding the DNA endonuclease is a ribonucleic acid (RNA).

In some embodiments, the RNA encoding said DNA endonuclease is an mRNA.

In some embodiments, the RNA encoding the DNA endonuclease is linked to the gRNA via a covalent bond.

In some embodiments, the donor template is encoded in an Adeno Associated Virus (AAV) vector.

In some embodiments, the donor template comprises a donor cassette comprising the nucleic acid sequence encoding a Factor VIII (FVIII) protein or functional derivative, and the donor cassette is flanked on one or both sides by a gRNA target site. In some embodiments, the donor cassette is flanked on both sides by a gRNA target site. In some embodiments, the gRNA target site is a target site for the gRNA of (a). In some embodiments, the gRNA target site of the donor template is the reverse complement of a gRNA target site in the cell genome for the gRNA of (a). In some embodiments,

In some embodiments, one or more of (a), (b) and (c) are formulated in a liposome or lipid nanoparticle.

In some embodiments, the DNA endonuclease or nucleic acid encoding the DNA endonuclease is formulated in a liposome or lipid nanoparticle.

In some embodiments, the liposome or lipid nanoparticle also comprises the gRNA.

In some embodiments, the DNA endonuclease is precomplexed with the gRNA, forming a Ribonucleoprotein (RNP) complex, prior to the provision to the cell.

In some embodiments, (a) and (b) are provided to the cell after (c) is provided to the cell.

In some embodiments, (a) and (b) are provided to the cell about 1 to 14 days after (c) is provided to the cell.

In some embodiments, the gRNA of (a) and the DNA endonuclease or nucleic acid encoding the DNA endonuclease of (b) are provided to the cell more than 4 days after the donor template of (c) is provided to the cell.

In some embodiments, the gRNA of (a) and the DNA endonuclease or nucleic acid encoding the DNA endonuclease of (b) are provided to the cell at least 14 days after (c) is provided to the cell.

In some embodiments, one or more additional doses of the gRNA of (a) and the DNA endonuclease or nucleic acid encoding the DNA endonuclease of (b) are provided to the cell following the first dose of the gRNA of (a) and the DNA endonuclease or nucleic acid encoding the DNA endonuclease of (b).

In some embodiments, one or more additional doses of the gRNA of (a) and the DNA endonuclease or nucleic acid encoding the DNA endonuclease of (b) are provided to the cell following the first dose of the gRNA of (a) and the DNA endonuclease or nucleic acid encoding the DNA endonuclease of (b) until a target level of targeted integration of the nucleic acid sequence encoding a Factor VIII (FVIII) protein or functional derivative and/or a target level of expression of the nucleic acid sequence encoding a Factor VIII (FVIII) protein or functional derivative is achieved.

In some embodiments, the nucleic acid sequence encoding a Factor VIII (FVIII) protein or functional derivative is inserted into a genomic sequence of the cell.

In some embodiments, the insertion is at, within, or near the albumin gene or albumin gene regulatory elements in the genome of the cell.

In some embodiments, the insertion is in the first intron of the albumin gene.

In some embodiments, the insertion is at least 37 bp downstream of the end of the first exon of the human albumin gene in the genome and at least 330 bp upstream of the start of the second exon of the human albumin gene in the genome.

In some embodiments, the nucleic acid sequence encoding a Factor VIII (FVIII) protein or functional derivative is expressed under the control of the endogenous albumin promoter.

In some embodiments, the cell is a hepatocyte.

In another aspect, provided herein is a genetically modified cell in which the genome of the cell is edited by any of the method described above.

In some embodiments, the nucleic acid sequence encoding a Factor VIII (FVIII) protein or functional derivative is inserted into a genomic sequence of the cell.

In some embodiments, the insertion is at, within, or near the albumin gene or albumin gene regulatory elements in the genome of the cell.

In some embodiments, the insertion is in the first intron of the albumin gene.

In some embodiments, the insertion is at least 37 bp downstream of the end of the first exon of the human albumin gene in the genome and at least 330 bp upstream of the start of the second exon of the human albumin gene in the genome.

In some embodiments, the nucleic acid sequence encoding a Factor VIII (FVIII) protein or functional derivative is expressed under the control of the endogenous albumin promoter.

In some embodiments, the nucleic acid sequence encoding a Factor VIII (FVIII) protein or functional derivative thereof is codon optimized.

In some embodiments, the cell is a hepatocyte.

In another aspect, provided herein is a method of treating Hemophilia A in a subject, the method comprising providing the following to a cell in the subject: (a) a gRNA comprising a spacer sequence from any one of SEQ ID NOs: 22, 21, 28, 30, 18-20, 23-27, 29, 31-44, and 104; (b) a DNA endonuclease or nucleic acid encoding said DNA endonuclease; and (c) a donor template comprising a nucleic acid sequence encoding a Factor VIII (FVIII) protein or functional derivative.

In some embodiments, the gRNA comprises a spacer sequence from any one of SEQ ID NOs: 22, 21, 28, and 30. In some embodiments, the gRNA comprises a spacer sequence from SEQ ID NO: 22. In some embodiments, the gRNA comprises a spacer sequence from SEQ ID NO: 21. In some embodiments, the gRNA comprises a spacer sequence from SEQ ID NO: 28. In some embodiments, the gRNA comprises a spacer sequence from SEQ ID NO: 30.

In some embodiments, the subject is a patient having or is suspected of having Hemophilia A.

In some embodiments, the subject is diagnosed with a risk of Hemophilia A.

In some embodiments, the DNA endonuclease is selected from the group consisting of a Cas1, Cas1B, Cas2, Cas3, Cas4, Cas5, Cas6, Cas7, Cas8, Cas9 (also known as Csn1 and Csx12), Cas100, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, or Cpf1 endonuclease; or a functional derivative thereof.

In some embodiments, the DNA endonuclease is Cas9. In some embodiments, the Cas9 is from Streptococcus pyogenes (spCas9). In some embodiments, the Cas9 is from Staphylococcus lugdunensis (SluCas9).

In some embodiments, the nucleic acid encoding said DNA endonuclease is codon optimized for expression in the cell.

In some embodiments, the nucleic acid sequence encoding a Factor VIII (FVIII) protein or functional derivative thereof is codon optimized for expression in the cell.

In some embodiments, the nucleic acid encoding said DNA endonuclease is a deoxyribonucleic acid (DNA).

In some embodiments, the nucleic acid encoding said DNA endonuclease is a ribonucleic acid (RNA). In some embodiments, the RNA encoding said DNA endonuclease is an mRNA.

In some embodiments, one or more of the gRNA of (a), the DNA endonuclease or nucleic acid encoding the DNA endonuclease of (b), and the donor template of (c) are formulated in a liposome or lipid nanoparticle.

In some embodiments, the donor template is encoded in an Adeno Associated Virus (AAV) vector.

In some embodiments, the donor template comprises a donor cassette comprising the nucleic acid sequence encoding a Factor VIII (FVIII) protein or functional derivative, and wherein the donor cassette is flanked on one or both sides by a gRNA target site. In some embodiments, the donor cassette is flanked on both sides by a gRNA target site. In some embodiments, the gRNA target site is a target site for the gRNA of (a). In some embodiments, the gRNA target site of the donor template is the reverse complement of the gRNA target site in the cell genome for the gRNA of (a).

In some embodiments, providing the donor template to the cell comprises administering the donor template to the subject. In some embodiments, the administration is via intravenous route.

In some embodiments, DNA endonuclease or nucleic acid encoding the DNA endonuclease is formulated in a liposome or lipid nanoparticle. In some embodiments, the liposome or lipid nanoparticle also comprises the gRNA.

In some embodiments, providing the gRNA and the DNA endonuclease or nucleic acid encoding the DNA endonuclease to the cell comprises administering the liposome or lipid nanoparticle to the subject. In some embodiments, the administration is via intravenous route.

In some embodiments, the method comprises providing to the cell the DNA endonuclease pre-complexed with the gRNA, forming a Ribonucleoprotein (RNP) complex.

In some embodiments, the gRNA of (a) and the DNA endonuclease or nucleic acid encoding the DNA endonuclease of (b) are provided to the cell more than 4 days after the donor template of (c) is provided to the cell. In some embodiments, the gRNA of (a) and the DNA endonuclease or nucleic acid encoding the DNA endonuclease of (b) are provided to the cell at least 14 days after the donor template of (c) is provided to the cell.

In some embodiments, one or more additional doses of the gRNA of (a) and the DNA endonuclease or nucleic acid encoding the DNA endonuclease of (b) are provided to the cell following the first dose of the gRNA of (a) and the DNA endonuclease or nucleic acid encoding the DNA endonuclease of (b). In some embodiments, one or more additional doses of the gRNA of (a) and the DNA endonuclease or nucleic acid encoding the DNA endonuclease of (b) are provided to the cell following the first dose of the gRNA of (a) and the DNA endonuclease or nucleic acid encoding the DNA endonuclease of (b) until a target level of targeted integration of the nucleic acid sequence encoding a Factor VIII (FVIII) protein or functional derivative and/or a target level of expression of the nucleic acid sequence encoding a Factor VIII (FVIII) protein or functional derivative is achieved.

In some embodiments, providing the gRNA of (a) and the DNA endonuclease or nucleic acid encoding the DNA endonuclease of (b) to the cell comprises administering to the subject a lipid nanoparticle comprising nucleic acid encoding the DNA endonuclease and the gRNA.

In some embodiments, providing the donor template of (c) to the cell comprises administering to the subject the donor template encoded in an AAV vector.

In some embodiments, the nucleic acid sequence encoding a Factor VIII (FVIII) protein or functional derivative is expressed under the control of the endogenous albumin promoter.

In some embodiments, the cell is a hepatocyte.

In some embodiments, the nucleic acid sequence encoding a Factor VIII (FVIII) protein or functional derivative is expressed in the liver of the subject.

In another aspect, provided herein is a method of treating Hemophilia A in a subject. The method comprises administering any of the above-mentioned genetically modified cells to the subject.

In some embodiments, the subject is a patient having or is suspected of having Hemophilia A.

In some embodiments, the subject is diagnosed with a risk of Hemophilia A.

In some embodiments, the genetically modified cell is autologous.

In some embodiments, the cell is a hepatocyte.

In some embodiments, the nucleic acid sequence encoding a Factor VIII (FVIII) protein or functional derivative is inserted into a genomic sequence of the cell.

In some embodiments, the insertion is at, within, or near the albumin gene or albumin gene regulatory elements in the genome of the cell.

In some embodiments, the insertion is in the first intron of the albumin gene.

In some embodiments, the insertion is at least 37 bp downstream of the end of the first exon of the human albumin gene in the genome and at least 330 bp upstream of the start of the second exon of the human albumin gene in the genome.

In some embodiments, the nucleic acid sequence encoding a Factor VIII (FVIII) protein or functional derivative is expressed under the control of the endogenous albumin promoter.

In some embodiments, the method further comprises obtaining a biological sample from the subject wherein the biological sample comprises a hepatocyte cell and editing the genome of the hepatocyte cell by inserting a nucleic acid sequence encoding a Factor VIII (FVIII) protein or functional derivative thereof into a genomic sequence of the cell, thereby producing the genetically modified cell.

In another aspect, provided herein is a method of treating Hemophilia A in a subject. The method comprises obtaining a biological sample from the subject wherein the biological sample comprises a hepatocyte cell, providing the following to the hepatocyte cell: (a) any of the gRNA described above; (b) a deoxyribonucleic acid (DNA) endonuclease or a nucleic acid encoding the DNA endonuclease; and (c) a donor template having a nucleic acid sequence encoding a Factor VIII (FVIII) protein or functional derivative, thereby producing a genetically modified cell, and administering the genetically modified cell to the subject.

In some embodiments, the subject is a patient having or is suspected of having Hemophilia A.

In some embodiments, the subject is diagnosed with a risk of Hemophilia A.

In some embodiments, the genetically modified cell is autologous.

In some embodiments, the gRNA comprises a sequence selected from those listed in Table 3 and variants thereof having at least 85% homology to any of those listed in Table 3.

In some embodiments, the DNA endonuclease is selected from the group consisting of a Cas1, Cas1B, Cas2, Cas3, Cas4, Cas5, Cas6, Cas7, Cas8, Cas9 (also known as Csn1 and Csx12), Cas100, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, or Cpf1 endonuclease, or a functional derivative thereof.

In some embodiments, the DNA endonuclease is Cas9. In some embodiments, the Cas9 is from Streptococcus pyogenes (spCas9). In some embodiments, the Cas9 is from Staphylococcus lugdunensis (SluCas9).

In some embodiments, the nucleic acid encoding the DNA endonuclease is codon optimized.

In some embodiments, the nucleic acid sequence encoding a Factor VIII (FVIII) protein or functional derivative thereof is codon optimized.

In some embodiments, the nucleic acid encoding the DNA endonuclease is a deoxyribonucleic acid (DNA) sequence.

In some embodiments, the nucleic acid encoding the DNA endonuclease is a ribonucleic acid (RNA) sequence.

In some embodiments, the RNA sequence encoding the DNA endonuclease is linked to the gRNA via a covalent bond.

In some embodiments, one or more of (a), (b) and (c) are formulated in a liposome or lipid nanoparticle.

In some embodiments, the donor template is encoded in an Adeno Associated Virus (AAV) vector.

In some embodiments, the DNA endonuclease is formulated in a liposome or lipid nanoparticle.

In some embodiments, the liposome or lipid nanoparticle also comprises the gRNA.

In some embodiments, the DNA endonuclease is precomplexed with the gRNA, forming a Ribonucleoprotein (RNP) complex, prior to the provision to the cell.

In some embodiments, (a) and (b) are provided to the cell after (c) is provided to the cell.

In some embodiments, (a) and (b) are provided to the cell about 1 to 14 days after (c) is provided to the cell.

In some embodiments, the nucleic acid sequence encoding a Factor VIII (FVIII) protein or functional derivative is inserted into a genomic sequence of the cell.

In some embodiments, the insertion is at, within, or near the albumin gene or albumin gene regulatory elements in the genome of the cell.

In some embodiments, the insertion is in the first intron of the albumin gene.

In some embodiments, the insertion is at least 37 bp downstream of the end of the first exon of the human albumin gene in the genome and at least 330 bp upstream of the start of the second exon of the human albumin gene in the genome.

In some embodiments, the nucleic acid sequence encoding a Factor VIII (FVIII) protein or functional derivative is expressed under the control of the endogenous albumin promoter.

In some embodiments, the cell is a hepatocyte.

In another aspect, provided herein is a method of treating Hemophilia A in a subject. The method comprises providing the following to a cell in the subject: (a) any of the gRNA described above; (b) a deoxyribonucleic acid (DNA) endonuclease or a nucleic acid encoding the DNA endonuclease; and (c) a donor template having a nucleic acid sequence encoding a Factor VIII (FVIII) protein or functional derivative.

In some embodiments, the subject is a patient having or is suspected of having Hemophilia A.

In some embodiments, the subject is diagnosed with a risk of Hemophilia A.

In some embodiments, the gRNA comprises a sequence selected from those listed in Table 3 and variants thereof having at least 85% homology to any of those listed in Table 3.

In some embodiments, the DNA endonuclease is selected from the group consisting of a Cas1, Cas1B, Cas2, Cas3, Cas4, Cas5, Cas6, Cas7, Cas8, Cas9 (also known as Csn1 and Csx12), Cas100, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, or Cpf1 endonuclease; or a functional derivative thereof.

In some embodiments, the DNA endonuclease is Cas9. In some embodiments, the Cas9 is from Streptococcus pyogenes (spCas9). In some embodiments, the Cas9 is from Staphylococcus lugdunensis (SluCas9).

In some embodiments, the nucleic acid encoding the DNA endonuclease is codon optimized.

In some embodiments, the nucleic acid sequence encoding a Factor VIII (FVIII) protein or functional derivative thereof is codon optimized.

In some embodiments, the nucleic acid encoding the DNA endonuclease is a deoxyribonucleic acid (DNA) sequence.

In some embodiments, the nucleic acid encoding the DNA endonuclease is a ribonucleic acid (RNA) sequence.

In some embodiments, the RNA sequence encoding the DNA endonuclease is linked to the gRNA via a covalent bond.

In some embodiments, one or more of (a), (b) and (c) are formulated in a liposome or lipid nanoparticle.

In some embodiments, the donor template is encoded in an Adeno Associated Virus (AAV) vector.

In some embodiments, the DNA endonuclease is formulated in a liposome or lipid nanoparticle.

In some embodiments, the liposome or lipid nanoparticle also comprises the gRNA.

In some embodiments, the DNA endonuclease is precomplexed with the gRNA, forming a Ribonucleoprotein (RNP) complex, prior to the provision to the cell.

In some embodiments, (a) and (b) are provided to the cell after (c) is provided to the cell.

In some embodiments, (a) and (b) are provided to the cell about 1 to 14 days after (c) is provided to the cell.

In some embodiments, the nucleic acid sequence encoding a Factor VIII (FVIII) protein or functional derivative is inserted into a genomic sequence of the cell.

In some embodiments, the insertion is at, within, or near the albumin gene or albumin gene regulatory elements in the genome of the cell.

In some embodiments, the insertion is in the first intron of the albumin gene in the genome of the cell.

In some embodiments, the insertion is at least 37 bp downstream of the end of the first exon of the human albumin gene in the genome and at least 330 bp upstream of the start of the second exon of the human albumin gene in the genome.

In some embodiments, the nucleic acid sequence encoding a Factor VIII (FVIII) protein or functional derivative is expressed under the control of the endogenous albumin promoter.

In some embodiments, the cell is a hepatocyte.

In some embodiments, the nucleic acid sequence encoding a Factor VIII (FVIII) protein or functional derivative is expressed in the liver of the subject.

In another aspect, provided herein is a kit comprising one or more elements of a system described above, and further comprising instructions for use.

### BRIEF DESCRIPTION OF THE DRAWINGS

An understanding of certain features and advantages of the present disclosure will be obtained by reference to the following detailed description that sets forth illustrative embodiments, in which the principles of the disclosure are utilized, and the accompanying drawings of which:

FIG. 1 shows multiple alignment of differently codon optimized FVIII-BDD coding sequences. Only the mature coding sequence is shown (signal peptide region is deleted). ClustalW algorithm was used.

FIG. 2 shows non-limiting, exemplary designs of DNA donor template.

FIG. 3 shows the results of TIDE analysis of cutting efficiency of mA1b gRNA-T1 in Hepa1-6 cells.

FIG. 4 shows the results of INDEL frequencies in the liver and spleen of mice 3 days after dosing with lipid nanoparticles (LNP) encapsulating Cas9 mRNA and mA1b gRNA_T1 at different doses or PBS control. N=5 mice per group, mean values are plotted.

FIG. 5 shows designs of DNA donor templates for targeted integration in to albumin intron 1 used in Example 4. SA; splice acceptor sequence, LHA; Left homology arm; RHA; right homology arm, pA; poly adenylation signal, gRNA site; target site for gRNA that mediates cutting by gRNA targeted Cas9 nuclease, delta furin; deletion of the furin site in FVIII, FVIII-BDD; coding sequence for human FVIII with B-domain deletion (BDD) in which the B-domain is replaced by the SQ link peptide.

FIG. 6 shows INDEL frequencies of 8 candidate gRNA targeting human albumin intron 1 in primary human hepatocytes from 4 donors. gRNA targeting the AAVS1 locus and unrelated human gene (C3) are included as controls.

FIG. 7 shows INDEL frequencies in non-human primate (Monkey) primary hepatocytes transfected with different albumin guide RNA and spCas9 mRNA.

FIG. 8 shows a schematic of an exemplary AAV-mSEAP donor cassette.

FIG. 9 shows a schematic of an exemplary FVIII donor cassette used for packaging into AAV.

FIG. 10 shows FVIII levels in the blood of hemophilia A mice over time after injection of AAV8-pCB056 followed by LNP encapsulating spCas9 mRNA and mAlbT1 guide RNA.

FIG. 11 shows FVIII levels in Hemophilia A mice at day 10 and day 17 after the LNP encapsulating spCas9 mRNA and gRNA was injected. LNP was dosed either 17 days or 4 days after AAV8-pCB056.

FIG. 12 shows a schematic of exemplary plasmid donors containing the human FVIII gene and different polyadenylation signal sequences.

FIG. 13 shows FVIII activity and FVIII activity/targeted integration ratios in mice after hydrodynamic injection of plasmid donors with 3 different polyA signals followed by LNP encapsulated Cas9mRNA and mAlbT1 gRNA. Groups 2, 3 and 4 were dosed with pCB065, pCB076 and pCB077 respectively. The table contains the values for FVIII activity on day 10, targeted integration frequency and FVIII activity/TI ratio (Ratio) for each individual mouse.

FIG. 14 shows a schematic of exemplary AAV donor cassettes used to evaluate targeted integration in primary human hepatocytes.

FIG. 15 shows SEAP activity in the media of primary human hepatocytes transduced with AAV-DJ-SEAP virus with or without lipofection of spCas9 mRNA and hALb4 gRNA. Two cell donors were tested (HJK, ONR) indicated by the black and white bars. The 3 pairs of bars on the left represent the SEAP activity in control conditions of cells transfected with only Cas9 and gRNA (first pair of bars), AAV-DJ-pCB0107 (SEAP virus) at 100,000 MOI alone (second pair of bars) or AAV-DJ-pCB0156 (FVIII virus) at 100,000 MOI alone (third pair of bars). The 4 pairs of bar on the right represent the SEAP activity in wells of cells transduced with the AAV-DJ-pCB0107 (SEAP virus) at various MOI and transfected with Cas9 mRNA and the hAlb T4 gRNA.

FIG. 16 shows FVIII activity in the media of primary human hepatocytes transduced with AAV-DJ-FVIII virus with or without lipofection of spCas9 mRNA and hALb4 gRNA. Two cell donors were tested (HJK, ONR) indicated by the black and white bars. The 2 pairs of bars on the left represent the FVIII activity in control conditions of cells transduced with AAV-DJ-pCB0107 (SEAP virus) at 100,000 MOI alone (first pair of bars) or AAV-DJ-pCB0156 (FVIII virus) at 100,000 MOI alone (second pair of bars). The 4 pairs of bar on the right represent the FVIII activity in media from wells of cells transduced with the AAV-DJ-pCB0156 (FVIII virus) at various MOI and transfected with Cas9 mRNA and the hAlb T4 gRNA.

### DETAILED DESCRIPTION

The disclosures provide, inter alia, compositions and methods for editing to modulate the expression, function or activity of a blood-clotting protein such as Factor VIII (FVIII) in a cell by genome editing. The disclosures also provide, inter alia, compositions and methods for treating a patient with Hemophilia A, both ex vivo and in vivo.

### Definitions

Unless defined otherwise, all technical and scientific terms used herein have the same meaning as is commonly understood by one of skill in the art to which the claimed subject matter belongs. It is to be understood that the detailed descriptions are exemplary and explanatory only and are not restrictive of any subject matter claimed. In this application, the use of the singular includes the plural unless specifically stated otherwise. It must be noted that, as used in the specification, the singular forms “a,” “an” and “the” include plural referents unless the context clearly dictates otherwise. In this application, the use of “or” means “and/or” unless stated otherwise. Furthermore, use of the term “including” as well as other forms, such as “include”, “includes,” and “included,” is not limiting.

Although various features of the disclosures may be described in the context of a single embodiment, the features may also be provided separately or in any suitable combination. Conversely, although the disclosures may be described herein in the context of separate embodiments for clarity, the disclosures may also be implemented in a single embodiment. Any published patent applications and any other published references, documents, manuscripts, and scientific literature cited herein are incorporated herein by reference for any purpose. In the case of conflict, the present specification, including definitions, will control. In addition, the materials, methods, and examples are illustrative only and not intended to be limiting.

As used herein, ranges and amounts can be expressed as “about” a particular value or range. About also includes the exact amount. Hence “about 5 μL” means “about 5 μL” and also “5 μL.” Generally, the term “about” includes an amount that would be expected to be within experimental error such as ±10%.

When a range of numerical values is presented herein, it is contemplated that each intervening value between the lower and upper limit of the range, the values that are the upper and lower limits of the range, and all stated values with the range are encompassed within the disclosure. All the possible sub-ranges within the lower and upper limits of the range are also contemplated by the disclosure.

The terms “polypeptide,” “polypeptide sequence,” “peptide,” “peptide sequence,” “protein,” “protein sequence” and “amino acid sequence” are used interchangeably herein to designate a linear series of amino acid residues connected one to the other by peptide bonds, which series may include proteins, polypeptides, oligopeptides, peptides, and fragments thereof. The protein may be made up of naturally occurring amino acids and/or synthetic (e.g., modified or non-naturally occurring) amino acids. Thus “amino acid”, or “peptide residue”, as used herein means both naturally occurring and synthetic amino acids. The terms “polypeptide”, “peptide”, and “protein” includes fusion proteins, including, but not limited to, fusion proteins with a heterologous amino acid sequence, fusions with heterologous and homologous leader sequences, with or without N-terminal methionine residues; immunologically tagged proteins; fusion proteins with detectable fusion partners, e.g., fusion proteins including as a fusion partner a fluorescent protein, β-galactosidase, luciferase, and the like. Furthermore, it should be noted that a dash at the beginning or end of an amino acid sequence indicates either a peptide bond to a further sequence of one or more amino acid residues or a covalent bond to a carboxyl or hydroxyl end group. However, the absence of a dash should not be taken to mean that such peptide bond or covalent bond to a carboxyl or hydroxyl end group is not present, as it is conventional in representation of amino acid sequences to omit such.

The term “polynucleotide,” “polynucleotide sequence,” “oligonucleotide,” “oligonucleotide sequence,” “oligomer,” “oligo,” “nucleic acid sequence” or “nucleotide sequence” used interchangeably herein, refer to a polymeric form of nucleotides of any length, either ribonucleotides or deoxyribonucleotides. Thus, this term includes, but is not limited to, single-, double-, or multi-stranded DNA or RNA, genomic DNA, cDNA, DNA-RNA hybrids, or a polymer having purine and pyrimidine bases or other natural, chemically or biochemically modified, non-natural, or derivatized nucleotide bases.

The terms “derivative” and “variant” refer without limitation to any compound such as nucleic acid or protein that has a structure or sequence derived from the compounds disclosed herein and whose structure or sequence is sufficiently similar to those disclosed herein such that it has the same or similar activities and utilities or, based upon such similarity, would be expected by one skilled in the art to exhibit the same or similar activities and utilities as the referenced compounds, thereby also interchangeably referred to “functionally equivalent” or as “functional equivalents.” Modifications to obtain “derivatives” or “variants” may include, for example, addition, deletion and/or substitution of one or more of the nucleic acids or amino acid residues.

The functional equivalent or fragment of the functional equivalent, in the context of a protein, may have one or more conservative amino acid substitutions. The term “conservative amino acid substitution” refers to substitution of an amino acid for another amino acid that has similar properties as the original amino acid. The groups of conservative amino acids are as follows:

| Group | Name of the amino acids |
|---|---|
| Aliphatic | Gly, Ala, Val, Leu, Ile |
| Hydroxyl or Sulfhydryl/Selenium-containing | Ser, Cys, Thr, Met |
| Cyclic | Pro |
| Aromatic | Phe, Tyr, Trp |
| Basic | His, Lys, Arg |
| Acidic and their Amide | Asp, Glu, Asn, Gln |

Conservative substitutions may be introduced in any position of a preferred predetermined peptide or fragment thereof. It may however also be desirable to introduce non-conservative substitutions, particularly, but not limited to, a non-conservative substitution in any one or more positions. A non-conservative substitution leading to the formation of a functionally equivalent fragment of the peptide would for example differ substantially in polarity, in electric charge, and/or in steric bulk while maintaining the functionality of the derivative or variant fragment.

“Percentage of sequence identity” is determined by comparing two optimally aligned sequences over a comparison window, wherein the portion of the polynucleotide or polypeptide sequence in the comparison window may have additions or deletions (i.e., gaps) as compared to the reference sequence (which does not have additions or deletions) for optimal alignment of the two sequences. In some cases the percentage can be calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

The terms “identical” or percent “identity” in the context of two or more nucleic acid or polypeptide sequences, refer to two or more sequences or subsequences that are the same or have a specified percentage of amino acid residues or nucleotides that are the same (e.g., 60%, 65%, 70%, 75%, 80%, 85%, 90%, 95%, 98%, or 99% identity over a specified region, e.g., the entire polypeptide sequences or individual domains of the polypeptides), when compared and aligned for maximum correspondence over a comparison window or designated region as measured using one of the following sequence comparison algorithms or by manual alignment and visual inspection. Such sequences are then said to be “substantially identical.” This definition also refers to the complement of a test sequence.

The term “complementary” or “substantially complementary,” interchangeably used herein, means that a nucleic acid (e.g. DNA or RNA) has a sequence of nucleotides that enables it to non-covalently bind, i.e. form Watson-Crick base pairs and/or G/U base pairs to another nucleic acid in a sequence-specific, antiparallel, manner (i.e., a nucleic acid specifically binds to a complementary nucleic acid). As is known in the art, standard Watson-Crick base-pairing includes: adenine (A) pairing with thymidine (T), adenine (A) pairing with uracil (U), and guanine (G) pairing with cytosine (C).

A DNA sequence that “encodes” a particular RNA is a DNA nucleic acid sequence that is transcribed into RNA. A DNA polynucleotide may encode an RNA (mRNA) that is translated into protein, or a DNA polynucleotide may encode an RNA that is not translated into protein (e.g. tRNA, rRNA, or a guide RNA; also called “non-coding” RNA or “ncRNA”). A “protein coding sequence or a sequence that encodes a particular protein or polypeptide, is a nucleic acid sequence that is transcribed into mRNA (in the case of DNA) and is translated (in the case of mRNA) into a polypeptide in vitro or in vivo when placed under the control of appropriate regulatory sequences.

As used herein, “codon” refers to a sequence of three nucleotides that together form a unit of genetic code in a DNA or RNA molecule. As used herein the term “codon degeneracy” refers to the nature in the genetic code permitting variation of the nucleotide sequence without affecting the amino acid sequence of an encoded polypeptide.

The term “codon-optimized” or “codon optimization” refers to genes or coding regions of nucleic acid molecules for transformation of various hosts, refers to the alteration of codons in the gene or coding regions of the nucleic acid molecules to reflect the typical codon usage of the host organism without altering the polypeptide encoded by the DNA. Such optimization includes replacing at least one, or more than one, or a significant number, of codons with one or more codons that are more frequently used in the genes of that organism. Codon usage tables are readily available, for example, at the “Codon Usage Database” available at www.kazusa.or.jp/codon/(visited Mar. 20, 2008). By utilizing the knowledge on codon usage or codon preference in each organism, one of ordinary skill in the art can apply the frequencies to any given polypeptide sequence, and produce a nucleic acid fragment of a codon-optimized coding region which encodes the polypeptide, but which uses codons optimal for a given species. Codon-optimized coding regions can be designed by various methods known to those skilled in the art.

The term “recombinant” or “engineered” when used with reference, for example, to a cell, a nucleic acid, a protein, or a vector, indicates that the cell, nucleic acid, protein or vector has been modified by or is the result of laboratory methods. Thus, for example, recombinant or engineered proteins include proteins produced by laboratory methods. Recombinant or engineered proteins can include amino acid residues not found within the native (non-recombinant or wild-type) form of the protein or can be include amino acid residues that have been modified, e.g., labeled. The term can include any modifications to the peptide, protein, or nucleic acid sequence. Such modifications may include the following: any chemical modifications of the peptide, protein or nucleic acid sequence, including of one or more amino acids, deoxyribonucleotides, or ribonucleotides; addition, deletion, and/or substitution of one or more of amino acids in the peptide or protein; and addition, deletion, and/or substitution of one or more of nucleic acids in the nucleic acid sequence.

The term “genomic DNA” or “genomic sequence” refers to the DNA of a genome of an organism including, but not limited to, the DNA of the genome of a bacterium, fungus, archea, plant or animal.

As used herein, “transgene,” “exogenous gene” or “exogenous sequence,” in the context of nucleic acid, refers to a nucleic acid sequence or gene that was not present in the genome of a cell but artificially introduced into the genome, e.g. via genome-edition.

As used herein, “endogenous gene” or “endogenous sequence,” in the context of nucleic acid, refers to a nucleic acid sequence or gene that is naturally present in the genome of a cell, without being introduced via any artificial means.

The term “vector” or “expression vector” means a replicon, such as plasmid, phage, virus, or cosmid, to which another DNA segment, i.e. an “insert”, may be attached so as to bring about the replication of the attached segment in a cell.

The term “expression cassette” refers to a vector having a DNA coding sequence operably linked to a promoter. “Operably linked” refers to a juxtaposition wherein the components so described are in a relationship permitting them to function in their intended manner. For instance, a promoter is operably linked to a coding sequence if the promoter affects its transcription or expression. The terms “recombinant expression vector,” or “DNA construct” are used interchangeably herein to refer to a DNA molecule having a vector and at least one insert. Recombinant expression vectors are usually generated for the purpose of expressing and/or propagating the insert(s), or for the construction of other recombinant nucleotide sequences. The nucleic acid(s) may or may not be operably linked to a promoter sequence and may or may not be operably linked to DNA regulatory sequences.

The term “operably linked” means that the nucleotide sequence of interest is linked to regulatory sequence(s) in a manner that allows for expression of the nucleotide sequence. The term “regulatory sequence” is intended to include, for example, promoters, enhancers and other expression control elements (e.g., polyadenylation signals). Such regulatory sequences are well known in the art and are described, for example, in Goeddel; Gene Expression Technology: Methods in Enzymology 185, Academic Press, San Diego, Calif. (1990). Regulatory sequences include those that direct constitutive expression of a nucleotide sequence in many types of host cells, and those that direct expression of the nucleotide sequence only in certain host cells (e.g., tissue-specific regulatory sequences). It will be appreciated by those skilled in the art that the design of the expression vector can depend on such factors as the choice of the target cell, the level of expression desired, and the like.

A cell has been “genetically modified” or “transformed” or “transfected” by exogenous DNA, e.g. a recombinant expression vector, when such DNA has been introduced inside the cell. The presence of the exogenous DNA results in permanent or transient genetic change. The transforming DNA may or may not be integrated (covalently linked) into the genome of the cell. The genetically modified (or transformed or transfected) cells that have therapeutic activity, e.g. treating Hemophilia A, can be used and referred to as therapeutic cells.

The term “concentration” used in the context of a molecule such as peptide fragment refers to an amount of molecule, e.g., the number of moles of the molecule, present in a given volume of solution.

The terms “individual,” “subject” and “host” are used interchangeably herein and refer to any subject for whom diagnosis, treatment or therapy is desired. In some aspects, the subject is a mammal. In some aspects, the subject is a human being. In some aspects, the subject is a human patient. In some aspects, the subject can have or is suspected of having Hemophilia A and/or has one or more symptoms of Hemophilia A. In some aspects, the subject is a human who is diagnosed with a risk of Hemophilia A at the time of diagnosis or later. In some cases, the diagnosis with a risk of Hemophilia A can be determined based on the presence of one or more mutations in the endogenous Factor VIII (FVIII) gene or genomic sequence near the Factor VIII (FVIII) gene in the genome that may affect the expression of FVIII gene.

The term “treatment” used referring to a disease or condition means that at least an amelioration of the symptoms associated with the condition afflicting an individual is achieved, where amelioration is used in a broad sense to refer to at least a reduction in the magnitude of a parameter, e.g., a symptom, associated with the condition (e.g., Hemophilia A) being treated. As such, treatment also includes situations where the pathological condition, or at least symptoms associated therewith, are completely inhibited, e.g., prevented from happening, or eliminated entirely such that the host no longer suffers from the condition, or at least the symptoms that characterize the condition. Thus, treatment includes: (i) prevention, that is, reducing the risk of development of clinical symptoms, including causing the clinical symptoms not to develop, e.g., preventing disease progression; (ii) inhibition, that is, arresting the development or further development of clinical symptoms, e.g., mitigating or completely inhibiting an active disease.

The terms “effective amount,” “pharmaceutically effective amount,” or “therapeutically effective amount” as used herein mean a sufficient amount of the composition to provide the desired utility when administered to a subject having a particular condition. In the context of ex vivo treatment of Hemophilia A, the term “effective amount” refers to the amount of a population of therapeutic cells or their progeny needed to prevent or alleviate at least one or more signs or symptoms of Hemophilia A, and relates to a sufficient amount of a composition having the therapeutic cells or their progeny to provide the desired effect, e.g., to treat symptoms of Hemophilia A of a subject. The term “therapeutically effective amount” therefore refers to an amount of therapeutic cells or a composition having therapeutic cells that is sufficient to promote a particular effect when administered to a subject in need of treatment, such as one who has or is at risk for Hemophilia A. An effective amount would also include an amount sufficient to prevent or delay the development of a symptom of the disease, alter the course of a symptom of the disease (for example but not limited to, slow the progression of a symptom of the disease), or reverse a symptom of the disease. In the context of in vivo treatment of Hemophilia A in a subject (e.g. patient) or genome edition done in a cell cultured in vitro, an effective amount refers to an amount of components used for genome edition such as gRNA, donor template and/or a site-directed polypeptide (e.g. DNA endonuclease) needed to edit the genome of the cell in the subject or the cell cultured in vitro. It is understood that for any given case, an appropriate “effective amount” can be determined by one of ordinary skill in the art using routine experimentation.

The term “pharmaceutically acceptable excipient” as used herein refers to any suitable substance that provides a pharmaceutically acceptable carrier, additive or diluent for administration of a compound(s) of interest to a subject. “Pharmaceutically acceptable excipient” can encompass substances referred to as pharmaceutically acceptable diluents, pharmaceutically acceptable additives, and pharmaceutically acceptable carriers.

### Nucleic Acids

### Genome-Targeting Nucleic Acid or Guide RNA

The present disclosure provides a genome-targeting nucleic acid that can direct the activities of an associated polypeptide (e.g., a site-directed polypeptide or DNA endonuclease) to a specific target sequence within a target nucleic acid. In some embodiments, the genome-targeting nucleic acid is an RNA. A genome-targeting RNA is referred to as a “guide RNA” or “gRNA” herein. A guide RNA has at least a spacer sequence that hybridizes to a target nucleic acid sequence of interest and a CRISPR repeat sequence. In Type II systems, the gRNA also has a second RNA called the tracrRNA sequence. In the Type II guide RNA (gRNA), the CRISPR repeat sequence and tracrRNA sequence hybridize to each other to form a duplex. In the Type V guide RNA (gRNA), the crRNA forms a duplex. In both systems, the duplex binds a site-directed polypeptide such that the guide RNA and site-direct polypeptide form a complex. The genome-targeting nucleic acid provides target specificity to the complex by virtue of its association with the site-directed polypeptide. The genome-targeting nucleic acid thus directs the activity of the site-directed polypeptide.

In some embodiments, the genome-targeting nucleic acid is a double-molecule guide RNA. In some embodiments, the genome-targeting nucleic acid is a single-molecule guide RNA. A double-molecule guide RNA has two strands of RNA. The first strand has in the 5′ to 3′ direction, an optional spacer extension sequence, a spacer sequence and a minimum CRISPR repeat sequence. The second strand has a minimum tracrRNA sequence (complementary to the minimum CRISPR repeat sequence), a 3′ tracrRNA sequence and an optional tracrRNA extension sequence. A single-molecule guide RNA (sgRNA) in a Type II system has, in the 5′ to 3′ direction, an optional spacer extension sequence, a spacer sequence, a minimum CRISPR repeat sequence, a single-molecule guide linker, a minimum tracrRNA sequence, a 3′ tracrRNA sequence and an optional tracrRNA extension sequence. The optional tracrRNA extension may have elements that contribute additional functionality (e.g., stability) to the guide RNA. The single-molecule guide linker links the minimum CRISPR repeat and the minimum tracrRNA sequence to form a hairpin structure. The optional tracrRNA extension has one or more hairpins. A single-molecule guide RNA (sgRNA) in a Type V system has, in the 5′ to 3′ direction, a minimum CRISPR repeat sequence and a spacer sequence.

By way of illustration, guide RNAs used in the CRISPR/Cas/Cpf1 system, or other smaller RNAs can be readily synthesized by chemical means as illustrated below and described in the art. While chemical synthetic procedures are continually expanding, purifications of such RNAs by procedures such as high performance liquid chromatography (HPLC, which avoids the use of gels such as PAGE) tends to become more challenging as polynucleotide lengths increase significantly beyond a hundred or so nucleotides. One approach used for generating RNAs of greater length is to produce two or more molecules that are ligated together. Much longer RNAs, such as those encoding a Cas9 or Cpf1 endonuclease, are more readily generated enzymatically. Various types of RNA modifications can be introduced during or after chemical synthesis and/or enzymatic generation of RNAs, e.g., modifications that enhance stability, reduce the likelihood or degree of innate immune response, and/or enhance other attributes, as described in the art.

### Spacer Extension Sequence

In some embodiments of genome-targeting nucleic acids, a spacer extension sequence can modify activity, provide stability and/or provide a location for modifications of a genome-targeting nucleic acid. A spacer extension sequence can modify on- or off-target activity or specificity. In some embodiments, a spacer extension sequence is provided. A spacer extension sequence can have a length of more than 1, 5, 10, 15, 20, 25, 30, 35, 40, 45, 50, 60, 70, 80, 90, 100, 120, 140, 160, 180, 200, 220, 240, 260, 280, 300, 320, 340, 360, 380, 400, 1000, 2000, 3000, 4000, 5000, 6000, or 7000 or more nucleotides. A spacer extension sequence can have a length of about 1, 5, 10, 15, 20, 25, 30, 35, 40, 45, 50, 60, 70, 80, 90, 100, 120, 140, 160, 180, 200, 220, 240, 260, 280, 300, 320, 340, 360, 380, 400, 1000, 2000, 3000, 4000, 5000, 6000, or 7000 or more nucleotides. A spacer extension sequence can have a length of less than 1, 5, 10, 15, 20, 25, 30, 35, 40, 45, 50, 60, 70, 80, 90, 100, 120, 140, 160, 180, 200, 220, 240, 260, 280, 300, 320, 340, 360, 380, 400, 1000, 2000, 3000, 4000, 5000, 6000, 7000 or more nucleotides. In some embodiments, a spacer extension sequence is less than 10 nucleotides in length. In some embodiments, a spacer extension sequence is between 10-30 nucleotides in length. In some embodiments, a spacer extension sequence is between 30-70 nucleotides in length.

In some embodiments, the spacer extension sequence has another moiety (e.g., a stability control sequence, an endoribonuclease binding sequence, a ribozyme). In some embodiments, the moiety decreases or increases the stability of a nucleic acid targeting nucleic acid. In some embodiments, the moiety is a transcriptional terminator segment (i.e., a transcription termination sequence). In some embodiments, the moiety functions in a eukaryotic cell. In some embodiments, the moiety functions in a prokaryotic cell. In some embodiments, the moiety functions in both eukaryotic and prokaryotic cells. Non-limiting examples of suitable moieties include: a 5′ cap (e.g., a 7-methylguanylate cap (m7 G)), a riboswitch sequence (e.g., to allow for regulated stability and/or regulated accessibility by proteins and protein complexes), a sequence that forms a dsRNA duplex (i.e., a hairpin), a sequence that targets the RNA to a subcellular location (e.g., nucleus, mitochondria, chloroplasts, and the like), a modification or sequence that provides for tracking (e.g., direct conjugation to a fluorescent molecule, conjugation to a moiety that facilitates fluorescent detection, a sequence that allows for fluorescent detection, etc.), and/or a modification or sequence that provides a binding site for proteins (e.g., proteins that act on DNA, including transcriptional activators, transcriptional repressors, DNA methyltransferases, DNA demethylases, histone acetyltransferases, histone deacetylases, and the like).

### Spacer Sequence

The spacer sequence hybridizes to a sequence in a target nucleic acid of interest. The spacer of a genome-targeting nucleic acid interacts with a target nucleic acid in a sequence-specific manner via hybridization (i.e., base pairing). The nucleotide sequence of the spacer thus varies depending on the sequence of the target nucleic acid of interest.

In a CRISPR/Cas system herein, the spacer sequence is designed to hybridize to a target nucleic acid that is located 5′ of a PAM of the Cas9 enzyme used in the system. The spacer can perfectly match the target sequence or can have mismatches. Each Cas9 enzyme has a particular PAM sequence that it recognizes in a target DNA. For example, S. pyogenes recognizes in a target nucleic acid a PAM that has the sequence 5′-NRG-3′, where R has either A or G, where N is any nucleotide and N is immediately 3′ of the target nucleic acid sequence targeted by the spacer sequence.

In some embodiments, the target nucleic acid sequence has 20 nucleotides. In some embodiments, the target nucleic acid has less than 20 nucleotides. In some embodiments, the target nucleic acid has more than 20 nucleotides. In some embodiments, the target nucleic acid has at least: 5, 10, 15, 16, 17, 18, 19, 20, 21, 22, 23, 24, 25, 30 or more nucleotides. In some embodiments, the target nucleic acid has at most: 5, 10, 15, 16, 17, 18, 19, 20, 21, 22, 23, 24, 25, 30 or more nucleotides. In some embodiments, the target nucleic acid sequence has 20 bases immediately 5′ of the first nucleotide of the PAM. For example, in a sequence having 5′-NNNNNNNNNNNNNNNNNNNNNRG-3′ (SEQ ID NO: 100), the target nucleic acid has the sequence that corresponds to the Ns, wherein N is any nucleotide, and the underlined NRG sequence (R is G or A) is the Streptococcus pyogenes Cas9 PAM. In some embodiments, the PAM sequence used in the compositions and methods of the present disclosure as a sequence recognized by S.p. Cas9 is NGG.

In some embodiments, the spacer sequence that hybridizes to the target nucleic acid has a length of at least about 6 nucleotides (nt). The spacer sequence can be at least about 6 nt, about 10 nt, about 15 nt, about 18 nt, about 19 nt, about 20 nt, about 25 nt, about 30 nt, about 35 nt or about 40 nt, from about 6 nt to about 80 nt, from about 6 nt to about 50 nt, from about 6 nt to about 45 nt, from about 6 nt to about 40 nt, from about 6 nt to about 35 nt, from about 6 nt to about 30 nt, from about 6 nt to about 25 nt, from about 6 nt to about 20 nt, from about 6 nt to about 19 nt, from about 10 nt to about 50 nt, from about 10 nt to about 45 nt, from about 10 nt to about 40 nt, from about 10 nt to about 35 nt, from about 10 nt to about 30 nt, from about 10 nt to about 25 nt, from about 10 nt to about 20 nt, from about 10 nt to about 19 nt, from about 19 nt to about 25 nt, from about 19 nt to about 30 nt, from about 19 nt to about 35 nt, from about 19 nt to about 40 nt, from about 19 nt to about 45 nt, from about 19 nt to about 50 nt, from about 19 nt to about 60 nt, from about 20 nt to about 25 nt, from about 20 nt to about 30 nt, from about 20 nt to about 35 nt, from about 20 nt to about 40 nt, from about 20 nt to about 45 nt, from about 20 nt to about 50 nt, or from about 20 nt to about 60 nt. In some embodiments, the spacer sequence has 20 nucleotides. In some embodiments, the spacer has 19 nucleotides. In some embodiments, the spacer has 18 nucleotides. In some embodiments, the spacer has 17 nucleotides. In some embodiments, the spacer has 16 nucleotides. In some embodiments, the spacer has 15 nucleotides.

In some embodiments, the percent complementarity between the spacer sequence and the target nucleic acid is at least about 30%, at least about 40%, at least about 50%, at least about 60%, at least about 65%, at least about 70%, at least about 75%, at least about 80%, at least about 85%, at least about 90%, at least about 95%, at least about 97%, at least about 98%, at least about 99%, or 100%. In some embodiments, the percent complementarity between the spacer sequence and the target nucleic acid is at most about 30%, at most about 40%, at most about 50%, at most about 60%, at most about 65%, at most about 70%, at most about 75%, at most about 80%, at most about 85%, at most about 90%, at most about 95%, at most about 97%, at most about 98%, at most about 99%, or 100%. In some embodiments, the percent complementarity between the spacer sequence and the target nucleic acid is 100% over the six contiguous 5′-most nucleotides of the target sequence of the complementary strand of the target nucleic acid. In some embodiments, the percent complementarity between the spacer sequence and the target nucleic acid is at least 60% over about 20 contiguous nucleotides. In some embodiments, the length of the spacer sequence and the target nucleic acid can differ by 1 to 6 nucleotides, which can be thought of as a bulge or bulges.

In some embodiments, the spacer sequence is designed or chosen using a computer program. The computer program can use variables, such as predicted melting temperature, secondary structure formation, predicted annealing temperature, sequence identity, genomic context, chromatin accessibility, % GC, frequency of genomic occurrence (e.g., of sequences that are identical or are similar but vary in one or more spots as a result of mismatch, insertion or deletion), methylation status, presence of SNPs, and the like.

### Minimum CRISPR Repeat Sequence

In some embodiments, a minimum CRISPR repeat sequence is a sequence with at least about 30%, about 40%, about 50%, about 60%, about 65%, about 70%, about 75%, about 80%, about 85%, about 90%, about 95%, or 100% sequence identity to a reference CRISPR repeat sequence (e.g., crRNA from S. pyogenes).

In some embodiments, a minimum CRISPR repeat sequence has nucleotides that can hybridize to a minimum tracrRNA sequence in a cell. The minimum CRISPR repeat sequence and a minimum tracrRNA sequence form a duplex, i.e. a base-paired double-stranded structure. Together, the minimum CRISPR repeat sequence and the minimum tracrRNA sequence bind to the site-directed polypeptide. At least a part of the minimum CRISPR repeat sequence hybridizes to the minimum tracrRNA sequence. In some embodiments, at least a part of the minimum CRISPR repeat sequence has at least about 30%, about 40%, about 50%, about 60%, about 65%, about 70%, about 75%, about 80%, about 85%, about 90%, about 95%, or 100% complementary to the minimum tracrRNA sequence. In some embodiments, at least a part of the minimum CRISPR repeat sequence has at most about 30%, about 40%, about 50%, about 60%, about 65%, about 70%, about 75%, about 80%, about 85%, about 90%, about 95%, or 100% complementary to the minimum tracrRNA sequence.

The minimum CRISPR repeat sequence can have a length from about 7 nucleotides to about 100 nucleotides. For example, the length of the minimum CRISPR repeat sequence is from about 7 nucleotides (nt) to about 50 nt, from about 7 nt to about 40 nt, from about 7 nt to about 30 nt, from about 7 nt to about 25 nt, from about 7 nt to about 20 nt, from about 7 nt to about 15 nt, from about 8 nt to about 40 nt, from about 8 nt to about 30 nt, from about 8 nt to about 25 nt, from about 8 nt to about 20 nt, from about 8 nt to about 15 nt, from about 15 nt to about 100 nt, from about 15 nt to about 80 nt, from about 15 nt to about 50 nt, from about 15 nt to about 40 nt, from about 15 nt to about 30 nt, or from about 15 nt to about 25 nt. In some embodiments, the minimum CRISPR repeat sequence is approximately 9 nucleotides in length. In some embodiments, the minimum CRISPR repeat sequence is approximately 12 nucleotides in length.

In some embodiments, the minimum CRISPR repeat sequence is at least about 60% identical to a reference minimum CRISPR repeat sequence (e.g., wild-type crRNA from S. pyogenes) over a stretch of at least 6, 7, or 8 contiguous nucleotides. For example, the minimum CRISPR repeat sequence is at least about 65% identical, at least about 70% identical, at least about 75% identical, at least about 80% identical, at least about 85% identical, at least about 90% identical, at least about 95% identical, at least about 98% identical, at least about 99% identical or 100% identical to a reference minimum CRISPR repeat sequence over a stretch of at least 6, 7, or 8 contiguous nucleotides.

Minimum tracrRNA Sequence

In some embodiments, a minimum tracrRNA sequence is a sequence with at least about 30%, about 40%, about 50%, about 60%, about 65%, about 70%, about 75%, about 80%, about 85%, about 90%, about 95%, or 100% sequence identity to a reference tracrRNA sequence (e.g., wild type tracrRNA from S. pyogenes).

In some embodiments, a minimum tracrRNA sequence has nucleotides that hybridize to a minimum CRISPR repeat sequence in a cell. A minimum tracrRNA sequence and a minimum CRISPR repeat sequence form a duplex, i.e. a base-paired double-stranded structure. Together, the minimum tracrRNA sequence and the minimum CRISPR repeat bind to a site-directed polypeptide. At least a part of the minimum tracrRNA sequence can hybridize to the minimum CRISPR repeat sequence. In some embodiments, the minimum tracrRNA sequence is at least about 30%, about 40%, about 50%, about 60%, about 65%, about 70%, about 75%, about 80%, about 85%, about 90%, about 95%, or 100% complementary to the minimum CRISPR repeat sequence.

The minimum tracrRNA sequence can have a length from about 7 nucleotides to about 100 nucleotides. For example, the minimum tracrRNA sequence can be from about 7 nucleotides (nt) to about 50 nt, from about 7 nt to about 40 nt, from about 7 nt to about 30 nt, from about 7 nt to about 25 nt, from about 7 nt to about 20 nt, from about 7 nt to about 15 nt, from about 8 nt to about 40 nt, from about 8 nt to about 30 nt, from about 8 nt to about 25 nt, from about 8 nt to about 20 nt, from about 8 nt to about 15 nt, from about 15 nt to about 100 nt, from about 15 nt to about 80 nt, from about 15 nt to about 50 nt, from about 15 nt to about 40 nt, from about 15 nt to about 30 nt or from about 15 nt to about 25 nt long. In some embodiments, the minimum tracrRNA sequence is approximately 9 nucleotides in length. In some embodiments, the minimum tracrRNA sequence is approximately 12 nucleotides. In some embodiments, the minimum tracrRNA consists of tracrRNA nt 23-48 described in Jinek et al. Science, 337(6096):816-821 (2012).

In some embodiments, the minimum tracrRNA sequence is at least about 60% identical to a reference minimum tracrRNA (e.g., wild type, tracrRNA from S. pyogenes) sequence over a stretch of at least 6, 7, or 8 contiguous nucleotides. For example, the minimum tracrRNA sequence is at least about 65% identical, about 70% identical, about 75% identical, about 80% identical, about 85% identical, about 90% identical, about 95% identical, about 98% identical, about 99% identical or 100% identical to a reference minimum tracrRNA sequence over a stretch of at least 6, 7, or 8 contiguous nucleotides.

In some embodiments, the duplex between the minimum CRISPR RNA and the minimum tracrRNA has a double helix. In some embodiments, the duplex between the minimum CRISPR RNA and the minimum tracrRNA has at least about 1, 2, 3, 4, 5, 6, 7, 8, 9, or 10 or more nucleotides. In some embodiments, the duplex between the minimum CRISPR RNA and the minimum tracrRNA has at most about 1, 2, 3, 4, 5, 6, 7, 8, 9, or 10 or more nucleotides.

In some embodiments, the duplex has a mismatch (i.e., the two strands of the duplex are not 100% complementary). In some embodiments, the duplex has at least about 1, 2, 3, 4, or 5 or mismatches. In some embodiments, the duplex has at most about 1, 2, 3, 4, or 5 or mismatches. In some embodiments, the duplex has no more than 2 mismatches.

### Bulges

In some embodiments, there is a “bulge” in the duplex between the minimum CRISPR RNA and the minimum tracrRNA. The bulge is an unpaired region of nucleotides within the duplex. In some embodiments, the bulge contributes to the binding of the duplex to the site-directed polypeptide. A bulge has, on one side of the duplex, an unpaired 5′-XXXY-3′ where X is any purine and Y has a nucleotide that can form a wobble pair with a nucleotide on the opposite strand, and an unpaired nucleotide region on the other side of the duplex. The number of unpaired nucleotides on the two sides of the duplex can be different.

In one example, the bulge has an unpaired purine (e.g., adenine) on the minimum CRISPR repeat strand of the bulge. In some embodiments, a bulge has an unpaired 5′-AAGY-3′ of the minimum tracrRNA sequence strand of the bulge, where Y has a nucleotide that can form a wobble pairing with a nucleotide on the minimum CRISPR repeat strand.

In some embodiments, a bulge on the minimum CRISPR repeat side of the duplex has at least 1, 2, 3, 4, or 5 or more unpaired nucleotides. In some embodiments, a bulge on the minimum CRISPR repeat side of the duplex has at most 1, 2, 3, 4, or 5 or more unpaired nucleotides. In some embodiments, a bulge on the minimum CRISPR repeat side of the duplex has 1 unpaired nucleotide.

In some embodiments, a bulge on the minimum tracrRNA sequence side of the duplex has at least 1, 2, 3, 4, 5, 6, 7, 8, 9, or 10 or more unpaired nucleotides. In some embodiments, a bulge on the minimum tracrRNA sequence side of the duplex has at most 1, 2, 3, 4, 5, 6, 7, 8, 9, or 10 or more unpaired nucleotides. In some embodiments, a bulge on a second side of the duplex (e.g., the minimum tracrRNA sequence side of the duplex) has 4 unpaired nucleotides.

In some embodiments, a bulge has at least one wobble pairing. In some embodiments, a bulge has at most one wobble pairing. In some embodiments, a bulge has at least one purine nucleotide. In some embodiments, a bulge has at least 3 purine nucleotides. In some embodiments, a bulge sequence has at least 5 purine nucleotides. In some embodiments, a bulge sequence has at least one guanine nucleotide. In some embodiments, a bulge sequence has at least one adenine nucleotide.

### Hairpins

In various embodiments, one or more hairpins are located 3′ to the minimum tracrRNA in the 3′ tracrRNA sequence.

In some embodiments, the hairpin starts at least about 1, 2, 3, 4, 5, 6, 7, 8, 9, 10, 15, or 20 or more nucleotides 3′ from the last paired nucleotide in the minimum CRISPR repeat and minimum tracrRNA sequence duplex. In some embodiments, the hairpin can start at most about 1, 2, 3, 4, 5, 6, 7, 8, 9 or 10 or more nucleotides 3′ of the last paired nucleotide in the minimum CRISPR repeat and minimum tracrRNA sequence duplex.

In some embodiments, a hairpin has at least about 1, 2, 3, 4, 5, 6, 7, 8, 9, 10, 15, or 20 or more consecutive nucleotides. In some embodiments, a hairpin has at most about 1, 2, 3, 4, 5, 6, 7, 8, 9, 10, 15, or more consecutive nucleotides.

In some embodiments, a hairpin has a CC dinucleotide (i.e., two consecutive cytosine nucleotides).

In some embodiments, a hairpin has duplexed nucleotides (e.g., nucleotides in a hairpin, hybridized together). For example, a hairpin has a CC dinucleotide that is hybridized to a GG dinucleotide in a hairpin duplex of the 3′ tracrRNA sequence.

One or more of the hairpins can interact with guide RNA-interacting regions of a site-directed polypeptide.

In some embodiments, there are two or more hairpins, and in some embodiments there are three or more hairpins.

3′ tracrRNA Sequence

In some embodiments, a 3′ tracrRNA sequence has a sequence with at least about 30%, about 40%, about 50%, about 60%, about 65%, about 70%, about 75%, about 80%, about 85%, about 90%, about 95%, or 100% sequence identity to a reference tracrRNA sequence (e.g., a tracrRNA from S. pyogenes).

In some embodiments, the 3′ tracrRNA sequence has a length from about 6 nucleotides to about 100 nucleotides. For example, the 3′ tracrRNA sequence can have a length from about 6 nucleotides (nt) to about 50 nt, from about 6 nt to about 40 nt, from about 6 nt to about 30 nt, from about 6 nt to about 25 nt, from about 6 nt to about 20 nt, from about 6 nt to about 15 nt, from about 8 nt to about 40 nt, from about 8 nt to about 30 nt, from about 8 nt to about 25 nt, from about 8 nt to about 20 nt, from about 8 nt to about 15 nt, from about 15 nt to about 100 nt, from about 15 nt to about 80 nt, from about 15 nt to about 50 nt, from about 15 nt to about 40 nt, from about 15 nt to about 30 nt, or from about 15 nt to about 25 nt. In some embodiments, the 3′ tracrRNA sequence has a length of approximately 14 nucleotides.

In some embodiments, the 3′ tracrRNA sequence is at least about 60% identical to a reference 3′ tracrRNA sequence (e.g., wild type 3′ tracrRNA sequence from S. pyogenes) over a stretch of at least 6, 7, or 8 contiguous nucleotides. For example, the 3′ tracrRNA sequence is at least about 60% identical, about 65% identical, about 70% identical, about 75% identical, about 80% identical, about 85% identical, about 90% identical, about 95% identical, about 98% identical, about 99% identical, or 100% identical, to a reference 3′ tracrRNA sequence (e.g., wild type 3′ tracrRNA sequence from S. pyogenes) over a stretch of at least 6, 7, or 8 contiguous nucleotides.

In some embodiments, a 3′ tracrRNA sequence has more than one duplexed region (e.g., hairpin, hybridized region). In some embodiments, a 3′ tracrRNA sequence has two duplexed regions.

In some embodiments, the 3′ tracrRNA sequence has a stem loop structure. In some embodiments, a stem loop structure in the 3′ tracrRNA has at least 1, 2, 3, 4, 5, 6, 7, 8, 9, 10, 15 or 20 or more nucleotides. In some embodiments, the stem loop structure in the 3′ tracrRNA has at most 1, 2, 3, 4, 5, 6, 7, 8, 9 or 10 or more nucleotides. In some embodiments, the stem loop structure has a functional moiety. For example, the stem loop structure can have an aptamer, a ribozyme, a protein-interacting hairpin, a CRISPR array, an intron, or an exon. In some embodiments, the stem loop structure has at least about 1, 2, 3, 4, or 5 or more functional moieties. In some embodiments, the stem loop structure has at most about 1, 2, 3, 4, or 5 or more functional moieties.

In some embodiments, the hairpin in the 3′ tracrRNA sequence has a P-domain. In some embodiments, the P-domain has a double-stranded region in the hairpin.

tracrRNA Extension Sequence

In some embodiments, a tracrRNA extension sequence can be provided whether the tracrRNA is in the context of single-molecule guides or double-molecule guides. In some embodiments, a tracrRNA extension sequence has a length from about 1 nucleotide to about 400 nucleotides. In some embodiments, a tracrRNA extension sequence has a length of more than 1, 5, 10, 15, 20, 25, 30, 35, 40, 45, 50, 60, 70, 80, 90, 100, 120, 140, 160, 180, 200, 220, 240, 260, 280, 300, 320, 340, 360, 380, or 400 nucleotides. In some embodiments, a tracrRNA extension sequence has a length from about 20 to about 5000 or more nucleotides. In some embodiments, a tracrRNA extension sequence has a length of more than 1000 nucleotides. In some embodiments, a tracrRNA extension sequence has a length of less than 1, 5, 10, 15, 20, 25, 30, 35, 40, 45, 50, 60, 70, 80, 90, 100, 120, 140, 160, 180, 200, 220, 240, 260, 280, 300, 320, 340, 360, 380, 400 or more nucleotides. In some embodiments, a tracrRNA extension sequence can have a length of less than 1000 nucleotides. In some embodiments, a tracrRNA extension sequence has less than 10 nucleotides in length. In some embodiments, a tracrRNA extension sequence is 10-30 nucleotides in length. In some embodiments, tracrRNA extension sequence is 30-70 nucleotides in length.

In some embodiments, the tracrRNA extension sequence has a functional moiety (e.g., a stability control sequence, ribozyme, endoribonuclease binding sequence). In some embodiments, the functional moiety has a transcriptional terminator segment (i.e., a transcription termination sequence). In some embodiments, the functional moiety has a total length from about 10 nucleotides (nt) to about 100 nucleotides, from about 10 nt to about 20 nt, from about 20 nt to about 30 nt, from about 30 nt to about 40 nt, from about 40 nt to about 50 nt, from about 50 nt to about 60 nt, from about 60 nt to about 70 nt, from about 70 nt to about 80 nt, from about 80 nt to about 90 nt, or from about 90 nt to about 100 nt, from about 15 nt to about 80 nt, from about 15 nt to about 50 nt, from about 15 nt to about 40 nt, from about 15 nt to about 30 nt, or from about 15 nt to about 25 nt. In some embodiments, the functional moiety functions in a eukaryotic cell. In some embodiments, the functional moiety functions in a prokaryotic cell. In some embodiments, the functional moiety functions in both eukaryotic and prokaryotic cells.

Non-limiting examples of suitable tracrRNA extension functional moieties include a 3′ poly-adenylated tail, a riboswitch sequence (e.g., to allow for regulated stability and/or regulated accessibility by proteins and protein complexes), a sequence that forms a dsRNA duplex (i.e., a hairpin), a sequence that targets the RNA to a subcellular location (e.g., nucleus, mitochondria, chloroplasts, and the like), a modification or sequence that provides for tracking (e.g., direct conjugation to a fluorescent molecule, conjugation to a moiety that facilitates fluorescent detection, a sequence that allows for fluorescent detection, etc.), and/or a modification or sequence that provides a binding site for proteins (e.g., proteins that act on DNA, including transcriptional activators, transcriptional repressors, DNA methyltransferases, DNA demethylases, histone acetyltransferases, histone deacetylases, and the like). In some embodiments, a tracrRNA extension sequence has a primer binding site or a molecular index (e.g., barcode sequence). In some embodiments, the tracrRNA extension sequence has one or more affinity tags.

### Single-Molecule Guide Linker Sequence

In some embodiments, the linker sequence of a single-molecule guide nucleic acid has a length from about 3 nucleotides to about 100 nucleotides. In Jinek et al., supra, for example, a simple 4 nucleotide “tetraloop” (-GAAA-) was used, Science, 337(6096):816-821 (2012). An illustrative linker has a length from about 3 nucleotides (nt) to about 90 nt, from about 3 nt to about 80 nt, from about 3 nt to about 70 nt, from about 3 nt to about 60 nt, from about 3 nt to about 50 nt, from about 3 nt to about 40 nt, from about 3 nt to about 30 nt, from about 3 nt to about 20 nt, from about 3 nt to about 10 nt. For example, the linker can have a length from about 3 nt to about 5 nt, from about 5 nt to about 10 nt, from about 10 nt to about 15 nt, from about 15 nt to about 20 nt, from about 20 nt to about 25 nt, from about 25 nt to about 30 nt, from about 30 nt to about 35 nt, from about 35 nt to about 40 nt, from about 40 nt to about 50 nt, from about 50 nt to about 60 nt, from about 60 nt to about 70 nt, from about 70 nt to about 80 nt, from about 80 nt to about 90 nt, or from about 90 nt to about 100 nt. In some embodiments, the linker of a single-molecule guide nucleic acid is between 4 and 40 nucleotides. In some embodiments, a linker is at least about 100, 500, 1000, 1500, 2000, 2500, 3000, 3500, 4000, 4500, 5000, 5500, 6000, 6500, or 7000 or more nucleotides. In some embodiments, a linker is at most about 100, 500, 1000, 1500, 2000, 2500, 3000, 3500, 4000, 4500, 5000, 5500, 6000, 6500, or 7000 or more nucleotides.

Linkers can have any of a variety of sequences, although in some embodiments, the linker will not have sequences that have extensive regions of homology with other portions of the guide RNA, which might cause intramolecular binding that could interfere with other functional regions of the guide. In Jinek et al., supra, a simple 4 nucleotide sequence -GAAA- was used, Science, 337(6096):816-821 (2012), but numerous other sequences, including longer sequences can likewise be used.

In some embodiments, the linker sequence has a functional moiety. For example, the linker sequence can have one or more features, including an aptamer, a ribozyme, a protein-interacting hairpin, a protein binding site, a CRISPR array, an intron, or an exon. In some embodiments, the linker sequence has at least about 1, 2, 3, 4, or 5 or more functional moieties. In some embodiments, the linker sequence has at most about 1, 2, 3, 4, or 5 or more functional moieties.

In some embodiments, a genomic location targeted by gRNAs in accordance with the preset disclosure can be at, within or near the endogenous albumin locus in a genome, e.g. human genome. Exemplary guide RNAs targeting such locations include the spacer sequences listed in Tables 3 or 4 (e.g., spacer sequences from any one of SEQ ID NOs: 18-44 and 104) and the associated Cas9 or Cpf1 cut site. For example, a gRNA including a spacer sequence from SEQ ID NO: 18 can include the spacer sequence UAAUUUUCUUUUGCGCACUA (SEQ ID NO: 105). As is understood by the person of ordinary skill in the art, each guide RNA is designed to include a spacer sequence complementary to its genomic target sequence. For example, each of the spacer sequences listed in Tables 3 or 4 can be put into a single RNA chimera or a crRNA (along with a corresponding tracrRNA). See Jinek et al., Science, 337, 816-821 (2012) and Deltcheva et al., Nature, 471, 602-607 (2011).

### Donor DNA or Donor Template

Site-directed polypeptides, such as a DNA endonuclease, can introduce double-strand breaks or single-strand breaks in nucleic acids, e.g., genomic DNA. The double-strand break can stimulate a cell's endogenous DNA-repair pathways (e.g., homology-dependent repair (HDR) or non-homologous end joining or alternative non-homologous end joining (A-NHEJ) or microhomology-mediated end joining (MMEJ). NHEJ can repair cleaved target nucleic acid without the need for a homologous template. This can sometimes result in small deletions or insertions (indels) in the target nucleic acid at the site of cleavage, and can lead to disruption or alteration of gene expression. HDR, which is also known as homologous recombination (HR) can occur when a homologous repair template, or donor, is available.

The homologous donor template has sequences that are homologous to sequences flanking the target nucleic acid cleavage site. The sister chromatid is generally used by the cell as the repair template. However, for the purposes of genome editing, the repair template is often supplied as an exogenous nucleic acid, such as a plasmid, duplex oligonucleotide, single-strand oligonucleotide, double-stranded oligonucleotide, or viral nucleic acid. With exogenous donor templates, it is common to introduce an additional nucleic acid sequence (such as a transgene) or modification (such as a single or multiple base change or a deletion) between the flanking regions of homology so that the additional or altered nucleic acid sequence also becomes incorporated into the target locus. MMEJ results in a genetic outcome that is similar to NHEJ in that small deletions and insertions can occur at the cleavage site. MMEJ makes use of homologous sequences of a few base pairs flanking the cleavage site to drive a favored end-joining DNA repair outcome. In some instances, it can be possible to predict likely repair outcomes based on analysis of potential microhomologies in the nuclease target regions.

Thus, in some cases, homologous recombination is used to insert an exogenous polynucleotide sequence into the target nucleic acid cleavage site. An exogenous polynucleotide sequence is termed a donor polynucleotide (or donor or donor sequence or polynucleotide donor template) herein. In some embodiments, the donor polynucleotide, a portion of the donor polynucleotide, a copy of the donor polynucleotide, or a portion of a copy of the donor polynucleotide is inserted into the target nucleic acid cleavage site. In some embodiments, the donor polynucleotide is an exogenous polynucleotide sequence, i.e., a sequence that does not naturally occur at the target nucleic acid cleavage site.

When an exogenous DNA molecule is supplied in sufficient concentration inside the nucleus of a cell in which the double strand break occurs, the exogenous DNA can be inserted at the double strand break during the NHEJ repair process and thus become a permanent addition to the genome. These exogenous DNA molecules are referred to as donor templates in some embodiments. If the donor template contains a coding sequence for a gene of interest such as a FVIII gene optionally together with relevant regulatory sequences such as promoters, enhancers, polyA sequences and/or splice acceptor sequences (also referred to herein as a “donor cassette”), the gene of interest can be expressed from the integrated copy in the genome resulting in permanent expression for the life of the cell. Moreover, the integrated copy of the donor DNA template can be transmitted to the daughter cells when the cell divides.

In the presence of sufficient concentrations of a donor DNA template that contains flanking DNA sequences with homology to the DNA sequence either side of the double strand break (referred to as homology arms), the donor DNA template can be integrated via the HDR pathway. The homology arms act as substrates for homologous recombination between the donor template and the sequences either side of the double strand break. This can result in an error free insertion of the donor template in which the sequences either side of the double strand break are not altered from that in the un-modified genome.

Supplied donors for editing by HDR vary markedly but generally contain the intended sequence with small or large flanking homology arms to allow annealing to the genomic DNA. The homology regions flanking the introduced genetic changes can be 30 bp or smaller, or as large as a multi-kilobase cassette that can contain promoters, cDNAs, etc. Both single-stranded and double-stranded oligonucleotide donors can be used. These oligonucleotides range in size from less than 100 nt to over many kb, though longer ssDNA can also be generated and used. Double-stranded donors are often used, including PCR amplicons, plasmids, and mini-circles. In general, it has been found that an AAV vector is a very effective means of delivery of a donor template, though the packaging limits for individual donors is <5 kb. Active transcription of the donor increased HDR three-fold, indicating the inclusion of promoter can increase conversion. Conversely, CpG methylation of the donor can decrease gene expression and HDR.

In some embodiments, the donor DNA can be supplied with the nuclease or independently by a variety of different methods, for example by transfection, nano-particle, micro-injection, or viral transduction. A range of tethering options can be used to increase the availability of the donors for HDR in some embodiments. Examples include attaching the donor to the nuclease, attaching to DNA binding proteins that bind nearby, or attaching to proteins that are involved in DNA end binding or repair.

In addition to genome editing by NHEJ or HDR, site-specific gene insertions can be conducted that use both the NHEJ pathway and HR. A combination approach can be applicable in certain settings, possibly including intron/exon borders. NHEJ can prove effective for ligation in the intron, while the error-free HDR can be better suited in the coding region.

In embodiments, an exogenous sequence that is intended to be inserted into a genome is a Factor VIII (FVIII) gene or functional derivative thereof. The exogenous gene can include a nucleotide sequence encoding a Factor VIII protein or functional derivative thereof. The functional derivative of a FVIII gene can include a nucleic acid sequence encoding a functional derivative of a FVIII protein that has a substantial activity of a wildtype FVIII protein such as. the wildtype human FVIII protein, e.g. at least about 30%, about 40%, about 50%, about 60%, about 70%, about 80%, about 90%, about 95% or about 100% of the activity that the wildtype FVIII protein exhibits. In some embodiments, the functional derivative of a FVIII protein can have at least about 30%, about 40%, about 50%, about 60%, about 70%, about 80%, about 85%, about 90%, about 95%, about 96%, about 97%, about 98% or about 99% amino acid sequence identity to the FVIII protein, e.g. the wildtype FVIII protein. In some embodiments, one having ordinary skill in the art can use a number of methods known in the field to test the functionality or activity of a compound, e.g. peptide or protein. The functional derivative of the FVIII protein can also include any fragment of the wildtype FVIII protein or fragment of a modified FVIII protein that has conservative modification on one or more of amino acid residues in the full length, wildtype FVIII protein. Thus, in some embodiments, the functional derivative of a nucleic acid sequence of a FVIII gene can have at least about 30%, about 40%, about 50%, about 60%, about 70%, about 80%, about 85%, about 90%, about 95%, about 96%, about 97%, about 98% or about 99% nucleic acid sequence identity to the FVIII gene, e g. the wildtype FVIII gene.

In some embodiments where the insertion of a Factor VIII (FVIII) gene or functional derivative thereof is concerned, a cDNA of Factor VIII gene or functional derivative thereof can be inserted into a genome of a patient having defective FVIII gene or its regulatory sequences. In such a case, a donor DNA or donor template can be an expression cassette or vector construct having the sequence encoding Factor VIII gene or functional derivative thereof, e.g. cDNA sequence. In some embodiments, the expression vector contains a sequence encoding a modified Factor VIII protein such as FVIII-BDD, which is described elsewhere in the disclosures, can be used.

In some embodiments, according to any of the donor templates described herein comprising a donor cassette, the donor cassette is flanked on one or both sides by a gRNA target site. For example, such a donor template may comprise a donor cassette with a gRNA target site 5′ of the donor cassette and/or a gRNA target site 3′ of the donor cassette. In some embodiments, the donor template comprises a donor cassette with a gRNA target site 5′ of the donor cassette. In some embodiments, the donor template comprises a donor cassette with a gRNA target site 3′ of the donor cassette. In some embodiments, the donor template comprises a donor cassette with a gRNA target site 5′ of the donor cassette and a gRNA target site 3′ of the donor cassette. In some embodiments, the donor template comprises a donor cassette with a gRNA target site 5′ of the donor cassette and a gRNA target site 3′ of the donor cassette, and the two gRNA target sites comprise the same sequence. In some embodiments, the donor template comprises at least one gRNA target site, and the at least one gRNA target site in the donor template comprises the same sequence as a gRNA target site in a target locus into which the donor cassette of the donor template is to be integrated. In some embodiments, the donor template comprises at least one gRNA target site, and the at least one gRNA target site in the donor template comprises the reverse complement of a gRNA target site in a target locus into which the donor cassette of the donor template is to be integrated. In some embodiments, the donor template comprises a donor cassette with a gRNA target site 5′ of the donor cassette and a gRNA target site 3′ of the donor cassette, and the two gRNA target sites in the donor template comprises the same sequence as a gRNA target site in a target locus into which the donor cassette of the donor template is to be integrated. In some embodiments, the donor template comprises a donor cassette with a gRNA target site 5′ of the donor cassette and a gRNA target site 3′ of the donor cassette, and the two gRNA target sites in the donor template comprises the reverse complement of a gRNA target site in a target locus into which the donor cassette of the donor template is to be integrated.

### Nucleic Acid Encoding a Site-Directed Polypeptide or DNA Endonuclease

In some embodiments, the methods of genome edition and compositions therefore can use a nucleic acid sequence (or oligonucleotide) encoding a site-directed polypeptide or DNA endonuclease. The nucleic acid sequence encoding the site-directed polypeptide can be DNA or RNA. If the nucleic acid sequence encoding the site-directed polypeptide is RNA, it can be covalently linked to a gRNA sequence or exist as a separate sequence. In some embodiments, a peptide sequence of the site-directed polypeptide or DNA endonuclease can be used instead of the nucleic acid sequence thereof.

### Vectors

In another aspect, the present disclosure provides a nucleic acid having a nucleotide sequence encoding a genome-targeting nucleic acid of the disclosure, a site-directed polypeptide of the disclosure, and/or any nucleic acid or proteinaceous molecule necessary to carry out the embodiments of the methods of the disclosure. In some embodiments, such a nucleic acid is a vector (e.g., a recombinant expression vector).

Expression vectors contemplated include, but are not limited to, viral vectors based on vaccinia virus, poliovirus, adenovirus, adeno-associated virus, SV40, herpes simplex virus, human immunodeficiency virus, retrovirus (e.g., Murine Leukemia Virus, spleen necrosis virus, and vectors derived from retroviruses such as Rous Sarcoma Virus, Harvey Sarcoma Virus, avian leukosis virus, a lentivirus, human immunodeficiency virus, myeloproliferative sarcoma virus, and mammary tumor virus) and other recombinant vectors. Other vectors contemplated for eukaryotic target cells include, but are not limited to, the vectors pXT1, pSG5, pSVK3, pBPV, pMSG, and pSVLSV40 (Pharmacia). Additional vectors contemplated for eukaryotic target cells include, but are not limited to, the vectors pCTx-1, pCTx-2, and pCTx-3. Other vectors can be used so long as they are compatible with the host cell.

In some embodiments, a vector has one or more transcription and/or translation control elements. Depending on the host/vector system utilized, any of a number of suitable transcription and translation control elements, including constitutive and inducible promoters, transcription enhancer elements, transcription terminators, etc. can be used in the expression vector. In some embodiments, the vector is a self-inactivating vector that either inactivates the viral sequences or the components of the CRISPR machinery or other elements.

Non-limiting examples of suitable eukaryotic promoters (i.e., promoters functional in a eukaryotic cell) include those from cytomegalovirus (CMV) immediate early, herpes simplex virus (HSV) thymidine kinase, early and late SV40, long terminal repeats (LTRs) from retrovirus, human elongation factor-1 promoter (EF1), a hybrid construct having the cytomegalovirus (CMV) enhancer fused to the chicken beta-actin promoter (CAG), murine stem cell virus promoter (MSCV), phosphoglycerate kinase-1 locus promoter (PGK), and mouse metallothionein-I.

For expressing small RNAs, including guide RNAs used in connection with Cas endonuclease, various promoters such as RNA polymerase III promoters, including for example U6 and H1, can be advantageous. Descriptions of and parameters for enhancing the use of such promoters are known in art, and additional information and approaches are regularly being described; see, e.g., Ma, H. et al., Molecular Therapy—Nucleic Acids 3, e161 (2014) doi:10.1038/mtna.2014.12.

The expression vector can also contain a ribosome binding site for translation initiation and a transcription terminator. The expression vector can also include appropriate sequences for amplifying expression. The expression vector can also include nucleotide sequences encoding non-native tags (e.g., histidine tag, hemagglutinin tag, green fluorescent protein, etc.) that are fused to the site-directed polypeptide, thus resulting in a fusion protein.

In some embodiments, a promoter is an inducible promoter (e.g., a heat shock promoter, tetracycline-regulated promoter, steroid-regulated promoter, metal-regulated promoter, estrogen receptor-regulated promoter, etc.). In some embodiments, a promoter is a constitutive promoter (e.g., CMV promoter, UBC promoter). In some embodiments, the promoter is a spatially restricted and/or temporally restricted promoter (e.g., a tissue specific promoter, a cell type specific promoter, etc.). In some embodiments, a vector does not have a promoter for at least one gene to be expressed in a host cell if the gene is going to be expressed, after it is inserted into a genome, under an endogenous promoter present in the genome.

### Site-Directed Polypeptide or DNA Endonuclease

The modifications of the target DNA due to NHEJ and/or HDR can lead to, for example, mutations, deletions, alterations, integrations, gene correction, gene replacement, gene tagging, transgene insertion, nucleotide deletion, gene disruption, translocations and/or gene mutation. The process of integrating non-native nucleic acid into genomic DNA is an example of genome editing.

A site-directed polypeptide is a nuclease used in genome editing to cleave DNA. The site-directed can be administered to a cell or a patient as either: one or more polypeptides, or one or more mRNAs encoding the polypeptide.

In the context of a CRISPR/Cas or CRISPR/Cpf1 system, the site-directed polypeptide can bind to a guide RNA that, in turn, specifies the site in the target DNA to which the polypeptide is directed. In embodiments of CRISPR/Cas or CRISPR/Cpf1 systems herein, the site-directed polypeptide is an endonuclease, such as a DNA endonuclease.

In some embodiments, a site-directed polypeptide has a plurality of nucleic acid-cleaving (i.e., nuclease) domains. Two or more nucleic acid-cleaving domains can be linked together via a linker. In some embodiments, the linker has a flexible linker. Linkers can have 1, 2, 3, 4, 5, 6, 7, 8, 9, 10, 11, 12, 13, 14, 15, 16, 17, 18, 19, 20, 21, 22, 23, 24, 25, 30, 35, 40 or more amino acids in length.

Naturally-occurring wild-type Cas9 enzymes have two nuclease domains, a HNH nuclease domain and a RuvC domain. Herein, the “Cas9” refers to both naturally-occurring and recombinant Cas9s. Cas9 enzymes contemplated herein have a HNH or HNH-like nuclease domain, and/or a RuvC or RuvC-like nuclease domain.

HNH or HNH-like domains have a McrA-like fold. HNH or HNH-like domains has two antiparallel β-strands and an a-helix. HNH or HNH-like domains has a metal binding site (e.g., a divalent cation binding site). HNH or HNH-like domains can cleave one strand of a target nucleic acid (e.g., the complementary strand of the crRNA targeted strand).

RuvC or RuvC-like domains have an RNaseH or RNaseH-like fold. RuvC/RNaseH domains are involved in a diverse set of nucleic acid-based functions including acting on both RNA and DNA. The RNaseH domain has 5 β-strands surrounded by a plurality of a-helices. RuvC/RNaseH or RuvC/RNaseH-like domains have a metal binding site (e.g., a divalent cation binding site). RuvC/RNaseH or RuvC/RNaseH-like domains can cleave one strand of a target nucleic acid (e.g., the non-complementary strand of a double-stranded target DNA).

In some embodiments, the site-directed polypeptide has an amino acid sequence having at least 10%, at least 15%, at least 20%, at least 30%, at least 40%, at least 50%, at least 60%, at least 70%, at least 75%, at least 80%, at least 85%, at least 90%, at least 95%, at least 99%, or 100% amino acid sequence identity to a wild-type exemplary site-directed polypeptide [e.g., Cas9 from S. pyogenes, US2014/0068797 Sequence ID No. 8 or Sapranauskas et al., Nucleic Acids Res, 39(21): 9275-9282 (2011)], and various other site-directed polypeptides).

In some embodiments, the site-directed polypeptide has an amino acid sequence having at least 10%, at least 15%, at least 20%, at least 30%, at least 40%, at least 50%, at least 60%, at least 70%, at least 75%, at least 80%, at least 85%, at least 90%, at least 95%, at least 99%, or 100% amino acid sequence identity to the nuclease domain of a wild-type exemplary site-directed polypeptide (e.g., Cas9 from S. pyogenes, supra).

In some embodiments, a site-directed polypeptide has at least 70, 75, 80, 85, 90, 95, 97, 99, or 100% identity to a wild-type site-directed polypeptide (e.g., Cas9 from S. pyogenes, supra) over 10 contiguous amino acids. In some embodiments, a site-directed polypeptide has at most: 70, 75, 80, 85, 90, 95, 97, 99, or 100% identity to a wild-type site-directed polypeptide (e.g., Cas9 from S. pyogenes, supra) over 10 contiguous amino acids. In some embodiments, a site-directed polypeptide has at least: 70, 75, 80, 85, 90, 95, 97, 99, or 100% identity to a wild-type site-directed polypeptide (e.g., Cas9 from S. pyogenes, supra) over 10 contiguous amino acids in a HNH nuclease domain of the site-directed polypeptide. In some embodiments, a site-directed polypeptide has at most: 70, 75, 80, 85, 90, 95, 97, 99, or 100% identity to a wild-type site-directed polypeptide (e.g., Cas9 from S. pyogenes, supra) over 10 contiguous amino acids in a HNH nuclease domain of the site-directed polypeptide. In some embodiments, a site-directed polypeptide has at least: 70, 75, 80, 85, 90, 95, 97, 99, or 100% identity to a wild-type site-directed polypeptide (e.g., Cas9 from S. pyogenes, supra) over 10 contiguous amino acids in a RuvC nuclease domain of the site-directed polypeptide. In some embodiments, a site-directed polypeptide has at most: 70, 75, 80, 85, 90, 95, 97, 99, or 100% identity to a wild-type site-directed polypeptide (e.g., Cas9 from S. pyogenes, supra) over 10 contiguous amino acids in a RuvC nuclease domain of the site-directed polypeptide.

In some embodiments, the site-directed polypeptide has a modified form of a wild-type exemplary site-directed polypeptide. The modified form of the wild-type exemplary site-directed polypeptide has a mutation that reduces the nucleic acid-cleaving activity of the site-directed polypeptide. In some embodiments, the modified form of the wild-type exemplary site-directed polypeptide has less than 90%, less than 80%, less than 70%, less than 60%, less than 50%, less than 40%, less than 30%, less than 20%, less than 10%, less than 5%, or less than 1% of the nucleic acid-cleaving activity of the wild-type exemplary site-directed polypeptide (e.g., Cas9 from S. pyogenes, supra). The modified form of the site-directed polypeptide can have no substantial nucleic acid-cleaving activity. When a site-directed polypeptide is a modified form that has no substantial nucleic acid-cleaving activity, it is referred to herein as “enzymatically inactive.”

In some embodiments, the modified form of the site-directed polypeptide has a mutation such that it can induce a single-strand break (SSB) on a target nucleic acid (e.g., by cutting only one of the sugar-phosphate backbones of a double-strand target nucleic acid). In some embodiments, the mutation results in less than 90%, less than 80%, less than 70%, less than 60%, less than 50%, less than 40%, less than 30%, less than 20%, less than 10%, less than 5%, or less than 1% of the nucleic acid-cleaving activity in one or more of the plurality of nucleic acid-cleaving domains of the wild-type site directed polypeptide (e.g., Cas9 from S. pyogenes, supra). In some embodiments, the mutation results in one or more of the plurality of nucleic acid-cleaving domains retaining the ability to cleave the complementary strand of the target nucleic acid, but reducing its ability to cleave the non-complementary strand of the target nucleic acid. In some embodiments, the mutation results in one or more of the plurality of nucleic acid-cleaving domains retaining the ability to cleave the non-complementary strand of the target nucleic acid, but reducing its ability to cleave the complementary strand of the target nucleic acid. For example, residues in the wild-type exemplary S. pyogenes Cas9 polypeptide, such as Asp10, His840, Asn854 and Asn856, are mutated to inactivate one or more of the plurality of nucleic acid-cleaving domains (e.g., nuclease domains). In some embodiments, the residues to be mutated correspond to residues Asp10, His840, Asn854 and Asn856 in the wild-type exemplary S. pyogenes Cas9 polypeptide (e.g., as determined by sequence and/or structural alignment). Non-limiting examples of mutations include D10A, H840A, N854A or N856A. One skilled in the art will recognize that mutations other than alanine substitutions are suitable.

In some embodiments, a D10A mutation is combined with one or more of H840A, N854A, or N856A mutations to produce a site-directed polypeptide substantially lacking DNA cleavage activity. In some embodiments, a H840A mutation is combined with one or more of D10A, N854A, or N856A mutations to produce a site-directed polypeptide substantially lacking DNA cleavage activity. In some embodiments, a N854A mutation is combined with one or more of H840A, D10A, or N856A mutations to produce a site-directed polypeptide substantially lacking DNA cleavage activity. In some embodiments, a N856A mutation is combined with one or more of H840A, N854A, or D10A mutations to produce a site-directed polypeptide substantially lacking DNA cleavage activity. Site-directed polypeptides that have one substantially inactive nuclease domain are referred to as “nickases”.

In some embodiments, variants of RNA-guided endonucleases, for example Cas9, can be used to increase the specificity of CRISPR-mediated genome editing. Wild type Cas9 is typically guided by a single guide RNA designed to hybridize with a specified ˜20 nucleotide sequence in the target sequence (such as an endogenous genomic locus). However, several mismatches can be tolerated between the guide RNA and the target locus, effectively reducing the length of required homology in the target site to, for example, as little as 13 nt of homology, and thereby resulting in elevated potential for binding and double-strand nucleic acid cleavage by the CRISPR/Cas9 complex elsewhere in the target genome—also known as off-target cleavage. Because nickase variants of Cas9 each only cut one strand, in order to create a double-strand break it is necessary for a pair of nickases to bind in close proximity and on opposite strands of the target nucleic acid, thereby creating a pair of nicks, which is the equivalent of a double-strand break. This requires that two separate guide RNAs—one for each nickase—must bind in close proximity and on opposite strands of the target nucleic acid. This requirement essentially doubles the minimum length of homology needed for the double-strand break to occur, thereby reducing the likelihood that a double-strand cleavage event will occur elsewhere in the genome, where the two guide RNA sites—if they exist—are unlikely to be sufficiently close to each other to enable the double-strand break to form. As described in the art, nickases can also be used to promote HDR versus NHEJ. HDR can be used to introduce selected changes into target sites in the genome through the use of specific donor sequences that effectively mediate the desired changes. Descriptions of various CRISPR/Cas systems for use in gene editing can be found, e.g., in international patent application publication number WO2013/176772, and in Nature Biotechnology 32, 347-355 (2014), and references cited therein.

In some embodiments, the site-directed polypeptide (e.g., variant, mutated, enzymatically inactive and/or conditionally enzymatically inactive site-directed polypeptide) targets nucleic acid. In some embodiments, the site-directed polypeptide (e.g., variant, mutated, enzymatically inactive and/or conditionally enzymatically inactive endoribonuclease) targets DNA. In some embodiments, the site-directed polypeptide (e.g., variant, mutated, enzymatically inactive and/or conditionally enzymatically inactive endoribonuclease) targets RNA.

In some embodiments, the site-directed polypeptide has one or more non-native sequences (e.g., the site-directed polypeptide is a fusion protein).

In some embodiments, the site-directed polypeptide has an amino acid sequence having at least 15% amino acid identity to a Cas9 from a bacterium (e.g., S. pyogenes), a nucleic acid binding domain, and two nucleic acid cleaving domains (i.e., a HNH domain and a RuvC domain).

In some embodiments, the site-directed polypeptide has an amino acid sequence having at least 15% amino acid identity to a Cas9 from a bacterium (e.g., S. pyogenes), and two nucleic acid cleaving domains (i.e., a HNH domain and a RuvC domain).

In some embodiments, the site-directed polypeptide has an amino acid sequence having at least 15% amino acid identity to a Cas9 from a bacterium (e.g., S. pyogenes), and two nucleic acid cleaving domains, wherein one or both of the nucleic acid cleaving domains have at least 50% amino acid identity to a nuclease domain from Cas9 from a bacterium (e.g., S. pyogenes).

In some embodiments, the site-directed polypeptide has an amino acid sequence having at least 15% amino acid identity to a Cas9 from a bacterium (e.g., S. pyogenes), two nucleic acid cleaving domains (i.e., a HNH domain and a RuvC domain), and non-native sequence (for example, a nuclear localization signal) or a linker linking the site-directed polypeptide to a non-native sequence.

In some embodiments, the site-directed polypeptide has an amino acid sequence having at least 15% amino acid identity to a Cas9 from a bacterium (e.g., S. pyogenes), two nucleic acid cleaving domains (i.e., a HNH domain and a RuvC domain), wherein the site-directed polypeptide has a mutation in one or both of the nucleic acid cleaving domains that reduces the cleaving activity of the nuclease domains by at least 50%.

In some embodiments, the site-directed polypeptide has an amino acid sequence having at least 15% amino acid identity to a Cas9 from a bacterium (e.g., S. pyogenes), and two nucleic acid cleaving domains (i.e., a HNH domain and a RuvC domain), wherein one of the nuclease domains has mutation of aspartic acid 10, and/or wherein one of the nuclease domains has mutation of histidine 840, and wherein the mutation reduces the cleaving activity of the nuclease domain(s) by at least 50%.

In some embodiments, the one or more site-directed polypeptides, e.g. DNA endonucleases, include two nickases that together effect one double-strand break at a specific locus in the genome, or four nickases that together effect two double-strand breaks at specific loci in the genome. Alternatively, one site-directed polypeptide, e.g. DNA endonuclease, affects one double-strand break at a specific locus in the genome.

In some embodiments, a polynucleotide encoding a site-directed polypeptide can be used to edit genome. In some of such embodiments, the polynucleotide encoding a site-directed polypeptide is codon-optimized according to methods standard in the art for expression in the cell containing the target DNA of interest. For example, if the intended target nucleic acid is in a human cell, a human codon-optimized polynucleotide encoding Cas9 is contemplated for use for producing the Cas9 polypeptide.

The following provides some examples of site-directed polypeptides that can be used in various embodiments of the disclosures.

### CRISPR Endonuclease System

A CRISPR (Clustered Regularly Interspaced Short Palindromic Repeats) genomic locus can be found in the genomes of many prokaryotes (e.g., bacteria and archaea). In prokaryotes, the CRISPR locus encodes products that function as a type of immune system to help defend the prokaryotes against foreign invaders, such as virus and phage. There are three stages of CRISPR locus function: integration of new sequences into the CRISPR locus, expression of CRISPR RNA (crRNA), and silencing of foreign invader nucleic acid. Five types of CRISPR systems (e.g., Type I, Type II, Type III, Type U, and Type V) have been identified.

A CRISPR locus includes a number of short repeating sequences referred to as “repeats.” When expressed, the repeats can form secondary hairpin structures (e.g., hairpins) and/or have unstructured single-stranded sequences. The repeats usually occur in clusters and frequently diverge between species. The repeats are regularly interspaced with unique intervening sequences referred to as “spacers,” resulting in a repeat-spacer-repeat locus architecture. The spacers are identical to or have high homology with known foreign invader sequences. A spacer-repeat unit encodes a crisprRNA (crRNA), which is processed into a mature form of the spacer-repeat unit. A crRNA has a “seed” or spacer sequence that is involved in targeting a target nucleic acid (in the naturally occurring form in prokaryotes, the spacer sequence targets the foreign invader nucleic acid). A spacer sequence is located at the 5′ or 3′ end of the crRNA.

A CRISPR locus also has polynucleotide sequences encoding CRISPR Associated (Cas) genes. Cas genes encode endonucleases involved in the biogenesis and the interference stages of crRNA function in prokaryotes. Some Cas genes have homologous secondary and/or tertiary structures.

### Type II CRISPR Systems

crRNA biogenesis in a Type II CRISPR system in nature requires a trans-activating CRISPR RNA (tracrRNA). The tracrRNA is modified by endogenous RNaseIII, and then hybridizes to a crRNA repeat in the pre-crRNA array. Endogenous RNaseIII is recruited to cleave the pre-crRNA. Cleaved crRNAs are subjected to exoribonuclease trimming to produce the mature crRNA form (e.g., 5′ trimming) The tracrRNA remains hybridized to the crRNA, and the tracrRNA and the crRNA associate with a site-directed polypeptide (e.g., Cas9). The crRNA of the crRNA-tracrRNA-Cas9 complex guides the complex to a target nucleic acid to which the crRNA can hybridize. Hybridization of the crRNA to the target nucleic acid activates Cas9 for targeted nucleic acid cleavage. The target nucleic acid in a Type II CRISPR system is referred to as a protospacer adjacent motif (PAM). In nature, the PAM is essential to facilitate binding of a site-directed polypeptide (e.g., Cas9) to the target nucleic acid. Type II systems (also referred to as Nmeni or CASS4) are further subdivided into Type II-A (CASS4) and II-B (CASS4a). Jinek et al., Science, 337(6096):816-821 (2012) showed that the CRISPR/Cas9 system is useful for RNA-programmable genome editing, and international patent application publication number WO 2013/176772 provides numerous examples and applications of the CRISPR/Cas endonuclease system for site-specific gene editing.

### Type V CRISPR Systems

Type V CRISPR systems have several important differences from Type II systems. For example, Cpf1 is a single RNA-guided endonuclease that, in contrast to Type II systems, lacks tracrRNA. In fact, Cpf1-associated CRISPR arrays are processed into mature crRNAS without the requirement of an additional trans-activating tracrRNA. The Type V CRISPR array is processed into short mature crRNAs of 42-44 nucleotides in length, with each mature crRNA beginning with 19 nucleotides of direct repeat followed by 23-25 nucleotides of spacer sequence. In contrast, mature crRNAs in Type II systems start with 20-24 nucleotides of spacer sequence followed by about 22 nucleotides of direct repeat. Also, Cpf1 utilizes a T-rich protospacer-adjacent motif such that Cpf1-crRNA complexes efficiently cleave target DNA preceded by a short T-rich PAM, which is in contrast to the G-rich PAM following the target DNA for Type II systems. Thus, Type V systems cleave at a point that is distant from the PAM, while Type II systems cleave at a point that is adjacent to the PAM. In addition, in contrast to Type II systems, Cpf1 cleaves DNA via a staggered DNA double-stranded break with a 4 or 5 nucleotide 5′ overhang. Type II systems cleave via a blunt double-stranded break. Similar to Type II systems, Cpf1 contains a predicted RuvC-like endonuclease domain, but lacks a second HNH endonuclease domain, which is in contrast to Type II systems.

### Cas Genes/Polypeptides and Protospacer Adjacent Motifs

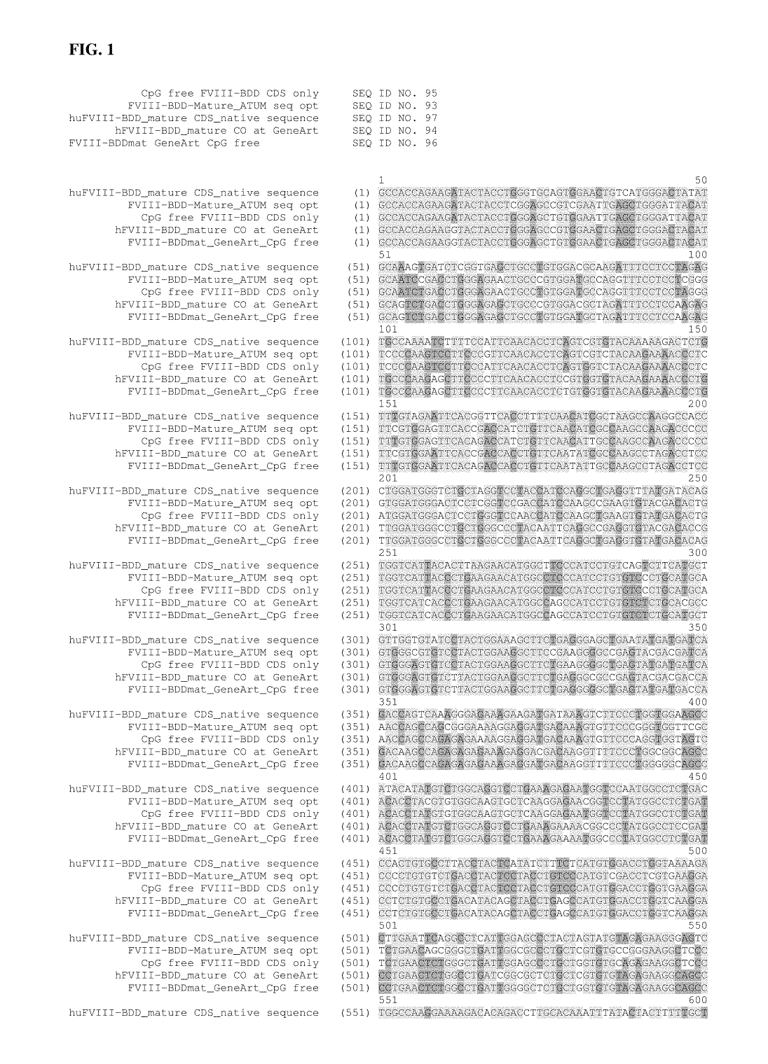

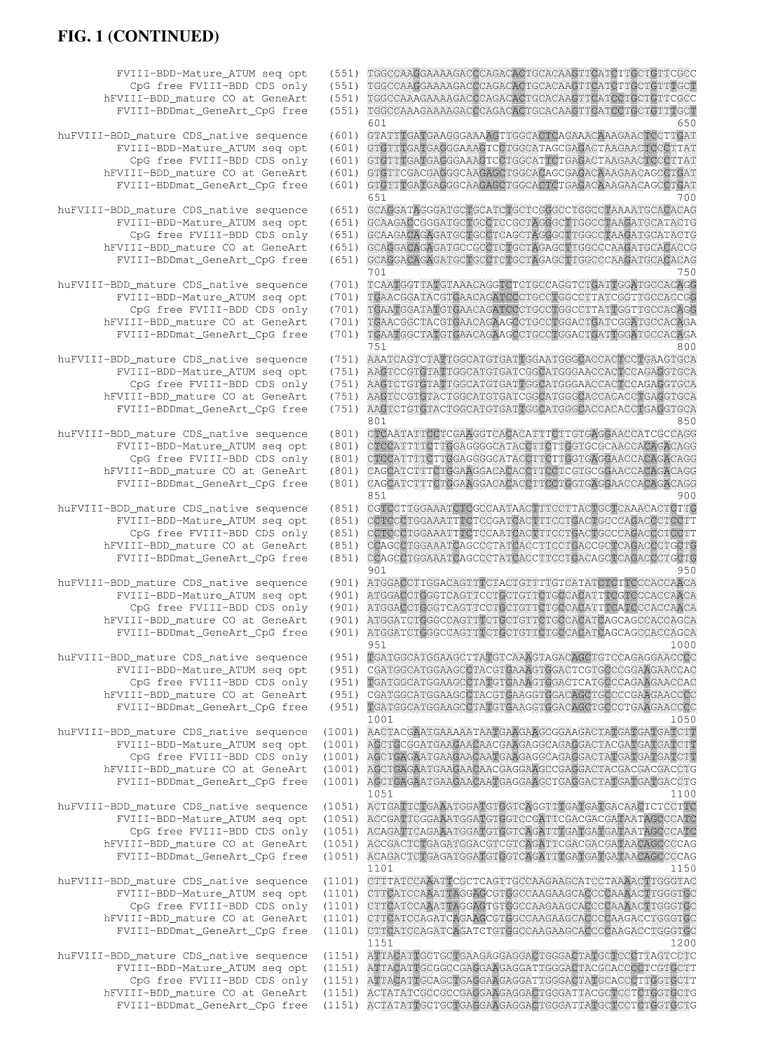

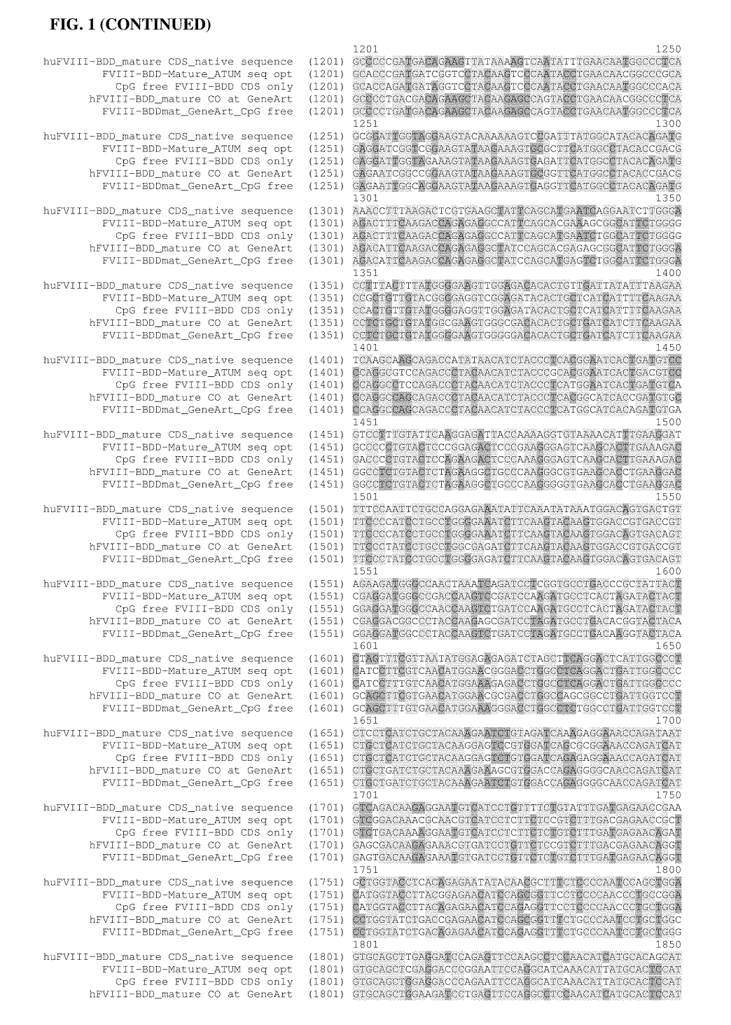

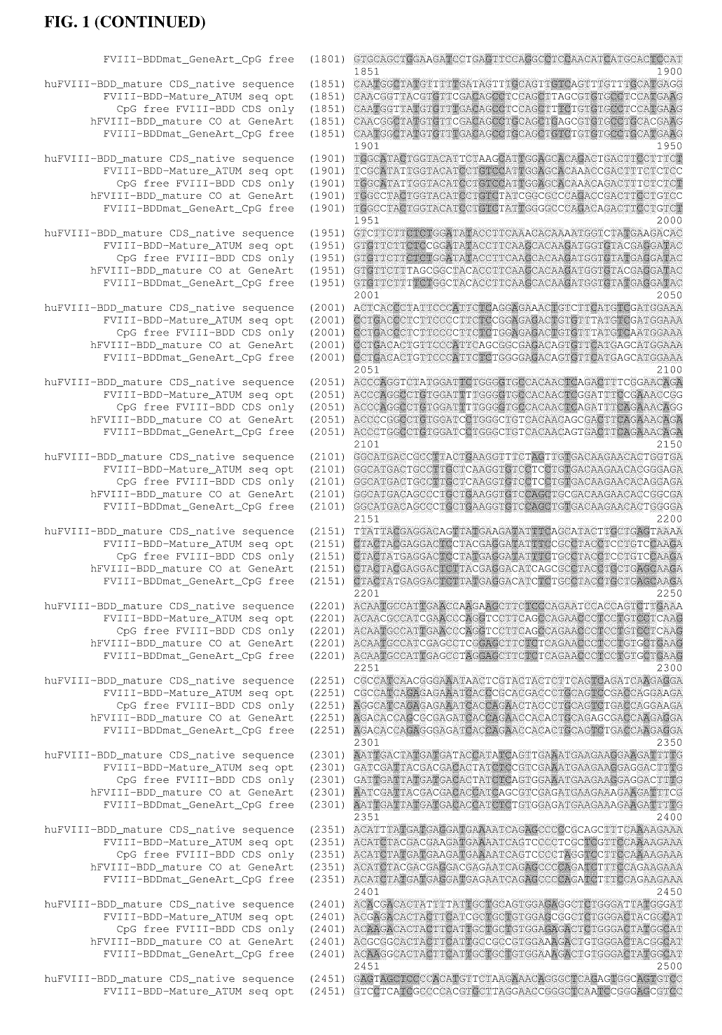

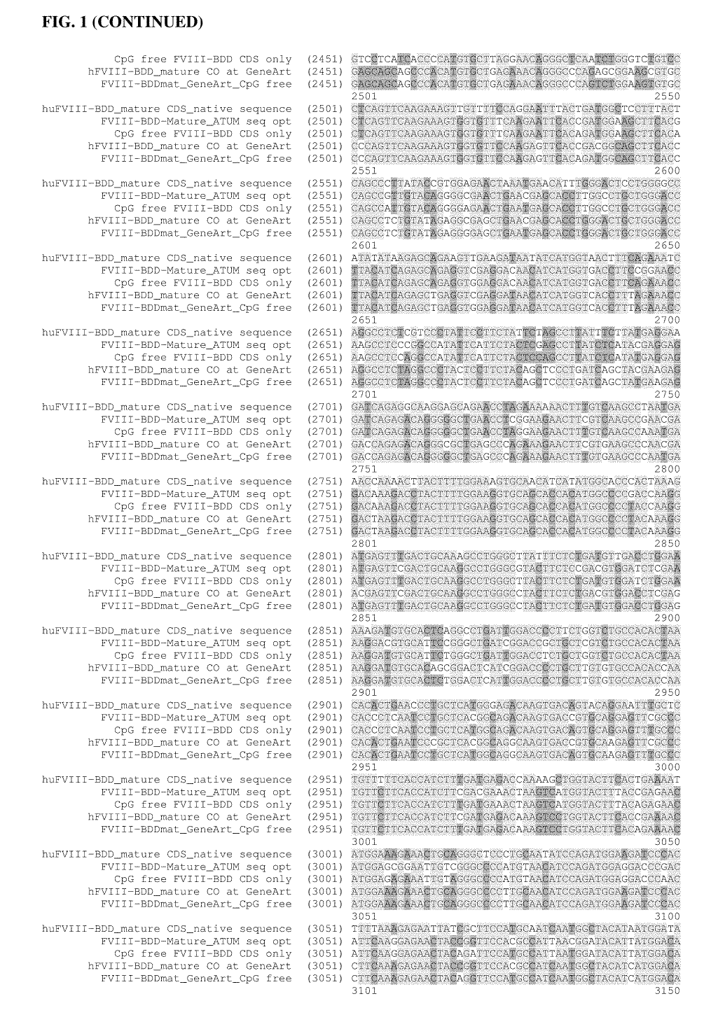

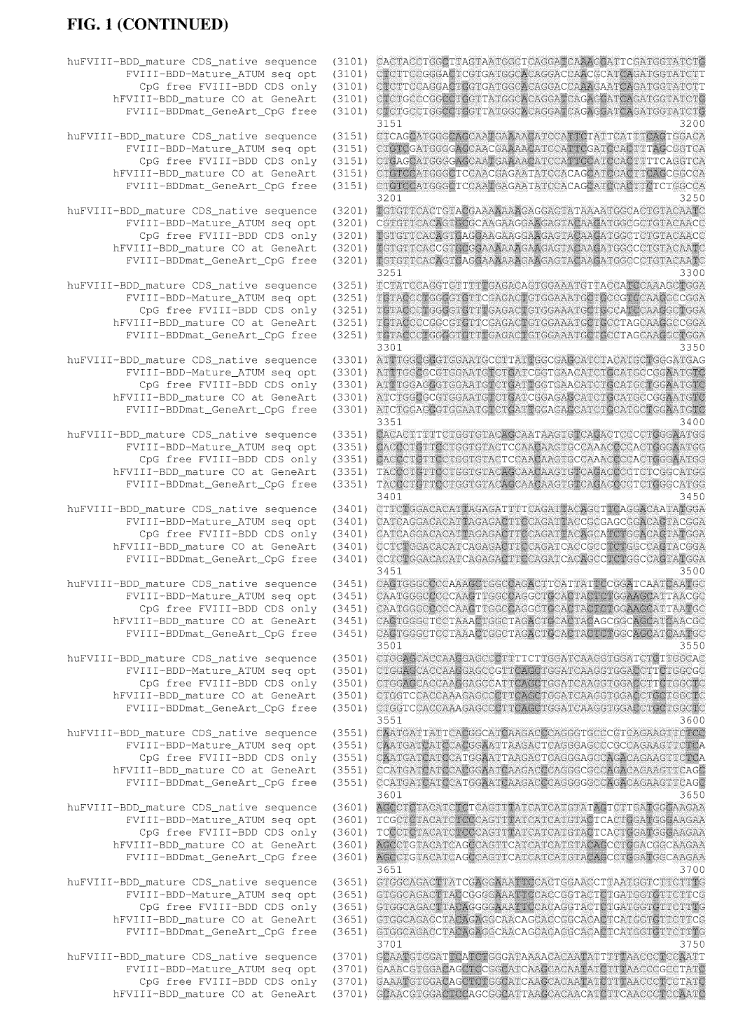

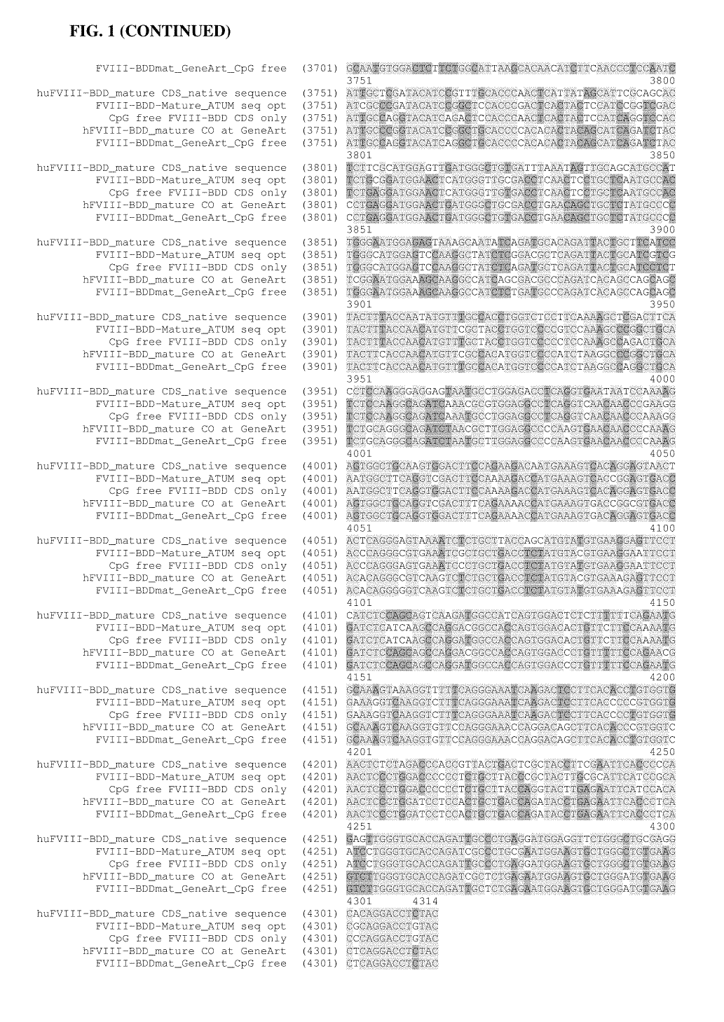

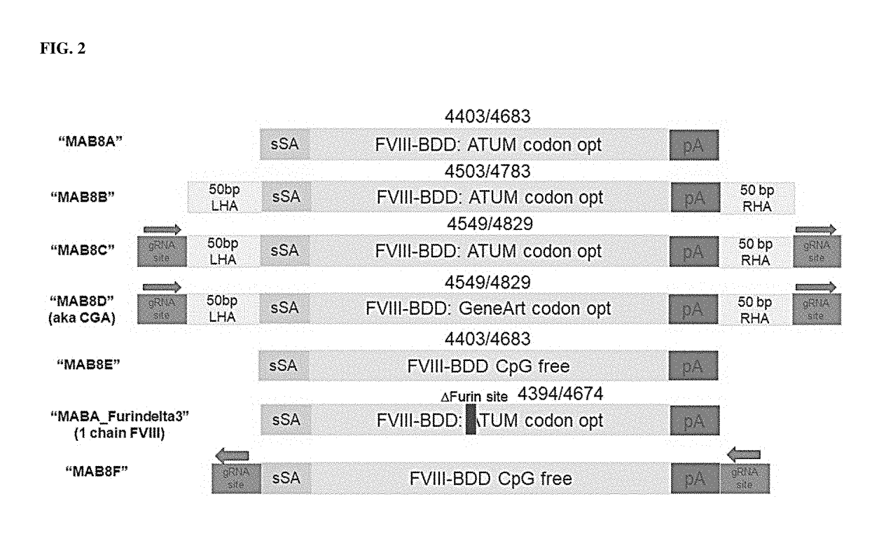

Exemplary CRISPR/Cas polypeptides include the Cas9 polypeptides in FIG. 1 of Fonfara et al., Nucleic Acids Research, 42: 2577-2590 (2014). The CRISPR/Cas gene naming system has undergone extensive rewriting since the Cas genes were discovered. FIG. 5 of Fonfara, supra, provides PAM sequences for the Cas9 polypeptides from various species.

### Complexes of a Genome-Targeting Nucleic Acid and a Site-Directed Polypeptide

A genome-targeting nucleic acid interacts with a site-directed polypeptide (e.g., a nucleic acid-guided nuclease such as Cas9), thereby forming a complex. The genome-targeting nucleic acid (e.g. gRNA) guides the site-directed polypeptide to a target nucleic acid.

As stated previously, in some embodiments the site-directed polypeptide and genome-targeting nucleic acid can each be administered separately to a cell or a patient. On the other hand, in some other embodiments the site-directed polypeptide can be pre-complexed with one or more guide RNAs, or one or more crRNA together with a tracrRNA. The pre-complexed material can then be administered to a cell or a patient. Such pre-complexed material is known as a ribonucleoprotein particle (RNP).

### Systems for Genome Editing

Provided herein are systems for genome editing, in particular, for inserting a Factor VIII (FVIII) gene or functional derivative thereof into the genome of a cell. These systems can be used in methods described herein, such as for editing the genome of a cell and for treating a subject, e.g. a patient of Hemophilia A.

In some embodiments, provided herein is a system comprising (a) a deoxyribonucleic acid (DNA) endonuclease or nucleic acid encoding said DNA endonuclease; (b) a guide RNA (gRNA) targeting the albumin locus in the genome of a cell; and (c) a donor template comprising a nucleic acid sequence encoding a Factor VIII (FVIII) protein or functional derivative thereof. In some embodiments, the gRNA targets intron 1 of the albumin gene. In some embodiments, the gRNA comprises a spacer sequence from any one of SEQ ID NOs: 18-44 and 104.

In some embodiments, provided herein is a system comprising (a) a deoxyribonucleic acid (DNA) endonuclease or nucleic acid encoding said DNA endonuclease; (b) a guide RNA (gRNA) comprising a spacer sequence from any one of SEQ ID NOs: 18-44 and 104; and (c) a donor template comprising a nucleic acid sequence encoding a Factor VIII (FVIII) protein or functional derivative thereof. In some embodiments, the gRNA comprises a spacer sequence from any one of SEQ ID NOs: 21, 22, 28, and 30. In some embodiments, the gRNA comprises a spacer sequence from SEQ ID NO: 21. In some embodiments, the gRNA comprises a spacer sequence from SEQ ID NO: 22. In some embodiments, the gRNA comprises a spacer sequence from SEQ ID NO: 28. In some embodiments, the gRNA comprises a spacer sequence from SEQ ID NO: 30.

In some embodiments, according to any of the systems described herein, the DNA endonuclease is selected from the group consisting of a Cas1, Cas1B, Cas2, Cas3, Cas4, Cas5, Cas6, Cas7, Cas8, Cas9 (also known as Csn1 and Csx12), Cas100, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, or Cpf1 endonuclease, or a functional derivative thereof. In some embodiments, the DNA endonuclease is Cas9. In some embodiments, the Cas9 is from Streptococcus pyogenes (spCas9). In some embodiments, the Cas9 is from Staphylococcus lugdunensis (SluCas9).

In some embodiments, according to any of the systems described herein, the nucleic acid sequence encoding a Factor VIII (FVIII) protein or functional derivative thereof is codon optimized for expression in a host cell. In some embodiments, the nucleic acid sequence encoding a Factor VIII (FVIII) protein or functional derivative thereof is codon optimized for expression in a human cell.

In some embodiments, according to any of the systems described herein, the system comprises a nucleic acid encoding the DNA endonuclease. In some embodiments, the nucleic acid encoding the DNA endonuclease is codon optimized for expression in a host cell. In some embodiments, the nucleic acid encoding the DNA endonuclease is codon optimized for expression in a human cell. In some embodiments, the nucleic acid encoding the DNA endonuclease is DNA, such as a DNA plasmid. In some embodiments, the nucleic acid encoding the DNA endonuclease is RNA, such as mRNA.

In some embodiments, according to any of the systems described herein, the donor template is encoded in an Adeno Associated Virus (AAV) vector. In some embodiments, the donor template comprises a donor cassette comprising the nucleic acid sequence encoding a Factor VIII (FVIII) protein or functional derivative, and the donor cassette is flanked on one or both sides by a gRNA target site. In some embodiments, the donor cassette is flanked on both sides by a gRNA target site. In some embodiments, the gRNA target site is a target site for a gRNA in the system. In some embodiments, the gRNA target site of the donor template is the reverse complement of a cell genome gRNA target site for a gRNA in the system.

In some embodiments, according to any of the systems described herein, the DNA endonuclease or nucleic acid encoding the DNA endonuclease is formulated in a liposome or lipid nanoparticle. In some embodiments, the liposome or lipid nanoparticle also comprises the gRNA. In some embodiments, the liposome or lipid nanoparticle is a lipid nanoparticle. In some embodiments, the system comprises a lipid nanoparticle comprising nucleic acid encoding the DNA endonuclease and the gRNA. In some embodiments, the nucleic acid encoding the DNA endonuclease is an mRNA encoding the DNA endonuclease.

In some embodiments, according to any of the systems described herein, the DNA endonuclease is complexed with the gRNA, forming a ribonucleoprotein (RNP) complex.

### Methods of Genome Edition

Provided herein is a method of genome editing, in particular, inserting a Factor VIII (FVIII) gene or functional derivative thereof into the genome of a cell. This method can be used to treat a subject, e.g. a patient of Hemophilia A and in such a case, a cell can be isolated from the patient or a separate donor. Then, the chromosomal DNA of the cell is edited using the materials and methods described herein.

In some embodiments, a knock-in strategy involves knocking-in a FVIII-encoding sequence, e.g. a wildtype FVIII gene (e.g. the wildtype human FVIII gene), a FVIII cDNA, a minigene (having natural or synthetic enhancer and promoter, one or more exons, and natural or synthetic introns, and natural or synthetic 3′UTR and polyadenylation signal) or a modified FVIII gene, into a genomic sequence. In some embodiments, the genomic sequence where the FVIII-encoding sequence is inserted is at, within or near the albumin locus.

Provided herein are methods to knock-in a FVIII gene or functional derivative thereof into a genome. In one aspect, the present disclosure provides insertion of a nucleic acid sequence of a FVIII gene, i.e. a nucleic acid sequence encoding a FVIII protein or functional derivative thereof into a genome of a cell. In embodiments, the FVIII gene can encode a wild-type FVIII protein. The functional derivative of a FVIII protein can include a peptide that has a substantial activity of the wildtype FVIII protein, e.g. at least about 30%, about 40%, about 50%, about 60%, about 70%, about 80%, about 90%, about 95% or about 100% of the activity that the wildtype FVIII protein exhibits. In some embodiments, one having ordinary skill in the art can use a number of methods known in the field to test the functionality or activity of a compound, e.g. peptide or protein. In some embodiments, the functional derivative of the FVIII protein can also include any fragment of the wildtype FVIII protein or fragment of a modified FVIII protein that has conservative modification on one or more of amino acid residues in the full length, wildtype FVIII protein. In some embodiments, the functional derivative of the FVIII protein can also include any modification(s), e.g. deletion, insertion and/or mutation of one or more amino acids that do not substantially negatively affect the functionality of the wildtype FVIII protein. Thus, in some embodiments, the functional derivative of a nucleic acid sequence of a FVIII gene can have at least about 30%, about 40%, about 50%, about 60%, about 70%, about 80%, about 85%, about 90%, about 95%, about 96%, about 97%, about 98% or about 99% nucleic acid sequence identity to the FVIII gene.

In some embodiments, a FVIII gene or functional derivative thereof is inserted into a genomic sequence in a cell. In some embodiments, the insertion site is at, or within the albumin locus in the genome of the cell. The insertion method uses one or more gRNAs targeting the first intron (or intron 1) of the albumin gene. In some embodiments, the donor DNA is single or double stranded DNA having a FVIII gene or functional derivative thereof.

In some embodiments, the genome editing methods utilize a DNA endonuclease such as a CRISPR/Cas system to genetically introduce (knock-in) a FVIII gene or functional derivative thereof. In some embodiments, the DNA endonuclease is a Cas1, Cas1B, Cas2, Cas3, Cas4, Cas5, Cas6, Cas7, Cas8, Cas9 (also known as Csn1 and Csx12), Cas100, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, or Cpf1 endonuclease, a homolog thereof, recombination of the naturally occurring molecule, codon-optimized, or modified version thereof, and combinations of any of the foregoing. In some embodiments, the DNA endonuclease is Cas9. In some embodiments, the Cas9 is from Streptococcus pyogenes (spCas9). In some embodiments, the Cas9 is from Staphylococcus lugdunensis (SluCas9).

In some embodiments, the cell subject to the genome-edition has one or more mutation(s) in the genome which results in reduction of the expression of endogenous FVIII gene as compared to the expression in a normal that does not have such mutation(s). The normal cell can be a healthy or control cell that is originated (or isolated) from a different subject who does not have FVIII gene defects. In some embodiments, the cell subject to the genome-edition can be originated (or isolated) from a subject who is in need of treatment of FVIII gene related condition or disorder, e.g. Hemophilia A. Therefore, in some embodiments the expression of endogenous FVIII gene in such cell is about 10%, about 20%, about 30%, about 40%, about 50%, about 60%, about 70%, about 80%, about 90% or about 100% reduced as compared to the expression of endogenous FVIII gene expression in the normal cell.

In some embodiments, the genome editing methods conducts targeted integration at non-coding region of the genome of a functional FVIII gene, e.g. a FVIII coding sequence that is operably linked to a supplied promoter so as to stably generate FVIII protein in vivo. In some embodiments, the targeted integration of a FVIII coding sequence occurs in an intron of the albumin gene that is highly expressed in the cell type of interest, e.g. hepatocytes or sinusoidal endothelial cells. In some embodiments, the FVIII coding sequence to be inserted can be a wildtype FVIII coding sequence, e.g. the wildtype human FVIII coding sequence. In some embodiments, the FVIII coding sequence can be a functional derivative of a wildtype FVIII coding sequence such as the wildtype human FVIII coding sequence.

In one aspect, the present disclosure proposes insertion of a nucleic acid sequence of a FVIII gene or functional derivative thereof into a genome of a cell. In embodiments, the FVIII coding sequence to be inserted is a modified FVIII coding sequence. In some embodiments, in the modified FVIII coding sequence the B-domain of the wildtype FVIII coding sequence is deleted and replaced with a linker peptide called the “SQ link” (amino acid sequence SFSQNPPVLKRHQR—SEQ ID NO: 1). This B-domain deleted FVIII (FVIII-BDD) is well known in the art and has equivalent biological activity as full length FVIII. In some embodiments, a B-domain deleted FVIII is preferable over a full length FVIII because of its smaller size (4371 bp vs 7053 bp). Thus, in some embodiments the FVIII-BDD coding sequence lacking the FVIII signal peptide and containing a splice acceptor sequence at its 5′ end (N-Terminus of the FVIII coding sequence) is integrated specifically in to intron 1 of the albumin gene in the hepatocytes of mammals, including humans. The transcription of this modified FVIII coding sequence from the albumin promoter can result in a pre-mRNA that contains exon 1 of albumin, part of intron 1 and the integrated FVIII-BDD gene sequence. When this pre-mRNA undergoes the natural splicing process to remove the introns, the splicing machinery can join the splice donor at the 3′ side of albumin exon 1 to the next available splice acceptor which will be the splice acceptor at the 5′ end of the FVIII-BDD coding sequence of the inserted DNA donor. This can result in a mature mRNA containing albumin exon 1 fused to the mature coding sequence for FVIII-BDD. Exon 1 of albumin encodes the signal peptide plus 2 additional amino acids and ⅓ of a codon that in humans normally encodes the protein sequence DAH at the N-terminus of albumin. Therefore, in some embodiments after the predicted cleavage of the albumin signal peptide during secretion from the cell a FVIII-BDD protein can be generated that has 3 additional amino acid residues added to the N-terminus resulting in the amino acid sequence—DAHATRRYY (SEQ ID NO: 98)—at the N-terminus of the FVIII-BDD protein. Because the 3^{rd} of these 3 amino acids (underlined) is encoded partly by the end of exon 1 and partly by the FVIII-BDD DNA donor template it is possible to select the identity of the 3^{rd} additional amino acid residue to be either Leu, Pro, His, Gln or Arg. Among these options Leu is preferable in some embodiments since Leu is the least molecularly complex and thus least likely to form a new T-cell epitope, resulting in the amino acid sequence—DALATRRYY—at the N-terminus of the FVIII-BDD protein. Alternatively, the DNA donor template can be designed to delete the 3′ residue resulting in the amino acid sequence DALTRRYY at the N-terminus of the FVIII-BDD protein. In some cases, adding additional amino acids to the sequence of a native protein can increase the immunogenicity risk. Therefore in some embodiments where an in silico analysis to predict the potential immunogenicity of the 2 potential options for the N-terminus of FVIII-BDD demonstrates that the deletion of 1 residue (DALTRRYY) has a lower immunogenicity score, this can be a preferred design at least in some embodiments.

In some embodiments, a DNA sequence encoding FVIII-BDD in which the codon usage has been optimized can be used so as to improve the expression in mammalian cells (so called codon optimization). Different computer algorithms are also available in the field for performing codon optimization and these generate distinct DNA sequences. Examples of commercially available codon optimization algorithms are those employed by companies ATUM and GeneArt (part of Thermo Fisher Scientific). Codon optimization the FVIII coding sequence was demonstrated to significantly improve the expression of FVIII after gene based delivery to mice (Nathwani A C, Gray J T, Ng C Y, et al. Blood. 2006; 107(7):2653-2661.; Ward N J, Buckley S M, Waddington S N, et al. Blood. 2011; 117(3):798-807.; Radcliffe P A, Sion C J, Wilkes F J, et al. Gene Ther. 2008; 15(4):289-297).

In some embodiments, the sequence homology or identity between FVIII-BDD coding sequence that was codon optimized by different algorithms and the native FVIII sequence (as present in the human genome) can range from about 30%, about 40%, about 50%, about 60%, about 65%, about 70%, about 75%, about 80%, about 85%, about 90%, about 95%, or 100%. In some embodiments, the codon-optimized FVIII-BDD coding sequence has between about 75% to about 79% of sequence homology or identity to the native FVIII sequence. In some embodiments, the codon-optimized FVIII-BDD coding sequence has about 70%, about 71%, about 72%, about 73%, about 74%, about 75%, about 76%, about 77%, about 78%, about 79% or about 80% of sequence homology or identity to the native FVIII sequence.

In some embodiments, a donor template or donor construct is prepared to contain a DNA sequence encoding FVIII-BDD. In some embodiments, a DNA donor template is designed to contain a codon optimized human FVIII-BDD coding sequence. In some embodiments, the codon-optimization is done in such a way that the sequence at the 5′ end encoding the signal peptide of FVIII has been deleted and replaced with a splice acceptor sequence, and in addition a polyadenylation signal is added to the 3′ end after the FVIII stop codon (MAB8A—SEQ ID NO: 87). The splice acceptor sequence can be selected from among known splice acceptor sequences from known genes or a consensus splice acceptor sequence can be used that is derived from an alignment of many splice acceptor sequences known in the field. In some embodiments, a splice acceptor sequence from highly expressed genes is used since such sequences are thought to provide optimal splicing efficiency. In some embodiments, the consensus splicing acceptor sequence is composed of a Branch site with the consensus sequence T/CNC/TT/CA/GAC/T (SEQ ID NO: 99) followed within 20 bp with a polypyrimidine tract (C or T) of 10 to 12 bases followed by AG>G/A in which the > is the location of the intron/exon boundary. In one preferred embodiment, a synthetic splice acceptor sequence

(ctgacctcttctcttcctcccacag-SEQ ID NO: 2)

is used. In another preferred embodiment, the native splice acceptor sequence from the albumin gene intron 1/exon 2 boundary of human

(TTAACAATCCTTTTTTTTCTTCCCTTGCCCAG-SEQ ID NO: 3)

or mouse

(ttaaatatgttgtgtggtttttctctccctgtttccacag-SEQ ID
NO: 4)

is used.

The polyadenylation sequence provides a signal for the cell to add a polyA tail which is essential for the stability of the mRNA within the cell. In some embodiments that the DNA-donor template is going to be packaged into AAV particles it is preferred to keep the size of the packaged DNA within the packaging limits for AAV which are preferably less than about 5 Kb and ideally not more than about 4.7 Kb. Thus, in some embodiments it is desirable to use as short a polyA sequence as possible, e.g. about 10-mer, about 20-mer, about 30-mer, about 40-mer, about 50-mer or about 60-mer or any intervening number of nucleotides of the foregoing. A consensus synthetic poly A signal sequence has been described in the literature (Levitt N, Briggs D, Gil A, Proudfoot N.J. Genes Dev. 1989; 3(7):1019-1025.) with the sequence AATAAAAGATCTTTATTTTCATTAGATCTGTGTGTTGGTTTTTTGTGTG (SEQ ID NO: 5) and is commonly used in numerous expression vectors.

In some embodiments, additional sequence elements can be added to the DNA donor template to improve the integration frequency. One such element is homology arms which are sequences identical to the DNA sequence either side of the double strand break in the genome at which integration is targeted to enable integration by HDR. A sequence from the left side of the double strand break (LHA) is appended to the 5′ (N-terminal to the FVIII coding sequence) end of the DNA donor template and a sequence from the right side of the double strand break (RHA) is appended to the 3′ (C-terminal of the FVIII coding sequence) end of the DNA donor template for example MAB8B (SEQ ID NO: 88).

An alternative DNA donor template design that is provided in some embodiments has a sequence complementary to the recognition sequence for the sgRNA that will be used to cleave the genomic site. MAB8C (SEQ ID NO: 89) represents an example of this type of DNA donor templates. By including the sgRNA recognition site the DNA donor template will be cleaved by the sgRNA/Cas9 complex inside the nucleus of the cell to which the DNA donor template and the sgRNA/Cas9 have been delivered. Cleavage of the donor DNA template in to linear fragments can increase the frequency of integration at a double strand break by the non-homologous end joining mechanism or by the HDR mechanism. This can be particularly beneficial in the case of delivery of donor DNA templates packaged in AAV because after delivery to the nucleus the AAV genomes are known to concatemerize to form larger circular double stranded DNA molecules (Nakai et al JOURNAL OF VIROLOGY 2001, vol 75 p. 6969-6976). Therefore, in some cases the circular concatemers can be less efficient donors for integration at double strand breaks, particularly by the NHEJ mechanism. It was reported previously that the efficiency of targeted integration using circular plasmid DNA donor templates could be increased by including zinc finger nuclease cut sites in the plasmid (Cristea et al Biotechnol. Bioeng. 2013; 110: 871-880). More recently this approach was also applied using the CRISPR/Cas9 nuclease (Suzuki et al 2017, Nature 540, 144-149). While a sgRNA recognition sequence is active when present on either strand of a double stranded DNA donor template, use of the reverse complement of the sgRNA recognition sequence that is present in the genome is predicted to favor stable integration because integration in the reverse orientation re-creates the sgRNA recognition sequence which can be recut thereby releasing the inserted donor DNA template. Integration of such a donor DNA template in the genome in the forward orientation by NHEJ is predicted to not re-create the sgRNA recognition sequence such that the integrated donor DNA template cannot be excised out of the genome. The benefit of including sgRNA recognition sequences in the donor with or without homology arms upon the efficiency of integration of FVIII donor DNA template can be tested and determined, e.g. in mice using AAV for delivery of the donor and LNP for delivery of the CRISPR-Cas9 components.

In some embodiments, the donor DNA template comprises the FVIII gene or functional derivative thereof in a donor cassette according to any of the embodiments described herein flanked on one or both sides by a gRNA target site. In some embodiments, the donor template comprises a gRNA target site 5′ of the donor cassette and/or a gRNA target site 3′ of the donor cassette. In some embodiments, the donor template comprises two flanking gRNA target sites, and the two gRNA target sites comprise the same sequence. In some embodiments, the donor template comprises at least one gRNA target site, and the at least one gRNA target site in the donor template is a target site for at least one of the one or more gRNAs targeting the first intron of the albumin gene. In some embodiments, the donor template comprises at least one gRNA target site, and the at least one gRNA target site in the donor template is the reverse complement of a target site for at least one of the one or more gRNAs in the first intron of the albumin gene. In some embodiments, the donor template comprises a gRNA target site 5′ of the donor cassette and a gRNA target site 3′ of the donor cassette, and the two gRNA target sites in the donor template are targeted by the one or more gRNAs targeting the first intron of the albumin gene. In some embodiments, the donor template comprises a gRNA target site 5′ of the donor cassette and a gRNA target site 3′ of the donor cassette, and the two gRNA target sites in the donor template are the reverse complement of a target site for at least one of the one or more gRNAs in the first intron of the albumin gene.

Insertion of a FVIII-encoding gene into a target site, i.e. a genomic location where the FVIII-encoding gene is inserted, can be in the endogenous albumin gene locus or neighboring sequences thereof. In some embodiments, the FVIII-encoding gene is inserted in a manner that the expression of the inserted gene is controlled by the endogenous promoter of the albumin gene. In some embodiments, the FVIII-encoding gene in inserted in one of introns of the albumin gene. In some embodiments, the FVIII-encoding gene is inserted in one of exons of the albumin gene. In some embodiments, the FVIII-encoding gene is inserted at a junction of intron:exon (or vice versa). In some embodiments, the insertion of the FVIII-encoding gene is in the first intron (or intron 1) of the albumin locus. In some embodiments, the insertion of the FVIII-encoding gene does not significantly affect, e.g. upregulate or downregulate the expression of the albumin gene.

In embodiments, the target site for the insertion of a FVIII-encoding gene is at, within, or near the endogenous albumin gene. In some embodiments, the target site is in an intergenic region that is upstream of the promoter of the albumin gene locus in the genome. In some embodiments, the target site is within the albumin gene locus. In some embodiments, the target site in one of the introns of the albumin gene locus. In some embodiments, the target site in one of the exons of the albumin gene locus. In some embodiments, the target site is in one of the junctions between an intron and exon (or vice versa) of the albumin gene locus. In some embodiments, the target site is in the first intron (or intron 1) of the albumin gene locus. In certain embodiments, the target site is at least, about or at most 0, 1, 5, 10, 20, 30, 40, 50, 100, 150, 200, 250, 300, 350, 400, 450 or 500 or 550 or 600 or 650 bp downstream of the first exon (i.e. from the last nucleic acid of the first exon) of the albumin gene. In some embodiments, the target site is at least, about or at most 0.1 kb, about 0.2 kb, about 0.3 kb, about 0.4 kb, about 0.5 kb, about 1 kb, about 1.5 kb, about 2 kb, about 2.5 kb, about 3 kb, about 3.5 kb, about 4 kb, about 4.5 kb or about 5 kb upstream of the first intron of the albumin gene. In some embodiments, the target site is anywhere within about 0 bp to about 100 bp upstream, about 101 bp to about 200 bp upstream, about 201 bp to about 300 bp upstream, about 301 bp to about 400 bp upstream, about 401 bp to about 500 bp upstream, about 501 bp to about 600 bp upstream, about 601 bp to about 700 bp upstream, about 701 bp to about 800 bp upstream, about 801 bp to about 900 bp upstream, about 901 bp to about 1000 bp upstream, about 1001 bp to about 1500 bp upstream, about 1501 bp to about 2000 bp upstream, about 2001 bp to about 2500 bp upstream, about 2501 bp to about 3000 bp upstream, about 3001 bp to about 3500 bp upstream, about 3501 bp to about 4000 bp upstream, about 4001 bp to about 4500 bp upstream or about 4501 bp to about 5000 bp upstream of the second exon of the albumin gene. In some embodiments, the target site is at least 37 bp downstream of the end (i.e. the 3′ end) of the first exon of the human albumin gene in the genome. In some embodiments, the target site is at least 330 bp upstream of the start (i.e. the 5′ start) of the second exon of the human albumin gene in the genome.

In some embodiments, provided herein is a method of editing a genome in a cell, the method comprising providing the following to the cell: (a) a guide RNA (gRNA) targeting the albumin locus in the cell genome; (b) a DNA endonuclease or nucleic acid encoding said DNA endonuclease; and (c) a donor template comprising a nucleic acid sequence encoding a Factor VIII (FVIII) protein or functional derivative. In some embodiments, the gRNA targets intron 1 of the albumin gene. In some embodiments, the gRNA comprises a spacer sequence from any one of SEQ ID NOs: 18-44 and 104.

In some embodiments, provided herein is a method of editing a genome in a cell, the method comprising providing the following to the cell: (a) a gRNA comprising a spacer sequence from any one of SEQ ID NOs: 18-44 and 104; (b) a DNA endonuclease or nucleic acid encoding said DNA endonuclease; and (c) a donor template comprising a nucleic acid sequence encoding a Factor VIII (FVIII) protein or functional derivative. In some embodiments, the gRNA comprises a spacer sequence from any one of SEQ ID NOs: 21, 22, 28, and 30. In some embodiments, the gRNA comprises a spacer sequence from SEQ ID NO: 21. In some embodiments, the gRNA comprises a spacer sequence from SEQ ID NO: 22. In some embodiments, the gRNA comprises a spacer sequence from SEQ ID NO: 28. In some embodiments, the gRNA comprises a spacer sequence from SEQ ID NO: 30. In some embodiments, the cell is a human cell, e.g., a human hepatocyte cell.

In some embodiments, according to any of the methods of editing a genome in a cell described herein, the DNA endonuclease is selected from the group consisting of a Cas1, Cas1B, Cas2, Cas3, Cas4, Cas5, Cas6, Cas7, Cas8, Cas9 (also known as Csn1 and Csx12), Cas100, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, or Cpf1 endonuclease, or a functional derivative thereof. In some embodiments, the DNA endonuclease is Cas9. In some embodiments, the Cas9 is from Streptococcus pyogenes (spCas9). In some embodiments, the Cas9 is from Staphylococcus lugdunensis (SluCas9).

In some embodiments, according to any of the methods of editing a genome in a cell described herein, the nucleic acid sequence encoding a Factor VIII (FVIII) protein or functional derivative thereof is codon optimized for expression in the cell. In some embodiments, the cell is a human cell.

In some embodiments, according to any of the methods of editing a genome in a cell described herein, the method employs a nucleic acid encoding the DNA endonuclease. In some embodiments, the nucleic acid encoding the DNA endonuclease is codon optimized for expression in the cell. In some embodiments, the cell is a human cell, e.g., a human hepatocyte cell. In some embodiments, the nucleic acid encoding the DNA endonuclease is DNA, such as a DNA plasmid. In some embodiments, the nucleic acid encoding the DNA endonuclease is RNA, such as mRNA.

In some embodiments, according to any of the methods of editing a genome in a cell described herein, the donor template is encoded in an Adeno Associated Virus (AAV) vector. In some embodiments, the donor template comprises a donor cassette comprising the nucleic acid sequence encoding a Factor VIII (FVIII) protein or functional derivative, and the donor cassette is flanked on one or both sides by a gRNA target site. In some embodiments, the donor cassette is flanked on both sides by a gRNA target site. In some embodiments, the gRNA target site is a target site for the gRNA of (a). In some embodiments, the gRNA target site of the donor template is the reverse complement of a cell genome gRNA target site for the gRNA of (a).

In some embodiments, according to any of the methods of editing a genome in a cell described herein, the DNA endonuclease or nucleic acid encoding the DNA endonuclease is formulated in a liposome or lipid nanoparticle. In some embodiments, the liposome or lipid nanoparticle also comprises the gRNA. In some embodiments, the liposome or lipid nanoparticle is a lipid nanoparticle. In some embodiments, the method employs a lipid nanoparticle comprising nucleic acid encoding the DNA endonuclease and the gRNA. In some embodiments, the nucleic acid encoding the DNA endonuclease is an mRNA encoding the DNA endonuclease.

In some embodiments, according to any of the methods of editing a genome in a cell described herein, the DNA endonuclease is pre-complexed with the gRNA, forming a ribonucleoprotein (RNP) complex.

In some embodiments, according to any of the methods of editing a genome in a cell described herein, the gRNA of (a) and the DNA endonuclease or nucleic acid encoding the DNA endonuclease of (b) are provided to the cell after the donor template of (c) is provided to the cell. In some embodiments, the gRNA of (a) and the DNA endonuclease or nucleic acid encoding the DNA endonuclease of (b) are provided to the cell more than 4 days after the donor template of (c) is provided to the cell. In some embodiments, the gRNA of (a) and the DNA endonuclease or nucleic acid encoding the DNA endonuclease of (b) are provided to the cell at least 14 days after the donor template of (c) is provided to the cell. In some embodiments, the gRNA of (a) and the DNA endonuclease or nucleic acid encoding the DNA endonuclease of (b) are provided to the cell at least 17 days after the donor template of (c) is provided to the cell. In some embodiments, (a) and (b) are provided to the cell as a lipid nanoparticle comprising nucleic acid encoding the DNA endonuclease and the gRNA. In some embodiments, the nucleic acid encoding the DNA endonuclease is an mRNA encoding the DNA endonuclease. In some embodiments, (c) is provided to the cell as an AAV vector encoding the donor template.

In some embodiments, according to any of the methods of editing a genome in a cell described herein, one or more additional doses of the gRNA of (a) and the DNA endonuclease or nucleic acid encoding the DNA endonuclease of (b) are provided to the cell following the first dose of the gRNA of (a) and the DNA endonuclease or nucleic acid encoding the DNA endonuclease of (b). In some embodiments, one or more additional doses of the gRNA of (a) and the DNA endonuclease or nucleic acid encoding the DNA endonuclease of (b) are provided to the cell following the first dose of the gRNA of (a) and the DNA endonuclease or nucleic acid encoding the DNA endonuclease of (b) until a target level of targeted integration of the nucleic acid sequence encoding a Factor VIII (FVIII) protein or functional derivative and/or a target level of expression of the nucleic acid sequence encoding a Factor VIII (FVIII) protein or functional derivative is achieved.

In some embodiments, according to any of the methods of editing a genome in a cell described herein, the nucleic acid sequence encoding a Factor VIII (FVIII) protein or functional derivative is expressed under the control of the endogenous albumin promoter.

In some embodiments, provided herein is a method of inserting a FVIII gene or functional derivative thereof into the albumin locus of a cell genome, comprising introducing into the cell (a) a Cas DNA endonuclease (e.g., Cas9) or nucleic acid encoding the Cas DNA endonuclease, (b) a gRNA or nucleic acid encoding the gRNA, wherein the gRNA is capable of guiding the Cas DNA endonuclease to cleave a target polynucleotide sequence in the albumin locus, and (c) a donor template according to any of the embodiments described herein comprising the FVIII gene or functional derivative thereof. In some embodiments, the method comprises introducing into the cell an mRNA encoding the Cas DNA endonuclease. In some embodiments, the method comprises introducing into the cell an LNP according to any of the embodiments described herein comprising i) an mRNA encoding the Cas DNA endonuclease and ii) the gRNA. In some embodiments, the donor template is an AAV donor template. In some embodiments, the donor template comprises a donor cassette comprising the FVIII gene or functional derivative thereof, wherein the donor cassette is flanked on one or both sides by a target site of the gRNA. In some embodiments, the gRNA target sites flanking the donor cassette are the reverse complement of the gRNA target site in the albumin locus. In some embodiments, the Cas DNA endonuclease or nucleic acid encoding the Cas DNA endonuclease and the gRNA or nucleic acid encoding the gRNA are introduced into the cell following introduction of the donor template into the cell. In some embodiments, the Cas DNA endonuclease or nucleic acid encoding the Cas DNA endonuclease and the gRNA or nucleic acid encoding the gRNA are introduced into the cell a sufficient time following introduction of the donor template into the cell to allow for the donor template to enter the cell nucleus. In some embodiments, the Cas DNA endonuclease or nucleic acid encoding the Cas DNA endonuclease and the gRNA or nucleic acid encoding the gRNA are introduced into the cell a sufficient time following introduction of the donor template into the cell to allow for the donor template to be converted from a single stranded AAV genome to a double stranded DNA molecule in the cell nucleus. In some embodiments, the Cas DNA endonuclease is Cas9.

In some embodiments, according to any of the methods of inserting a FVIII gene or functional derivative thereof into the albumin locus of a cell genome described herein, the target polynucleotide sequence is in intron 1 of the albumin gene. In some embodiments, the gRNA comprises a spacer sequence listed in Table 3 or 4. In some embodiments, the gRNA comprises a spacer sequence from any one of SEQ ID NOs: 18-44 and 104. In some embodiments, the gRNA comprises a spacer sequence from any one of SEQ ID NOs: 21, 22, 28, and 30. In some embodiments, the gRNA comprises a spacer sequence from SEQ ID NO: 21. In some embodiments, the gRNA comprises a spacer sequence from SEQ ID NO: 22. In some embodiments, the gRNA comprises a spacer sequence from SEQ ID NO: 28. In some embodiments, the gRNA comprises a spacer sequence from SEQ ID NO: 30.

In some embodiments, provided herein is a method of inserting a FVIII gene or functional derivative thereof into the albumin locus of a cell genome, comprising introducing into the cell (a) an LNP according to any of the embodiments described herein comprising i) an mRNA encoding a Cas9 DNA endonuclease and ii) a gRNA, wherein the gRNA is capable of guiding the Cas9 DNA endonuclease to cleave a target polynucleotide sequence in the albumin locus, and (b) an AAV donor template according to any of the embodiments described herein comprising the FVIII gene or functional derivative thereof. In some embodiments, the donor template comprises a donor cassette comprising the FVIII gene or functional derivative thereof, wherein the donor cassette is flanked on one or both sides by a target site of the gRNA. In some embodiments, the gRNA target sites flanking the donor cassette are the reverse complement of the gRNA target site in the albumin locus. In some embodiments, the LNP is introduced into the cell following introduction of the AAV donor template into the cell. In some embodiments, the LNP is introduced into the cell a sufficient time following introduction of the AAV donor template into the cell to allow for the donor template to enter the cell nucleus. In some embodiments, the LNP is introduced into the cell a sufficient time following introduction of the AAV donor template into the cell to allow for the donor template to be converted from a single stranded AAV genome to a double stranded DNA molecule in the cell nucleus. In some embodiments, one or more (such as 2, 3, 4, 5, or more) additional introductions of the LNP into the cell are performed following the first introduction of the LNP into the cell. In some embodiments, the gRNA comprises a spacer sequence from any one of SEQ ID NOs: 18-44 and 104. In some embodiments, the gRNA comprises a spacer sequence from any one of SEQ ID NOs: 21, 22, 28, and 30. In some embodiments, the gRNA comprises a spacer sequence from SEQ ID NO: 21. In some embodiments, the gRNA comprises a spacer sequence from SEQ ID NO: 22. In some embodiments, the gRNA comprises a spacer sequence from SEQ ID NO: 28. In some embodiments, the gRNA comprises a spacer sequence from SEQ ID NO: 30.

### Target Sequence Selection

In some embodiments, shifts in the location of the 5′ boundary and/or the 3′ boundary relative to particular reference loci are used to facilitate or enhance particular applications of gene editing, which depend in part on the endonuclease system selected for the editing, as further described and illustrated herein.

In a first, non-limiting aspect of such target sequence selection, many endonuclease systems have rules or criteria that guide the initial selection of potential target sites for cleavage, such as the requirement of a PAM sequence motif in a particular position adjacent to the DNA cleavage sites in the case of CRISPR Type II or Type V endonucleases.

In another, non-limiting aspect of target sequence selection or optimization, the frequency of “off-target” activity for a particular combination of target sequence and gene editing endonuclease (i.e. the frequency of DSBs occurring at sites other than the selected target sequence) is assessed relative to the frequency of on-target activity. In some cases, cells that have been correctly edited at the desired locus can have a selective advantage relative to other cells. Illustrative, but non-limiting, examples of a selective advantage include the acquisition of attributes such as enhanced rates of replication, persistence, resistance to certain conditions, enhanced rates of successful engraftment or persistence in vivo following introduction into a patient, and other attributes associated with the maintenance or increased numbers or viability of such cells. In other cases, cells that have been correctly edited at the desired locus can be positively selected for by one or more screening methods used to identify, sort or otherwise select for cells that have been correctly edited. Both selective advantage and directed selection methods can take advantage of the phenotype associated with the correction. In some embodiments, cells can be edited two or more times in order to create a second modification that creates a new phenotype that is used to select or purify the intended population of cells. Such a second modification could be created by adding a second gRNA for a selectable or screenable marker. In some cases, cells can be correctly edited at the desired locus using a DNA fragment that contains the cDNA and also a selectable marker.

In embodiments, whether any selective advantage is applicable or any directed selection is to be applied in a particular case, target sequence selection is also guided by consideration of off-target frequencies in order to enhance the effectiveness of the application and/or reduce the potential for undesired alterations at sites other than the desired target. As described further and illustrated herein and in the art, the occurrence of off-target activity is influenced by a number of factors including similarities and dissimilarities between the target site and various off-target sites, as well as the particular endonuclease used. Bioinformatics tools are available that assist in the prediction of off-target activity, and frequently such tools can also be used to identify the most likely sites of off-target activity, which can then be assessed in experimental settings to evaluate relative frequencies of off-target to on-target activity, thereby allowing the selection of sequences that have higher relative on-target activities. Illustrative examples of such techniques are provided herein, and others are known in the art.

Another aspect of target sequence selection relates to homologous recombination events. Sequences sharing regions of homology can serve as focal points for homologous recombination events that result in deletion of intervening sequences. Such recombination events occur during the normal course of replication of chromosomes and other DNA sequences, and also at other times when DNA sequences are being synthesized, such as in the case of repairs of double-strand breaks (DSBs), which occur on a regular basis during the normal cell replication cycle but can also be enhanced by the occurrence of various events (such as UV light and other inducers of DNA breakage) or the presence of certain agents (such as various chemical inducers). Many such inducers cause DSBs to occur indiscriminately in the genome, and DSBs are regularly being induced and repaired in normal cells. During repair, the original sequence can be reconstructed with complete fidelity, however, in some cases, small insertions or deletions (referred to as “indels”) are introduced at the DSB site.

DSBs can also be specifically induced at particular locations, as in the case of the endonucleases systems described herein, which can be used to cause directed or preferential gene modification events at selected chromosomal locations. The tendency for homologous sequences to be subject to recombination in the context of DNA repair (as well as replication) can be taken advantage of in a number of circumstances, and is the basis for one application of gene editing systems, such as CRISPR, in which homology directed repair is used to insert a sequence of interest, provided through use of a “donor” polynucleotide, into a desired chromosomal location.

Regions of homology between particular sequences, which can be small regions of “microhomology” that can have as few as ten base pairs or less, can also be used to bring about desired deletions. For example, a single DSB is introduced at a site that exhibits microhomology with a nearby sequence. During the normal course of repair of such DSB, a result that occurs with high frequency is the deletion of the intervening sequence as a result of recombination being facilitated by the DSB and concomitant cellular repair process.

In some circumstances, however, selecting target sequences within regions of homology can also give rise to much larger deletions, including gene fusions (when the deletions are in coding regions), which can or cannot be desired given the particular circumstances.

The examples provided herein further illustrate the selection of various target regions for the creation of DSBs designed to insert a FVIII-encoding gene, as well as the selection of specific target sequences within such regions that are designed to minimize off-target events relative to on-target events.

### Targeted Integration

In some embodiments, the method provided herein is to integrate a FVIII encoding gene or a functional FVIII gene at a specific location in the genome of the hepatocytes which is referred to as “targeted integration”. In some embodiments, targeted integration is enabled by using a sequence specific nuclease to generate a double stranded break in the genomic DNA.

The CRISPR-Cas system used in some embodiments has the advantage that a large number of genomic targets can be rapidly screened to identify an optimal CRISPR-Cas design. The CRISPR-Cas system uses a RNA molecule called a single guide RNA (sgRNA) that targets an associated Cas nuclease (for example the Cas9 nuclease) to a specific sequence in DNA. This targeting occurs by Watson-Crick based pairing between the sgRNA and the sequence of the genome within the approximately 20 bp targeting sequence of the sgRNA. Once bound at a target site the Cas nuclease cleaves both strands of the genomic DNA creating a double strand break. The only requirement for designing a sgRNA to target a specific DNA sequence is that the target sequence must contain a protospacer adjacent motif (PAM) sequence at the 3′ end of the sgRNA sequence that is complementary to the genomic sequence. In the case of the Cas9 nuclease the PAM sequence is NRG (where R is A or G and N is any base), or the more restricted PAM sequence NGG Therefore, sgRNA molecules that target any region of the genome can be designed in silico by locating the 20 bp sequence adjacent to all PAM motifs. PAM motifs occur on average very 15 bp in the genome of eukaryotes. However, sgRNA designed by in silico methods will generate double strand breaks in cells with differeing efficiencies and it is not possible to predict the cutting efficiencies of a series of sgRNA molecule using in silico methods. Because sgRNA can be rapidly synthesized in vitro this enables the rapid screening of all potential sgRNA sequences in a given genomic region to identify the sgRNA that results in the most efficient cutting. Typically when a series of sgRNA within a given genomic region are tested in cells a range of cleavage efficiencies between 0 and 90% is observed. In silico algorithms as well as laboratory experiments can also be used to determine the off-target potential of any given sgRNA. While a perfect match to the 20 bp recognition sequence of a sgRNA will primarily occur only once in most eukaryotic genomes there will be a number of additional sites in the genome with 1 or more base pair mismatches to the sgRNA. These sites can be cleaved at variable frequencies which are often not predictable based on the number or location of the mismatches. Cleavage at additional off-target sites that were not identified by the in silico analysis can also occur. Thus, screening a number of sgRNA in a relevant cell type to identify sgRNA that have the most favorable off-target profile is a critical component of selecting an optimal sgRNA for therapeutic use. A favorable off target profile will take into account not only the number of actual off-target sites and the frequency of cutting at these sites, but also the location in the genome of these sites. For example, off-target sites close to or within functionally important genes, particularly oncogenes or anti-oncogenes would be considered as less favorable than sites in intergenic regions with no known function. Thus, the identification of an optimal sgRNA cannot be predicted simply by in silico analysis of the genomic sequence of an organism but requires experimental testing. While in silico analysis can be helpful in narrowing down the number of guides to test it cannot predict guides that have high on target cutting or predict guides with low desirable off-target cutting. Experimental data indicates that the cutting efficiency of sgRNA that each has a perfect match to the genome in a region of interest (such as the albumin intron 1) varies from no cutting to >90% cutting and is not predictable by any known algorithm. The ability of a given sgRNA to promote cleavage by a Cas enzyme can relate to the accessibility of that specific site in the genomic DNA which can be determined by the chromatin structure in that region. While the majority of the genomic DNA in a quiescent differentiated cell, such as a hepatocyte, exists in highly condensed heterochromatin, regions that are actively transcribed exists in more open chromatin states that are known to be more accessible to large molecules such as proteins like the Cas protein. Even within actively transcribed genes some specific regions of the DNA are more accessible than others due to the presence or absence of bound transcription factors or other regulatory proteins. Predicting sites in the genome or within a specific genomic locus or region of a genomic locus such as an intron, and such as albumin intron 1 is not possible and therefore would need to be determined experimentally in a relevant cell type. Once some sites are selected as potential sites for insertion, it can be possible to add some variations to such a site, e.g. by moving a few nucleotides upstream or downstream from the selected sites, with or without experimental tests.

In some embodiments, gRNAs that can be used in the methods disclosed herein are one or more listed from Table 3 or any derivatives thereof having at least about 85% nucleotide sequence identity to those from Table 3.

### Nucleic Acid Modifications

In some embodiments, polynucleotides introduced into cells have one or more modifications that can be used individually or in combination, for example, to enhance activity, stability or specificity, alter delivery, reduce innate immune responses in host cells, or for other enhancements, as further described herein and known in the art.

In certain embodiments, modified polynucleotides are used in the CRISPR/Cas9/Cpf1 system, in which case the guide RNAs (either single-molecule guides or double-molecule guides) and/or a DNA or an RNA encoding a Cas or Cpf1 endonuclease introduced into a cell can be modified, as described and illustrated below. Such modified polynucleotides can be used in the CRISPR/Cas9/Cpf1 system to edit any one or more genomic loci.

Using the CRISPR/Cas9/Cpf1 system for purposes of non-limiting illustrations of such uses, modifications of guide RNAs can be used to enhance the formation or stability of the CRISPR/Cas9/Cpf1 genome editing complex having guide RNAs, which can be single-molecule guides or double-molecule, and a Cas or Cpf1 endonuclease. Modifications of guide RNAs can also or alternatively be used to enhance the initiation, stability or kinetics of interactions between the genome editing complex with the target sequence in the genome, which can be used, for example, to enhance on-target activity. Modifications of guide RNAs can also or alternatively be used to enhance specificity, e.g., the relative rates of genome editing at the on-target site as compared to effects at other (off-target) sites.

Modifications can also or alternatively be used to increase the stability of a guide RNA, e.g., by increasing its resistance to degradation by ribonucleases (RNases) present in a cell, thereby causing its half-life in the cell to be increased. Modifications enhancing guide RNA half-life can be particularly useful in embodiments in which a Cas or Cpf1 endonuclease is introduced into the cell to be edited via an RNA that needs to be translated in order to generate endonuclease, because increasing the half-life of guide RNAs introduced at the same time as the RNA encoding the endonuclease can be used to increase the time that the guide RNAs and the encoded Cas or Cpf1 endonuclease co-exist in the cell.

Modifications can also or alternatively be used to decrease the likelihood or degree to which RNAs introduced into cells elicit innate immune responses. Such responses, which have been well characterized in the context of RNA interference (RNAi), including small-interfering RNAs (siRNAs), as described below and in the art, tend to be associated with reduced half-life of the RNA and/or the elicitation of cytokines or other factors associated with immune responses.

One or more types of modifications can also be made to RNAs encoding an endonuclease that are introduced into a cell, including, without limitation, modifications that enhance the stability of the RNA (such as by increasing its degradation by RNAses present in the cell), modifications that enhance translation of the resulting product (i.e. the endonuclease), and/or modifications that decrease the likelihood or degree to which the RNAs introduced into cells elicit innate immune responses.

Combinations of modifications, such as the foregoing and others, can likewise be used. In the case of CRISPR/Cas9/Cpf1, for example, one or more types of modifications can be made to guide RNAs (including those exemplified above), and/or one or more types of modifications can be made to RNAs encoding Cas endonuclease (including those exemplified above).

By way of illustration, guide RNAs used in the CRISPR/Cas9/Cpf1 system, or other smaller RNAs can be readily synthesized by chemical means, enabling a number of modifications to be readily incorporated, as illustrated below and described in the art. While chemical synthetic procedures are continually expanding, purifications of such RNAs by procedures such as high performance liquid chromatography (HPLC, which avoids the use of gels such as PAGE) tends to become more challenging as polynucleotide lengths increase significantly beyond a hundred or so nucleotides. One approach used for generating chemically-modified RNAs of greater length is to produce two or more molecules that are ligated together. Much longer RNAs, such as those encoding a Cas9 endonuclease, are more readily generated enzymatically. While fewer types of modifications are generally available for use in enzymatically produced RNAs, there are still modifications that can be used to, e g, enhance stability, reduce the likelihood or degree of innate immune response, and/or enhance other attributes, as described further below and in the art; and new types of modifications are regularly being developed.

By way of illustration of various types of modifications, especially those used frequently with smaller chemically synthesized RNAs, modifications can have one or more nucleotides modified at the 2′ position of the sugar, in some embodiments a 2′-O-alkyl, 2′-O-alkyl-O-alkyl, or 2′-fluoro-modified nucleotide. In some embodiments, RNA modifications include 2′-fluoro, 2′-amino or 2′ O-methyl modifications on the ribose of pyrimidines, abasic residues, or an inverted base at the 3′ end of the RNA. Such modifications are routinely incorporated into oligonucleotides and these oligonucleotides have been shown to have a higher Tm (i.e., higher target binding affinity) than 2′-deoxyoligonucleotides against a given target.

A number of nucleotide and nucleoside modifications have been shown to make the oligonucleotide into which they are incorporated more resistant to nuclease digestion than the native oligonucleotide; these modified oligos survive intact for a longer time than unmodified oligonucleotides. Specific examples of modified oligonucleotides include those having modified backbones, for example, phosphorothioates, phosphotriesters, methyl phosphonates, short chain alkyl or cycloalkyl intersugar linkages or short chain heteroatomic or heterocyclic intersugar linkages. Some oligonucleotides are oligonucleotides with phosphorothioate backbones and those with heteroatom backbones, particularly CH₂—NH—O—CH₂, CH, ˜N(CH₃)˜O˜CH₂ (known as a methylene(methylimino) or MMI backbone), CH₂—O—N(CH₃)—CH₂, CH₂—N(CH₃)—N (CH₃)—CH₂ and O—N(CH₃)—CH₂—CH₂ backbones, wherein the native phosphodiester backbone is represented as O—P—O—CH,); amide backbones [see De Mesmaeker et al., Ace. Chem. Res., 28:366-374 (1995)]; morpholino backbone structures (see Summerton and Weller, U.S. Pat. No. 5,034,506); peptide nucleic acid (PNA) backbone (wherein the phosphodiester backbone of the oligonucleotide is replaced with a polyamide backbone, the nucleotides being bound directly or indirectly to the aza nitrogen atoms of the polyamide backbone, see Nielsen et al., Science 1991, 254, 1497). Phosphorus-containing linkages include, but are not limited to, phosphorothioates, chiral phosphorothioates, phosphorodithioates, phosphotriesters, aminoalkylphosphotriesters, methyl and other alkyl phosphonates having 3′alkylene phosphonates and chiral phosphonates, phosphinates, phosphoramidates having 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those having inverted polarity wherein the adjacent pairs of nucleoside units are linked 3′-5′ to 5′-3′ or 2′-5′ to 5′-2′; see U.S. Pat. Nos. 3,687,808; 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,306; 5,550,111; 5,563,253; 5,571,799; 5,587,361; and 5,625,050.

Morpholino-based oligomeric compounds are described in Braasch and David Corey, Biochemistry, 41(14): 4503-4510 (2002); Genesis, Volume 30, Issue 3, (2001); Heasman, Dev. Biol., 243: 209-214 (2002); Nasevicius et al., Nat. Genet., 26:216-220 (2000); Lacerra et al., Proc. Natl. Acad. Sci., 97: 9591-9596 (2000); and U.S. Pat. No. 5,034,506, issued Jul. 23, 1991.

Cyclohexenyl nucleic acid oligonucleotide mimetics are described in Wang et al., J. Am. Chem. Soc., 122: 8595-8602 (2000).

Modified oligonucleotide backbones that do not include a phosphorus atom therein have backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These have those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S, and CH₂ component parts; see U.S. Pat. Nos. 5,034,506; 5,166,315; 5,185,444; 5,214,134; 5,216,141; 5,235,033; 5,264,562; 5,264,564; 5,405,938; 5,434,257; 5,466,677; 5,470,967; 5,489,677; 5,541,307; 5,561,225; 5,596,086; 5,602,240; 5,610,289; 5,602,240; 5,608,046; 5,610,289; 5,618,704; 5,623,070; 5,663,312; 5,633,360; 5,677,437; and 5,677,439, each of which is herein incorporated by reference.

One or more substituted sugar moieties can also be included, e.g., one of the following at the 2′ position: OH, SH, SCH₃, F, OCN, OCH₃OCH₃, OCH₃O(CH₂)ₙCH₃, O(CH₂)ₙNH₂, or O(CH₂)ₙCH₃, where n is from 1 to about 10; C1 to C10 lower alkyl, alkoxyalkoxy, substituted lower alkyl, alkaryl or aralkyl; Cl; Br; CN; CF₃; OCF₃; O-, S-, or N-alkyl; O-, S-, or N-alkenyl; SOCH₃; SO₂CH₃; ONO₂; NO₂; N₃; NH₂; heterocycloalkyl; heterocycloalkaryl; aminoalkylamino; polyalkylamino; substituted silyl; an RNA cleaving group; a reporter group; an intercalator; a group for improving the pharmacokinetic properties of an oligonucleotide; or a group for improving the pharmacodynamic properties of an oligonucleotide and other substituents having similar properties. In some embodiments, a modification includes 2′-methoxyethoxy (2′-O—CH₂CH₂OCH₃, also known as 2′-O-(2-methoxyethyl)) (Martin et al, Helv. Chim Acta, 1995, 78, 486). Other modifications include 2′-methoxy (2′-O—CH₃), 2′-propoxy (2′-OCH₂CH₂CH₃) and 2′-fluoro (2′-F). Similar modifications can also be made at other positions on the oligonucleotide, particularly the 3′ position of the sugar on the 3′ terminal nucleotide and the 5′ position of 5′ terminal nucleotide. Oligonucleotides can also have sugar mimetics, such as cyclobutyls in place of the pentofuranosyl group.

In some embodiments, both a sugar and an internucleoside linkage, i.e., the backbone, of the nucleotide units are replaced with novel groups. The base units are maintained for hybridization with an appropriate nucleic acid target compound. One such oligomeric compound, an oligonucleotide mimetic that has been shown to have excellent hybridization properties, is referred to as a peptide nucleic acid (PNA). In PNA compounds, the sugar-backbone of an oligonucleotide is replaced with an amide containing backbone, for example, an aminoethylglycine backbone. The nucleobases are retained and are bound directly or indirectly to aza nitrogen atoms of the amide portion of the backbone. Representative United States patents that teach the preparation of PNA compounds have, but are not limited to, U.S. Pat. Nos. 5,539,082; 5,714,331; and 5,719,262. Further teaching of PNA compounds can be found in Nielsen et al, Science, 254: 1497-1500 (1991).

In some embodiments, guide RNAs can also include, additionally or alternatively, nucleobase (often referred to in the art simply as “base”) modifications or substitutions. As used herein, “unmodified” or “natural” nucleobases include adenine (A), guanine (G), thymine (T), cytosine (C), and uracil (U). Modified nucleobases include nucleobases found only infrequently or transiently in natural nucleic acids, e.g., hypoxanthine, 6-methyladenine, 5-Me pyrimidines, particularly 5-methylcytosine (also referred to as 5-methyl-2′ deoxycytosine and often referred to in the art as 5-Me-C), 5-hydroxymethylcytosine (HMC), glycosyl HMC and gentobiosyl HMC, as well as synthetic nucleobases, e.g., 2-aminoadenine, 2-(methylamino)adenine, 2-(imidazolylalkyl)adenine, 2-(aminoalklyamino)adenine or other heterosubstituted alkyladenines, 2-thiouracil, 2-thiothymine, 5-bromouracil, 5-hydroxymethyluracil, 8-azaguanine, 7-deazaguanine, N6 (6-aminohexyl)adenine, and 2,6-diaminopurine. Kornberg, A., DNA Replication, W. H. Freeman & Co., San Francisco, pp 75-77 (1980); Gebeyehu et al., Nucl. Acids Res. 15:4513 (1997). A “universal” base known in the art, e.g., inosine, can also be included. 5-Me—C substitutions have been shown to increase nucleic acid duplex stability by 0.6-1.2° C. (Sanghvi, Y. S., in Crooke, S. T. and Lebleu, B., eds., Antisense Research and Applications, CRC Press, Boca Raton, 1993, pp. 276-278) and are embodiments of base substitutions.

In some embodiments, modified nucleobases include other synthetic and natural nucleobases, such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudo-uracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylquanine and 7-methyladenine, 8-azaguanine and 8-azaadenine, 7-deazaguanine and 7-deazaadenine, and 3-deazaguanine and 3-deazaadenine.

Further, nucleobases include those disclosed in U.S. Pat. No. 3,687,808, those disclosed in ‘The Concise Encyclopedia of Polymer Science And Engineering’, pages 858-859, Kroschwitz, J. I., ed. John Wiley & Sons, 1990, those disclosed by Englisch et al., Angewandle Chemie, International Edition’, 1991, 30, page 613, and those disclosed by Sanghvi, Y. S., Chapter 15, Antisense Research and Applications', pages 289-302, Crooke, S. T. and Lebleu, B. ea., CRC Press, 1993. Certain of these nucleobases are particularly useful for increasing the binding affinity of the oligomeric compounds of the disclosure. These include 5-substituted pyrimidines, 6-azapyrimidines and N-2, N-6 and 0-6 substituted purines, having 2-aminopropyladenine, 5-propynyluracil and 5-propynylcytosine. 5-methylcytosine substitutions have been shown to increase nucleic acid duplex stability by 0.6-1.2° C. (Sanghvi, Y. S., Crooke, S. T. and Lebleu, B., eds, ‘Antisense Research and Applications,’ CRC Press, Boca Raton, 1993, pp. 276-278) and are embodiments of base substitutions, even more particularly when combined with 2′-O-methoxyethyl sugar modifications. Modified nucleobases are described in U.S. Pat. No. 3,687,808, as well as U.S. Pat. Nos. 4,845,205; 5,130,302; 5,134,066; 5,175,273; 5,367,066; 5,432,272; 5,457,187; 5,459,255; 5,484,908; 5,502,177; 5,525,711; 5,552,540; 5,587,469; 5,596,091; 5,614,617; 5,681,941; 5,750,692; 5,763,588; 5,830,653; 6,005,096; and U.S. Patent Application Publication 2003/0158403.

In some embodiments, the guide RNAs and/or mRNA (or DNA) encoding an endonuclease are chemically linked to one or more moieties or conjugates that enhance the activity, cellular distribution, or cellular uptake of the oligonucleotide. Such moieties include, but are not limited to, lipid moieties such as a cholesterol moiety [Letsinger et al., Proc. Natl. Acad. Sci. USA, 86: 6553-6556 (1989)]; cholic acid [Manoharan et al., Bioorg. Med. Chem. Let., 4: 1053-1060 (1994)]; a thioether, e.g., hexyl-S-tritylthiol [Manoharan et al, Ann. N. Y. Acad. Sci., 660: 306-309 (1992) and Manoharan et al., Bioorg. Med. Chem. Let., 3: 2765-2770 (1993)]; a thiocholesterol [Oberhauser et al., Nucl. Acids Res., 20: 533-538 (1992)]; an aliphatic chain, e.g., dodecandiol or undecyl residues [Kabanov et al., FEBS Lett., 259: 327-330 (1990) and Svinarchuk et al., Biochimie, 75: 49-54 (1993)]; a phospholipid, e.g., di-hexadecyl-rac-glycerol or triethylammonium 1,2-di-O-hexadecyl- rac-glycero-3-H-phosphonate [Manoharan et al., Tetrahedron Lett., 36: 3651-3654 (1995) and Shea et al., Nucl. Acids Res., 18: 3777-3783 (1990)]; a polyamine or a polyethylene glycol chain [Mancharan et al., Nucleosides & Nucleotides, 14: 969-973 (1995)]; adamantane acetic acid [Manoharan et al., Tetrahedron Lett., 36: 3651-3654 (1995)]; a palmityl moiety [(Mishra et al., Biochim Biophys. Acta, 1264: 229-237 (1995)]; or an octadecylamine or hexylamino-carbonyl-t oxycholesterol moiety [Crooke et al., J. Pharmacol. Exp. Ther., 277: 923-937 (1996)]. See also U.S. Pat. Nos. 4,828,979; 4,948,882; 5,218,105; 5,525,465; 5,541,313; 5,545,730; 5,552,538; 5,578,717, 5,580,731; 5,580,731; 5,591,584; 5,109,124; 5,118,802; 5,138,045; 5,414,077; 5,486,603; 5,512,439; 5,578,718; 5,608,046; 4,587,044; 4,605,735; 4,667,025; 4,762,779; 4,789,737; 4,824,941; 4,835,263; 4,876,335; 4,904,582; 4,958,013; 5,082,830; 5,112,963; 5,214,136; 5,082,830; 5,112,963; 5,214,136; 5,245,022; 5,254,469; 5,258,506; 5,262,536; 5,272,250; 5,292,873; 5,317,098; 5,371,241, 5,391,723; 5,416,203, 5,451,463; 5,510,475; 5,512,667; 5,514,785; 5,565,552; 5,567,810; 5,574,142; 5,585,481; 5,587,371; 5,595,726; 5,597,696; 5,599,923; 5,599, 928 and 5,688,941.

In some embodiments, sugars and other moieties can be used to target proteins and complexes having nucleotides, such as cationic polysomes and liposomes, to particular sites. For example, hepatic cell directed transfer can be mediated via asialoglycoprotein receptors (ASGPRs); see, e.g., Hu, et al., Protein Pept Lett. 21(10):1025-30 (2014). Other systems known in the art and regularly developed can be used to target biomolecules of use in the present case and/or complexes thereof to particular target cells of interest.

In some embodiments, these targeting moieties or conjugates can include conjugate groups covalently bound to functional groups, such as primary or secondary hydroxyl groups. Conjugate groups of the disclosure include intercalators, reporter molecules, polyamines, polyamides, polyethylene glycols, polyethers, groups that enhance the pharmacodynamic properties of oligomers, and groups that enhance the pharmacokinetic properties of oligomers. Typical conjugate groups include cholesterols, lipids, phospholipids, biotin, phenazine, folate, phenanthridine, anthraquinone, acridine, fluoresceins, rhodamines, coumarins, and dyes. Groups that enhance the pharmacodynamic properties, in the context of this disclosure, include groups that improve uptake, enhance resistance to degradation, and/or strengthen sequence-specific hybridization with the target nucleic acid. Groups that enhance the pharmacokinetic properties, in the context of this disclosure, include groups that improve uptake, distribution, metabolism or excretion of the compounds of the present disclosure. Representative conjugate groups are disclosed in International Patent Application No. PCT/US92/09196, filed Oct. 23, 1992, and U.S. Pat. No. 6,287,860, which are incorporated herein by reference. Conjugate moieties include, but are not limited to, lipid moieties such as a cholesterol moiety, cholic acid, a thioether, e.g., hexyl-5-tritylthiol, a thiocholesterol, an aliphatic chain, e.g., dodecandiol or undecyl residues, a phospholipid, e.g., di-hexadecyl-rac- glycerol or triethylammonium 1,2-di-O-hexadecyl-rac-glycero-3-H-phosphonate, a polyamine or a polyethylene glycol chain, or adamantane acetic acid, a palmityl moiety, or an octadecylamine or hexylamino-carbonyl-oxy cholesterol moiety. See, e.g., U.S. Pat. Nos. 4,828,979; 4,948,882; 5,218,105; 5,525,465; 5,541,313; 5,545,730; 5,552,538; 5,578,717, 5,580,731; 5,580,731; 5,591,584; 5,109,124; 5,118,802; 5,138,045; 5,414,077; 5,486,603; 5,512,439; 5,578,718; 5,608,046; 4,587,044; 4,605,735; 4,667,025; 4,762,779; 4,789,737; 4,824,941; 4,835,263; 4,876,335; 4,904,582; 4,958,013; 5,082,830; 5,112,963; 5,214,136; 5,082,830; 5,112,963; 5,214,136; 5,245,022; 5,254,469; 5,258,506; 5,262,536; 5,272,250; 5,292,873; 5,317,098; 5,371,241, 5,391,723; 5,416,203, 5,451,463; 5,510,475; 5,512,667; 5,514,785; 5,565,552; 5,567,810; 5,574,142; 5,585,481; 5,587,371; 5,595,726; 5,597,696; 5,599,923; 5,599,928 and 5,688,941.

Longer polynucleotides that are less amenable to chemical synthesis and are typically produced by enzymatic synthesis can also be modified by various means. Such modifications can include, for example, the introduction of certain nucleotide analogs, the incorporation of particular sequences or other moieties at the 5′ or 3′ ends of molecules, and other modifications. By way of illustration, the mRNA encoding Cas9 is approximately 4 kb in length and can be synthesized by in vitro transcription. Modifications to the mRNA can be applied to, e.g., increase its translation or stability (such as by increasing its resistance to degradation with a cell), or to reduce the tendency of the RNA to elicit an innate immune response that is often observed in cells following introduction of exogenous RNAs, particularly longer RNAs such as that encoding Cas9.

Numerous such modifications have been described in the art, such as polyA tails, 5′ cap analogs (e.g., Anti Reverse Cap Analog (ARCA) or m7G (5′) ppp (5′) G (mCAP)), modified 5′ or 3′ untranslated regions (UTRs), use of modified bases (such as Pseudo-UTP, 2-Thio-UTP, 5-Methylcytidine-5′-Triphosphate (5-Methyl-CTP) or N6-Methyl-ATP), or treatment with phosphatase to remove 5′ terminal phosphates. These and other modifications are known in the art, and new modifications of RNAs are regularly being developed.

There are numerous commercial suppliers of modified RNAs, including for example, TriLink Biotech, AxoLabs, Bio-Synthesis Inc., Dharmacon and many others. As described by TriLink, for example, 5-Methyl-CTP can be used to impart desirable characteristics, such as increased nuclease stability, increased translation or reduced interaction of innate immune receptors with in vitro transcribed RNA. 5-Methylcytidine-5′-Triphosphate (5-Methyl-CTP), N6-Methyl-ATP, as well as Pseudo-UTP and 2-Thio-UTP, have also been shown to reduce innate immune stimulation in culture and in vivo while enhancing translation, as illustrated in publications by Kormann et al. and Warren et al. referred to below.

It has been shown that chemically modified mRNA delivered in vivo can be used to achieve improved therapeutic effects; see, e.g., Kormann et al., Nature Biotechnology 29, 154-157 (2011). Such modifications can be used, for example, to increase the stability of the RNA molecule and/or reduce its immunogenicity. Using chemical modifications such as Pseudo-U, N6-Methyl-A, 2-Thio-U and 5-Methyl-C, it was found that substituting just one quarter of the uridine and cytidine residues with 2-Thio-U and 5-Methyl-C respectively resulted in a significant decrease in toll-like receptor (TLR) mediated recognition of the mRNA in mice. By reducing the activation of the innate immune system, these modifications can be used to effectively increase the stability and longevity of the mRNA in vivo; see, e.g., Kormann et al., supra.

It has also been shown that repeated administration of synthetic messenger RNAs incorporating modifications designed to bypass innate anti-viral responses can reprogram differentiated human cells to pluripotency. See, e.g., Warren, et al., Cell Stem Cell, 7(5):618-30 (2010). Such modified mRNAs that act as primary reprogramming proteins can be an efficient means of reprogramming multiple human cell types. Such cells are referred to as induced pluripotency stem cells (iPSCs), and it was found that enzymatically synthesized RNA incorporating 5-Methyl-CTP, Pseudo-UTP and an Anti Reverse Cap Analog (ARCA) could be used to effectively evade the cell's antiviral response; see, e.g., Warren et al., supra.

Other modifications of polynucleotides described in the art include, for example, the use of polyA tails, the addition of 5′ cap analogs (such as m7G (5′) ppp (5′) G (mCAP)), modifications of 5′ or 3′ untranslated regions (UTRs), or treatment with phosphatase to remove 5′ terminal phosphates—and new approaches are regularly being developed.

A number of compositions and techniques applicable to the generation of modified RNAs for use herein have been developed in connection with the modification of RNA interference (RNAi), including small-interfering RNAs (siRNAs). siRNAs present particular challenges in vivo because their effects on gene silencing via mRNA interference are generally transient, which can require repeat administration. In addition, siRNAs are double-stranded RNAs (dsRNA) and mammalian cells have immune responses that have evolved to detect and neutralize dsRNA, which is often a by-product of viral infection. Thus, there are mammalian enzymes such as PKR (dsRNA-responsive kinase), and potentially retinoic acid-inducible gene I (RIG-I), that can mediate cellular responses to dsRNA, as well as Toll-like receptors (such as TLR3, TLR7 and TLR8) that can trigger the induction of cytokines in response to such molecules; see, e.g., the reviews by Angart et al., Pharmaceuticals (Basel) 6(4): 440-468 (2013); Kanasty et al., Molecular Therapy 20(3): 513-524 (2012); Burnett et al., Biotechnol J. 6(9):1130-46 (2011); Judge and MacLachlan, Hum Gene Ther 19(2):111-24 (2008); and references cited therein.

A large variety of modifications have been developed and applied to enhance RNA stability, reduce innate immune responses, and/or achieve other benefits that can be useful in connection with the introduction of polynucleotides into human cells, as described herein; see, e.g., the reviews by Whitehead K A et al., Annual Review of Chemical and Biomolecular Engineering, 2: 77-96 (2011); Gaglione and Messere, Mini Rev Med Chem, 10(7):578-95 (2010); Chernolovskaya et al, Curr Opin Mol Ther., 12(2):158-67 (2010); Deleavey et al., Curr Protoc Nucleic Acid Chem Chapter 16:Unit 16.3 (2009); Behlke, Oligonucleotides 18(4):305-19 (2008); Fucini et al., Nucleic Acid Ther 22(3): 205-210 (2012); Bremsen et al., Front Genet 3:154 (2012).

As noted above, there are a number of commercial suppliers of modified RNAs, many of which have specialized in modifications designed to improve the effectiveness of siRNAs. A variety of approaches are offered based on various findings reported in the literature. For example, Dharmacon notes that replacement of a non-bridging oxygen with sulfur (phosphorothioate, PS) has been extensively used to improve nuclease resistance of siRNAs, as reported by Kole, Nature Reviews Drug Discovery 11:125-140 (2012). Modifications of the 2′-position of the ribose have been reported to improve nuclease resistance of the internucleotide phosphate bond while increasing duplex stability (Tm), which has also been shown to provide protection from immune activation. A combination of moderate PS backbone modifications with small, well-tolerated 2′-substitutions (2′-O-Methyl, 2′-Fluoro, 2′-Hydro) have been associated with highly stable siRNAs for applications in vivo, as reported by Soutschek et al. Nature 432:173-178 (2004); and 2′-O-Methyl modifications have been reported to be effective in improving stability as reported by Volkov, Oligonucleotides 19:191-202 (2009). With respect to decreasing the induction of innate immune responses, modifying specific sequences with 2′-O-Methyl, 2′-Fluoro, 2′-Hydro have been reported to reduce TLR7/TLR8 interaction while generally preserving silencing activity; see, e.g., Judge et al., Mol. Ther. 13:494-505 (2006); and Cekaite et al., J. Mol. Biol. 365:90-108 (2007). Additional modifications, such as 2-thiouracil, pseudouracil, 5-methylcytosine, 5-methyluracil, and N6-methyladenosine have also been shown to minimize the immune effects mediated by TLR3, TLR7, and TLR8; see, e.g., Kariko, K. et al., Immunity 23:165-175 (2005).

As is also known in the art, and commercially available, a number of conjugates can be applied to polynucleotides, such as RNAs, for use herein that can enhance their delivery and/or uptake by cells, including for example, cholesterol, tocopherol and folic acid, lipids, peptides, polymers, linkers and aptamers; see, e.g., the review by Winkler, Ther. Deliv. 4:791-809 (2013), and references cited therein.

### Delivery

In some embodiments, any nucleic acid molecules used in the methods provided herein, e.g. a nucleic acid encoding a genome-targeting nucleic acid of the disclosure and/or a site-directed polypeptide are packaged into or on the surface of delivery vehicles for delivery to cells. Delivery vehicles contemplated include, but are not limited to, nanospheres, liposomes, quantum dots, nanoparticles, polyethylene glycol particles, hydrogels, and micelles. As described in the art, a variety of targeting moieties can be used to enhance the preferential interaction of such vehicles with desired cell types or locations.

Introduction of the complexes, polypeptides, and nucleic acids of the disclosure into cells can occur by viral or bacteriophage infection, transfection, conjugation, protoplast fusion, lipofection, electroporation, nucleofection, calcium phosphate precipitation, polyethyleneimine (PEI)-mediated transfection, DEAE-dextran mediated transfection, liposome-mediated transfection, particle gun technology, calcium phosphate precipitation, direct micro-injection, nanoparticle-mediated nucleic acid delivery, and the like.

In embodiments, guide RNA polynucleotides (RNA or DNA) and/or endonuclease polynucleotide(s) (RNA or DNA) can be delivered by viral or non-viral delivery vehicles known in the art. Alternatively, endonuclease polypeptide(s) can be delivered by viral or non-viral delivery vehicles known in the art, such as electroporation or lipid nanoparticles. In some embodiments, the DNA endonuclease can be delivered as one or more polypeptides, either alone or pre-complexed with one or more guide RNAs, or one or more crRNA together with a tracrRNA.

In embodiments, polynucleotides can be delivered by non-viral delivery vehicles including, but not limited to, nanoparticles, liposomes, ribonucleoproteins, positively charged peptides, small molecule RNA-conjugates, aptamer-RNA chimeras, and RNA-fusion protein complexes. Some exemplary non-viral delivery vehicles are described in Peer and Lieberman, Gene Therapy, 18: 1127-1133 (2011) (which focuses on non-viral delivery vehicles for siRNA that are also useful for delivery of other polynucleotides).

In embodiments, polynucleotides, such as guide RNA, sgRNA, and mRNA encoding an endonuclease, can be delivered to a cell or a patient by a lipid nanoparticle (LNP).

While several non-viral delivery methods for nucleic acids have been tested both in animal models and in humans the most well developed system is lipid nanoparticles. Lipid nanoparticles (LNP) are generally composed of an ionizable cationic lipid and 3 or more additional components, typically cholesterol, DOPE and a Polyethylene Glycol (PEG) containing lipid, see, e.g. Example 2. The cationic lipid can bind to the positively charged nucleic acid forming a dense complex that protects the nucleic from degradation. During passage through a micro fluidics system the components self-assemble to form particles in the size range of 50 to 150 nM in which the nucleic acid is encapsulated in the core complexed with the cationic lipid and surrounded by a lipid bilayer like structure. After injection in to the circulation of a subject these particles can bind to apolipoprotein E (apoE). ApoE is a ligand for the LDL receptor and mediates uptake in to the hepatocytes of the liver via receptor mediated endocytosis. LNP of this type have been shown to efficiently deliver mRNA and siRNA to the hepatocytes of the liver of rodents, primates and humans. After endocytosis, the LNP are present in endosomes. The encapsulated nucleic acid undergoes a process of endosomal escape mediate by the ionizable nature of the cationic lipid. This delivers the nucleic acid into the cytoplasm where mRNA can be translated in to the encoded protein. Thus, in some embodiments encapsulation of gRNA and mRNA encoding Cas9 in to a LNP is used to efficiently deliver both components to the hepatocytes after IV injection. After endosomal escape the Cas9 mRNA is translated in to Cas9 protein and can form a complex with the gRNA. In some embodiments, inclusion of a nuclear localization signal in to the Cas9 protein sequence promotes translocation of the Cas9 protein/gRNA complex to the nucleus. Alternatively, the small gRNA crosses the nuclear pore complex and form complexes with Cas9 protein in the nucleus. Once in the nucleus the gRNA/Cas9 complex scan the genome for homologous target sites and generate double strand breaks preferentially at the desired target site in the genome. The half-life of RNA molecules in vivo is short on the order of hours to days. Similarly, the half-life of proteins tends to be short, on the order of hours to days. Thus, in some embodiments delivery of the gRNA and Cas9 mRNA using an LNP can result in only transient expression and activity of the gRNA/Cas9 complex. This can provide the advantage of reducing the frequency of off-target cleavage and thus minimize the risk of genotoxicity in some embodiments. LNP are generally less immunogenic than viral particles. While many humans have preexisting immunity to AAV there is no pre-existing immunity to LNP. In additional and adaptive immune response against LNP is unlikely to occur which enables repeat dosing of LNP.

Several different ionizable cationic lipids have been developed for use in LNP. These include C12-200 (Love et al (2010), PNAS vol. 107, 1864-1869), MC3, LN16, MD1 among others. In one type of LNP a GalNac moiety is attached to the outside of the LNP and acts as a ligand for uptake in to the liver via the asialyloglycoprotein receptor. Any of these cationic lipids are used to formulate LNP for delivery of gRNA and Cas9 mRNA to the liver.

In some embodiments, a LNP refers to any particle having a diameter of less than 1000 nm, 500 nm, 250 nm, 200 nm, 150 nm, 100 nm, 75 nm, 50 nm, or 25 nm. Alternatively, a nanoparticle can range in size from 1-1000 nm, 1-500 nm, 1-250 nm, 25-200 nm, 25-100 nm, 35-75 nm, or 25-60 nm.

LNPs can be made from cationic, anionic, or neutral lipids. Neutral lipids, such as the fusogenic phospholipid DOPE or the membrane component cholesterol, can be included in LNPs as ‘helper lipids’ to enhance transfection activity and nanoparticle stability. Limitations of cationic lipids include low efficacy owing to poor stability and rapid clearance, as well as the generation of inflammatory or anti-inflammatory responses. LNPs can also have hydrophobic lipids, hydrophilic lipids, or both hydrophobic and hydrophilic lipids.

Any lipid or combination of lipids that are known in the art can be used to produce a LNP. Examples of lipids used to produce LNPs are: DOTMA, DOSPA, DOTAP, DMRIE, DC-cholesterol, DOTAP-cholesterol, GAP-DMORIE-DPyPE, and GL67A-DOPE-DMPE-polyethylene glycol (PEG). Examples of cationic lipids are: 98N12-5, C12-200, DLin-KC2-DMA (KC2), DLin-MC3-DMA (MC3), XTC, MD1, and 7C1. Examples of neutral lipids are: DPSC, DPPC, POPC, DOPE, and SM. Examples of PEG-modified lipids are: PEG-DMG, PEG-CerC14, and PEG-CerC20.

In embodiments, the lipids can be combined in any number of molar ratios to produce a LNP. In addition, the polynucleotide(s) can be combined with lipid(s) in a wide range of molar ratios to produce a LNP.

In embodiments, the site-directed polypeptide and genome-targeting nucleic acid can each be administered separately to a cell or a patient. On the other hand, the site-directed polypeptide can be pre-complexed with one or more guide RNAs, or one or more crRNA together with a tracrRNA. The pre-complexed material can then be administered to a cell or a patient. Such pre-complexed material is known as a ribonucleoprotein particle (RNP).

RNA is capable of forming specific interactions with RNA or DNA. While this property is exploited in many biological processes, it also comes with the risk of promiscuous interactions in a nucleic acid-rich cellular environment. One solution to this problem is the formation of ribonucleoprotein particles (RNPs), in which the RNA is pre-complexed with an endonuclease. Another benefit of the RNP is protection of the RNA from degradation.

In some embodiments, the endonuclease in the RNP can be modified or unmodified. Likewise, the gRNA, crRNA, tracrRNA, or sgRNA can be modified or unmodified. Numerous modifications are known in the art and can be used.

The endonuclease and sgRNA can be generally combined in a 1:1 molar ratio. Alternatively, the endonuclease, crRNA and tracrRNA can be generally combined in a 1:1:1 molar ratio. However, a wide range of molar ratios can be used to produce a RNP.

In some embodiments, a recombinant adeno-associated virus (AAV) vector can be used for delivery. Techniques to produce rAAV particles, in which an AAV genome to be packaged that includes the polynucleotide to be delivered, rep and cap genes, and helper virus functions are provided to a cell are standard in the art. Production of rAAV requires that the following components are present within a single cell (denoted herein as a packaging cell): a rAAV genome, AAV rep and cap genes separate from (i.e., not in) the rAAV genome, and helper virus functions. The AAV rep and cap genes can be from any AAV serotype for which recombinant virus can be derived, and can be from a different AAV serotype than the rAAV genome ITRs, including, but not limited to, AAV serotypes AAV-1, AAV-2, AAV-3, AAV-4, AAV-5, AAV-6, AAV-7, AAV-8, AAV-9, AAV-10, AAV-11, AAV-12, AAV-13 and AAV rh.74. Production of pseudotyped rAAV is disclosed in, for example, international patent application publication number WO 01/83692. See Table 1.

TABLE 1
AAV serotype and Genbank Accession No. of some selected AAVs.

| AAV Serotype | Genbank Accession No. |
|---|---|
| AAV-1 | NC_002077.1 |
| AAV-2 | NC_001401.2 |
| AAV-3 | NC_001729.1 |
| AAV-3B | AF028705.1 |
| AAV-4 | NC_001829.1 |
| AAV-5 | NC_006152.1 |
| AAV-6 | AF028704.1 |
| AAV-7 | NC_006260.1 |
| AAV-8 | NC_006261.1 |
| AAV-9 | AX753250.1 |
| AAV-10 | AY631965.1 |
| AAV-11 | AY631966.1 |
| AAV-12 | DQ813647.1 |
| AAV-13 | EU285562.1 |

In some embodiments, a method of generating a packaging cell involves creating a cell line that stably expresses all of the necessary components for AAV particle production. For example, a plasmid (or multiple plasmids) having a rAAV genome lacking AAV rep and cap genes, AAV rep and cap genes separate from the rAAV genome, and a selectable marker, such as a neomycin resistance gene, are integrated into the genome of a cell. AAV genomes have been introduced into bacterial plasmids by procedures such as GC tailing (Samulski et al., 1982, Proc. Natl. Acad. S6. USA, 79:2077-2081), addition of synthetic linkers containing restriction endonuclease cleavage sites (Laughlin et al., 1983, Gene, 23:65-73) or by direct, blunt-end ligation (Senapathy & Carter, 1984, J. Biol. Chem., 259:4661-4666). The packaging cell line is then infected with a helper virus, such as adenovirus. The advantages of this method are that the cells are selectable and are suitable for large-scale production of rAAV. Other examples of suitable methods employ adenovirus or baculovirus, rather than plasmids, to introduce rAAV genomes and/or rep and cap genes into packaging cells.

General principles of rAAV production are reviewed in, for example, Carter, 1992, Current Opinions in Biotechnology, 1533-539; and Muzyczka, 1992, Curr. Topics in Microbial. and Immunol., 158:97-129). Various approaches are described in Ratschin et al., Mol. Cell. Biol. 4:2072 (1984); Hermonat et al., Proc. Natl. Acad. Sci. USA, 81:6466 (1984); Tratschin et al., Mol. Cell. Biol. 5:3251 (1985); McLaughlin et al., J. Virol., 62:1963 (1988); and Lebkowski et al., 1988 Mol. Cell. Biol., 7:349 (1988). Samulski et al. (1989, J. Virol., 63:3822-3828); U.S. Pat. No. 5,173,414; WO 95/13365 and corresponding U.S. Pat. No. 5,658,776; WO 95/13392; WO 96/17947; PCT/US98/18600; WO 97/09441 (PCT/US96/14423); WO 97/08298 (PCT/US96/13872); WO 97/21825 (PCT/US96/20777); WO 97/06243 (PCT/FR96/01064); WO 99/11764; Perrin et al. (1995) Vaccine 13:1244-1250; Paul et al. (1993) Human Gene Therapy 4:609-615; Clark et al. (1996) Gene Therapy 3:1124-1132; U.S. Pat. Nos. 5,786,211; 5,871,982; and 6,258,595.

AAV vector serotypes can be matched to target cell types. For example, the following exemplary cell types can be transduced by the indicated AAV serotypes among others. For example, the serotypes of AAV vectors suitable to liver tissue/cell type include, but not limited to, AAV3, AAV5, AAV8 and AAV9.

In addition to adeno-associated viral vectors, other viral vectors can be used. Such viral vectors include, but are not limited to, lentivirus, alphavirus, enterovirus, pestivirus, baculovirus, herpesvirus, Epstein Barr virus, papovavirusr, poxvirus, vaccinia virus, and herpes simplex virus.

In some embodiments, Cas9 mRNA, sgRNA targeting one or two loci in albumin genes, and donor DNA are each separately formulated into lipid nanoparticles, or are all co-formulated into one lipid nanoparticle, or co-formulated into two or more lipid nanoparticles.

In some embodiments, Cas9 mRNA is formulated in a lipid nanoparticle, while sgRNA and donor DNA are delivered in an AAV vector. In some embodiments, Cas9 mRNA and sgRNA are co-formulated in a lipid nanoparticle, while donor DNA is delivered in an AAV vector.

Options are available to deliver the Cas9 nuclease as a DNA plasmid, as mRNA or as a protein. The guide RNA can be expressed from the same DNA, or can also be delivered as an RNA. The RNA can be chemically modified to alter or improve its half-life, or decrease the likelihood or degree of immune response. The endonuclease protein can be complexed with the gRNA prior to delivery. Viral vectors allow efficient delivery; split versions of Cas9 and smaller orthologs of Cas9 can be packaged in AAV, as can donors for HDR. A range of non-viral delivery methods also exist that can deliver each of these components, or non-viral and viral methods can be employed in tandem. For example, nano-particles can be used to deliver the protein and guide RNA, while AAV can be used to deliver a donor DNA.

In some embodiments that are related to deliver genome-editing components for therapeutic treatments, at least two components are delivered in to the nucleus of a cell to be transformed, e.g. hepatocytes; a sequence specific nuclease and a DNA donor template. In some embodiments, the donor DNA template is packaged in to an Adeno Associated Virus (AAV) with tropism for the liver. In some embodiments, the AAV is selected from the serotypes AAV8, AAV9, AAVrh10, AAV5, AAV6 or AAV-DJ. In some embodiments, the AAV packaged DNA donor template is administered to a subject, e.g. a patient first by peripheral IV injection followed by the sequence specific nuclease. The advantage of delivering an AAV packaged donor DNA template first is that the delivered donor DNA template will be stably maintained in the nucleus of the transduced hepatocytes which allows for the subsequent administration of the sequence specific nuclease which will create a double strand break in the genome with subsequent integration of the DNA donor by HDR or NHEJ. It is desirable in some embodiments that the sequence specific nuclease remain active in the target cell only for the time required to promote targeted integration of the transgene at sufficient levels for the desired therapeutic effect. If the sequence specific nuclease remains active in the cell for an extended duration this will result in an increased frequency of double strand breaks at off-target sites. Specifically, the frequency of off target cleavage is a function of the off-target cutting efficiency multiplied by the time over which the nuclease is active. Delivery of a sequence specific nuclease in the form of a mRNA results in a short duration of nuclease activity in the range of hours to a few days because the mRNA and the translated protein are short lived in the cell. Thus, delivery of the sequence specific nuclease in to cells that already contain the donor template is expected to result in the highest possible ratio of targeted integration relative to off-target integration. In addition, AAV mediated delivery of a donor DNA template to the nucleus of hepatocytes after peripheral IV injection takes time, typically on the order of 1 to 14 days due to the requirement for the virus to infect the cell, escape the endosomes and then transit to the nucleus and conversion of the single stranded AAV genome to a double stranded DNA molecule by host components. Thus, it is preferable at least in some embodiments to allow the process of delivery of the donor DNA template to the nucleus to be completed before supplying the CRISPR-Cas9 components since these nuclease components will only be active for about 1 to 3 days.

In some embodiments, the sequence specific nuclease is CRISPR-Cas9 which is composed of a sgRNA directed to a DNA sequence within intron 1 of the albumin gene together with a Cas9 nuclease. In some embodiments, the Cas9 nuclease is delivered as a mRNA encoding the Cas9 protein operably fused to one or more nuclear localization signals (NLS). In some embodiments, the sgRNA and the Cas9 mRNA are delivered to the hepatocytes by packaging into a lipid nanoparticle. In some embodiments, the lipid nanoparticle contains the lipid C12-200 (Love et al 2010, PNAS vol 107 1864-1869). In some embodiments, the ratio of the sgRNA to the Cas9 mRNA that is packaged in the LNP is 1:1 (mass ratio) to result in maximal DNA cleavage in vivo in mice. In alternative embodiments, different mass ratios of the sgRNA to the Cas9 mRNA that is packaged in the LNP can be used, for example, 10:1, 9:1, 8:1, 7:1, 6:1, 5:1, 4:1, 3:1 or 2:1 or reverse ratios. In some embodiments, the Cas9 mRNA and the sgRNA are packaged into separate LNP formulations and the Cas9 mRNA containing LNP is delivered to the patient about 1 to about 8 hr before the LNP containing the sgRNA to allow optimal time for the Cas9 mRNA to be translated prior to delivery of the sgRNA.

In some embodiments, a LNP formulation encapsulating a gRNA and a Cas9 mRNA (“the LNP-nuclease formulation”) is administered to a subject, e.g. a patient, that previously was administered a DNA donor template packaged in to an AAV. In some embodiments, the LNP-nuclease formulation is administered to the subject within 1 day to 28 days or within 7 days to 28 days or within 7 days to 14 days after administration of the AAV-donor DNA template. The optimal timing of delivery of the LNP-nuclease formulation relative to the AAV-donor DNA template can be determined using the techniques known in the art, e.g. studies done in animal models including mice and monkeys.

In some embodiments, a DNA-donor template is delivered to the hepatocytes of a subject, e.g. a patient using a non-viral delivery method. While some patients (typically 30%) have pre-existing neutralizing antibodies directed to most commonly used AAV serotypes that prevents the efficacious gene delivery by said AAV, all patients will be treatable with a non-viral delivery method. Several non-viral delivery methodologies have been known in the field. In particular lipid nanoparticles (LNP) are known to efficiently deliver their encapsulated cargo to the cytoplasm of hepatocytes after intravenous injection in animals and humans. These LNP are actively taken up by the liver through a process of receptor mediated endocytosis resulting in preferential uptake in to the liver.

In some embodiments, in order to promote nuclear localization of a donor template, DNA sequence that can promote nuclear localization of plasmids, e.g. a 366 bp region of the simian virus 40 (SV40) origin of replication and early promoter can be added to the donor template. Other DNA sequences that bind to cellular proteins can also be used to improve nuclear entry of DNA.

In some embodiments, a level of expression or activity of introduced FVIII gene is measured in the blood of a subject, e.g. a patient, following the first administration of a LNP-nuclease formulation, e.g. containing gRNA and Cas9 nuclease or mRNA encoding Cas9 nuclease, after the AAV-donor DNA template. If the FVIII level is not sufficient to cure the disease as defined for example as FVIII levels of at least 5 to 50%, in particular 5 to 20% of normal levels, then a second or third administration of the LNP-nuclease formulation can be given to promote additional targeted integration in to the albumin intron 1 site. The feasibility of using multiple doses of the LNP-nuclease formulation to obtain the desired therapeutic levels of FVIII can be tested and optimized using the techniques known in the field, e.g. tests using animal models including the mouse and the monkey.

In some embodiments, according to any of the methods described herein comprising administration of i) an AAV-donor DNA template comprising a donor cassette and ii) an LNP-nuclease formulation to a subject, an initial dose of the LNP-nuclease formulation is administered to the subject within 1 day to 28 days after administration of the AAV-donor DNA template to the subject. In some embodiments, the initial dose of the LNP-nuclease formulation is administered to the subject after a sufficient time to allow delivery of the donor DNA template to the nucleus of a target cell. In some embodiments, the initial dose of the LNP-nuclease formulation is administered to the subject after a sufficient time to allow conversion of the single stranded AAV genome to a double stranded DNA molecule in the nucleus of a target cell. In some embodiments, one or more (such as 2, 3, 4, 5, or more) additional doses of the LNP-nuclease formulation are administered to the subject following administration of the initial dose. In some embodiments, one or more doses of the LNP-nuclease formulation are administered to the subject until a target level of targeted integration of the donor cassette and/or a target level of expression of the donor cassette is achieved. In some embodiments, the method further comprises measuring the level of targeted integration of the donor cassette and/or the level of expression of the donor cassette following each administration of the LNP-nuclease formulation, and administering an additional dose of the LNP-nuclease formulation if the target level of targeted integration of the donor cassette and/or the target level of expression of the donor cassette is not achieved. In some embodiments, the amount of at least one of the one or more additional doses of the LNP-nuclease formulation is the same as the initial dose. In some embodiments, the amount of at least one of the one or more additional doses of the LNP-nuclease formulation is less than the initial dose. In some embodiments, the amount of at least one of the one or more additional doses of the LNP-nuclease formulation is more than the initial dose.

### Genetically Modified Cells and Cell Populations

In one aspect, the disclosures herewith provide a method of editing a genome in a cell, thereby creating a genetically modified cell. In some aspects, a population of genetically modified cells are provided. The genetically modified cell therefore refers to a cell that has at least one genetic modification introduced by genome editing (e.g., using the CRISPR/Cas9/Cpf1 system). In some embodiments, the genetically modified cell is a genetically modified hepatocyte cell. A genetically modified cell having an exogenous genome-targeting nucleic acid and/or an exogenous nucleic acid encoding a genome-targeting nucleic acid is contemplated herein.

In some embodiments, the genome of a cell can be edited by inserting a nucleic acid sequence of a FVIII gene or functional derivative thereof into a genomic sequence of the cell. In some embodiments, the cell subject to the genome-edition has one or more mutation(s) in the genome which results in reduction of the expression of endogenous FVIII gene as compared to the expression in a normal that does not have such mutation(s). The normal cell can be a healthy or control cell that is originated (or isolated) from a different subject who does not have FVIII gene defects. In some embodiments, the cell subject to the genome-edition can be originated (or isolated) from a subject who is in need of treatment of FVIII gene related condition or disorder. Therefore, in some embodiments the expression of endogenous FVIII gene in such cell is about 10%, about 20%, about 30%, about 40%, about 50%, about 60%, about 70%, about 80%, about 90% or about 100% reduced as compared to the expression of endogenous FVIII gene expression in the normal cell.

Upon successful insertion of the transgene, e.g. a nucleic acid encoding a FVIII gene or functional fragment thereof, the expression of the introduced FVIII gene or functional derivative thereof in the cell can be at least about 10%, about 20%, about 30%, about 40%, about 50%, about 60%, about 70%, about 80%, about 90%, about 100%, about 200%, about 300%, about 400%, about 500%, about 600%, about 700%, about 800%, about 900%, about 1,000%, about 2,000%, about 3,000%, about 5,000%, about 10,000% or more as compared to the expression of endogenous FVIII gene of the cell. In some embodiments, the activity of introduced FVIII gene products including the functional fragment of FVIII in the genome-edited cell can be at least about 10%, about 20%, about 30%, about 40%, about 50%, about 60%, about 70%, about 80%, about 90%, about 100%, about 200%, about 300%, about 400%, about 500%, about 600%, about 700%, about 800%, about 900%, about 1,000%, about 2,000%, about 3,000%, about 5,000%, about 10,000% or more as compared to the expression of endogenous FVIII gene of the cell. In some embodiments, the expression of the introduced FVIII gene or functional derivative thereof in the cell is at least about 2 folds, about 3 folds, about 4 folds, about 5 folds, about 6 folds, about 7 folds, about 8 folds, about 9 folds, about 10 folds, about 15 folds, about 20 folds, about 30 folds, about 50 folds, about 100 folds, about 1000 folds or more of the expression of endogenous FVIII gene of the cell. Also, in some embodiments, the activity of introduced FVIII gene products including the functional fragment of FVIII in the genome-edited cell can be comparable to or more than the activity of FVIII gene products in a normal, healthy cell.

In embodiments where treating or ameliorating Hemophilia A is concerned, the principal targets for gene editing are human cells. For example, in the ex vivo methods and the in vivo methods, the human cells are hepatocytes. In some embodiments, by performing gene editing in autologous cells that are derived from and therefore already completely matched with the patient in need, it is possible to generate cells that can be safely re-introduced into the patient, and effectively give rise to a population of cells that will be effective in ameliorating one or more clinical conditions associated with the patient's disease. In some embodiments for such treatments, hepatocyte cells can be isolated according to any method known in the art and used to create genetically modified, therapeutically effective cells. In one embodiment liver stem cells are genetically modified ex vivo and then re-introduced into the patient where they will give rise to genetically modified hepatocytes or sinusoidal endothelial cells that express the inserted FVIII gene.

### Therapeutic Approach

In one aspect, provided herein is a gene therapy approach for treating Hemophilia A in a patient by editing the genome of the patient. In some embodiments, the gene therapy approach integrates a functional FVIII gene in to the genome of a relevant cell type in patients and this can provide a permanent cure for Hemophilia A. In some embodiments, a cell type subject to the gene therapy approach in which to integrate the FVIII gene is the hepatocyte because these cells efficiently express and secrete many proteins in to the blood. In addition, this integration approach using hepatocytes can be considered for pediatric patients whose livers are not fully grown because the integrated gene would be transmitted to the daughter cells as the hepatocytes divide.

In another aspect, provided herein are cellular, ex vivo and in vivo methods for using genome engineering tools to create permanent changes to the genome by knocking-in a FVIII-encoding gene or functional derivative thereof into a gene locus into a genome and restoring FVIII protein activity. Such methods use endonucleases, such as CRISPR-associated (CRISPR/Cas9, Cpf1 and the like) nucleases, to permanently delete, insert, edit, correct, or replace any sequences from a genome or insert an exogenous sequence, e.g. a FVIII-encoding gene in a genomic locus. In this way, the examples set forth in the present disclosure restore the activity of FVIII gene with a single treatment (rather than deliver potential therapies for the lifetime of the patient).

In some embodiments, an ex vivo cell-based therapy is done using a hepatocyte that is isolated from a patient. Next, the chromosomal DNA of these cells is edited using the materials and methods described herein. Finally, the edited cells are implanted into the patient.

One advantage of an ex vivo cell therapy approach is the ability to conduct a comprehensive analysis of the therapeutic prior to administration. All nuclease-based therapeutics have some level of off-target effects. Performing gene correction ex vivo allows one to fully characterize the corrected cell population prior to implantation. Aspects of the disclosure include sequencing the entire genome of the corrected cells to ensure that the off-target cuts, if any, are in genomic locations associated with minimal risk to the patient. Furthermore, populations of specific cells, including clonal populations, can be isolated prior to implantation.

Another embodiment of such method is an in vivo based therapy. In this method, the chromosomal DNA of the cells in the patient is corrected using the materials and methods described herein. In some embodiments, the cells are hepatocytes.

An advantage of in vivo gene therapy is the ease of therapeutic production and administration. The same therapeutic approach and therapy can be used to treat more than one patient, for example a number of patients who share the same or similar genotype or allele. In contrast, ex vivo cell therapy typically uses a patient's own cells, which are isolated, manipulated and returned to the same patient.

In some embodiments, the subject who is in need of the treatment method accordance with the disclosures is a patient having symptoms of Hemophilia A. In some embodiments, the subject can be a human suspected of having Hemophilia A. Alternatively, the subject can be a human diagnosed with a risk of Hemophilia A. In some embodiments, the subject who is in need of the treatment can have one or more genetic defects (e.g. deletion, insertion and/or mutation) in the endogenous FVIII gene or its regulatory sequences such that the activity including the expression level or functionality of the FVIII protein is substantially reduced compared to a normal, healthy subject.

In some embodiments, provided herein is a method of treating Hemophilia A in a subject, the method comprising providing the following to a cell in the subject: (a) a guide RNA (gRNA) targeting the albumin locus in the cell genome; (b) a DNA endonuclease or nucleic acid encoding said DNA endonuclease; and (c) a donor template comprising a nucleic acid sequence encoding a Factor VIII (FVIII) protein or functional derivative. In some embodiments, the gRNA targets intron 1 of the albumin gene. In some embodiments, the gRNA comprises a spacer sequence from any one of SEQ ID NOs: 18-44 and 104.

In some embodiments, provided herein is a method of treating Hemophilia A in a subject, the method comprising providing the following to a cell in the subject: (a) a gRNA comprising a spacer sequence from any one of SEQ ID NOs: 18-44 and 104; (b) a DNA endonuclease or nucleic acid encoding said DNA endonuclease; and (c) a donor template comprising a nucleic acid sequence encoding a Factor VIII (FVIII) protein or functional derivative. In some embodiments, the gRNA comprises a spacer sequence from any one of SEQ ID NOs: 21, 22, 28, and 30. In some embodiments, the gRNA comprises a spacer sequence from SEQ ID NO: 21. In some embodiments, the gRNA comprises a spacer sequence from SEQ ID NO: 22. In some embodiments, the gRNA comprises a spacer sequence from SEQ ID NO: 28. In some embodiments, the gRNA comprises a spacer sequence from SEQ ID NO: 30. In some embodiments, the cell is a human cell, e.g., a human hepatocyte cell. In some embodiments, the subject is a patient having or is suspected of having Hemophilia A. In some embodiments, the subject is diagnosed with a risk of Hemophilia A.

In some embodiments, according to any of the methods of treating Hemophilia A described herein, the DNA endonuclease is selected from the group consisting of a Cas1, Cas1B, Cas2, Cas3, Cas4, Cas5, Cas6, Cas7, Cas8, Cas9 (also known as Csn1 and Csx12), Cas100, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, or Cpf1 endonuclease, or a functional derivative thereof. In some embodiments, the DNA endonuclease is Cas9. In some embodiments, the Cas9 is from Streptococcus pyogenes (spCas9). In some embodiments, the Cas9 is from Staphylococcus lugdunensis (SluCas9).

In some embodiments, according to any of the methods of treating Hemophilia A described herein, the nucleic acid sequence encoding a Factor VIII (FVIII) protein or functional derivative thereof is codon optimized for expression in the cell. In some embodiments, the cell is a human cell.

In some embodiments, according to any of the methods of treating Hemophilia A described herein, the method employs a nucleic acid encoding the DNA endonuclease. In some embodiments, the nucleic acid encoding the DNA endonuclease is codon optimized for expression in the cell. In some embodiments, the cell is a human cell, e.g., a human hepatocyte cell. In some embodiments, the nucleic acid encoding the DNA endonuclease is DNA, such as a DNA plasmid. In some embodiments, the nucleic acid encoding the DNA endonuclease is RNA, such as mRNA.

In some embodiments, according to any of the methods of treating Hemophilia A described herein, the donor template is encoded in an Adeno Associated Virus (AAV) vector. In some embodiments, the donor template comprises a donor cassette comprising the nucleic acid sequence encoding a Factor VIII (FVIII) protein or functional derivative, and the donor cassette is flanked on one or both sides by a gRNA target site. In some embodiments, the donor cassette is flanked on both sides by a gRNA target site. In some embodiments, the gRNA target site is a target site for the gRNA of (a). In some embodiments, the gRNA target site of the donor template is the reverse complement of a cell genome gRNA target site for the gRNA of (a). In some embodiments, providing the donor template to the cell comprises administering the donor template to the subject. In some embodiments, the administration is via intravenous route.

In some embodiments, according to any of the methods of treating Hemophilia A described herein, the DNA endonuclease or nucleic acid encoding the DNA endonuclease is formulated in a liposome or lipid nanoparticle. In some embodiments, the liposome or lipid nanoparticle also comprises the gRNA. In some embodiments, providing the gRNA and the DNA endonuclease or nucleic acid encoding the DNA endonuclease to the cell comprises administering the liposome or lipid nanoparticle to the subject. In some embodiments, the administration is via intravenous route. In some embodiments, the liposome or lipid nanoparticle is a lipid nanoparticle. In some embodiments, the method employs a lipid nanoparticle comprising nucleic acid encoding the DNA endonuclease and the gRNA. In some embodiments, the nucleic acid encoding the DNA endonuclease is an mRNA encoding the DNA endonuclease.

In some embodiments, according to any of the methods of treating Hemophilia A described herein, the DNA endonuclease is pre-complexed with the gRNA, forming a ribonucleoprotein (RNP) complex.

In some embodiments, according to any of the methods of treating Hemophilia A described herein, the gRNA of (a) and the DNA endonuclease or nucleic acid encoding the DNA endonuclease of (b) are provided to the cell after the donor template of (c) is provided to the cell. In some embodiments, the gRNA of (a) and the DNA endonuclease or nucleic acid encoding the DNA endonuclease of (b) are provided to the cell more than 4 days after the donor template of (c) is provided to the cell. In some embodiments, the gRNA of (a) and the DNA endonuclease or nucleic acid encoding the DNA endonuclease of (b) are provided to the cell at least 14 days after the donor template of (c) is provided to the cell. In some embodiments, the gRNA of (a) and the DNA endonuclease or nucleic acid encoding the DNA endonuclease of (b) are provided to the cell at least 17 days after the donor template of (c) is provided to the cell. In some embodiments, providing (a) and (b) to the cell comprises administering (such as by intravenous route) to the subject a lipid nanoparticle comprising nucleic acid encoding the DNA endonuclease and the gRNA. In some embodiments, the nucleic acid encoding the DNA endonuclease is an mRNA encoding the DNA endonuclease. In some embodiments, providing (c) to the cell comprises administering (such as by intravenous route) to the subject the donor template encoded in an AAV vector.

In some embodiments, according to any of the methods of treating Hemophilia A described herein, one or more additional doses of the gRNA of (a) and the DNA endonuclease or nucleic acid encoding the DNA endonuclease of (b) are provided to the cell following the first dose of the gRNA of (a) and the DNA endonuclease or nucleic acid encoding the DNA endonuclease of (b). In some embodiments, one or more additional doses of the gRNA of (a) and the DNA endonuclease or nucleic acid encoding the DNA endonuclease of (b) are provided to the cell following the first dose of the gRNA of (a) and the DNA endonuclease or nucleic acid encoding the DNA endonuclease of (b) until a target level of targeted integration of the nucleic acid sequence encoding a Factor VIII (FVIII) protein or functional derivative and/or a target level of expression of the nucleic acid sequence encoding a Factor VIII (FVIII) protein or functional derivative is achieved. In some embodiments, providing (a) and (b) to the cell comprises administering (such as by intravenous route) to the subject a lipid nanoparticle comprising nucleic acid encoding the DNA endonuclease and the gRNA. In some embodiments, the nucleic acid encoding the DNA endonuclease is an mRNA encoding the DNA endonuclease.

In some embodiments, according to any of the methods of treating Hemophilia A described herein, the nucleic acid sequence encoding a Factor VIII (FVIII) protein or functional derivative is expressed under the control of the endogenous albumin promoter.

In some embodiments, according to any of the methods of treating Hemophilia A described herein, the nucleic acid sequence encoding a Factor VIII (FVIII) protein or functional derivative is expressed in the liver of the subject.

Implanting Cells into a Subject

In some embodiments, the ex vivo methods of the disclosure involve implanting the genome-edited cells into a subject who is in need of such method. This implanting step can be accomplished using any method of implantation known in the art. For example, the genetically modified cells can be injected directly in the subject's blood or otherwise administered to the subject.

In some embodiments, the methods disclosed herein include administering, which can be interchangeably used with “introducing” and “transplanting,” genetically-modified, therapeutic cells into a subject, by a method or route that results in at least partial localization of the introduced cells at a desired site such that a desired effect(s) is produced. The therapeutic cells or their differentiated progeny can be administered by any appropriate route that results in delivery to a desired location in the subject where at least a portion of the implanted cells or components of the cells remain viable. The period of viability of the cells after administration to a subject can be as short as a few hours, e.g., twenty-four hours, to a few days, to as long as several years, or even the life time of the patient, i.e., long-term engraftment.

When provided prophylactically, the therapeutic cells described herein can be administered to a subject in advance of any symptom of Hemophilia A. Accordingly, in some embodiments the prophylactic administration of a genetically modified hepatocyte cell population serves to prevent the occurrence of Hemophilia A symptoms.

When provided therapeutically in some embodiments, genetically modified hepatocyte cells are provided at (or after) the onset of a symptom or indication of Hemophilia A, e.g., upon the onset of disease.

In some embodiments, a therapeutic hepatocyte cell population being administered according to the methods described herein has allogeneic hepatocyte cells obtained from one or more donors. “Allogeneic” refers to a hepatocyte cell or biological samples having hepatocyte cells obtained from one or more different donors of the same species, where the genes at one or more loci are not identical. For example, a hepatocyte cell population being administered to a subject can be derived from one more unrelated donor subjects, or from one or more non-identical siblings. In some embodiments, syngeneic hepatocyte cell populations can be used, such as those obtained from genetically identical animals, or from identical twins. In other embodiments, the hepatocyte cells are autologous cells; that is, the hepatocyte cells are obtained or isolated from a subject and administered to the same subject, i.e., the donor and recipient are the same.

In one embodiment, an effective amount refers to the amount of a population of therapeutic cells needed to prevent or alleviate at least one or more signs or symptoms of Hemophilia A, and relates to a sufficient amount of a composition to provide the desired effect, e.g., to treat a subject having Hemophilia A. In embodiments, a therapeutically effective amount therefore refers to an amount of therapeutic cells or a composition having therapeutic cells that is sufficient to promote a particular effect when administered to a typical subject, such as one who has or is at risk for Hemophilia A. An effective amount would also include an amount sufficient to prevent or delay the development of a symptom of the disease, alter the course of a symptom of the disease (for example but not limited to, slow the progression of a symptom of the disease), or reverse a symptom of the disease. It is understood that for any given case, an appropriate effective amount can be determined by one of ordinary skill in the art using routine experimentation.

For use in the various embodiments described herein, an effective amount of therapeutic cells, e.g. genome-edited hepatocyte cells can be at least 10² cells, at least 5×10² cells, at least 10³ cells, at least 5×10³ cells, at least 10⁴ cells, at least 5×10⁴ cells, at least 10⁵ cells, at least 2×10⁵ cells, at least 3×10⁵ cells, at least 4×10⁵ cells, at least 5×10⁵ cells, at least 6×10⁵ cells, at least 7×10⁵ cells, at least 8×10⁵ cells, at least 9×10⁵ cells, at least 1×10⁶ cells, at least 2×10⁶ cells, at least 3×10⁶ cells, at least 4×10⁶ cells, at least 5×10⁶ cells, at least 6×10⁶ cells, at least 7×10⁶ cells, at least 8×10⁶ cells, at least 9×10⁶ cells, or multiples thereof. The therapeutic cells can be derived from one or more donors, or are obtained from an autologous source. In some embodiments described herein, the therapeutic cells are expanded in culture prior to administration to a subject in need thereof.

In some embodiments, modest and incremental increases in the levels of functional FVIII expressed in cells of patients having Hemophilia A can be beneficial for ameliorating one or more symptoms of the disease, for increasing long-term survival, and/or for reducing side effects associated with other treatments. Upon administration of such cells to human patients, the presence of therapeutic cells that are producing increased levels of functional FVIII is beneficial. In some embodiments, effective treatment of a subject gives rise to at least about 1%, 3%, 5% or 7% functional FVIII relative to total FVIII in the treated subject. In some embodiments, functional FVIII is at least about 10% of total FVIII. In some embodiments, functional FVIII is at least, about or at most 20%, 30%, 40%, 50%, 60%, 70%, 80%, 90% or 100% of total FVIII. Similarly, the introduction of even relatively limited subpopulations of cells having significantly elevated levels of functional FVIII can be beneficial in various patients because in some situations normalized cells will have a selective advantage relative to diseased cells. However, even modest levels of therapeutic cells with elevated levels of functional FVIII can be beneficial for ameliorating one or more aspects of Hemophilia A in patients. In some embodiments, about 10%, about 20%, about 30%, about 40%, about 50%, about 60%, about 70%, about 80%, about 90% or more of the therapeutic in patients to whom such cells are administered are producing increased levels of functional FVIII.

In embodiments, the delivery of a therapeutic cell composition into a subject by a method or route results in at least partial localization of the cell composition at a desired site. A cell composition can be administered by any appropriate route that results in effective treatment in the subject, i.e. administration results in delivery to a desired location in the subject where at least a portion of the composition delivered, i.e. at least 1×10⁴ cells are delivered to the desired site for a period of time. Modes of administration include injection, infusion, instillation, or ingestion. “Injection” includes, without limitation, intravenous, intramuscular, intra-arterial, intrathecal, intraventricular, intracapsular, intraorbital, intracardiac, intradermal, intraperitoneal, transtracheal, subcutaneous, subcuticular, intraarticular, sub capsular, subarachnoid, intraspinal, intracerebro spinal, and intrasternal injection and infusion. In some embodiments, the route is intravenous. For the delivery of cells, administration by injection or infusion can be made.

In one embodiment, the cells are administered systemically, in other words a population of therapeutic cells are administered other than directly into a target site, tissue, or organ, such that it enters, instead, the subject's circulatory system and, thus, is subject to metabolism and other like processes.

The efficacy of a treatment having a composition for the treatment of Hemophilia A can be determined by the skilled clinician. However, a treatment is considered effective treatment if any one or all of the signs or symptoms of, as but one example, levels of functional FVIII are altered in a beneficial manner (e.g., increased by at least 10%), or other clinically accepted symptoms or markers of disease are improved or ameliorated. Efficacy can also be measured by failure of an individual to worsen as assessed by hospitalization or need for medical interventions (e.g., progression of the disease is halted or at least slowed). Methods of measuring these indicators are known to those of skill in the art and/or described herein. Treatment includes any treatment of a disease in an individual or an animal (some non-limiting examples include a human, or a mammal) and includes: (1) inhibiting the disease, e.g., arresting, or slowing the progression of symptoms; or (2) relieving the disease, e.g., causing regression of symptoms; and (3) preventing or reducing the likelihood of the development of symptoms.

### Composition

In one aspect, the present disclosure provides compositions for carrying out the methods disclosed herein. A composition can include one or more of the following: a genome-targeting nucleic acid (e.g. gRNA); a site-directed polypeptide (e.g. DNA endonuclease) or a nucleotide sequence encoding the site-directed polypeptide; and a polynucleotide to be inserted (e.g. a donor template) to effect the desired genetic modification of the methods disclosed herein.

In some embodiments, a composition has a nucleotide sequence encoding a genome-targeting nucleic acid (e.g. gRNA).

In some embodiments, a composition has a site-directed polypeptide (e.g. DNA endonuclease). In some embodiments, a composition has a nucleotide sequence encoding the site-directed polypeptide.

In some embodiments, a composition has a polynucleotide (e.g. a donor template) to be inserted into a genome.

In some embodiments, a composition has (i) a nucleotide sequence encoding a genome-targeting nucleic acid (e.g. gRNA) and (ii) a site-directed polypeptide (e.g. DNA endonuclease) or a nucleotide sequence encoding the site-directed polypeptide.

In some embodiments, a composition has (i) a nucleotide sequence encoding a genome-targeting nucleic acid (e.g. gRNA) and (ii) a polynucleotide (e.g. a donor template) to be inserted into a genome.

In some embodiments, a composition has (i) a site-directed polypeptide (e.g. DNA endonuclease) or a nucleotide sequence encoding the site-directed polypeptide and (ii) a polynucleotide (e.g. a donor template) to be inserted into a genome.

In some embodiments, a composition has (i) a nucleotide sequence encoding a genome-targeting nucleic acid (e.g. gRNA), (ii) a site-directed polypeptide (e.g. DNA endonuclease) or a nucleotide sequence encoding the site-directed polypeptide and (iii) a polynucleotide (e.g. a donor template) to be inserted into a genome.

In some embodiments of any of the above compositions, the composition has a single-molecule guide genome-targeting nucleic acid. In some embodiments of any of the above compositions, the composition has a double-molecule genome-targeting nucleic acid. In some embodiments of any of the above compositions, the composition has two or more double-molecule guides or single-molecule guides. In some embodiments, the composition has a vector that encodes the nucleic acid targeting nucleic acid. In some embodiments, the genome-targeting nucleic acid is a DNA endonuclease, in particular, Cas9.

In some embodiments, a composition can contain composition that includes one or more gRNA that can be used for genome-edition, in particular, insertion of a FVIII gene or derivative thereof into a genome of a cell. The gRNA for the composition can target a genomic site at, within, or near the endogenous albumin gene. Therefore, in some embodiments, the gRNA can have a spacer sequence complementary to a genomic sequence at, within, or near the albumin gene.

In some embodiments, a gRNA for a composition is a sequence selected from those listed in Table 3 and variants thereof having at least about 50%, about 55%, about 60%, about 65%, about 70%, about 75%, about 80%, about 85%, about 90% or about 95% identity or homology to any of those listed in Table 3. In some embodiments, the variants of gRNA for the kit have at least about 85% homology to any of those listed in Table 3.

In some embodiments, a gRNA for a composition has a spacer sequence that is complementary to a target site in the genome. In some embodiments, the spacer sequence is 15 bases to 20 bases in length. In some embodiments, a complementarity between the spacer sequence to the genomic sequence is at least 80%, at least 85%, at least 90%, at least 95%, at least 96%, at least 97%, at least 98%, at least 99% or at least 100%.

In some embodiments, a composition can have a DNA endonuclease or a nucleic acid encoding the DNA endonuclease and/or a donor template having a nucleic acid sequence of a FVIII gene or functional derivative thereof. In some embodiments, the DNA endonuclease is Cas9. In some embodiments, the nucleic acid encoding the DNA endonuclease is DNA or RNA.

In some embodiments, one or more of any oligonucleotides or nucleic acid sequences for the kit can be encoded in an Adeno Associated Virus (AAV) vector. Therefore, in some embodiments, a gRNA can be encoded in an AAV vector. In some embodiments, a nucleic acid encoding a DNA endonuclease can be encoded in an AAV vector. In some embodiments, a donor template can be encoded in an AAV vector. In some embodiments, two or more oligonucleotides or nucleic acid sequences can be encoded in a single AAV vector. Thus, in some embodiments, a gRNA sequence and a DNA endonuclease-encoding nucleic acid can be encoded in a single AAV vector.

In some embodiments, a composition can have a liposome or a lipid nanoparticle. Therefore, in some embodiments, any compounds (e.g. a DNA endonuclease or a nucleic acid encoding thereof, gRNA and donor template) of the composition can be formulated in a liposome or lipid nanoparticle. In some embodiments, one or more such compounds are associated with a liposome or lipid nanoparticle via a covalent bond or non-covalent bond. In some embodiments, any of the compounds can be separately or together contained in a liposome or lipid nanoparticle. Therefore, in some embodiments, each of a DNA endonuclease or a nucleic acid encoding thereof, gRNA and donor template is separately formulated in a liposome or lipid nanoparticle. In some embodiments, a DNA endonuclease is formulated in a liposome or lipid nanoparticle with gRNA. In some embodiments, a DNA endonuclease or a nucleic acid encoding thereof, gRNA and donor template are formulated in a liposome or lipid nanoparticle together.

In some embodiments, a composition described above further has one or more additional reagents, where such additional reagents are selected from a buffer, a buffer for introducing a polypeptide or polynucleotide into a cell, a wash buffer, a control reagent, a control vector, a control RNA polynucleotide, a reagent for in vitro production of the polypeptide from DNA, adaptors for sequencing and the like. A buffer can be a stabilization buffer, a reconstituting buffer, a diluting buffer, or the like. In some embodiments, a composition can also include one or more components that can be used to facilitate or enhance the on-target binding or the cleavage of DNA by the endonuclease, or improve the specificity of targeting.

In some embodiments, any components of a composition are formulated with pharmaceutically acceptable excipients such as carriers, solvents, stabilizers, adjuvants, diluents, etc., depending upon the particular mode of administration and dosage form. In embodiments, guide RNA compositions are generally formulated to achieve a physiologically compatible pH, and range from a pH of about 3 to a pH of about 11, about pH 3 to about pH 7, depending on the formulation and route of administration. In some embodiments, the pH is adjusted to a range from about pH 5.0 to about pH 8. In some embodiments, the composition has a therapeutically effective amount of at least one compound as described herein, together with one or more pharmaceutically acceptable excipients. Optionally, the composition can have a combination of the compounds described herein, or can include a second active ingredient useful in the treatment or prevention of bacterial growth (for example and without limitation, anti-bacterial or anti-microbial agents), or can include a combination of reagents of the disclosure. In some embodiments, gRNAs are formulated with other one or more oligonucleotides, e.g. a nucleic acid encoding DNA endonuclease and/or a donor template. Alternatively, a nucleic acid encoding DNA endonuclease and a donor template, separately or in combination with other oligonucleotides, are formulated with the method described above for gRNA formulation.

Suitable excipients can include, for example, carrier molecules that include large, slowly metabolized macromolecules such as proteins, polysaccharides, polylactic acids, polyglycolic acids, polymeric amino acids, amino acid copolymers, and inactive virus particles. Other exemplary excipients include antioxidants (for example and without limitation, ascorbic acid), chelating agents (for example and without limitation, EDTA), carbohydrates (for example and without limitation, dextrin, hydroxyalkylcellulose, and hydroxyalkylmethylcellulose), stearic acid, liquids (for example and without limitation, oils, water, saline, glycerol and ethanol), wetting or emulsifying agents, pH buffering substances, and the like.

In some embodiments, any compounds (e.g. a DNA endonuclease or a nucleic acid encoding thereof, gRNA and donor template) of a composition can be delivered via transfection such as electroporation. In some exemplary embodiments, a DNA endonuclease can be precomplexed with a gRNA, forming a Ribonucleoprotein (RNP) complex, prior to the provision to the cell and the RNP complex can be electroporated. In such embodiments, the donor template can delivered via electroporation.

In some embodiments, a composition refers to a therapeutic composition having therapeutic cells that are used in an ex vivo treatment method.

In embodiments, therapeutic compositions contain a physiologically tolerable carrier together with the cell composition, and optionally at least one additional bioactive agent as described herein, dissolved or dispersed therein as an active ingredient. In some embodiments, the therapeutic composition is not substantially immunogenic when administered to a mammal or human patient for therapeutic purposes, unless so desired.

In general, the genetically-modified, therapeutic cells described herein are administered as a suspension with a pharmaceutically acceptable carrier. One of skill in the art will recognize that a pharmaceutically acceptable carrier to be used in a cell composition will not include buffers, compounds, cryopreservation agents, preservatives, or other agents in amounts that substantially interfere with the viability of the cells to be delivered to the subject. A formulation having cells can include e.g., osmotic buffers that permit cell membrane integrity to be maintained, and optionally, nutrients to maintain cell viability or enhance engraftment upon administration. Such formulations and suspensions are known to those of skill in the art and/or can be adapted for use with the progenitor cells, as described herein, using routine experimentation.

In some embodiments, a cell composition can also be emulsified or presented as a liposome composition, provided that the emulsification procedure does not adversely affect cell viability. The cells and any other active ingredient can be mixed with excipients that are pharmaceutically acceptable and compatible with the active ingredient, and in amounts suitable for use in the therapeutic methods described herein.

Additional agents included in a cell composition can include pharmaceutically acceptable salts of the components therein. Pharmaceutically acceptable salts include the acid addition salts (formed with the free amino groups of the polypeptide) that are formed with inorganic acids, such as, for example, hydrochloric or phosphoric acids, or such organic acids as acetic, tartaric, mandelic and the like. Salts formed with the free carboxyl groups can also be derived from inorganic bases, such as, for example, sodium, potassium, ammonium, calcium or ferric hydroxides, and such organic bases as isopropylamine, trimethylamine, 2-ethylamino ethanol, histidine, procaine and the like.

Physiologically tolerable carriers are well known in the art. Exemplary liquid carriers are sterile aqueous solutions that contain no materials in addition to the active ingredients and water, or contain a buffer such as sodium phosphate at physiological pH value, physiological saline or both, such as phosphate-buffered saline. Still further, aqueous carriers can contain more than one buffer salt, as well as salts such as sodium and potassium chlorides, dextrose, polyethylene glycol and other solutes. Liquid compositions can also contain liquid phases in addition to and to the exclusion of water. Exemplary of such additional liquid phases are glycerin, vegetable oils such as cottonseed oil, and water-oil emulsions. The amount of an active compound used in the cell compositions that is effective in the treatment of a particular disorder or condition will depend on the nature of the disorder or condition, and can be determined by standard clinical techniques.

### Kit

Some embodiments provide a kit that contains any of the above-described compositions, e.g. a composition for genome edition or a therapeutic cell composition and one or more additional components.

In some embodiments, a kit can have one or more additional therapeutic agents that can be administered simultaneously or in sequence with the composition for a desired purpose, e.g. genome edition or cell therapy.

In some embodiments, a kit can further include instructions for using the components of the kit to practice the methods. The instructions for practicing the methods are generally recorded on a suitable recording medium. For example, the instructions can be printed on a substrate, such as paper or plastic, etc. The instructions can be present in the kits as a package insert, in the labeling of the container of the kit or components thereof (i.e., associated with the packaging or subpackaging), etc. The instructions can be present as an electronic storage data file present on a suitable computer readable storage medium, e.g. CD-ROM, diskette, flash drive, etc. In some instances, the actual instructions are not present in the kit, but means for obtaining the instructions from a remote source (e.g. via the Internet), can be provided. An example of this embodiment is a kit that includes a web address where the instructions can be viewed and/or from which the instructions can be downloaded. As with the instructions, this means for obtaining the instructions can be recorded on a suitable substrate.

### Other Possible Therapeutic Approaches

Gene editing can be conducted using nucleases engineered to target specific sequences. To date there are four major types of nucleases: meganucleases and their derivatives, zinc finger nucleases (ZFNs), transcription activator like effector nucleases (TALENs), and CRISPR-Cas9 nuclease systems. The nuclease platforms vary in difficulty of design, targeting density and mode of action, particularly as the specificity of ZFNs and TALENs is through protein-DNA interactions, while RNA-DNA interactions primarily guide Cas9. Cas9 cleavage also requires an adjacent motif, the PAM, which differs between different CRISPR systems. Cas9 from Streptococcus pyogenes cleaves using a NRG PAM, CRISPR from Neisseria meningitidis can cleave at sites with PAMs including NNNNGATT (SEQ ID NO: 101), NNNNNGTTT (SEQ ID NO: 102) and NNNNGCTT (SEQ ID NO: 103). A number of other Cas9 orthologs target protospacer adjacent to alternative PAMs.

CRISPR endonucleases, such as Cas9, can be used in various embodiments of the methods of the disclosure. However, the teachings described herein, such as therapeutic target sites, could be applied to other forms of endonucleases, such as ZFNs, TALENs, HEs, or MegaTALs, or using combinations of nucleases. However, in order to apply the teachings of the present disclosure to such endonucleases, one would need to, among other things, engineer proteins directed to the specific target sites.

Additional binding domains can be fused to the Cas9 protein to increase specificity. The target sites of these constructs would map to the identified gRNA specified site, but would require additional binding motifs, such as for a zinc finger domain. In the case of Mega-TAL, a meganuclease can be fused to a TALE DNA-binding domain. The meganuclease domain can increase specificity and provide the cleavage. Similarly, inactivated or dead Cas9 (dCas9) can be fused to a cleavage domain and require the sgRNA/Cas9 target site and adjacent binding site for the fused DNA-binding domain. This likely would require some protein engineering of the dCas9, in addition to the catalytic inactivation, to decrease binding without the additional binding site.

In some embodiments, the compositions and methods of editing genome in accordance with the present disclosures (e.g. insertion of a FVIII-encoding sequence into the albumin locus) can utilize or be done using any of the following approaches.

### Zinc Finger Nucleases

Zinc finger nucleases (ZFNs) are modular proteins having an engineered zinc finger DNA binding domain linked to the catalytic domain of the type II endonuclease Fold. Because FokI functions only as a dimer, a pair of ZFNs must be engineered to bind to cognate target “half-site” sequences on opposite DNA strands and with precise spacing between them to enable the catalytically active FokI dimer to form. Upon dimerization of the FokI domain, which itself has no sequence specificity per se, a DNA double-strand break is generated between the ZFN half-sites as the initiating step in genome editing.

The DNA binding domain of each ZFN typically has 3-6 zinc fingers of the abundant Cys2-His2 architecture, with each finger primarily recognizing a triplet of nucleotides on one strand of the target DNA sequence, although cross-strand interaction with a fourth nucleotide also can be important. Alteration of the amino acids of a finger in positions that make key contacts with the DNA alters the sequence specificity of a given finger. Thus, a four-finger zinc finger protein will selectively recognize a 12 bp target sequence, where the target sequence is a composite of the triplet preferences contributed by each finger, although triplet preference can be influenced to varying degrees by neighboring fingers. An important aspect of ZFNs is that they can be readily re-targeted to almost any genomic address simply by modifying individual fingers, although considerable expertise is required to do this well. In most applications of ZFNs, proteins of 4-6 fingers are used, recognizing 12-18 bp respectively. Hence, a pair of ZFNs will typically recognize a combined target sequence of 24-36 bp, not including the 5-7 bp spacer between half-sites. The binding sites can be separated further with larger spacers, including 15-17 bp. A target sequence of this length is likely to be unique in the human genome, assuming repetitive sequences or gene homologs are excluded during the design process. Nevertheless, the ZFN protein-DNA interactions are not absolute in their specificity so off-target binding and cleavage events do occur, either as a heterodimer between the two ZFNs, or as a homodimer of one or the other of the ZFNs. The latter possibility has been effectively eliminated by engineering the dimerization interface of the FokI domain to create “plus” and “minus” variants, also known as obligate heterodimer variants, which can only dimerize with each other, and not with themselves. Forcing the obligate heterodimer prevents formation of the homodimer. This has greatly enhanced specificity of ZFNs, as well as any other nuclease that adopts these Fold variants.

A variety of ZFN-based systems have been described in the art, modifications thereof are regularly reported, and numerous references describe rules and parameters that are used to guide the design of ZFNs; see, e.g., Segal et al., Proc Natl Acad Sci USA 96(6):2758-63 (1999); Dreier B et al., J Mol Biol. 303(4):489-502 (2000); Liu Q et al., J Biol Chem. 277(6):3850-6 (2002); Dreier et al., J Biol Chem 280(42):35588-97 (2005); and Dreier et al., J Biol Chem. 276(31):29466-78 (2001).

### Transcription Activator-Like Effector Nucleases (TALENs)

TALENs represent another format of modular nucleases whereby, as with ZFNs, an engineered DNA binding domain is linked to the FokI nuclease domain, and a pair of TALENs operate in tandem to achieve targeted DNA cleavage. The major difference from ZFNs is the nature of the DNA binding domain and the associated target DNA sequence recognition properties. The TALEN DNA binding domain derives from TALE proteins, which were originally described in the plant bacterial pathogen Xanthomonas sp. TALEs have tandem arrays of 33-35 amino acid repeats, with each repeat recognizing a single base pair in the target DNA sequence that is typically up to 20 bp in length, giving a total target sequence length of up to 40 bp. Nucleotide specificity of each repeat is determined by the repeat variable diresidue (RVD), which includes just two amino acids at positions 12 and 13. The bases guanine, adenine, cytosine and thymine are predominantly recognized by the four RVDs: Asn-Asn, Asn-Ile, His-Asp and Asn-Gly, respectively. This constitutes a much simpler recognition code than for zinc fingers, and thus represents an advantage over the latter for nuclease design. Nevertheless, as with ZFNs, the protein-DNA interactions of TALENs are not absolute in their specificity, and TALENs have also benefitted from the use of obligate heterodimer variants of the Fold domain to reduce off-target activity.

Additional variants of the FokI domain have been created that are deactivated in their catalytic function. If one half of either a TALEN or a ZFN pair contains an inactive FokI domain, then only single-strand DNA cleavage (nicking) will occur at the target site, rather than a DSB. The outcome is comparable to the use of CRISPR/Cas9/Cpf1 “nickase” mutants in which one of the Cas9 cleavage domains has been deactivated. DNA nicks can be used to drive genome editing by HDR, but at lower efficiency than with a DSB. The main benefit is that off-target nicks are quickly and accurately repaired, unlike the DSB, which is prone to NHEJ-mediated mis-repair.

A variety of TALEN-based systems have been described in the art, and modifications thereof are regularly reported; see, e.g., Boch, Science 326(5959):1509-12 (2009); Mak et al., Science 335(6069):716-9 (2012); and Moscou et al., Science 326(5959):1501 (2009). The use of TALENs based on the “Golden Gate” platform, or cloning scheme, has been described by multiple groups; see, e.g., Cermak et al., Nucleic Acids Res. 39(12):e82 (2011); Li et al., Nucleic Acids Res. 39(14):6315-25(2011); Weber et al., PLoS One. 6(2):e16765 (2011); Wang et al., J Genet Genomics 41(6):339-47, Epub 2014 Can 17 (2014); and Cermak T et al., Methods Mol Biol. 1239:133-59 (2015).

### Homing Endonucleases

Homing endonucleases (HEs) are sequence-specific endonucleases that have long recognition sequences (14-44 base pairs) and cleave DNA with high specificity—often at sites unique in the genome. There are at least six known families of HEs as classified by their structure, including LAGLIDADG (SEQ ID NO:6), GIY-YIG, His-Cis box, H—N—H, PD-(D/E)xK, and Vsr-like that are derived from a broad range of hosts, including eukarya, protists, bacteria, archaea, cyanobacteria and phage. As with ZFNs and TALENs, HEs can be used to create a DSB at a target locus as the initial step in genome editing. In addition, some natural and engineered HEs cut only a single strand of DNA, thereby functioning as site-specific nickases. The large target sequence of HEs and the specificity that they offer have made them attractive candidates to create site-specific DSBs.

A variety of HE-based systems have been described in the art, and modifications thereof are regularly reported; see, e.g., the reviews by Steentoft et al., Glycobiology 24(8):663-80 (2014); Belfort and Bonocora, Methods Mol Biol. 1123:1-26 (2014); Hafez and Hausner, Genome 55(8):553-69 (2012); and references cited therein.

### MegaTAL/Tev-mTALEN/MegaTev

As further examples of hybrid nucleases, the MegaTAL platform and Tev-mTALEN platform use a fusion of TALE DNA binding domains and catalytically active HEs, taking advantage of both the tunable DNA binding and specificity of the TALE, as well as the cleavage sequence specificity of the HE; see, e.g., Boissel et al., NAR 42: 2591-2601 (2014); Kleinstiver et al., G3 4:1155-65 (2014); and Boissel and Scharenberg, Methods Mol. Biol. 1239: 171-96 (2015).

In a further variation, the MegaTev architecture is the fusion of a meganuclease (Mega) with the nuclease domain derived from the GIY-YIG homing endonuclease I-TevI (Tev). The two active sites are positioned ˜30 bp apart on a DNA substrate and generate two DSBs with non-compatible cohesive ends; see, e.g., Wolfs et al., NAR 42, 8816-29 (2014). It is anticipated that other combinations of existing nuclease-based approaches will evolve and be useful in achieving the targeted genome modifications described herein.

dCas9-FokI or dCpf1-FokI and Other Nucleases

Combining the structural and functional properties of the nuclease platforms described above offers a further approach to genome editing that can potentially overcome some of the inherent deficiencies. As an example, the CRISPR genome editing system typically uses a single Cas9 endonuclease to create a DSB. The specificity of targeting is driven by a 20 or 22 nucleotide sequence in the guide RNA that undergoes Watson-Crick base-pairing with the target DNA (plus an additional 2 bases in the adjacent NAG or NGG PAM sequence in the case of Cas9 from S. pyogenes). Such a sequence is long enough to be unique in the human genome, however, the specificity of the RNA/DNA interaction is not absolute, with significant promiscuity sometimes tolerated, particularly in the 5′ half of the target sequence, effectively reducing the number of bases that drive specificity. One solution to this has been to completely deactivate the Cas9 or Cpf1 catalytic function—retaining only the RNA-guided DNA binding function—and instead fusing a Fold domain to the deactivated Cas9; see, e.g., Tsai et al., Nature Biotech 32: 569-76 (2014); and Guilinger et al., Nature Biotech. 32: 577-82 (2014). Because FokI must dimerize to become catalytically active, two guide RNAs are required to tether two FokI fusions in close proximity to form the dimer and cleave DNA. This essentially doubles the number of bases in the combined target sites, thereby increasing the stringency of targeting by CRISPR-based systems.

As further example, fusion of the TALE DNA binding domain to a catalytically active HE, such as I-TevI, takes advantage of both the tunable DNA binding and specificity of the TALE, as well as the cleavage sequence specificity of I-TevI, with the expectation that off-target cleavage can be further reduced.

The details of one or more embodiments of the disclosure are set forth in the accompanying description below. Although any materials and methods similar or equivalent to those described herein can be used in the practice or testing of the present disclosure, the preferred materials and methods are now described. Other features, objects and advantages of the disclosure will be apparent from the description. In the description, the singular forms also include the plural unless the context clearly dictates otherwise. Unless defined otherwise, all technical and scientific terms used herein have the same meaning as commonly understood by one of ordinary skill in the art to which this disclosure belongs. In the case of conflict, the present description will control.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

Some embodiments of the disclosures provided herewith are further illustrated by the following non-limiting examples.

### EXEMPLARY EMBODIMENTS

### Embodiment 1

A system comprising:

a deoxyribonucleic acid (DNA) endonuclease or nucleic acid encoding said DNA endonuclease;

guide RNA (gRNA) comprising a spacer sequence from any one of SEQ ID NOs: 22, 21, 28, 30, 18-20, 23-27, 29, 31-44, and 104; and

a donor template comprising a nucleic acid sequence encoding a Factor VIII (FVIII) protein or functional derivative thereof.

### Embodiment 2

The system of embodiment 1, wherein the gRNA comprises a spacer sequence from any one of SEQ ID NOs: 22, 21, 28, and 30.

### Embodiment 3

The system of embodiment 2, wherein the gRNA comprises a spacer sequence from SEQ ID NO: 22.

### Embodiment 4

The system of embodiment 2, wherein the gRNA comprises a spacer sequence from SEQ ID NO: 21.

### Embodiment 5

The system of embodiment 2, wherein the gRNA comprises a spacer sequence from SEQ ID NO: 28.

### Embodiment 6

The system of embodiment 2, wherein the gRNA comprises a spacer sequence from SEQ ID NO: 30.

### Embodiment 7

The system of any one of embodiments 1-6, wherein said DNA endonuclease is selected from the group consisting of a Cast, Cas1B, Cas2, Cas3, Cas4, Cas5, Cas6, Cas7, Cas8, Cas9 (also known as Csn1 and Csx12), Cas100, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, or Cpf1 endonuclease, or a functional derivative thereof.

### Embodiment 8

The system of any one of embodiments 1-7, wherein said DNA endonuclease is Cas9.

### Embodiment 9

The system of any one of embodiments 1-8, wherein the nucleic acid encoding said DNA endonuclease is codon optimized for expression in a host cell.

### Embodiment 10

The system of any one of embodiments 1-9, wherein the nucleic acid sequence encoding a Factor VIII (FVIII) protein or functional derivative thereof is codon optimized for expression in a host cell.

### Embodiment 11

The system of any one of embodiments 1-10, wherein the nucleic acid encoding said DNA endonuclease is a deoxyribonucleic acid (DNA).

### Embodiment 12

The system of any one of embodiments 1-10, wherein the nucleic acid encoding said DNA endonuclease is a ribonucleic acid (RNA).

### Embodiment 13

The system of embodiment 12, wherein the RNA encoding said DNA endonuclease is an mRNA.

### Embodiment 14

The system of any one of embodiments 1-13, wherein the donor template is encoded in an Adeno Associated Virus (AAV) vector.

### Embodiment 15

The system of embodiment 14, wherein the donor template comprises a donor cassette comprising the nucleic acid sequence encoding a Factor VIII (FVIII) protein or functional derivative, and wherein the donor cassette is flanked on one or both sides by a gRNA target site.

### Embodiment 16

The system of embodiment 15, wherein the donor cassette is flanked on both sides by a gRNA target site.

### Embodiment 17

The system of embodiment 15 or 16, wherein the gRNA target site is a target site for a gRNA in the system.

### Embodiment 18

The system of embodiment 17, wherein the gRNA target site of the donor template is the reverse complement of a genomic gRNA target site for a gRNA in the system.

### Embodiment 19

The system of any one of embodiments 1-18, wherein said DNA endonuclease or nucleic acid encoding the DNA endonuclease is formulated in a liposome or lipid nanoparticle.

### Embodiment 20

The system of embodiment 19, wherein said liposome or lipid nanoparticle also comprises the gRNA.

### Embodiment 21

The system of any one of embodiments 1-20, comprising the DNA endonuclease precomplexed with the gRNA, forming a Ribonucleoprotein (RNP) complex.

### Embodiment 22

A method of editing a genome in a cell, the method comprising providing the following to the cell:
(a) a gRNA comprising a spacer sequence from any one of SEQ ID NOs: 22, 21, 28, 30, 18-20, 23-27, 29, 31-44, and 104;
(b) a DNA endonuclease or nucleic acid encoding said DNA endonuclease; and
(c) a donor template comprising a nucleic acid sequence encoding a Factor VIII (FVIII) protein or functional derivative.

### Embodiment 23

The method of embodiment 22, wherein the gRNA comprises a spacer sequence from any one of SEQ ID NOs: 22, 21, 28, and 30.

### Embodiment 24

The method of embodiment 23, wherein the gRNA comprises a spacer sequence from SEQ ID NO: 21.

### Embodiment 25

The method of embodiment 23, wherein the gRNA comprises a spacer sequence from SEQ ID NO: 22.

### Embodiment 26

The method of embodiment 23, wherein the gRNA comprises a spacer sequence from SEQ ID NO: 28.

### Embodiment 27

The method of embodiment 23, wherein the gRNA comprises a spacer sequence from SEQ ID NO: 30.

### Embodiment 28

The method of any one of embodiments 22-27, wherein said DNA endonuclease is selected from the group consisting of a Cas1, Cas1B, Cas2, Cas3, Cas4, Cas5, Cas6, Cas7, Cas8, Cas9 (also known as Csn1 and Csx12), Cas100, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, or Cpf1 endonuclease; or a functional derivative thereof.

### Embodiment 29

The method of any one of embodiments 22-28, wherein said DNA endonuclease is Cas9.

### Embodiment 30

The method of any one of embodiments 22-29, wherein the nucleic acid encoding said DNA endonuclease is codon optimized for expression in the cell.

### Embodiment 31

The method of any one of embodiments 22-30, wherein the nucleic acid sequence encoding a Factor VIII (FVIII) protein or functional derivative thereof is codon optimized for expression in the cell.

### Embodiment 32

The method of any one of embodiments 22-31, wherein the nucleic acid encoding said DNA endonuclease is a deoxyribonucleic acid (DNA).

### Embodiment 33

The method of any one of embodiments 22-31, wherein the nucleic acid encoding said DNA endonuclease is a ribonucleic acid (RNA).

### Embodiment 34

The method of embodiment 33, wherein the RNA encoding said DNA endonuclease is an mRNA.

### Embodiment 35

The method of any one of embodiments 22-34, wherein the donor template is encoded in an Adeno Associated Virus (AAV) vector.

### Embodiment 36

The method of any one of embodiments 22-35, wherein the donor template comprises a donor cassette comprising the nucleic acid sequence encoding a Factor VIII (FVIII) protein or functional derivative, and wherein the donor cassette is flanked on one or both sides by a gRNA target site.

### Embodiment 37

The method of embodiment 36, wherein the donor cassette is flanked on both sides by a gRNA target site.

### Embodiment 38

The method of embodiment 36 or 37, wherein the gRNA target site is a target site for the gRNA of (a).

### Embodiment 39

The method of embodiment 38, wherein the gRNA target site of the donor template is the reverse complement of a gRNA target site in the cell genome for the gRNA of (a).

### Embodiment 40

The method of any one of embodiments 22-39, wherein said DNA endonuclease or nucleic acid encoding the DNA endonuclease is formulated in a liposome or lipid nanoparticle.

### Embodiment 41

The method of embodiment 40, wherein said liposome or lipid nanoparticle also comprises the gRNA.

### Embodiment 42

The method of any one of embodiments 22-41, comprising providing to the cell the DNA endonuclease precomplexed with the gRNA, forming a Ribonucleoprotein (RNP) complex.

### Embodiment 43

The method of any one of embodiments 22-42, wherein the gRNA of
(a) and the DNA endonuclease or nucleic acid encoding the DNA endonuclease of (b) are provided to the cell more than 4 days after the donor template of (c) is provided to the cell.

### Embodiment 44

The method of any one of embodiments 22-43, wherein the gRNA of (a) and the DNA endonuclease or nucleic acid encoding the DNA endonuclease of (b) are provided to the cell at least 14 days after (c) is provided to the cell.

### Embodiment 45

The method of embodiment 43 or 44, wherein one or more additional doses of the gRNA of (a) and the DNA endonuclease or nucleic acid encoding the DNA endonuclease of (b) are provided to the cell following the first dose of the gRNA of (a) and the DNA endonuclease or nucleic acid encoding the DNA endonuclease of (b).

### Embodiment 46

The method of embodiment 45, wherein one or more additional doses of the gRNA of (a) and the DNA endonuclease or nucleic acid encoding the DNA endonuclease of (b) are provided to the cell following the first dose of the gRNA of (a) and the DNA endonuclease or nucleic acid encoding the DNA endonuclease of (b) until a target level of targeted integration of the nucleic acid sequence encoding a Factor VIII (FVIII) protein or functional derivative and/or a target level of expression of the nucleic acid sequence encoding a Factor VIII (FVIII) protein or functional derivative is achieved.

### Embodiment 47

The method of any one of embodiments 22-46, wherein the nucleic acid sequence encoding a Factor VIII (FVIII) protein or functional derivative is expressed under the control of the endogenous albumin promoter.

### Embodiment 48

The method of any one of embodiments 22-47, wherein said cell is a hepatocyte.

### Embodiment 49

A genetically modified cell in which the genome of the cell is edited by the method of any one of embodiments 22-48.

### Embodiment 50

The genetically modified cell of embodiment 49, wherein the nucleic acid sequence encoding a Factor VIII (FVIII) protein or functional derivative is expressed under the control of the endogenous albumin promoter.

### Embodiment 51

The genetically modified cell of embodiment 49 or 50, wherein the nucleic acid sequence encoding a Factor VIII (FVIII) protein or functional derivative thereof is codon optimized for expression in the cell.

### Embodiment 52

The genetically modified cell of any one of embodiments 49-51, wherein said cell is a hepatocyte.

### Embodiment 53

A method of treating Hemophilia A in a subject, the method comprising providing the following to a cell in the subject:
(a) a gRNA comprising a spacer sequence from any one of SEQ ID NOs: 22, 21, 28, 30, 18-20, 23-27, 29, 31-44, and 104;
(b) a DNA endonuclease or nucleic acid encoding said DNA endonuclease; and
(c) a donor template comprising a nucleic acid sequence encoding a Factor VIII (FVIII) protein or functional derivative.

### Embodiment 54

The method of embodiment 53, wherein the gRNA comprises a spacer sequence from any one of SEQ ID NOs: 22, 21, 28, and 30.

### Embodiment 55

The method of embodiment 54, wherein the gRNA comprises a spacer sequence from SEQ ID NO: 22.

### Embodiment 56

The method of embodiment 54, wherein the gRNA comprises a spacer sequence from SEQ ID NO: 21.

### Embodiment 57

The method of embodiment 54, wherein the gRNA comprises a spacer sequence from SEQ ID NO: 28.

### Embodiment 58

The method of embodiment 54, wherein the gRNA comprises a spacer sequence from SEQ ID NO: 30.

### Embodiment 59

The method of any one of embodiments 53-58, wherein said subject is a patient having or is suspected of having Hemophilia A.

### Embodiment 60

The method of any one of embodiments 53-58, wherein said subject is diagnosed with a risk of Hemophilia A.

### Embodiment 61

The method of any one of embodiments 53-60, wherein said DNA endonuclease is selected from the group consisting of a Cas1, Cas1B, Cas2, Cas3, Cas4, Cas5, Cas6, Cas7, Cas8, Cas9 (also known as Csn1 and Csx12), Cas100, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, or Cpf1 endonuclease; or a functional derivative thereof.

### Embodiment 62

The method of any one of embodiments 53-61, wherein said DNA endonuclease is Cas9.

### Embodiment 63

The method of any one of embodiments 53-62, wherein the nucleic acid encoding said DNA endonuclease is codon optimized for expression in the cell.

### Embodiment 64

The method of any one of embodiments 53-63, wherein the nucleic acid sequence encoding a Factor VIII (FVIII) protein or functional derivative thereof is codon optimized for expression in the cell.

### Embodiment 65

The method of any one of embodiments 53-64, wherein the nucleic acid encoding said DNA endonuclease is a deoxyribonucleic acid (DNA).

### Embodiment 66

The method of any one of embodiments 53-64, wherein the nucleic acid encoding said DNA endonuclease is a ribonucleic acid (RNA).

### Embodiment 67

The method of embodiment 66, wherein the RNA encoding said DNA endonuclease is an mRNA.

### Embodiment 68

The method of any one of embodiments 53-67, wherein one or more of the gRNA of (a), the DNA endonuclease or nucleic acid encoding the DNA endonuclease of (b), and the donor template of (c) are formulated in a liposome or lipid nanoparticle.

### Embodiment 69

The method of any one of embodiments 53-68, wherein the donor template is encoded in an Adeno Associated Virus (AAV) vector.

### Embodiment 70

The method of any one of embodiments 53-69, wherein the donor template comprises a donor cassette comprising the nucleic acid sequence encoding a Factor VIII (FVIII) protein or functional derivative, and wherein the donor cassette is flanked on one or both sides by a gRNA target site.

### Embodiment 71

The method of embodiment 70, wherein the donor cassette is flanked on both sides by a gRNA target site.

### Embodiment 72

The method of embodiment 70 or 71, wherein the gRNA target site is a target site for the gRNA of (a).

### Embodiment 73

The method of embodiment 72, wherein the gRNA target site of the donor template is the reverse complement of the gRNA target site in the cell genome for the gRNA of (a).

### Embodiment 74

The method of any one of embodiments 53-73, wherein providing the donor template to the cell comprises administering the donor template to the subject.

### Embodiment 75

The method of embodiment 74, wherein the administration is via intravenous route.

### Embodiment 76

The method of any one of embodiments 53-75, wherein said DNA endonuclease or nucleic acid encoding the DNA endonuclease is formulated in a liposome or lipid nanoparticle.

### Embodiment 77

The method of embodiment 76, wherein said liposome or lipid nanoparticle also comprises the gRNA.

### Embodiment 78

The method of embodiment 77, wherein providing the gRNA and the DNA endonuclease or nucleic acid encoding the DNA endonuclease to the cell comprises administering the liposome or lipid nanoparticle to the subject.

### Embodiment 79

The method of embodiment 78, wherein the administration is via intravenous route.

### Embodiment 80

The method of any one of embodiments 53-79, comprising providing to the cell the DNA endonuclease pre-complexed with the gRNA, forming a Ribonucleoprotein (RNP) complex.

### Embodiment 81

The method of any one of embodiments 53-80, wherein the gRNA of (a) and the DNA endonuclease or nucleic acid encoding the DNA endonuclease of (b) are provided to the cell more than 4 days after the donor template of (c) is provided to the cell.

### Embodiment 82

The method of any one of embodiments 53-81, wherein the gRNA of (a) and the DNA endonuclease or nucleic acid encoding the DNA endonuclease of (b) are provided to the cell at least 14 days after the donor template of (c) is provided to the cell.

### Embodiment 83

The method of embodiment 81 or 82, wherein one or more additional doses of the gRNA of (a) and the DNA endonuclease or nucleic acid encoding the DNA endonuclease of (b) are provided to the cell following the first dose of the gRNA of (a) and the DNA endonuclease or nucleic acid encoding the DNA endonuclease of (b).

### Embodiment 84

The method of embodiment 83, wherein one or more additional doses of the gRNA of (a) and the DNA endonuclease or nucleic acid encoding the DNA endonuclease of (b) are provided to the cell following the first dose of the gRNA of (a) and the DNA endonuclease or nucleic acid encoding the DNA endonuclease of (b) until a target level of targeted integration of the nucleic acid sequence encoding a Factor VIII (FVIII) protein or functional derivative and/or a target level of expression of the nucleic acid sequence encoding a Factor VIII (FVIII) protein or functional derivative is achieved.

### Embodiment 85

The method of any one of embodiments 81-84, wherein providing the gRNA of (a) and the DNA endonuclease or nucleic acid encoding the DNA endonuclease of (b) to the cell comprises administering to the subject a lipid nanoparticle comprising nucleic acid encoding the DNA endonuclease and the gRNA.

### Embodiment 86

The method of any one of embodiments 81-85, wherein providing the donor template of (c) to the cell comprises administering to the subject the donor template encoded in an AAV vector.

### Embodiment 87

The method of any one of embodiments 53-86, wherein the nucleic acid sequence encoding a Factor VIII (FVIII) protein or functional derivative is expressed under the control of the endogenous albumin promoter.

### Embodiment 88

The method of any one of embodiments 53-87, wherein said cell is a hepatocyte.

### Embodiment 89

The method of any one of embodiments 53-88, wherein the nucleic acid sequence encoding a Factor VIII (FVIII) protein or functional derivative is expressed in the liver of the subject.

### Embodiment 90

A method of treating Hemophilia A in a subject comprising:

administering the genetically modified cell of any one of embodiments 49-52 to the subject.

### Embodiment 91

The method of embodiment 90, wherein said genetically modified cell is autologous to the subject.

### Embodiment 92

The method of embodiment 90 or 91 further comprising:

obtaining a biological sample from the subject wherein the biological sample comprises a hepatocyte cell, wherein the genetically modified cell is prepared from the hepatocyte.

### Embodiment 93

A kit comprising one or more elements of the system of any one of embodiments 1-21, and further comprising instructions for use.

### EXAMPLES

### Example 1: Identification of gRNAs that Direct Cleavage by Cas9 Nuclease in Intron 1 of the Mouse Albumin Gene in Hepa1-6 Cells In Vitro

For purposes of evaluation in relevant pre-clinical animal models, gRNA molecules that direct efficient cleavage by Cas9 nuclease in the intron 1 of albumin from relevant pre-clinical animal species were tested. Mouse models of Hemophilia A are well established (Bi L, Lawler A M, Antonarakis S E, High K A, Gearhart J D, Kazazian H H., Jr Targeted disruption of the mouse factor VIII gene produces a model of hemophilia A. (Nat Genet. 1995; 10:119-21. doi: 10.1038/ng0595-119) and represent a valuable model system for testing new therapeutic approaches for this disease. To identify gRNA with potential to cut in intron 1 of mouse albumin the sequence of the intron was analyzed using algorithms (for example CCTOP; https://crispr.cos.uni-heidelberg.de/) that identify all possible gRNA target sequences utilising a NOG PAM sequence that would be potential targets for cleavage by the Streptococcus pyogenes Cas9 (spCas9) in the sequence of interest, and all related sequences in the mouse genome. Each gRNA was then ranked based on the frequency of exact or related sequences in the mouse genome to identify gRNA with the least theoretical risk of off-target cutting. Based on an analysis of this type a gRNA called mALbgRNA_T1 was selected for testing.

The mAlbgRNA_T1exhibited homology to only 4 other sites in the mouse genome, each of which exhibits 4 nucleotide mismatches as shown in Table 2 below.

TABLE 2
Potential off target sites for gRNA mAlb_T1 in the mouse genome (MM = number of
mismatches)

| Chromo- | | | | | | gene |
|---|---|---|---|---|---|---|
| some | strand | MM | target_seq | Alignment | position | name |
| chr5 | + | 4 | TGCCTTTACCCCATCGTTAC | \|\|\|\|--\|-[\|\|\|-\|\|\|\|\|\|\|\|]PAM | Exonic | Tmprss11g |
| | | | (SEQ ID NO: 7) | | | |
| chr11 | - | 4 | TGCCTCCTCCCGATAGTTAC | \|\|\|\|---\|[\|\|\|\|\|\|-\|\|\|\|\|]PAM | Intronic | Dhrs7c |
| | | | (SEQ ID NO: 8) | | | |
| chr1 | + | 4 | GGACAGTTCCTGATTGTTAC | -\|-\|\|\|\|\|[\|\|-\|\|\|-\|\|\|\|\|]PAM | Exonic | Gm37600 |
| | | | (SEQ ID NO: 9) | | | |
| chrX | + | 4 | TGCCTTTTCCCGATTGTTAA | \|\|\|\|--\|\|[\|\|\|\|\|\|-\|\|\|\|-]PAM | Intergenic | Zfp280c |
| | | | (SEQ ID NO: 10) | | | |

To evaluate the efficiency of mALbgRNA_T1 to promote cleavage by Cas9 in mouse cells, the mouse liver cell derived cell line Hepa1-6 was used. Hepa1-6 cells were cultured in DMEM+10% FBS in a 5% CO₂ incubator. A ribonuclear-protein complex (RNP) composed of the gRNA bound to Streptococcus pyogenes Cas9 (spCas9) protein was pre-formed by mixing 2.4 μl of spCas9 (0.8 μg/μl) and 3 μl of the synthetic gRNA (20 μMolar) and 7 μl of PBS (1:5 spCas9: gRNA ratio) and incubated at room temperature for 10 minutes. For nucleofection the entire vial of SF supplement reagent (Lonza) was added to the SF Nucleofector reagent (Lonza) to prepare the complete nucleofection reagent. For each nucleofection 1×10⁵ Hepa1-6 cells were re-suspended in 20 μl of the complete nucleofection reagent, added to the RNP then transferred to a nucleofection cuvette (16 well strip) that was placed in the 4D nuclefection device (Lonza) and nucleofected using program EH-100. After allowing the cells to rest for 10 mins they were transferred to an appropriately sized plate with fresh complete media. 48 hrs post nucleofection the cells were collected genomic DNA was extracted and purified using the Qiagen DNeasy kit (cat 69506).

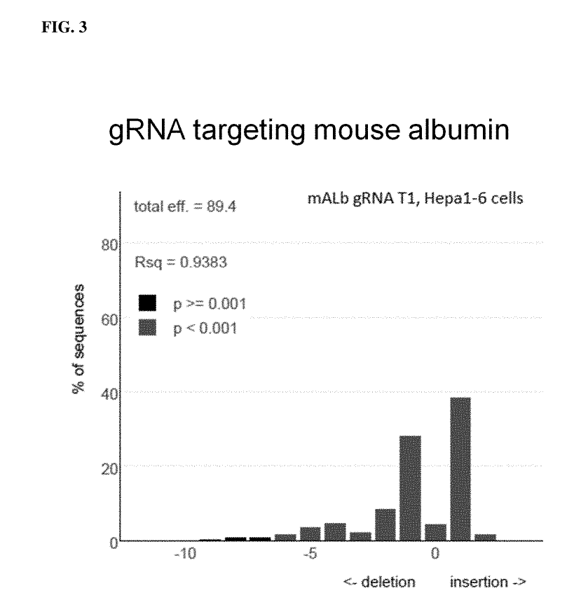

To evaluate the frequency of Cas9/gRNA mediated cutting at the target site in albumin intron 1 a pair of primers (MALBF3; 5′ TTATTACGGTCTCATAGGGC 3′ (SEQ ID NO: 11) and MALBRS: AGTCTTTCTGTCAATGCACAC 3′ (SEQ ID NO: 12)) flanking the target site were used in a polymerase chain reaction (PCR) using a 52° C. annealing temperature to amplify a 609 bp region from the genomic DNA. The PCR product was purified using the Qiagen PCR Purification Kit (Cat no. 28106) and sequenced directly using Sanger sequencing with the same primers used for the PCR reaction. The sequence data was analyzed by an algorithm called Tracking of Indels by Decomposition (TIDES) that determined the frequency of insertions and deletions (INDELS) present at the predicted cut site for the gRNA/Cas9 complex (Brinkman et al (2104); Nucleic Acids Research, 2014, 1). The overall frequency of INDEL generation for mAlbgRNA_T1 was between 85 and 95% when tested in 3 independent experiments indicating efficient cutting by the gRNA/Cas9 in the genome of these cells. An example of TIDES analysis in Hepa1-6 cells nucleofected with the mAlb gRNA-T1 is shown in FIG. 3. Most insertions and deletions consist of 1 bp insertions and 1 bp deletions with smaller numbers of deletions of up to 6 bp.

### Example 2: Evaluation of Cleavage Efficiency of mA1bgRNA_T1 In Vivo in Mice

To deliver Cas9 and the mAlbgRNA-T1 to the hepatocytes of mice a lipid nanoparticle (LNP) delivery vehicle was used. The sgRNA was chemically synthesized incorporating chemically modified nucleotides to improve resistance to nucleases. The gRNA in one example is composed of the following structure: 5′ usgscsCAGUUCCCGAUCGUUACGUUUUAGAgcuaGAAAuagcAAGUUAAAAUAAGGCU AGUCCGUUAUCaacuuGAAAaaguggcaccgagucggugcusususU-3′ (SEQ ID NO: 13), where “A, G, U, C” are native RNA nucleotides, “a, g, u, c” are 2′-O-methyl nucleotides, and “s” represents a phosphorothioate backbone. The mouse albumin targeting sequence of the gRNA is underlined, the remainder of the gRNA sequence is the common scaffold sequence. The spCas9 mRNA was designed to encode the spCas9 protein fused to a nuclear localization domain (NLS) which is required to transport the spCas9 protein in to the nuclear compartment where cleavage of genomic DNA can occur. Additional components of the Cas9 mRNA are a KOZAK sequence at the 5′ end prior to the first codon to promote ribosome binding, and a polyA tail at the 3′ end composed of a series of A residues. An example of the sequence of a spCas9 mRNA with NLS sequences is shown in SEQ ID NO: 81. The mRNA can be produced by different methods well known in the art. One of such methods used herein is in vitro transcription using T7 polymerase in which the sequence of the mRNA is encoded in a plasmid that contains a T7 polymerase promoter. Briefly, upon incubation of the plasmid in an appropriate buffer containing T7 polymerase and ribonucleotides a RNA molecule was produced that encodes the amino acid sequence of the desired protein. Either natural ribonucleotides or chemically modified ribonucleotides in the reaction mixture was used to generate mRNA molecules with either natural chemical structure or with modified chemical structures that may have advantages in terms of expression, stability or immunogenicity. In addition, the sequence of the spCas9 coding sequence was optimized for codon usage by utilizing the most frequently used codon for each amino acid. Additionally, the coding sequence was optimized to remove cryptic ribosome binding sites and upstream open reading frames in order to promote the most efficient translation of the mRNA in to spCas9 protein.

A primary component of the LNP used in these studies is the lipid C12-200 (Love et al (2010), PNAS vol. 107, 1864-1869). The C12-200 lipid forms a complex with the highly-charged RNA molecules. The C12-200 was combined with 1,2-Dioleoyl-sn-glycero-3-phosphoethanolamine (DOPE), DMPE-mPEG2000 and cholesterol. When mixed under controlled conditions for example in a NanoAssemblr device (Precision NanoSystems) with nucleic acids such as gRNA and mRNA, a self-assembly of LNP occurred in which the nucleic acid was encapsulated inside the LNP. To assemble the gRNA and the Cas9 mRNA in the LNP, ethanol and lipid stocks were pipetted into glass vials as appropriate. The ratio of C12-200 to DOPE, DMPE-mPEG2000 and cholesterol was adjusted to optimize the formulation. A typical ratio was composed of C12-200, DOPE, cholesterol and mPEG2000-DMG at a molar ratio of 50:10:38.5:1.5. The gRNA and mRNA were diluted in 100 mM Na Citrate pH 3.0 and 300 mM NaCl in RNase free tubes. The NanoAssemblr cartridge (Precision NanoSystems) was washed with ethanol on the lipid side and with water on the RNA side. The working stock of lipids were pulled into a syringe, air removed from the syringe and inserted in the cartridge. The same procedure was used for loading a syringe with the mixture of gRNA and Cas9 mRNA. The Nanoassemblr run was then performed under standard conditions. The LNP suspension was then dialyzed using a 20 Kd cutoff dialysis cartridges in 4 liters of PBS for 4 h and then concentrated using centrifugation through 20 Kd cutoff spin cartridges (Amicon) including washing three times in PBS during centrifugation. Finally, the LNP suspension was sterile filtered through 0.2 μM syringe filter. Endotoxin levels were checked using commercial endotoxin kit (LAL assay) and particle size distribution was determined by dynamic light scattering. The concentration of encapsulated RNA was determined using a ribogreen assay (Thermo Fisher). Alternatively, the gRNA and the Cas9 mRNA were formulated separately into LNP and then mixed together prior to treatment of cells in culture or injection in to animals. Using separately formulated gRNA and Cas9 mRNA allowed specific ratios of gRNA and Cas9 mRNA to be tested.

Alternative LNP formulations that utilized alternative cationic lipid molecules were also used for in vivo delivery of the gRNA and Cas9 mRNA. Freshly prepared LNP encapsulating the mALB gRNA T1 and Cas9 mRNA were mixed at a 1:1 mass ratio of the RNA and injected in to the tail vein (TV injection) of Hemophilia A mice. Alternatively, the LNP was dosed by retro orbital (RO) injection. The dose of LNP given to mice ranged from 0.5 to 2 mg of RNA per kg of body weight. Three days after injection of the LNP the mice were sacrificed and a piece of the left and right lobes of the liver and a piece of the spleen were collected and genomic DNA was purified from each. The genomic DNA was then subjected to TIDES analysis to measure the cutting frequency and cleavage profile at the target site in albumin intron 1. An example of the results is sown in FIG. 4, where on average 25% of the alleles were cleaved at a dose of 2 mg/kg. A dose response was seen with 0.5 mg/kg dose resulting in about 5% cutting and 1 mg/kg resulting in about 10% cutting. Mice injected with PBS buffer alone showed a low signal of about 1 to 2% in the TIDES assay which is a measure of the background of the TIDES assay itself.

### Example 3: Evaluating Indel Frequencies of SGRNAS Targeted to Intron 1 of Human Albumin

All potential gRNA sequences utilizing a NGG PAM sequence that would be targets for cleavage by the Streptococcus pyogenes Cas9 (spCas9) within intron 1 of the human albumin gene were identified using a proprietary algorithm called “Guido” that is based on the published algorithm called “CCTop” (see, e.g. https://crispr.cos.uni-heidelberg.de/). This algorithm identifies potential off-target sites in the human genome and ranks each gRNA based on predicted off-target cutting potential. The identified gRNA sequences are provided in the table below.

TABLE 3
Human albumin intron 1 gRNA sequences

| gRNA name | gRNA sequence (with PAM) |
|---|---|
| Human Albumin Intron-1_T1 | TAATTTTCTTTTGCGCACTAAGG (SEQ ID NO: 18) |
| Human Albumin Intron-1_T2 | TAGTGCAATGGATAGGTCTTTGG (SEQ ID NO: 19) |
| Human Albumin Intron-1_T3 | AGTGCAATGGATAGGTCTTTGGG (SEQ ID NO: 20) |
| Human Albumin Intron-1_T4 | TAAAGCATAGTGCAATGGATAGG (SEQ DI NO: 21) |
| Human Albumin Intron-1_T5 | ATTTATGAGATCAACAGCACAGG (SEQ ID NO: 22) |
| Human Albumin Intron-1_T6 | TGATTCCTACAGAAAAACTCAGG (SEQ ID NO: 23) |
| Human Albumin Intron-1_T7 | TGTATTTGTGAAGTCTTACAAGG (SEQ ID NO: 24) |
| Human Albumin Intron-1_T8 | GACTGAAACTTCACAGAATAGGG (SEQ ID NO: 25) |
| Human Albumin Intron-1_T9 | AATGCATAATCTAAGTCAAATGG (SEQ ID NO: 26) |
| Human Albumin Intron-1_T10 | TGACTGAAACTTCACAGAATAGG (SEQ ID NO: 27) |
| Human Albumin Intron-1_T11 | TTAAATAAAGCATAGTGCAATGG (SEQ ID NO: 28) |
| Human Albumin Intron-1_T12 | GATCAACAGCACAGGTTTTGTGG (SEQ ID NO: 29) |
| Human Albumin Intron-1_T13 | TAATAAAATTCAAACATCCTAGG (SEQ ID NO: 30) |
| Human Albumin Intron-1_T14 | TTCATTTTAGTCTGTCTTCTTGG (SEQ ID NO: 31) |
| Human Albumin Intron-1_T15 | ATTATCTAAGTTTGAATATAAGG (SEQ ID NO: 32) |
| Human Albumin Intron-1_T16 | ATCATCCTGAGTTTTTCTGTAGG (SEQ ID NO: 33) |
| Human Albumin Intron-1_T17 | GCATCTTTAAAGAATTATTTTGG (SEQ ID NO: 34) |
| Human Albumin Intron-1_T18 | TACTAAAACTTTATTTTACTGGG (SEQ ID NO: 35) |
| Human Albumin Intron-1_T19 | TGAATTATTCTTCTGTTTAAAGG (SEQ ID NO: 36) |
| Human Albumin Intron-1_T20 | AATTTTTAAAATAGTATTCTTGG (SEQ ID NO: 37) |
| Human Albumin Intron-1_T21 | ATGCATTTGTTTCAAAATATTGG (SEQ ID NO: 38) |
| Human Albumin Intron-1_T22 | TTTGGCATTTATTTCTAAAATGG (SEQ ID NO: 39) |
| Human Albumin Intron-1_T23 | AAAGTTGAACAATAGAAAAATGG (SEQ ID NO: 40) |
| Human Albumin Intron-1_T24 | TTACTAAAACTTTATTTTACTGG (SEQ ID NO: 41) |
| Human Albumin Intron-1_T25 | ACCTTTTTTTTTTTTTACCTAGG (SEQ ID NO: 104) |
| Human Albumin Intron-1_T26 | TGCATTTGTTTCAAAATATTGGG (SEQ ID NO: 42) |
| Human Albumin Intron-1_T27 | TGGGCAAGGGAAGAAAAAAAAGG (SEQ ID NO: 43) |
| Human Albumin Intron-1_T28 | TCCTAGGTAAAAAAAAAAAAAGG (SEQ ID NO: 44) |

Cas9 nuclease protein (Platinum™, GeneArt™) at 5 μg/μl was purchased from Thermo Fisher Scientific (catalog number A27865, Carlsbad, Calif.), then diluted 1:6 to a working concentration of 0.83 μg/μl or 5.2 μM. Chemically-modified synthetic single guide RNA (sgRNA) (Synthego Corp, Menlo Park, Calif.) was re-suspended at 100 μM with TE buffer as a stock solution. Alternatively, the gRNA used can be produced by in vitro transcription (IVT). This solution was diluted with nuclease-free water to a working concentration of 20 μM.

To make ribonucleoprotein complexes, Cas9 protein (12.5 pmol) and sgRNA (60 pmol) were incubated for 10-20 minutes at room temperature. During this incubation, HepG2 cells (American Type Culture Collection, Manassas, Va.) or HuH7 Cells (American Type Tissue Culture Collection, Manassas, Va.) were dissociated using Trypsin-EDTA at 0.25% (Thermo Fisher Scientific) for 5 minutes at 37° C. Each transfection reaction contained 1×10⁵ cells, and the appropriate number of cells per experiment were centrifuged at 350×G for 3 minutes, then re-suspended in 20 μl of Lonza SF nucleofection plus supplement solution (catalog number V4XC-2032, Basel, Switzerland) per transfection reaction. Re-suspended cells in 20 μl of nucleofection solution were added to each tube of RNP and the entire volume was transferred to one well of a 16-well nucleofection strip. HepG2 or HuH7 cells were transfected using the EH-100 program on the Amaxa 4D-Nucleofector System (Lonza). HepG2 and HuH7 are human hepatocyte cell lines that are therefore relevant for evaluating gRNA that is be used to cleave a gene in the liver. After transfection, cells were incubated in the nucleofection strip for 10 minutes, transferred into a 48-well plate containing warm medium, consisting of Eagle's Minimum Essential Medium (catalog number 10-009-CV, Corning, Corning, N.Y.) supplemented with 10% fetal bovine serum (catalog number 10438026, Thermo Fisher Scientific). Cells were re-fed with fresh medium the next day.

At 48 hours after transfection, HepG2 or HuH7 cells were dissociated and genomic DNA was extracted using the Qiagen DNeasy kit (catalog number 69506, Hilden, Germany). PCR was performed using extracted genomic DNA with the Platinum SuperFi Green PCR Master Mix (Thermo Fisher Scientific) and the following primers at 0.2 μM: Albumin forward: 5′-CCCTCCGTTTGTCCTAGCTT-3′ (SEQ ID NO: 14); Albumin reverse: 5′-TCTACGAGGCAGCACTGTT-3′ (SEQ ID NO: 15); AAVS1 forward: 5′-AACTGCTTCTCCTCTTGGGAAGT-3′ (SEQ ID NO: 16); AAVS1 reverse: 5′-CCTCTCCATCCTCTTGCTTTCTTTG-3′(SEQ ID NO: 17). PCR conditions were 2 minutes at 98° C. (1×), followed by 30 seconds at 98° C., 30s at 62.5° C. and 1 min at 72° C. (35×). The correct PCR product was confirmed using a 1.2% E-Gel (Thermo Fisher Scientific) and purified using the Qiagen PCR purification kit (catalog number 28106). Purified PCR products were subjected to Sanger sequencing using either the forward or reverse primer for the corresponding PCR product. The frequencies of insertions or deletions at the predicted cleavage site for the gRNA/Cas9 were determined using the TIDE analysis algorithm as described by Brinkman, et al. (Brinkman, E. K., Chen, T., Amendola, M, and van Steensel, B. Easy quantitative assessment of genome editing by sequence trace decomposition. Nucleic Acids Research, 2014, Vol. 42, No. 22 e168). Briefly, the chromatogram sequencing files were compared to a control chromatogram derived from non-treated cells to determine the relative abundance of aberrant nucleotides. The results are summarized in Table 4. It is also of interest to identify gRNA sequences in the human that are homologous in relevant pre-clinical species such as non-human primates. Alignment of the potential gRNA sequences identified in human albumin intron 1 with the albumin intron 1 sequences of the primates Macaca fascicularis and Macaca mulatta identified several gRNA molecules with perfect matches or 1 to 2 nucleotide mis-matches as shown in Table 4. INDEL frequencies generated using IVT guides were measured in HuH7 cells, and INDEL frequencies generated with synthetic guides were measured in HepG2 cells. The INDEL frequencies generated by the different guides in HuH7 cells ranged from 0.3% to 64% demonstrating that a gRNA that efficiently cleaves in intron 1 of albumin could not be selected purely based open a sequence based in silico algorithm. Based on the INDEL frequencies of the IVT gRNA in HuH7 and the synthetic gRNA in HepG2 cells, several gRNA with cleavage frequencies greater than 40% were identified. Of particular interest are gRNA T5 and T12 that exhibited 46% and 43% cutting as synthetic guides, and are 100% identical in human and primate.

TABLE 4
Cleavage efficiencies of sgRNA candidates in human albumin intron 1 and their
homology to primate. sgRNA = synthetic gRNA, IVT gRNA = gRNA made by in vitro
transcription. *Sequence alignment to Macaca fascicularis and Macaca mulatta with up to 2
mismatches in bold and underlined. INDEL data for IVT gRNA N = 1-2; Synthetic sgRNA, N = 2-3

| | | | Indel | Indel | | |
|---|---|---|---|---|---|---|
| | | | frequency- | frequency- | | |
| | | | IVT gRNA | synthetic sgRNA | | |
| sgRNA | Sequence | Strand | (%± SEM) | (%±SEM) | Macaca fascicularis* | Macaca mulatta* |
| T1 | TAATTTTCTTTTGCGCACTAAGG | + | 6.8 ± 1.7 | | TAATTTTCTTTTGCCCACTAAGG | TAATTTTCTTTTGCCCACTAAGG |
| | (SEQ ID NO: 18) | | | | (SEQ ID NO: 45) | (SEQ ID NO: 62) |
| T2 | TAGTGCAATGGATAGGTCTTTGG | - | 9.9 ± 2.2 | | TAGTGCAATGGATAGGTCTTAGG | TAGTGCAATGGATAGGTCTTA |
| | (SEQ ID NO: 19) | | | | (SEQ ID NO: 46) | GG (SEQ ID NO: 63) |
| T3 | AGTGCAATGGATAGGTCTTTGGG | - | 28.6 ± 20.5 | 52.3 ± 5.4 | AGTGCAATGGATAGGTCTTAGGG | AGTGCAATGGATAGGTCTTAG |
| | (SEQ ID NO: 20) | | | | (SEQ ID NO: 47) | GG (SEQ ID NO: 64) |
| T4 | TAAAGCATAGTGCAATGGATAGG | - | 26.3 ± 10.8 | 38.1 ± 11.9 | TAAAGCATAGTGCAATGGATAGG | TAAAGCATAGTGCAATGGATA |
| | (SEQ ID NO: 21) | | | | (SEQ ID NO: 48) | GG (SEQ ID NO: 65) |
| T5 | ATTTATGAGATCAACAGCACAGG | - | 64.4 ± 6.4 | 46.1 ± 3.4 | ATTTATGAGATCAACAGCACAGG | ATTTATGAGATCAACAGCACAG |
| | (SEQ ID NO: 22) | | | | (SEQ ID NO: 49) | G (SEQ ID NO: 66) |
| T6 | TGATTCCTACAGAAAAACTCAGG | + | 43.1 ± 13.3 | 37.7 ± 6.8 | TGATTCCTACAGAAAAAGTCAGG | TGATTCCTACAGAAAAAGTCAG |
| | (SEQ ID NO: 23) | | | | (SEQ ID NO: 50) | G (SEQ ID NO: 67) |
| T7 | TGTATTTGTGAAGTCTTACAAGG | + | 28.5 ± 17.2 | 38.2 ± 0.55 | | |
| | (SEQ ID NO: 24) | | | | | |
| TS | GACTGAAACTTCACAGAATAGGG | + | 45.7 ± 24.6 | 38.5 ± 3.7 | | |
| | (SEQ ID NO: 25) | | | | | |
| T9 | AATGCATAATCTAAGTCAAATGG | + | 5.6 ± 0.1 | | AATGCATAATCTAAGTCAAATGG | AATGCATAATCTAAGTCAAATG |
| | (SEQ ID NO: 26) | | | | (SEQ ID NO: 51) | G (SEQ ID NO: 68) |
| T10 | TGACTGAAACTTCACAGAATAGG | - | 24.4 ± 14.0 | | | |
| | (SEQ ID NO: 27) | | | | | |
| T11 | TTAAATAAAGCATAGTGCAATGG | - | 20.3 ± 9.4 | 61.3 | TTAAATAAAGCATAGTGCAATGG | TTAAATAAAGCATAGTGCAATG |
| | (SEQ ID NO: 28) | | | | (SEQ ID NO: 52) | G (SEQ ID NO: 69) |
| T12 | GATCAACAGCACAGGTTTTGTGG | + | 60.0 ± 0.6 | 43.5 ± 5.9 | ATTTATGAGATCAACAGCACAGG | ATTTATGAGATCAACAGCACAG |
| | (SEQ ID NO: 29) | | | | (SEQ ID NO: 53) | G (SEQ ID NO: 70) |
| T13 | TAATAAAATTCAAACATCCTAGG | + | 13.9 ± 9.3 | 38.5 ± 2.7 | TAATAAAATTCAAACATCCTAGG | TAATAAAATTCAAACATCCTAG |
| | (SEQ ID NO: 30) | | | | (SEQ ID NO: 54) | G (SEQ ID NO: 71) |
| T14 | TTCATTTTAGTCTGTCTTCTTGG | + | 1.1 ± 0.3 | | | |
| | (SEQ ID NO: 31) | | | | | |
| T15 | ATTATCTAAGTTTGAATATAAGG | + | 14.6 ± 3.9 | | | |
| | (SEQ ID NO: 32) | | | | | |
| T16 | ATCATCCTGAGTTTTTCTGTAGG | + | 14.7 ± 6.4 | | ATTATCCTGACTTTTTCTGTAGG | ATTATCCTGACTTTTTCTGTAGG |
| | (SEQ ID NO: 33) | | | | (SEQ ID NO: 55) | (SEQ ID NO: 72) |
| T17 | GCATCTTTAAAGAATTATTTTGG | + | 39.2 ± 25.9 | 22.5 ± 2.7 | | |
| | (SEQ ID NO: 34) | | | | | |
| T18 | TACTAAAACTTTATTTTACTGGG | - | 2.1 ± 0.2 | | TACTAAAACTTTATTTTACTTGG | TACTAAAACTTTATTTTACTTGG |
| | (SEQ ID NO: 35) | | | | (SEQ ID NO: 56) | (SEQ ID NO: 73) |
| T19 | TGAATTATTCTTCTGTTTAAAGG | + | 2.4 ± 1.1 | | TGAATTATTCCTCTGTTTAAAGG | TGAATTATTCCTCTGTTTAAAG |
| | (SEQ ID NO: 36) | | | | (SEQ ID NO: 57) | G (SEQ ID NO: 74) |
| T20 | AATTTTTAAAATAGTATTCTTGG | + | 0.3 | | | |
| | (SEQ ID NO: 37) | | | | | |
| T21 | ATGCATTTGTTTCAAAATATTGG | - | 2.2 ± 0.1 | | ATGCATTTGTTTCAAAATATTGG | ATGCATTTGTTTCAAAATATTG |
| | (SEQ ID NO: 38) | | | | (SEQ ID NO: 58) | G (SEQ ID NO: 75) |
| T22 | TTTGGCATTTATTTCTAAAATGG | + | 1.7 ± 0.6 | | TTTGGCATTTATTTCTAAAATGG | TTTGGCATTTATTTCTAAAATG |
| | (SEQ ID NO: 39) | | | | (SEQ ID NO: 59) | G (SEQ ID NO: 76) |
| T23 | AAAGTTGAACAATAGAAAAATGG | - | 4.7 ± 0.3 | | AAAGTTGAACAATAGAAAAATGG | AAAGTTGAACAATAGAAAAAT |
| | (SEQ ID NO: 40) | | | | (SEQ ID NO: 60) | GG (SEQ ID NO: 77) |
| T24 | TTACTAAAACTTTATTTTACTGG | - | 1.4 ± 0.7 | | | |
| | (SEQ ID NO: 41) | | | | | |
| T26 | TGCATTTGTTTCAAAATATTGGG | - | 3.2 ± 0.0 | | TGCATTTGTTTCAAAATATTGGG | TGCATTTGTTTCAAAATATTGG |
| | (SEQ ID NO: 42) | | | | (SEQ ID NO: 61) | G (SEQ ID NO: 78) |
| T27 | TGGGCAAGGGAAGAAAAAAAAGG | - | 32.0 ± 4.9 | 48.4 ± 13.3 | | TGGGGAAGGGGAGAAAAAAA |
| | (SEQ ID NO: 43) | | | | | AGG (SEQ ID NO: 79) |

### Example 4: Targeted Integration of a Therapeutic Gene of Interest at Mouse Albumin Intron 1

An approach to express a therapeutic protein required to treat a disease is the targeted integration of the cDNA or coding sequence of the gene encoding that protein in to the albumin locus in the liver in vivo. Targeted integration is a process by which a donor DNA template is integrated in to the genome of an organism at the site of a double strand break, such integration occurring either by HDR or NHEJ. This approach uses the introduction into the cells of the organism a sequence specific DNA nuclease and a donor DNA template encoding the therapeutic gene. We evaluated if a CRISPR-Cas9 nuclease targeted to albumin intron 1 was capable of promoting targeted integration of a donor DNA template. The donor DNA template is delivered in an AAV virus, preferably a AAV8 virus in the case of mice, which preferentially transduces the hepatocytes of the liver after intravenous injection. The sequence specific gRNA mAlb_T1 and the Cas9 mRNA are delivered to the hepatocytes of the liver of the same mice by intravenous or RO injection of a LNP formulation encapsulating the gRNA and Cas9 mRNA. In one case the AAV8-donor template is injected in to the mice before the LNP since it is known that transduction of the hepatocytes by AAV takes several hours to days and the delivered donor DNA is stably maintained in the nuclei of the hepatocytes for weeks to months. In contrast the gRNA and mRNA delivered by a LNP will persist in the hepatocytes for only 1 to 4 days due to the inherent instability of RNA molecules. In another case the LNP is injected into the mice between 1 day and 7 days after the AAV-donor template. The donor DNA template incorporates several design features with the goal of (i) maximizing integration and (ii) maximizing expression of the encoded therapeutic protein.

For integration to occur via HDR homology arms need to be included either side of the therapeutic gene cassette. These homology arms are composed of the sequences either side of the gRNA cut site in the mouse albumin intron 1. While longer homology arms generally promote more efficient HDR the length of the homology arms can be limited by the packaging limit for the AAV virus of about 4.7 to 5.0 Kb. Thus, identifying the optimal length of homology arm requires testing. Integration can also occur via NHEJ mechanisms in which the free ends of a double stranded DNA donor are joined to the ends of a double strand break. In this case homology arms are not required. However, incorporating gRNA cut sites either side of the gene cassette can improve the efficiency of integration by generating linear double strand fragments. By using gRNA cleavage sites in the reverse orientation, integration in the desired forward orientation can be favored. Introduction of a mutation in the furin cleavage site of FVIII can generate a FVIII protein that cannot be cleaved by furin during expression of the protein resulting in a one chain FVIII polypeptide that has been shown to have improved stability in the plasma while maintaining full functionality.

Exemplary DNA donors designed to integrate a FVIII gene at albumin intron 1 are shown in FIG. 5. Sequences of specific donor designs are in sequence from SEQ ID NOs: 87-92.

Production of AAV8 or other AAV serotype virus packaged with the FVIII donor DNA is accomplished using well established viral packaging methods. In one such method HEK293 cells are transfected with 3 plasmids, one encoding the AAV packaging proteins, the second encoding Adenovirus helper proteins and the 3^{rd} containing the FVIII donor DNA sequence flanked by AAV ITR sequences. The transfected cells give rise to AAV particles of the serotype specified by the composition of the AAV capsid proteins encoded on the first plasmid. These AAV particles are collected from the cell supernatant or the supernatant and the lysed cells and purified over a CsCl gradient or an Iodixanol gradient or by other methods as desired. The purified viral particles are quantified by measuring the number of genome copies of the donor DNA by quantitative PCR (Q-PCR).

In vivo delivery of the gRNA and the Cas9 mRNA are accomplished by various methods. In the first case, the gRNA and Cas9 protein are expressed from an AAV viral vector. In this case the transcription of the gRNA is driven off a U6 promoter and the Cas9 mRNA transcription is driven from either a ubiquitous promoter like EF1-alpha or preferably a liver specific promoter and enhancer such as the transthyretin promoter/enhancer. The size of the spCas9 gene (4.4 Kb) precludes inclusion of the spCas9 and the gRNA cassettes in a single AAV, thereby requiring separate AAV to deliver the gRNA and spCas9. In a second case, an AAV vector that has sequence elements that promote self-inactivation of the viral genome is used. In this case, including cleavage sites for the gRNA in the vector DNA results in cleavage of the vector DNA in vivo. By including cleavage sites in locations that blocks expression of the Cas9 when cleaved, Cas9 expression is limited to a shorter time period. In the third, alternative approach to deliver the gRNA and Cas9 to cells in vivo, a non-viral delivery method is used. In one example, lipid nanoparticles (LNP) are used as a non-viral delivery method. Several different ionizable cationic lipids are available for use in LNP. These include C12-200 (Love et al (2010), PNAS vol. 107, 1864-1869), MC3, LN16, MD1 among others. In one type of LNP a GalNac moiety is attached to the outside of the LNP and acts as a ligand for uptake in to the liver via the asialyloglycoprotein receptor. Any of these cationic lipids are used to formulate LNP for delivery of gRNA and Cas9 mRNA to the liver.

To evaluate targeted integration and expression of FVIII, Hemophilia A mice are first injected intravenously with a AAV virus, preferentially a AAV8 virus that encapsulates the FVIII donor DNA template. The dose of AAV ranges from 10¹⁰ to 10¹² vector genomes (VG) per mouse equivalent to 4×10¹¹ to 4×10¹³ VG/kg. Between 1 h and 7 days after injection of the AAV-donor the same mice are given iv injections of a LNP encapsulating the gRNA and the Cas9 mRNA. The Cas9 mRNA and gRNA are encapsulated in to separate LNP and then mixed prior to injection at a RNA mass ratio of 1:1. The dose of LNP given ranges from 0.25 to 2 mg of RNA per kg of body weight. The LNP is dosed by tail vein injection or by retroorbital injection. The impact of the time of LNP injection relative to AAV injection upon the efficiency of targeted integration and FVIII protein expression is evaluated by testing times of 1 hr, 24 h, 48 h, 72 h, 96 h, 120 h, 144 h and 168 h after AAV dosing.

In another example, the donor DNA template is delivered in vivo using a non-viral delivery system which is an LNP. DNA molecules are encapsulated in to similar LNP particles as those described above and delivered to the hepatocytes in the liver after iv injection. While escape of the DNA from the endosome to the cytoplasm occurs relatively efficiently, translocation of large charged DNA molecules into the nucleus is not efficient. In one case the way to improve the delivery of DNA to the nucleus is mimicing the AAV genome by incorporation of the AAV ITR in to the donor DNA template. In this case, the ITR sequences stabilize the DNA or otherwise improve nuclear translocation. The removal of CG dinucleotides (CpG sequences) form the donor DNA template sequence also improves nuclear delivery. DNA containing CG dinucleotides is recognized by the innate immune system and eliminated. Removal of CpG sequences that are present in artificial DNA sequences improves the persistence of DNA delivered by non-viral and viral vectors. The process of codon optimization typically increases the content of CG dinucleotides because the most frequent codons in many cases have a C residue in the 3^{rd} position which increases the chance of creating a CG when the next codon starts with a G. A combination of LNP delivery of the donor DNA template followed 1 h to 5 days later with a LNP containing the gRNA and Cas9 mRNA is evaluated in Hemophilia A mice

To evaluate the effectiveness of in vivo delivery of gRNA/Cas9 and donor DNA templates the injected Hemophilia mice are evaluated for FVIII levels in the blood at different times starting about 7 days after dosing the second component. Blood samples are collected by RO bleeding and the plasma is separated and assayed for FVIII activity using a chromogenic assay (Diapharma). FVIII protein standards are used to calibrate the assay and calculate the units per ml of FVIII activity in the blood.

The expression of FVIII mRNA is also measured in the livers of the mice at the end of the study. Total RNA extracted from the livers of the mice is assayed for the levels of albumin mRNA and FVIII mRNA using Q-PCR. The ratio of FVIII mRNA to albumin mRNA when compared to untreated mice is an indication of the % of albumin transcripts that have been co-opted to produce a hybrid albumin-FVIII mRNA.

The genomic DNA from the livers of treated mice is evaluated for targeted integration events at the target site of the gRNA, specifically in albumin intron 1. PCR primers pairs are designed to amplify the junction fragments at either end of the predicted targeted integration. These primers are designed to detect integration in both the forward and reverse orientations. Sequencing of the PCR products confirms if the expected integration event has occurred. To quantify the percentage of albumin alleles that have undergone targeted integration a standard is synthesized that corresponds to the expected junction fragments. When spiked in to genomic DNA from untreated mice at different concentrations and then subjected to the same PCR reaction a standard curve is generated and used to calculate the copy number of alleles with integration events in the samples from treated mice.

### Example 5: Targeted Integration in to Primate Albumin Intron 1

The same methodologies described in Example 4 for the mouse are applied to primate species using a gRNA that targets albumin intron 1 of the primate. Either AAV8 or a LNP is used to first deliver the donor DNA template by iv injection. The doses used are based upon those found to be successful in the mouse. Subsequently the same primates are given iv injections of LNP encapsulating the gRNA and Cas9 mRNA. The same LNP formulation and doses found to be effective in the mice are used. Because a Hemophilia model of primates does not exist, FVIII protein needs to be measured using a human FVIII specific ELISA assay. The same molecular analyses of targeted integration and FVIII mRNA levels described in Example 4 are performed in the primate. The primate is a good pre-clinical model to enable translational to clinical evaluation.

### Example 6: Evaluation of on and Off-Target Cleavage by GRNA/Cas9 and Targeted Integration in Human Primary Hepatocytes

Primary human hepatocytes are the most relevant cell type for evaluation of potency and off-target cleavage of a gRNA/Cas9 that will be delivered to the liver of patients. These cells are grown in culture as adherent monolayers for a limited duration. Methods have been established for transfection of adherent cells with mRNA, for example Message Max (Thermo Fisher). After transfection with a mixture of Cas9 mRNA and gRNA the on-target cleavage efficiency is measured using TIDES analysis. The same samples of genomic DNA are subjected to off-target analysis to identify additional sites in the genome that were cleaved by the gRNA/Cas9 complex. One such method is “GuideSeq” (Tsai et al Nat Biotechnol. 2015 February; 33(2):187-197). Other methods include deep sequencing, whole genome Sequencing, ChIP-seq (Nature Biotechnology 32677-083 2014), BLESS (2013 Crosetto et al. doi:10.1038/nmeth.2408), high-throughput, genome-wide, translocation sequencing (HTGTS) as described in 2015 Frock et al. doi:10.1038/nbt.3101, Digenome-seq (2015 Kim et al. doi:10.1038/nmeth.3284), and IDLY (2014 Wang et al. doi:10.1038/nbt.3127).

Primary human hepatocytes are also transduced by AAV viruses containing the donor DNA template. In particular, AAV6 or AAVDJ serotypes are particularly efficient at transducing cells in culture. Between 1 and 48 h after transduction by the AAV-DNA donor, the cells are then transfected with the gRNA and Cas9 mRNA to induce targeted integration. Targeted integration events are measured using the same PCR based approaches described in Example 4.

### Example 7: Identification and Selection of Guide RNA that Cleave Efficiently at Human Albumin Intron 1 in Primary Human Hepatocytes in Culture

Four gRNA (T4, T5, T11, T13) were selected, based on having perfect homology to the non-human primate and the screening for cutting efficiency in HuH7 and HepG2 cells (Table 4), for evaluation of cutting efficiency in primary human hepatocytes. Primary human hepatocytes (obtained from BioIVT) were thawed, transferred to Cryopreserved Hepatocyte Recovery Medium (CHRM) (Gibco), pelleted at low speed then plated in InVitroGRO™ CP Medium (BioIVT) plus Torpedo™ Antibiotic Mix (BioIVT) at a density of 0.7×10⁶ cells/ml in 24-well plates pre-coated with Collagen IV (Corning). Plates were incubated in 5% CO2 at 37° C. After the cells have adhered (3-4 hours after plating) dead cells that have not adhered to the plate were washed out with fresh warm complete medium was added then cells were incubated in 5% CO2 at 37° C. To transfect the cells, Cas9 mRNA (Trilink) and guide RNA (Synthego Corp, Menlo Park, Calif.) were thawed on ice then added to 30 ul OptiMem media (Gibco) at 0.6 ug mRNA and 0.2 ug guide per well. MessengerMax (ThermoFisher) diluted in 30 ul in OptiMem at a 2:1 volume to total nucleic acid weight was incubated with the Cas9 mRNA/gRNA OptiMem solution at room temperature for 20 minutes. This mixture was added dropwise to the 500 ul of hepatocyte plating medium per well of cultured hepatocytes in a 24-well plate and the cells incubated in 5% CO2 at 37° C. The cells were washed and re-fed the next morning and 48 h post transfection cells were collected for genomic DNA extraction by adding 200 ul of warm 0.25% Trypsin-EDTA (Gibco) to each well and incubating 5-10 minutes at 37° C. Once cells were dislodged, 200 ul FBS (Gibco) was added to inactivate trypsin. After adding to 1 ml PBS (Gibco) the cells were pelleted at 1200 rpm for 3 minutes then resuspended in 50 ul PBS. Genomic DNA was extracted using the MagMAX DNA Multi-Sample Ultra 2.0 Kit (Applied Biosytems) following the instructions in the kit. The genomic DNA quality and concentration was analyzed using a spectrophotometer. For TIDE analysis the genomic DNA was PCR amplified using primers flanking the predicted on-target cleavage site (AlbF: CCCTCCGTTTGTCCTAGCTTTTC, SEQ ID NO: 178, and AlbR: CCAGATACAGAATATCTTCCTCAACGCAGA, SEQ ID NO: 179) and Platinum PCR SuperMix High Fidelity (Invitrogen) using 35 cycles of PCR and an annealing temperature of 55° C. PCR products were first analyzed by agarose gel electrophoresis to confirm that the right sized product (1053 bp) had been generated then purified and sequenced using primers (For: CCTTTGGCACAATGAAGTGG, SEQ ID NO: 180, rev: GAATCTGAACCCTGATGACAAG, SEQ ID NO: 181). Sequence data was then analyzed using a modified version of the TIDES algorithm (Brinkman et al (2104); Nucleic Acids Research, 2014, 1) called Tsunami. This determines the frequency of insertions and deletions (INDELS) present at the predicted cut site for the gRNA/Cas9 complex.

Guide RNA containing either the standard 20 nucleotide target sequence or a 19 nucleotide target sequence (1 bp shorter at the 5′ end) of the T4, T5, T11, and T13 guides (chemically synthesized at AxoLabs, Kulmbach Germany, or Synthego Corp, Menlo Park, Calif.) were tested. A 19 nucleotide gRNA may be more sequence specific but a shorter guide may have lower potency. Control guides targeting human AAVS1 locus and human complement factor were included for comparison across donors. INDEL frequency at the target site in albumin intron 1 was measured 48 h after transfection using the TIDES method. FIG. 6 summarizes the results from transfections of primary hepatocyte from 4 different human donors. The results demonstrate cutting efficiencies ranging from to 20% to 80% for the different guides. The 20 nucleotide version of each albumin gRNA was consistently more potent than the 19 nucleotide variant. The superior potency of the 20 nucleotide gRNA may off-set any potential benefit a 19 nucleotide gRNA may have in terms of off-target cutting. Guide RNA T4 exhibited the most consistent cutting across the 4 cell donors with INDEL frequencies of about 60%. The gRNA T4, T5, T11 and T13 were selected for off-target analysis.

### Example 8: Identification of Off-Target Sites for Human Albumin Guide RNA

Two approaches for identification of off-target sites for CRISPR/Cas9 are ab initio prediction and empirical detection. Specification of the Cas9 cleavage site by the guide RNA is an imperfect process as Cas9 cleavage tolerates mismatching between the guide RNA sequence and the genome. It is important to know the spectrum of Cas9 cleavage sites to understand the safety risk of different guides and select guides with the most favorable off-target profile. The predictive method is based on Guido, a software tool adapted from the CCTop algorithm for off-target prediction (Stemmer et al., 2015). Guido uses the Bowtie 1 algorithm to identify potential off-target cleavage sites by searching for homology between the guide RNA and the entire GRCh38/hg38 build of the human genome (Langmead et al., 2009). Guido detects sequences with up to 5 mismatches to the guide RNA, prioritizing PAM-proximal homology and a correctly positioned NGG PAM. Sites were ranked by the number and position of their mismatches. For each run, the guide sequence as well as the genomic PAM are concatenated and run with default parameters. Top hits with three or fewer mismatches are shown in Tables 5-8 below for the albumin guides T4, T5, T11 and T13. The first line in each table shows the on-target site in the human genome, the lines below that show the predicted off-target sites.

TABLE 5
Guido predicted off target sites for hALB T4

| | | | | Mis- | | |
|---|---|---|---|---|---|---|
| Chr. | Position | Gene | Type | matches | Sequence | PAM |
| 4 | 73404720 | ALB | Intronic | 0 | TAAAGCATAGTGCAATGGAT | AGG |
| | | | | | (SEQ ID NO: 106) | |
| 1 | 105184629 | | Intergenic | 2 | GAAAGCATGGTGCAATGGAT | TGG |
| | | | | | (SEQ ID NO: 107) | |
| 20 | 51270388 | | Intergenic | 3 | TATTGCACAGTGCAATGGAT | GGG |
| | | | | | (SEQ ID NO: 108) | |
| 4 | 30923943 | PCDH7 | Intronic | 3 | TGATGCATATTGCAATGGAT | TGG |
| | | | | | (SEQ ID NO: 109) | |
| 1 | 58844572 | RP11- | Intronic | 3 | TAATGAATAGGGCAATGGAT | TGG |
| | | 63G10.2 | | | (SEQ ID NO: 110) | |
| 1 | 107412556 | NTNG1 | Intronic | 3 | TAAGGCACAGTGTAATGGAT | TGG |
| | | | | | (SEQ ID NO: 111) | |
| 8 | 10123839 | MSRA | Intronic | 3 | AAAAGCATAGACCAATGGAT | TGG |
| | | | | | (SEQ ID NO: 112) | |
| Y | 10935087 | | Intergenic | 3 | TAGAGTATAGTGCAGTGGAT | TGG |
| | | | | | (SEQ ID NO: 113) | |
| X | 21813781 | | Intergenic | 3 | CAAAGCAAAGTGCAATTGAT | GGG |
| | | | | | (SEQ ID NO: 114) | |
| 3 | 31414024 | | Intergenic | 3 | GGAAGCATAGTGCAATGGTT | GGG |
| | | | | | (SEQ ID NO: 115) | |
| 2 | 177957869 | AC011998.1 | Intronic | 3 | TAAAGGATAGAGCAATGTAT | AGG |
| | | | | | (SEQ ID NO: 116) | |
| Y | 10775325 | | Intergenic | 3 | TAGAGTATAGTGCAATGGAG | TGG |
| | | | | | (SEQ ID NO: 117) | |
| 8 | 116113757 | LINC00536 | Intronic | 3 | TAAAGAATAGTGAAATGGTT | TGG |
| | | | | | (SEQ ID NO: 118) | |

TABLE 6
Guido predicted off target sites for hALB T5

| | | | | Mis- | | |
|---|---|---|---|---|---|---|
| Chr. | Position | Gene | Type | matches | Sequence | PAM |
| 4 | 73404759 | ALB | Intronic | 0 | ATTTATGAGATCAACAGCAC | AGG |
| | | | | | (SEQ ID NO: 119) | |
| 19 | 31798902 | | Intergenic | 2 | ATTTATGATATCATCAGCAC | CGG |
| | | | | | (SEQ ID NO: 120) | |
| 11 | 98512684 | | Intergenic | 3 | AAATATGACATCAACAGCAC | AGG |
| | | | | | (SEQ ID NO: 121) | |
| 17 | 12093264 | MAP2K4 | Intronic | 3 | ATCTTTGAGATCATCAGCAC | TGG |
| | | | | | (SEQ ID NO: 122) | |
| 21 | 35820764 | RUNX1 | Intronic | 3 | ATGTATCAGATCATCAGCAC | GGG |
| | | | | | (SEQ ID NO: 123) | |
| 19 | 29334372 | CTC- | Intronic | 3 | AATTATGAGATTCACAGCAC | AGG |
| | | 525D6.1 | | | (SEQ ID NO: 124) | |
| 2 | 116633233 | | Intergenic | 3 | ATTTATGTGTTCAACCGCAC | AGG |
| | | | | | (SEQ ID NO: 125) | |
| 9 | 90654432 | | Intergenic | 3 | ATATATGACATCAACAGAAC | AGG |
| | | | | | (SEQ ID NO: 126) | |
| 6 | 17047800 | | Intergenic | 3 | ACTTATGATATCAACAGCAT | TGG |
| | | | | | (SEQ ID NO: 127) | |

TABLE 7
Guido predicted off target sites for hALB T11

| | | | | Mis- | | |
|---|---|---|---|---|---|---|
| Chr. | Position | Gene | Type | matches | Sequence | PAM |
| 4 | 73404725 | ALB | Intronic | 0 | TTAAATAAAGCATAGTGCAA | TGG |
| | | | | | (SEQ ID NO: 128) | |
| 2 | 229867834 | TRIP12 | Intronic | 1 | TAAAATAAAGCATAGTGCAA | AGG |
| | | | | | (SEQ ID NO: 129) | |
| 14 | 91174270 | C14orf159 | Intronic | 2 | TTAAATAAAGGATATTGCAA | AGG |
| | | | | | (SEQ ID NO: 130) | |
| 16 | 73177850 | | Intergenic | 2 | TTAAATAAAGCATTGAGCAA | GGG |
| | | | | | (SEQ ID NO: 131) | |
| 4 | 1839915 | LETM1 | Intronic | 3 | TACTATAAAGCATAGTGCAA | AGG |
| | | | | | (SEQ ID NO: 132) | |
| 4 | 82950298 | LIN54 | Intronic | 3 | TACTATAAAGCATAGTGCAA | GGG |
| | | | | | (SEQ ID NO: 133) | |
| 3 | 133084865 | TMEM108 | Intronic | 3 | TTAAGGAAACCATAGTGCAA | AGG |
| | | | | | (SEQ ID NO: 134) | |
| 8 | 5026909 | | Intergenic | 3 | ATAAATATATCATAGTGCAA | AGG |
| | | | | | (SEQ ID NO: 135) | |
| 8 | 59960346 | | Intergenic | 3 | CTAAATAGAGAATAGTGCAA | TGG |
| | | | | | (SEQ ID NO: 136) | |
| 21 | 18677763 | MIR548X | Intronic | 3 | TTAAAGAAATTATAGTGCAA | GGG |
| | | | | | (SEQ ID NO: 137) | |
| X | 66550751 | | Intergenic | 3 | TTAAATATATAATAGTGCAA | GGG |
| | | | | | (SEQ ID NO: 138) | |
| X | 109390455 | GUCY2F | Intronic | 3 | TTAAAAACAGCACAGTGCAA | AGG |
| | | | | | (SEQ ID NO: 139) | |
| 10 | 20767685 | | Intergenic | 3 | TTAAAATAAGCATGGTGCAA | GGG |
| | | | | | (SEQ ID NO: 140) | |
| 15 | 54261380 | UNC13C | Intronic | 3 | TTTGATAAAGCATAGGGCAA | TGG |
| | | | | | (SEQ ID NO: 141) | |
| 1 | 230563372 | | Intergenic | 3 | TTTTATAAAGCATAGTCCAA | AGG |
| | | | | | (SEQ ID NO: 142) | |
| 15 | 56985313 | TCF12 | Intronic | 3 | TTAAATGAAGAATATTGCAA | AGG |
| | | | | | (SEQ ID NO: 143) | |
| 3 | 153332862 | | Intergenic | 3 | ATAAATAAAGAATAGAGCAA | GGG |
| | | | | | (SEQ ID NO: 144) | |
| 14 | 31932077 | | Intergenic | 3 | TTGAATAAAGCAGAGTGGAA | GGG |
| | | | | | (SEQ ID NO: 145) | |
| 12 | 38399588 | | Intergenic | 3 | TTAATTAATGCATAGTGCCA | GGG |
| | | | | | (SEQ ID NO: 146) | |
| 7 | 141092721 | TMEM178B | Intronic | 3 | TTAGATAAAGCTTAGTGCTA | AGG |
| | | | | | (SEQ ID NO: 147) | |
| 4 | 60292980 | | Intergenic | 3 | TTAGATAAAGCATACTGGAA | TGG |
| | | | | | (SEQ ID NO: 148) | |
| 2 | 155632685 | | Intergenic | 3 | TTAAAGAAAGCATGGTGCAG | TGG |
| | | | | | (SEQ ID NO: 149) | |
| 8 | 19144500 | RP11- | Intronic | 3 | TTACATAAAGCATACTGCAT | GGG |
| | | 1080G15.2 | | | (SEQ ID NO: 150) | |
| 22 | 44584358 | | Intergenic | 3 | TTATATAAAGCATAGAGCAG | GGG |
| | | | | | (SEQ ID NO: 151) | |
| 20 | 47604347 | NCOA3 | Intronic | 3 | TTAAATGAAGCATAGTGAAG | AGG |
| | | | | | (SEQ ID NO: 152) | |

TABLE 8
Guido predicted off target sites for hALB T13

| | | | | Mis- | | |
|---|---|---|---|---|---|---|
| Chr. | Position | Gene | Type | matches | Sequence | PAM |
| 4 | 73404562 | ALB | Intronic | 0 | TAATAAAATTCAAACATCCT | AGG |
| | | | | | (SEQ ID NO: 153) | |
| 10 | 33567530 | | Intergenic | 2 | GAATAAAATTCTAACATCCT | TGG |
| | | | | | (SEQ ID NO: 154) | |
| 2 | 53855928 | GPR75 | Intronic | 2 | TAATATAATTCCAACATCCT | TGG |
| | | | | | (SEQ ID NO: 155) | |
| 10 | 7439135 | | Intergenic | 2 | AAATAAAATTCAAACTTCCT | TGG |
| | | | | | (SEQ ID NO: 156) | |
| 11 | 106969296 | GUCY1A2 | Intronic | 3 | GAGTTAAATTCAAACATCCT | GGG |
| | | | | | (SEQ ID NO: 157) | |
| 14 | 52353218 | | Intergenic | 3 | TTTTAAAAATCAAACATCCT | GGG |
| | | | | | (SEQ ID NO: 158) | |
| 3 | 25222362 | RARB | Intronic | 3 | AAATGAAAGTCAAACATCCT | TGG |
| | | | | | (SEQ ID NO: 159) | |
| 18 | 29352071 | CTD- | Intronic | 3 | GATTAAAATTTAAACATCCT | TGG |
| | | 2515C13.2 | | | (SEQ ID NO: 160) | |
| 1 | 48069696 | RP4- | Intronic | 3 | TCTTAAAATTCCAACATCCT | AGG |
| | | 683M8.2 | | | (SEQ ID NO: 161) | |
| 20 | 22206955 | | Intergenic | 3 | AAAAAAAATTCCAACATCCT | TGG |
| | | | | | (SEQ ID NO: 162) | |
| 2 | 145716708 | | Intergenic | 3 | TACTGAAATTCTAACATCCT | AGG |
| | | | | | (SEQ ID NO: 163) | |
| 4 | 135277467 | | Intergenic | 3 | TACAAAAATTCACACATCCT | GGG |
| | | | | | (SEQ ID NO: 164) | |
| 2 | 114502757 | Clostridiales-1 | Intronic | 3 | TATTAGAATTCAGACATCCT | TGG |
| | | | | | (SEQ ID NO: 165) | |
| 12 | 65459700 | MSRB3 | Intronic | 3 | TAATAAAGCCCAAACATCCT | AGG |
| | | | | | (SEQ ID NO: 166) | |
| 6 | 6201132 | F13A1 | Intronic | 3 | TATTTAAATTCAAATATCCT | TGG |
| | | | | | (SEQ ID NO: 167) | |
| 2 | 213458045 | SPAG16 | Intronic | 3 | AAATAAAGTTCAAAGATCCT | GGG |
| | | | | | (SEQ ID NO: 168) | |
| 5 | 4307376 | | Intergenic | 3 | TACAAAAATTCAAACTTCCT | TGG |
| | | | | | (SEQ ID NO: 169) | |
| 2 | 201942075 | | Intergenic | 3 | GAATAAAATTTAAATATCCT | AGG |
| | | | | | (SEQ ID NO: 170) | |
| 8 | 141400355 | CTD- | Intronic | 3 | TATAAAAATTCAAACAGCCT | GGG |
| | | 3064M3.4 | | | (SEQ ID NO: 171) | |
| 14 | 39294628 | CTAGE5 | Intronic | 3 | TACTAAAATTTAAACTTCCT | GGG |
| | | | | | (SEQ ID NO: 172) | |
| 14 | 72580607 | RP3- | Intronic | 3 | TAATAACCTTCAAACATTCT | TGG |
| | | 514A23.2 | | | (SEQ ID NO: 173) | |
| 12 | 3628277 | CRACR2A | Intronic | 3 | TAGTAAAATTCAAATGTCCT | AGG |
| | | | | | (SEQ ID NO: 174) | |
| 21 | 42611948 | AP001626.1 | Intronic | 3 | CAATAAAATTCAACCATCAT | GGG |
| | | | | | (SEQ ID NO: 175) | |
| 7 | 16480457 | GS1- | Intronic | 3 | GAATAAAATTCAAACTTCTT | TGG |
| | | 166A23.1 | | | (SEQ ID NO: 176) | |
| 12 | 108648894 | CORO1C | Intronic | 3 | AAATAAAATTCAAAAATCCC | AGG |
| | | | | | (SEQ ID NO: 177) | |

In addition, off-target sites for human albumin gRNA T4, T5, T11, T13 in human liver cells were identified using a method called GUIDE-seq. GUIDE-seq (Tsai et al. 2015) is an empirical method to find off-target cleavage sites. GUIDE-seq relies on the spontaneous capture of an oligonucleotide at the site of a double-strand break in chromosomal DNA. In brief, following transfection of relevant cells with the gRNA/Cas9 complex and double stranded oligonucleotide genomic DNA is purified from the cells, sonicated and a series of adapter ligations performed to create a library. The oligonucleotide-containing libraries are subjected to high-throughput DNA sequencing and the output processed with the default GUIDE-seq software to identify site of oligonucleotide capture.

In detail, the double stranded GUIDEseq oligo was generated by annealing two complementary single stranded oligonucleotides by heating to 89° C. then cooling slowly to room temperature. Ribonuclear protein complexes (RNP) were prepared by mixing 240 pmol of guide RNA (Synthego Corp, Menlo Park, Calif.) and 48 pmol of 20 uMolar Cas9 TruCut (ThermoFisher Scientific) in a final volume of 4.8 uL. In a separate tube 4 ul of the 10 uMolar GUIDeseq double stranded oligonucleotide was mixed with 1.2 ul of the RNP mix then added to a Nucleofection cassette (Lonza). To this was added 16.4 ul of Nucleofector SF solution (Lonza) and 3.6 ul of Supplement (Lonza). HepG2 cells grown as adherent cultures were treated with trypsin to release them from the plate then after deactivation of the trypsin were pelleted and resuspended at 12.5 e6 cells/ml in Nucleofector solution and 20 ul (2.5 e5 cells) added to each nucleofection cuvette. Nucleofection was performed with the EH-100 cell program in the 4-D Nucleofector Unit (Lonza). After incubation at room temperature for 10 minutes 80-ul of complete HepG2 media was added and the cell suspension placed in a well of a 24 well plate and incubated at 37° C. in 5% CO₂ for 48 hours. The cells were released with trypsin, pelleted by centrifugation (300 g 10 mins) then genomic DNA was extracted using the DNAeasy Blood and Tissue Kit (Qiagen). The human Albumin intron 1 region was PCR amplified using primers AlbF (CCCTCCGTTTGTCCTAGCTTTTC, SEQ ID NO: 178) and AlbR (CCAGATACAGAATATCTTCCTCAACGCAGA, SEQ ID NO: 179) and Platinum PCR SuperMix High Fidelity (Invitrogen) using 35 cycles of PC and an annealing temperature of 55° C. PCR products were first analyzed by agarose gel electrophoresis to confirm that the right sized product (1053 bp) had been generated then directly sequenced using primers (For: CCTTTGGCACAATGAAGTGG, SEQ ID NO: 180, rev: GAATCTGAACCCTGATGACAAG, SEQ ID NO: 181). Sequence data was then analyzed using a modified version of the TIDES algorithm (Brinkman et al (2104); Nucleic Acids Research, 2014, 1) called Tsunami. This determines the frequency of insertions and deletions (INDELS) present at the predicted cut site for the gRNA/Cas9 complex. Compared to the protocol described by Tsai et al. we performed GUIDE-seq with 40 pmol (˜1.67 μM) capture oligonucleotide to increase the sensitivity of off-target cleavage site identification. In order to achieve a sensitivity of approximately 0.01% we defined a minimum of 10,000 unique on-target sequence reads per transfection with a minimum of 50% on-target cleavage. Samples without transfection of RNPs were processed in parallel. Sites (+/−1 kb) found in both RNP-containing and RNP-naive samples are excluded from further analysis.

GUIDE-seq was performed in the human hepatoma cell line HepG2. In HepG2 the capture of the GUIDE-seq oligonucleotide at the on-target sites was in the range of 70%-200% of the NHEJ frequency demonstrating efficient oligo capture.

The Y-adapter was prepared by annealing the Common Adapter to each of the sample barcode adapters (A01-A16) that contain the 8-mer molecular index. Genomic DNA extracted from the HepG2 cells that had been nucleofected with RNP and the GUIDEDseq oligo were quantified using Qubit and all samples normalized to 400 ng in 120 uL volume TE Buffer. The genomic DNA was sheared to an average length of 200 bp according to the standard operating procedure for the Covaris 5220 sonicator. To confirm average fragment length, 1 uL of the sample was analyzed on a TapeStation according to manufacturer protocol. Samples of sheared DNA were cleaned up using AMPure XP SPRI beads according to manufacturer protocol and eluted in 17 uL of TE Buffer. The end repair reaction was performed on the genomic DNA by mixing 1.2 ul of dNTP mix (5 mM each dNTP), 3 ul of 10×T4 DNA Ligase Buffer, 2.4 ul of End-Repair Mix, 2.4 ul of 10× Platinum Taq Buffer (Mg2+ free), and 0.6 ul of Taq Polymerase (non-hotstart) and 14 uL sheared DNA sample (from previous step) for a total volume of 22.5 uL per tube and incubated in a thermocycler (12° C. 15 min; 37° C. 15 min; 72° C. 15 min; 4° C. hold). To this was added 1 ul annealed Y Adapter (10 uM), 2 ul T4 DNA Ligase and the mixture incubated in a thermocycler (16° C., 30 min; 22° C., 30 min; 4° C. hold). The sample was cleaned up using a AMPure XP SPRI beads according to manufacturer protocol and eluted in 23 uL of TE Buffer. 1 uL of sample was run on a TapeStation according to manufacturer protocol to confirm ligation of adapters to fragments. To prepare the GUIDEseq library a reaction was prepared containing 14 ul nuclease-free H₂O, 3.6 ul 10× Platinum Taq Buffer, 0.7 ul dNTP mix (10 mM each), 1.4 ul MgCl₂, 50 mM, 0.36 ul Platinum Taq Polymerase, 1.2 ul sense or antisense gene specific primer (10 uM), 1.8 ul TMAC (0.5M), 0.6 ul P5_1 (10 uM) and 10 ul of the sample from the previous step. This mix was incubated in a thermocycler (95° C. 5 min, then 15 cycles of 95° C. 30 sec, 70° C. (minus 1° C. per cycle) for 2 min, 72° C. 30 sec, followed by 10 cycles of 95° C. 30 sec, 55° C. 1 min, 72° C. 30 sec, followed by 72° C. 5 mins) The PCR reaction was cleaned up using AMPure XP SPRI beads according to manufacturer protocol and eluted in 15 uL of TE Buffer. 1 uL of sample was checked on TapeStation according to manufacturer protocol to track sample progress. A second PCR was performed by mixing 6.5 ul Nuclease-free H₂O, 3.6 ul 10× Platinum Taq Buffer (Mg2+ free), 0.7 ul dNTP mix (10 mM each), 1.4 ul MgCl₂ (50 mM), 0.4 ul Platinum Taq Polymerase, 1.2 ul of Gene Specific Primer (GSP) 2 (sense; + or antisense; −), 1.8 ul TMAC (0.5M), 0.6 ul P5_2 (10 uM) and 15 ul of the PCR product from the previous step. If GSP1+ was used in the first PCR then GSP2+ was used in PCR2. If GSP1− primer was used in the first PCR reaction then GSP2− primer was used in this second PCR reaction. After adding 1.5 ul of P7 (10 uM) the reaction was incubated in a thermocycler with the following program: 95° C. 5 min, then 15 cycles of 95° C. 30 sec, 70° C. (minus 1° C. per cycle) for 2 min, 72° C. 30 sec, followed by 10 cycles of 95° C. 30 sec, 55° C. 1 min, 72° C. 30 sec, followed by 72° C. 5 mins. The PCR reaction was cleaned up using AMPure XP SPRI beads according to manufacturer protocol and eluted in 30 uL of TE Buffer and 1 uL analyzed on a TapeStation according to manufacturer protocol to confirm amplification. The library of PCR products was quantitated using Kapa Biosystems kit for Illumina Library Quantification, according to manufacturer supplied protocol and subjected to next generation sequencing on the Illumina system to determine the sites at which the oligonucleotide had become integrated.

The results of GUIDE-seq are listed in Tables 9 to 12. It is important to take in to account the predicted target sequence identified by GUIDE-seq. If the predicted target sequence lacks a PAM or lacks significant homology to the gRNA, for example more than 5 mismatches (mm), then these genomic sites are not considered to be true off-target sites but background signals from the assay. The GUIDE-seq approach resulted in a high frequency of oligo capture in HepG2 cells indicating that this method is appropriate in this cell type. On-target read counts met the pre-set criteria of a minimum of 10,000 on target reads for 3 of the 4 guides. A small number of off-target sites for the 4 lead gRNA candidates were identified. The number of true off-target sites (meaning containing a PAM and having significant homology to the gRNA) ranged from 0 to 6 for the 4 gRNA. The T4 guide exhibited 2 off-target sites that appear real. The frequency of these events in GUIDE-seq as judged by the sequencing read count was 2% and 0.6% of the on-target cleavage frequency. Both the T13 and the T5 guides exhibited no off-target sites by GUIDE-seq that have homology to the gRNA and contain a PAM, and thus appear to have the most desirable off-target profile of the 4 guides tested. gRNA T11 exhibited one off-target site with a relatively high read count that was 23% of the on-target read count which suggest that this guide is less attractive for therapeutic use.

TABLE 9
hAlb T4 (The top ten potential off-target cleavage sites in HepG2 cells)

| Chr. | Position | Gene | Type | Predicted Target Sequence | Reads | Comment |
|---|---|---|---|---|---|---|
| 4 | 74270442 | ALB | Exonic | TAAAGCATAGTGCAATGGAT (SEQ ID | 25960 | Target site |
| | | | | NO: 106) | | |
| 14 | 55327909 | GCH1 | Intronic | TAAAGCATAGTGCCAATGGAT (SEQ ID | 515 | 1 nt bulge |
| | | | | NO: 182) | | |
| 6 | 132002620 | ENPP3 | Intronic | GAAAGCATAATAGCAATGGAT (SEQ | 158 | 2 mm, bulge |
| | | | | ID NO: 183) | | |
| 9 | 135937394 | CEL | Exonic | CTCACCATGGGGCGCCTGCAACTGGTT | 142 | No PAM |
| | | | | (SEQ ID NO: 184) | | |
| 1 | 107955183 | NTNG1 | Exonic | TAAGGCACAGTGTAATGGAT (SEQ ID | 58 | 3mm, alt. |
| | | | | NO: 111) | | spliceform |
| 3 | 31455533 | | Intergenic | GGAAGCATAGTGCAATGGTT (SEQ ID | 50 | 3 mm |
| | | | | NO: 115) | | |
| 2 | 148438664 | | Intergenic | | 37 | No PAM, homology |
| 6 | 31941379 | STK19 | Intronic | | 35 | No PAM, homology |
| 1 | 121485148 | | Intergenic | TAAAGGATCGTTCAACTCTGTGAGT | 37 | No PAM |
| | | | | (SEQ ID NO: 185) | | |
| 21 | 28069029 | | Intergenic | CCAAGCATAGGTAATGGAT (SEQ ID | 15 | 3 mm, anti-bulge |
| | | | | NO: 186) | | |
| 1 | 245132152 | | Intergenic | AAAAGCATAGTGAATGAAT (SEQ ID | 12 | 2 mm, anti-bulge |
| | | | | NO: 187) | | |

TABLE 10
hAlb T5 (The top ten potential off-target cleavage sites in HepG2 cells)

| Chr. | Position | Gene | Type | Predicted Target Sequence | Reads | Comment |
|---|---|---|---|---|---|---|
| 4 | 74270481 | ALB | Exonic | ATTTATGAGATCAACAGCAC (SEQ ID | 15407 | Target site |
| | | | | NO: 119) | | |
| 5 | 171126779 | | Intergenic | | 114 | No PAM, no |
| | | | | | | homology |
| 11 | 5271381 | | Intergenic | | 51 | No PAM, no |
| | | | | | | homology |
| 1 | 121485232 | | Intergenic | CTTCGTATAGAAACAAGACAG (SEQ | 40 | No PAM, repeat |
| | | | | ID NO: 188) | | |
| 128137 | LOC101928428 | | Intronic | GAGAGAGAGAGAAAGAGACAG (SEQ | 23 | Simple repeat |
| | | | | ID NO: 189) | | |
| 12 | 25600072 | | Intergenic | ATTCTAGAGGCATAGAGAGTTCAACC | 20 | No PAM, homology |
| | | | | T (SEQ ID NO: 190) | | |
| 1 | 121484872 | | Intergenic | TTTTCTGCCATTGACCTTAAAGCGC | 19 | No PAM, repeat |
| | | | | (SEQ ID NO: 191) | | |
| 118041 | | | Intergenic | ATCTGTGGGATTATGACTGAAC (SEQ | 14 | No PAM, homology |
| | | | | ID NO: 192) | | |
| 6 | 58778779 | | Intergenic | CTTCTCATAAAACCTAGACAG (SEQ ID | 13 | No PAM, homology |
| | | | | NO: 193) | | |
| 11 | 65429541 | RELA | Exonic | ATGTGGAGATCATTGAGCA (SEQ ID | 12 | No PAM, homology |
| | | | | NO: 194) | | |
| 12 | 19817224 | | Intergenic | ATTAATATGGTATCATGGGAGCAGGA | 9 | No homology |
| | | | | C (SEQ ID NO: 195) | | |

The two entries without a chromosome listed map to GL000220.1, an unplaced 161 kb contig.

TABLE 11
hAlb T11 (The top ten potential off-target cleavage sites in HepG2 cells)

| Chr | Position | Gene | Type | Predicted Target Sequence | Reads | Comment |
|---|---|---|---|---|---|---|
| 4 | 74270447 | ALB | Exonic | TTAAATAAAGCATAGTGCAA (SEQ | 20997 | Target site |
| | | | | ID NO: 128) | | |
| 2 | 230732566 | | Intronic | TTAAAATAAAGCATAGTGCAA | 4918 | Bulge |
| | | | | (SEQ ID NO: 196) | | |
| | 131491 | | Intergenic | | 100 | No PAM, no |
| | | | | | | homology |
| 1 | 121485377 | | Intergenic | TTCCAACGAAGGCCTCAA (SEQ ID | 86 | No PAM, homology |
| | | | | NO: 197) | | |
| 4 | 83871456 | LIN54 | Intronic | TTACTATAAAGCATAGTGCAA (SEQ | 86 | 1 mm, bulge |
| | | | | ID NO: 198) | | |
| 4 | 158301206 | | Intergenic | AAAAAAAAAGAAAAGAAAAGAAA | 37 | No PAM, homology |
| | | | | (SEQ ID NO: 199) | | |
| MT | 14042 | NDS | Exonic | TTACCTAAAACAATTTCACA (SEQ | 32 | No PAM, homology |
| | | | | ID NO: 200) | | |
| 9 | 13017306 | | Intergenic | TTAATACTGGGCCCTGAAGCCAAA | 28 | No PAM, homology |
| | | | | TACAGTT (SEQ ID NO: 201) | | |
| | 117905 | RNA45 | Exonic | GGCAACAACACATCATCAGTAGGG | 26 | No homology |
| | | SN4 | | TAA (SEQ ID NO: 202) | | |
| 14 | 105307026 | | Intergenic | TTCAGAAATAGAAAAGCTGATCCT | 21 | No PAM, homology |
| | | | | CAA (SEQ ID NO: 203) | | |
| 1 | 121484870 | | Intergenic | TTAAAGCGCTTGAAATCTACACTTG | 19 | No homology |
| | | | | CAA (SEQ ID NO: 204) | | |

The two entries without a chromosome listed map to GL000220.1, an unplaced 161 kb contig.

TABLE 12
hAlb T13 (The top ten potential off-target cleavage sites in HepG2 cells)

| Chr | Position | Gene | Type | Predicted Target Sequence | Reads | Comment |
|---|---|---|---|---|---|---|
| 4 | 74270295 | ALB | Exonic | TAATAAAATTCAAACATCCT (SEQ ID | 6620 | Target site |
| | | | | NO: 153) | | |
| 1 | 37943291 | Z3H12A | Intronic | TGATAGGATGTGTGTGTAGAAGAC | 206 | No homology |
| | | | | TCC (SEQ ID NO: 205) | | |
| 11 | 5276345 | | Intergenic | CCATAGAAGATACCAGGACTTCTT | 36 | No PAM, homology |
| | | | | (SEQ ID NO: 206) | | |
| | 124450 | | Intergenic | | 29 | No homology |
| 14 | 102377003 | PPP2R5C | Intronic | | 11 | No homology |
| 19 | 917722 | KISS1R | Exonic | ATGTAGAAGTTGGTCACGGTCCGC | 10 | No PAM, homology |
| | | | | ATCGGCT (SEQ ID NO: 207) | | |
| 3 | 80519749 | | Intergenic | AAATAGAATACCTCAGCATTTCT | 5 | No PAM, homology |
| | | | | (SEQ ID NO: 208) | | |
| 6 | 113169934 | | Intergenic | AGATGAAAATCTATCAATGGCACCA | 5 | No PAM, homology |
| | | | | GCGCCT (SEQ ID NO: 209) | | |
| 7 | 98360198 | | Intergenic | TAAAAAAGGGCTGAGCATAGTGGC | 5 | No PAM, homology |
| | | | | TCACACCT (SEQ ID NO: 210) | | |
| 1 | 121485138 | | Intergenic | TATTCAACTCACAGAGTTGAACGAT | 4 | No PAM, homology |
| | | | | CCT (SEQ ID NO: 211) | | |
| 1 | 121485228 | | Intergenic | | 3 | No homology |

The entry without a chromosome listed map to GL000220.1, an unplaced 161 kb contig.

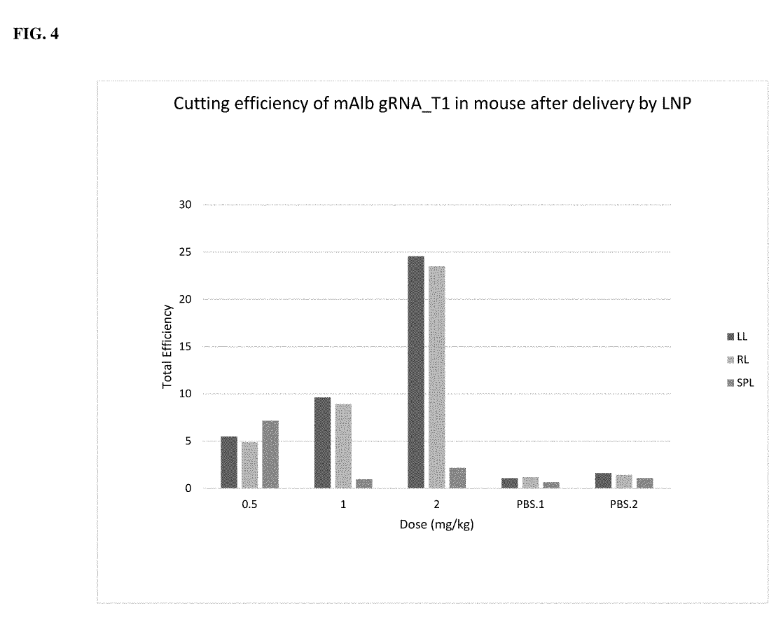

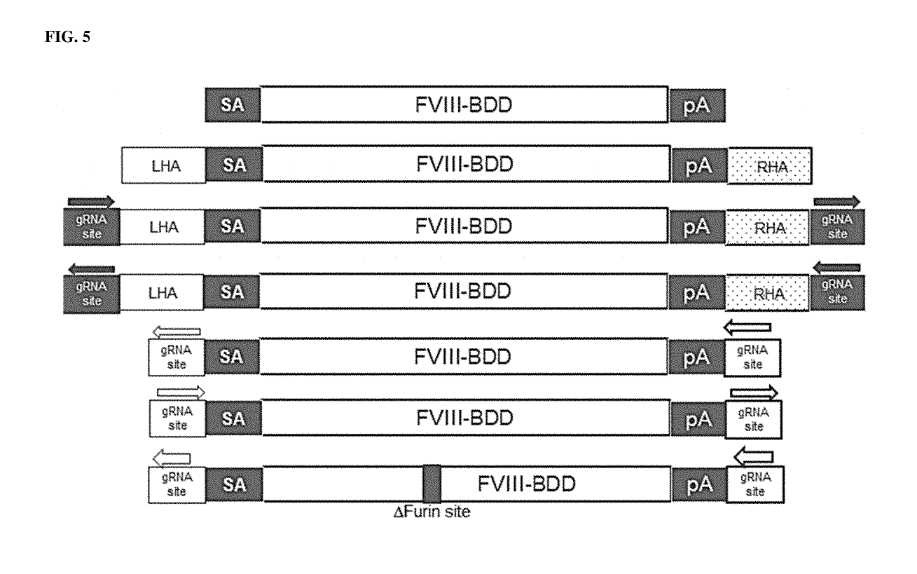

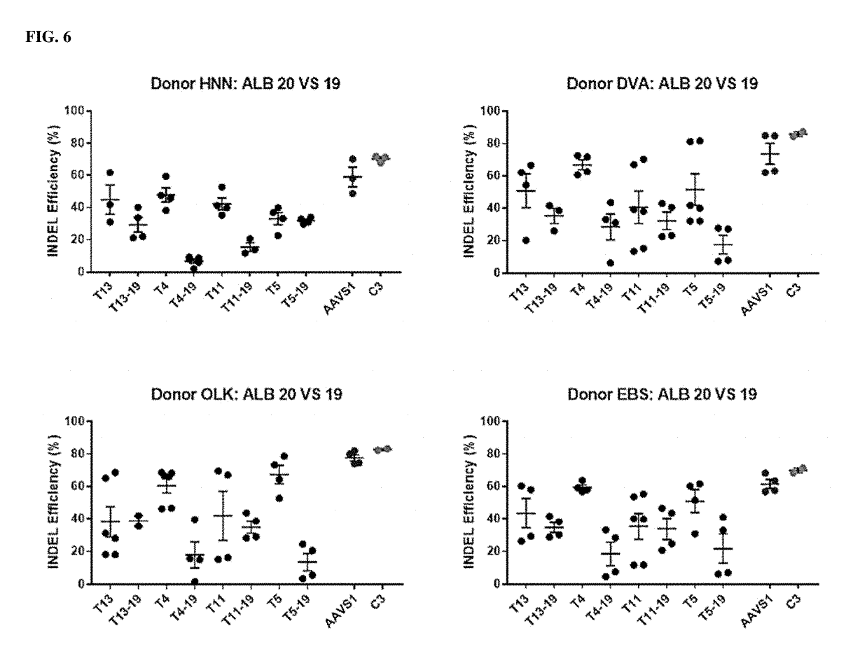

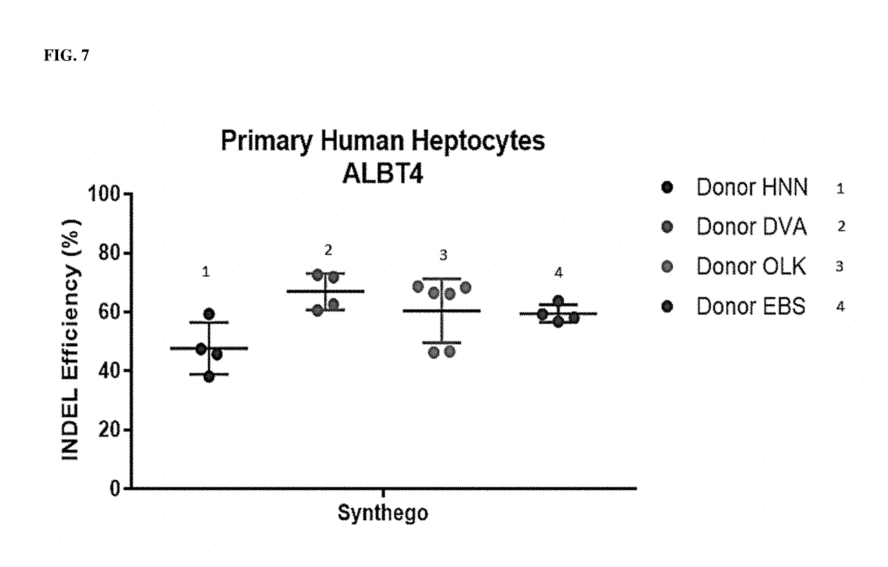

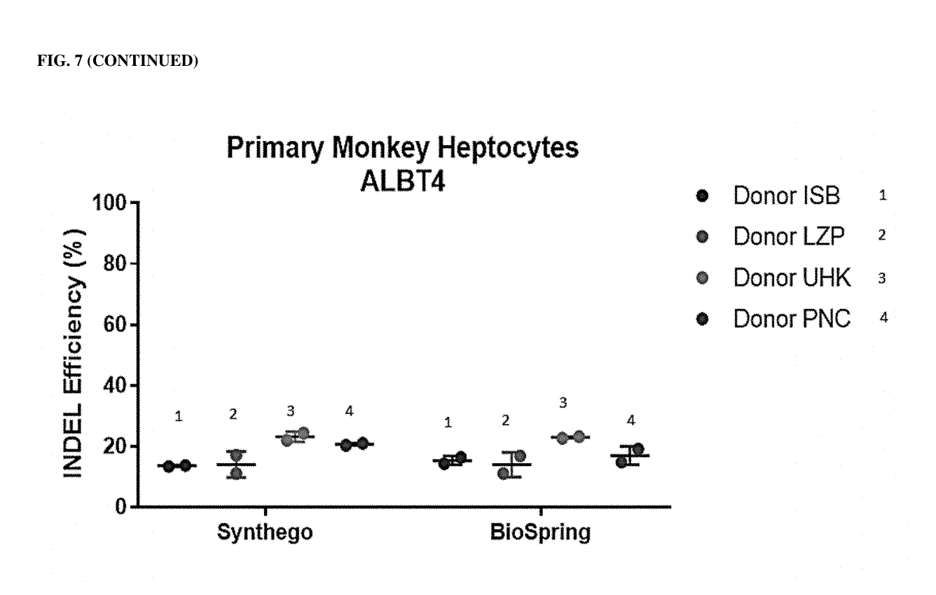

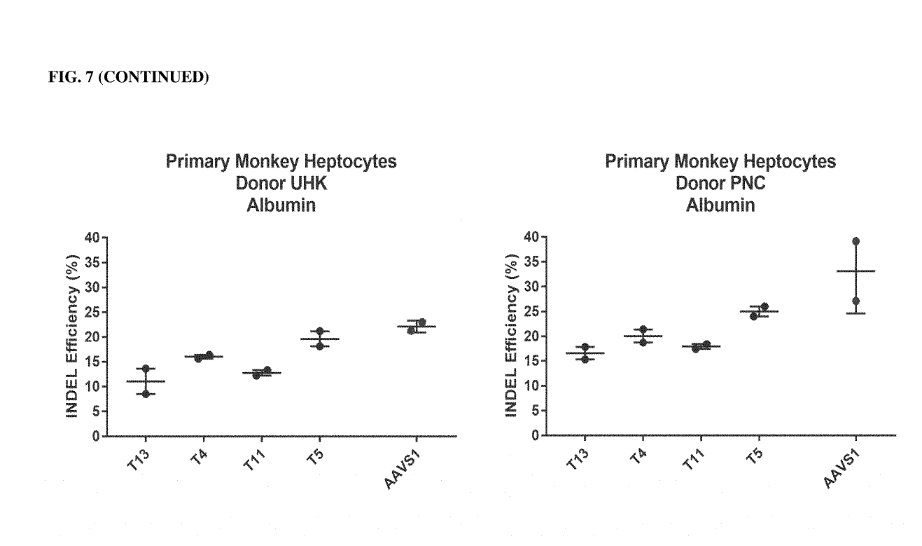

Therapeutic drug candidates are often evaluated in non-human primates in order to predict their potency and safety for human use. In the case of gene editing using the CRISPR-Cas9 system the sequence specificity of the guide RNA dictates that the same target sequence should be present in both humans and the non-human primate in order to test a guide that will be potentially used in humans Guides targeting human albumin intron 1 were screened in silico to identify those that matched the corresponding genomic sequence in Cynomologus macaques (see Table 4). However, the ability of these guides to cut the genome of non-human primates and the relative efficiency with which they cut at the predicted on-target site needs to be determined in a relevant cell system. Primary hepatocytes from Cynomolgus monkeys (obtained from BioIVT, Westbury, N.Y.) were transfected with albumin guide RNAs T4, T5, T11 or T13 and spCas9 mRNA using the same experimental protocol described above for primary human hepatocytes. The frequency of INDELS was then determined using the same TIDES protocol described above but using PCR primers specific to Cynomologus albumin intron 1. The results are summarized in FIG. 7. The corresponding data for Guide RNA T4 in human primary hepatocytes is shown in the same figure for comparison. All 4 guides promoted cleavage at the expected site in albumin intron 1 in Cynomologus hepatocytes from two different animal donors at frequencies ranging from 10% to 25%. The rank order of cutting efficiency was T5>T4>T11=T13. The T5 guide RNA was the most potent of the 4 guides and cut 20% and 25% of the target alleles in the 2 donors. The cutting efficiency was lower than the corresponding guides in human cells which may be due to differences in transfection efficiency. Alternatively, these guides and/or the spCas9 enzyme may be inherently less potent in primate cells. Nevertheless, the finding that T5 was the most potent of the 4 guides together with its favorable off-target profile by GUIDEseq makes T5 attractive for testing in NHP as well as in humans.

### Example 9: Targeted Integration of a SEAP Reporter Gene Donor in to Mouse Albumin Intron 1 Mediated by CRISPR/Cas9 Results in Expression of SEAP and Secretion into the Blood

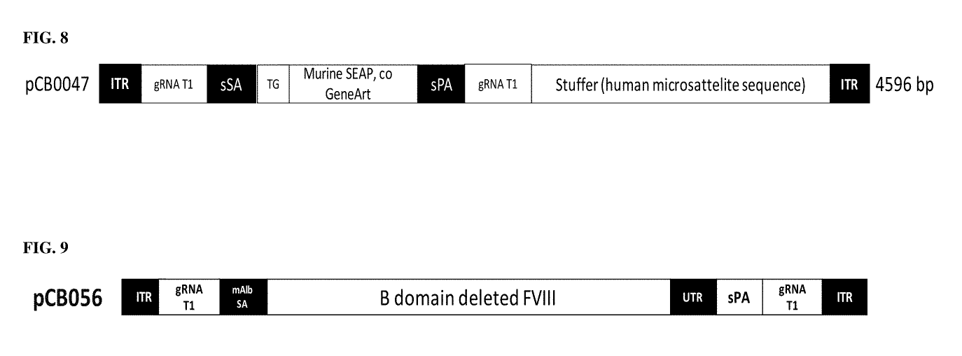

To evaluate the potential to use sequence specific cleavage by CRISPR/Cas9 to mediate integration of a donor template sequence encoding a gene of interest at the double strand break created by the Cas9/gRNA complex we designed and constructed a donor template encoding the reporter gene murine secreted alkaline phosphatase (mSEAP). The mSEAP gene is non-immunogenic in mice enabling the expression of the encoded mSEAP protein to be monitored without interference from an immune response to the protein. In addition, mSEAP is readily secreted in to the blood when an appropriate signal peptide is included at the 5′ end of the coding sequence and the protein is readily detectable using an assay that measures the activity of the protein. A mSEAP construct for packaging into Adeno Associated Virus (AAV) was designed as shown in FIG. 8 for targeted integration in to intron 1 of mouse albumin via cleavage with spCas9 and the guide RNA mALbT1 (tgccagttcccgatcgttacagg, SEQ ID NO: 80). The mSEAP coding sequence from which the signal peptide was removed was codon optimized for mouse and preceeded by two base pairs (TG) required to maintain the correct reading frame after splicing to endogenous mouse albumin exon 1. A splice acceptor consisting of the consensus splice acceptor sequence and a polypyrimidine tract (CTGACCTCTTCTCTTCCTCCCACAG, SEQ ID NO: 2) was added at the 5′ end of the coding sequence and a polyadenylation signal (sPA) was added at the 3′ end of the coding sequence (AATAAAAGATCTTTATTTTCATTAGATCTGTGTGTTGGTTTTTTGTGTG, SEQ ID NO: 5). The reverse complement of the target site for the mAlbT1 guide RNA present in the genome (TGCCAGTTCCCGATCGTTACAGG, SEQ ID NO: 80) was included on either side of this cassette. We hypothesized that by adding cut sites for the guide RNA the AAV genome should be cleaved in vivo inside the nucleus of the cells to which it was delivered thereby generating linear DNA fragments that are optimal templates for integration at a double stranded break by the non-homologous end joining (NHEJ) pathway. To enable efficient packaging into AAV capsids a stuffer fragment derived from human micro-satellite sequence was added to achieve on overall size including the ITR of 4596 bp. If this donor cassette becomes integrated in the forward orientation in to the double strand break in albumin intron 1 created by the Cas9/mALbT1 guide RNA complex, transcription from the albumin promoter is predicted to generate a primary transcript which can undergo splicing from the splice donor of albumin exon 1 to the consensus splice acceptor and generate a mature mRNA in which albumin exon1 is fused in frame to the mSEAP coding sequence. Translation of this mRNA will produce a mSEAP protein preceded by the signal peptide of mouse albumin (which is encoded in albumin exon 1). The signal peptide will direct secretion of mSEAP into the circulation and be cleaved off in the process of secretion leaving mature mSEAP protein. Because mouse albumin exon 1 encodes the signal peptide and the pro-peptide followed by 7 bp encoding the N-terminus of the mature albumin protein (encoding Glu-Ala plus 1 bp (C)), after the cleavage of the pro-peptide the SEAP protein is predicted to contain 3 additional amino acids at the N-terminus, namely Glu-Ala-Leu (Leu is generated by the last C base of albumin exon 1 that is spliced to TG from the integrated SEAP gene cassette). We chose to encode Leucine (Leu) as the 3^{rd} of the 3 additional amino acids added at the N-terminus because leucine is uncharged and non-polar and thus unlikely to interfere with the function of the SEAP protein. This SEAP donor cassette, designated pCB0047, was packaged in to the AAV8 serotype capsid using a HEK293 based transfection system and standard methods for virus purification (Vector Biolabs Inc). The virus was titered using quantitative PCR with primers and probe located within the mSEAP coding sequence.

The pCB0047 virus was injected in to the tail vein of mice on day 0 at a dose of 2e12 vg/kg followed 4 days later by a lipid nanoparticle (LNP) encapsulating the mALbT1 guide RNA (Guide RNA sequence 5′ TGCCAGTTCCCGATCGTTACAGG 3′, PAM underlined, SEQ ID NO: 80) and spCas9 mRNA. The single guide RNA was chemically synthesized and incorporated chemically modified bases essentially as described (Hendel et al, Nat Biotechnol. 2015 33(9): 985-989) and used a standard tracr RNA sequence. The spCas9 mRNA was synthesized using standard techniques and included nucleotide sequences that add a nuclear localization signal at both the N-terminus and the C-terminus of the protein. The nuclear localization signal is required to direct the spCas9 protein to the nucleus after the mRNA has been delivered to the cytoplasm of the cells of interest by the LNP and then translated in to spCas9 protein. The use of NLS sequences to direct Cas9 proteins to the nucleus is well known in the art for example see Jinek et al (eLife 2013; 2:e00471. DOI: 10.7554/eLife.00471). The spCas9 mRNA also contained a polyA tail and was capped at the 5′ end to improve stability and translation efficiency. To package the gRNA and Cas9 mRNA in LNP we used a protocol essentially as described by Kaufmann et al (Nano Lett. 15(11):7300-6) to assemble LNP based on the ionizable lipid C12-200 (purchased from AxoLabs). The other components of the LNP are cis-4,7,10,13,16,19-Docosahexaenoic acid (DHA, purchased from Sigma), 1,2-dilinoleoyl-sn-glycero-3-phosphocholine (DLPC, purchased from Avanti), 1,2-dimyristoyl-sn-glycero-3-phosphoethanolamine-N-[methoxy(polyethylene glycol)-2000] (DMPE-mPEG200, purchased from Avanti) and Cholestrol (purchased form Avanti). The LNP was produced using the Nanoassembler Benchtop instrument (Precision Nanosystems) in which the LNP self-assemble when the lipid and nucleic acid components are mixed under controlled conditions in a microfluidic chamber. The spCas9 mRNA and guide RNA were encapsulated in separate LNP. The LNP were concentrated by dialysis into phosphate buffered saline and stored at 4° C. for up to 1 week before use. The LNP were characterized using dynamic light scattering and typically had a size in the range of 50 to 60 nM. The concentration of RNA in the LNP was measured using the Ribogreen assay kit (Thermofisher Scientific) and used to determine the dose given to mice. For dosing mice, the spCas9 and guide RNA LNP were mixed at a 1:1 mass ratio of RNA immediately prior to injection. The ability of these LNP to deliver the spCas9 mRNA and guide RNA to the liver of mice was demonstrated by injecting mice IV with a range of LNP doses and measuring cleavage of the mouse genome at the on-target site in albumin intron 1 in the liver using the TIDES procedure (Brinkman et al, Nucleic Acids Res. 2014 Dec. 16; 42(22): e168). See Example 2 (FIG. 4) for a typical result where up to 25% of the alleles were cleaved at the on-target site.

Two cohorts of 5 mice were injected in the tail vein with 2e12 vg/kg of AAV8-CB0047 virus. Three days later one of the cohorts was injected with LNP encapsulating spCas9 mRNA and mAlbT1 guide RNA at a total RNA dose of 2 mg/kg (1:1 ratio of spCas9 and gRNA). Blood samples were collected weekly and the plasma was assayed for SEAP activity using a commercial kit (InvivoGen). The results (see Table 13) demonstrate that no SEAP activity was detectable in the mice that received only the AAV8-pCB0047 virus. Mice that received the AAV8-pCB0047 virus followed by the LNP had SEAP activity in the plasma that remained stable until the last time point at 4 weeks post dosing. The finding that SEAP was only expressed when mice received both the AAV8 donor SEAP gene and the CRISPR-Cas9 gene editing components suggests that the SEAP protein was being expressed from copies of the SEAP gene integrated in to the target site in albumin intron 1. Because the SEAP gene in pCB047 lacks a signal peptide or a promoter it cannot be expressed and secreted unless it is operably linked to a promoter and a signal peptide that is in- frame with the SEAP coding sequence. It is unlikely that this would happen if the pCB047 gene cassette was integrated in to a random site in the genome.

To confirm that the SEAP gene cassette from pCB0047 was integrated in intron 1 of albumin we used Droplet Digital PCR (DD-PCR) to measure the integration frequency in genomic DNA extracted from the livers of the mice at the end of the study. DD-PCR is a method to accurately quantify the number of copies of a nucleic acid sequence in a complex mixture. A pair of PCR primers were designed with one located in the mouse albumin genomic sequence on the 5′ side of the target site for mAlbT1 guide (the predicted site for targeted integration) and the other primer located at the 5′ end of the SEAP gene in pCB0047. This “in-out” PCR will amplify the junction between the mouse albumin genomic sequence and the integrated SEAP cassette when the SEAP cassette is integrated in the desired forward orientation. A fluorescent probe was designed that hybridizes to the DNA sequence amplified by these 2 primers. As an internal control for the DD-PCR assay a primer probe set that detects the mouse albumin gene was used. Using this DD-PCR assay we measured a targeted integration frequency of 0.24+/−0.07% (0.24 copies per 100 copies of the albumin gene) thereby confirming that the SEAP cassette was integrated at albumin intron 1.

TABLE 13
SEAP activity in the plasma of mice injected with the pCB0047 AAV8
virus alone or followed 3 days later with LNP encapsulating spCas9
mRNA and mAlbT1 guide RNA

| | SEAP activity (micro U/ml of plasma) | |
|---|---|---|
| Time after AAV | AAV-SEAP only | AAV-SEAP and LNP on day 3 |
| Week 1 | −19.9 ± 8.1 | 2264 ± 766 |
| Week 2 | −31.2 ± 14.6 | 3470 ± 1480 |
| Week 3 | −423.8 ± 7.6 | 4575 ± 1737 |
| Week 4 | −415.2 ± 8.1 | 2913 ± 1614 |

### Example 10: Targeted Integration of a Human FVIII Gene Donor in to Mouse Albumin Intron 1 Mediated by CRISPR/Cas9 Results in Expression of FVIII in the Blood

Hemophilia A is an extensively studied disease (Coppola et al, J Blood Med. 2010; 1: 183-195) in which patients have mutations in the Factor VIII gene that results in low levels of functional Factor VIII protein in their blood. Factor VIII is a critical component of the coagulation cascade and in the absence of sufficient amounts of FVIII the blood fails to form a stable clot at sites of injury resulting in excessive bleeding. Hemophilia A patients that are not effectively treated experience bleeding in to joints resulting in joint destruction. Intracranial bleeding can also occur and can sometimes be fatal.

To evaluate if this gene editing strategy could be used to treat Hemophilia A we used a mouse model in which the mouse FVIII gene is inactivated. These Hemophilia A mice have no detectable FVIII in their blood which makes it possible to measure exogenously supplied FVIII using a FVIII activity assay assay (Diapharma, Chromogenix Coatest SP Factor FVIII, cat# K824086kit). As standards in this assay we used Kogenate (Bayer), a recombinant human FVIII used in the treatment of hemophilia patients. The results of the assay are reported as percentage of normal human FVIII activity which is defined as 1 IU/ml. A human FVIII donor template was constructed based on a B-domain deleted FVIII coding sequence that had been shown to function when delivered to mice with an AAV vector under the control of a strong liver specific promoter (McIntosh et al, 2013; Blood; 121(17):3335-3344). The DNA sequence encoding the native signal peptide was removed from this FVIII coding sequence and replaced with two base pairs (TG) required to maintain the correct reading frame after splicing to mouse albumin exon 1. A splice acceptor sequence derived from mouse albumin intron 1 was inserted immediately 5′ of this FVIII coding sequence. A 3′ untranslated sequence from the human globin gene followed by a synthetic polyadenylation signal sequence was inserted on the 3′ side of the FVIII coding sequence. The synthetic polyadenylation signal is a short 49 bp sequence shown to effectively direct polyadenylation (Levitt et al, 1989; GENES & DEVELOPMENT 3:1019-1025). The 3′ UTR sequence was taken from the B-globin gene and may function to further improve polyadenylation efficiency. The reverse complement of the target sites for the mAlbT1 guide RNA were placed either site of this FVIII gene cassette to create a vector called pCB056 containing the ITR sequences of AAV2 as shown in FIG. 9. This plasmid was packaged in to AAV8 capsids to generated AAV8-pCB056 virus.

A cohort of 5 hemophilia A mice (Group 2; G2) were injected in the tail vein with AAV8-pCB056 virus at a dose of 1 e13 vg/kg and 19 days later the same mice were injected in the tail vein with a mixture of two C12-200 based LNP encapsulating spCas9 mRNA and mAlbT1 guide RNA, each at a dose of 1 mg RNA/kg. The LNP were formulated as described in Example 2 above. A separate cohort of 5 hemophilia A mice (Group 6; G6) were injected in the tail vein with AAV8-pCB056 virus at a dose of 1 e13 vg/kg and FVIII activity was monitored over the following 4 weeks. When only the AAV was injected no FVIII activity was measurable in the blood of the mice (G6 in FIG. 9). Mice that received the AAV8-pCB056 virus followed by the CRISPR/Cas9 gene editing components in a LNP had FVIII activity in their blood that ranged from 25% to 60% of normal human levels of FVIII activity. Severe Hemophilia patients have FVIII activity levels less than 1% of normal, moderate Hemophilia A patients have FVIII levels between 1 and 5% of normal and mild patients have levels between 6% and 30% of normal. An analysis of Hemophilia A patients taking FVIII replacement protein therapy reported that at predicted FVIII trough levels of 3%, 5%, 10%, 15% and 20% the frequency at which no bleeds occurred was 71%, 79%, 91%, 97%, and 100% respectively (Spotts et al Blood 2014 124:689), suggesting that when FVIII levels are maintained above a minimum level of 15 to 20% the rate of bleeding events was reduced to close to zero. While a precise FVIII level required to cure Hemophilia A has not been defined and likely varies between patients, levels of between 5% and 30% are likely to provide a significant reduction in bleeding events. Thus, in the Hemophilia A mouse model described above the FVIII levels that were achieved (25 to 60%) are in a therapeutically relevant range expected to be curative.

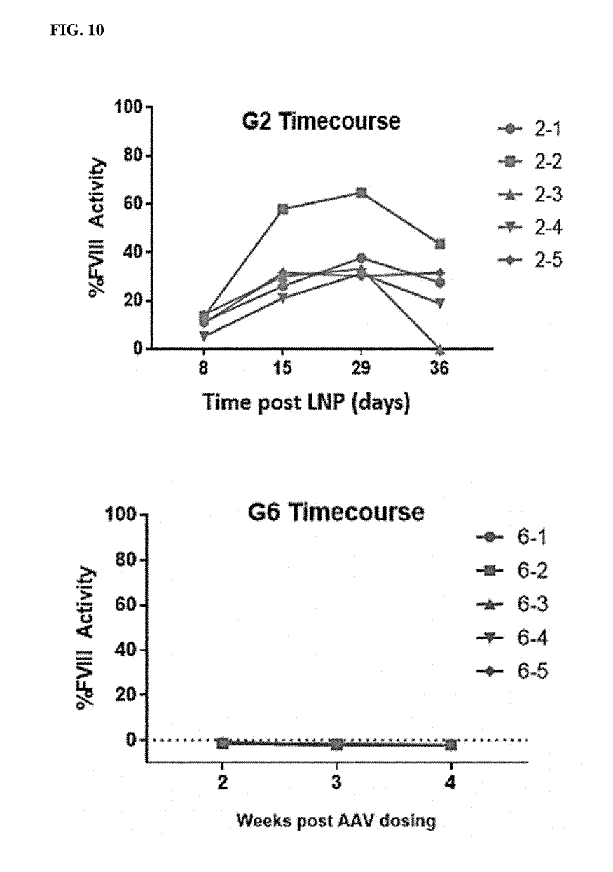

Four of the five mice in FIG. 10 exhibited stable FVIII levels (within normal variability of the assay and the variation in mouse physiology) up to the end of the study at day 36. FVIII activity in one of the mice (2-3) dropped to undetectable levels at day 36 and this was likely due to an immune response against the human FVIII protein that can be recognized as a foreign protein in mice (Meeks et al, 2012 Blood 120(12): 2512-2520). The observation that no FVIII protein was expressed in the mice when only the AAV-FVIII donor template was injected demonstrates that expression of FVIII required the provision of the CRISPR/Cas9 gene editing components. Because the FVIII donor cassette does not have a promoter or a signal peptide it is unlikely that FVIII would be made by integration of the cassette into random sites in the genome or by some other undefined mechanism. To confirm that the FVIII donor cassette was integrated in to intron 1 of albumin we used in-out PCR in a DD-PCR format. The whole livers of the mice in group 2 were homogenized and genomic DNA was extracted and assayed by DD-PCR using one primer located in the mouse albumin gene at a position 5′ of the cut site for the mAlbT1 gRNA at which on-target integration is predicted to have occurred. The second PCR primer was located at the 5′ end of the FVIII coding sequence within the pCB056 cassette. A fluorescent probe used for detection was designed to hybridize to a sequence between the two PCR primers. PCR using these 2 primers will amplify the 5′ junction of integration events in which the FVIII cassette was integrated at the mAlbT1 gRNA cut site in the forward orientation that would be capable of expressing the FVIII protein. A DD-PCR assay against a region within the mouse albumin gene was used as a control to measure the copy number of mouse genomes in the assay. This assay detected between 0.46 and 1.28 targeted integration events per 100 haploid mouse genomes (average of 1.0). There was a correlation between the targeted integration frequency and peak FVIII levels consistent with FVIII being produced from the integrated FVIII gene cassette. Assuming that about 70% of the cells in the mouse liver are hepatocytes and that both AAV8 and LNP are primarily taken up by hepatocytes it can be estimated that 1.4% (1.0*(1/0.7)) of the hepatocyte albumin alleles contained an integrated FVIII cassette in the forward orientation. These results demonstrate that CRSIPR/Cas9 can be used to integrate an appropriately designed FVIII gene cassette into albumin intron 1 of mice resulting in the expression and secretion of therapeutic levels of functional FVIII protein into the blood. The delivery modalities employed in this study, namely an AAV virus delivering the FVIII donor template and a LNP delivering the CRISPR/Cas9 components are potentially amenable to in vivo delivery to patients. Because the Cas9 was delivered as an mRNA that has a short life span in vivo (in the range of 1 to 3 days) the CRISPR/Cas9 gene editing complex will only be active for a short time which limits the time for off-target cleavage events to occur, thus providing a predicted safety benefit. These data demonstrate that although the CRISPR/Cas9 was active for only a short time this was sufficient to induce targeted integration at a frequency sufficient to produce therapeutically relevant levels of FVIII activity in mice.

TABLE 14
Targeted integration frequencies and FVIII levels in HemA mice from
Group 2 that were injected with both AAV8-pCB056 and LNP

| | | | Peak |
|---|---|---|---|
| | Targeted | FVIII activity at day 36 | FVIII activity |
| Mouse ID | Integration (%) | (% of normal) | (% of normal) |
| 2-1 | 0.97 | 27 | 38 |
| 2-2 | 1.28 | 43 | 62 |
| 2-3 | 0.46 | 0 | 25 |
| 2-4 | 1.01 | 19 | 30 |
| 2-5 | 1.28 | 32 | 32 |
| Naive HemA | 0.00 | 0 | 0 |
| mouse | | | |

### Example 11: The Timing of Dosing the Guide RNA and Cas9 mRNA in a LNP Relative to the AAV Donor Impacts the Levels of Gene Expression

To evaluate whether the time between injecting the AAV donor template and dosing of the LNP encapsulating the Cas9 mRNA and guide RNA had an impact on the level of expression of the gene encoded on the donor template we injected two cohorts of 5 mice each with AAV8-pCB0047 that encodes mSEAP. Four days after the AAV was injected one cohort of mice (group 3) was injected with C12-200 based LNP encapsulating spCas9 mRNA and mAlbT1 gRNA (1 mg/kg of each) and SEAP activity was measured in the plasma weekly for the next 4 weeks. The SEAP activity was monitored in the second cohort of mice for 4 weeks during which no SEAP was detected. At 28 days after the AAV had been injected the mice in group 4 were dosed with C12-200 based LNP encapsulating spCas9 mRNA and mAlbT1 gRNA (1 mg/kg of each) and SEAP activity was measured in the plasma weekly for the next 3 weeks. The SEAP data are summarized in Table 15. In group 3 that received LNP encapsulated spCas9/gRNA 4 days after the AAV the SEAP activity was on average 3306 microU/ml. In group 4 that received LNP encapsulated spCas9/gRNA 28 days after the AAV the SEAP activity was on average 13389 microU/ml which is 4-fold higher than that in group 3. These data demonstrated that dosing the LNP encapsulated spCas9/gRNA 28 days after the LNP results in 4-fold higher expression from the gene integrated in the genome than if the LNP encapsulated spCas9/gRNA is dosed just 4 days after the AAV-donor template. This improved expression is likely due to a higher frequency of integration of full length donor encoded gene cassettes into albumin intron 1.

TABLE 15
SEAP activity in the plasma from mice injected with AAV8-pCB0047
and LNP either 4 days or 28 days later

| | Group: Mean ± SD | |
|---|---|---|
| | 3 | 4 |
| | Mean SEAP activity in | |
| | plasma (microU/ml) in 5 mice | |
| | AAV8-SEAP + | AAV8-SEAP + |
| | LNP 4 days later | LNP 28 days later |
| Week 1 | 2264 ± 766 | −19.9 ± 8.2 |
| Week 2 | 3470 ± 1480 | −31.2 ± 14.6 |
| Week 3 | 4575 ± 1737 | −423.8 ± 7.6 |
| Week 4 | 2913 ± 1614 | −415.2 ± 8.1 |
| Week 5 | | 19856 ± 12732 |
| Week 6 | | 10657 ± 9642 |
| Week 7 | | 9680 ± 3678 |
| Mean SEAP after LNP dosing | 3306 ± 848 | 13389 ± 4584 |
| (average from 3 or 4 time points) | | |

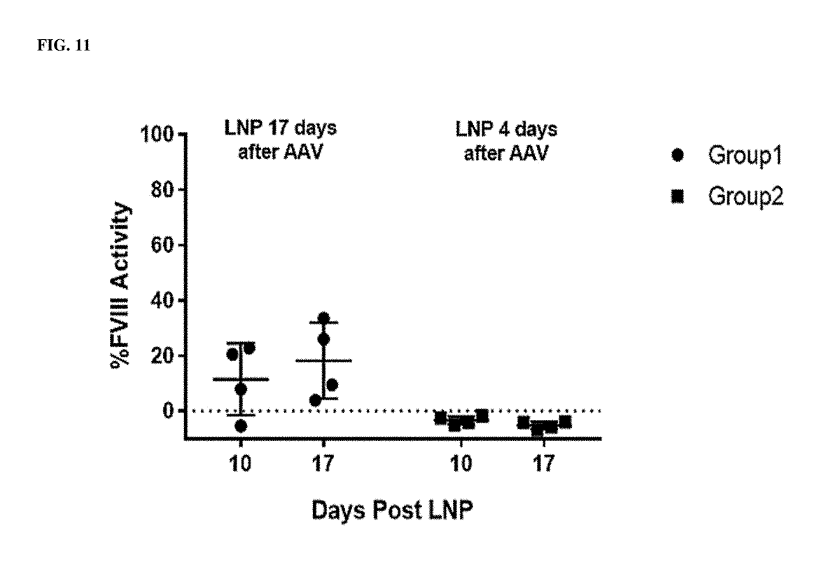

The impact of the timing of AAV-donor and LNP encapsulated Cas9/gRNA dosing was also evaluated using the Factor VIII gene as an example of a gene of therapeutic relevance. Two cohorts of hemophilia A mice were injected with AAV8-pCB056 which encodes a human FVIII donor cassette at a dose of 2e12 vg/kg on day 0. One of the cohorts was injected 4 days later with C12-200 based LNP encapsulating spCas9 mRNA and mAlbT1 gRNA (1 mg/kg each) while the second cohort was dosed 17 days later with C12-200 based LNP encapsulating spCas9 mRNA and mAlbT1 gRNA (1 mg/kg each). The dosing of the AAV8-pCB056 was staggered so that the same batch of LNP encapsulating spCas9 mRNA and guide RNA was used for both groups on the same day. The FVIII activity in the blood of the mice was measured at day 10 and day 17 after the LNP was dosed and the results are shown in FIG. 11. The mice that received LNP 4 days after the AAV had no detectable FVIII in their blood while the all 4 of the mice in the group that was injected with the LNP 17 days after the AAV had detectable FVIII activity that ranged from 2% to 30% of normal on day 17. These results demonstrate that for a AAV donor encoding FVIII, dosing the CRISPR/Cas9 components at least 17 days after the AAV donor results in therapeutically relevant levels of FVIII while dosing 4 days after the AAV did not lead to FVIII expression.

The process by which AAV infects cells, including the cells of the liver, involves escape from the endosome, virus uncoating and the transport of the AAV genome to the nucleus. In the case of the AAV used in these studies in which single stranded genomes are packaged in the virus, the single stranded genomes undergo a process of second strand DNA synthesis to form double stranded DNA genomes. The time required for complete conversion of single stranded genomes to double stranded genomes is not well established, but it is considered to be a rate limiting step (Ferrari et al 1996; J Virol. 70: 3227-3234). The double stranded linear genomes then become concatemerized in to multimeric circular forms composed of monomers joined head to tail and tail to head (Sun et al 2010; Human Gene Therapy 21:750-762). Because the AAV donor templates used in our studies do not contain homology arms they will not be templates for HDR and can therefore only integrate via the NEHJ pathway. Only double stranded linear DNA fragments are templates for NHEJ mediated integration at a double strand break. Thus, we hypothesize that delivering the CRISPR-Cas9 components to the liver cells soon after the AAV donor might lead to a low frequency of integration because the majority of the AAV genomes are in a single strand form and under these circumstances most of the double strand breaks in the genome will be repaired with small insertions and deletions without integration of a donor template. Delivering the CRISPR/Cas9 gene editing components at a later time after the AAV-donor template allows time for the formation of double strand AAV genomes which are templates for NHEJ mediated targeted integration. However, waiting too long after the AAV donor was delivered may result in the conversion of double stranded linear forms to circular (concatemeric) forms that will not be templates for NHEJ mediated targeted integration. The inclusion of cut sites for the guide RNA/Cas9 in the donor template will result in cleavage of circular forms to generate linear forms. Any remaining linear forms will also be cleaved to release short fragments containing the AAV ITR sequence. The inclusion of either 1 or 2 guide RNA cut sites in the AAV donor template will generate a variety of linear fragments from concatemeric forms of the AAV genome. The types of linear fragments will vary depending on the number of cut sites in the AAV genome and the number of multimers in each concatemer and on their relative orientation and is thus difficult to predict. A single gRNA site placed at the 5′ end of the cassette in AAV will release monomeric double stranded templates from both monomeric circles and head to tail concatemers (head to tail means the 5′ end of one AAV genome joined to the 3′ end of the next AAV genome). However, a single gRNA site at the 5′ end will not release a monomeric double stranded linear template from head to head concatemers (head to head concatemers consist of the 5′ end of one AAV genome joined to the 5′ end of the next AAV genome). A possible advantage of using a single gRNA site at the 5′ end is that it will only release short ITR containing double strand fragments from head to head concatemers but not from head to tail concatemers. With a single gRNA cut site at the 5′ end of the AAV genome the ITR will remain at the 3′ end of the linear monomeric gene cassettes and therefore will be integrated in the genome. When the donor cassette in AAV contains two gRNA sites (flanking the cassette) this will result in the release of monomeric double stranded templates from all forms of double strand DNA and therefore may liberate more template for targeted integration, especially if a mix of head to tail and tail to head concatemers are present. A potential disadvantage of including 2 gRNA target sites flanking the cassette is that this will release small (about 150 base pair) double stranded linear fragments that contain the AAV ITR sequence. Two of these small (about 150 base pair) fragments will be generated for each copy of the gene cassette containing the therapeutic gene of interest. The short ITR containing fragments are expected to also be templates for NHEJ mediated targeted integration at the double stranded break in the genome and will therefore compete with the fragment containing the gene cassette for integration in the double strand break in the genome and thereby reduce the frequency at which the desired event of integration of the therapeutic gene cassette in to the genome of the host cell occurs. Given the complexity of this biological system in which many parameters such as the kinetics of concatemer formation and the molecular composition of the concatemers (content of head to tail and tail to head concatemers and the number monomeric units in the concatemers) is not known, it is not possible to predict with any certainty whether 0, 1 or 2 guide cut sites in the donor cassette will achieve the highest targeted integration of the desired donor cassette containing the therapeutic gene or how this is effected by the timing of delivery of the CRISPR/Cas9 gene editing components. Our data support that inclusion of 2 guide RNA cut sites leads to measurable targeted integration in a setting where the CRISPR/Cas9 gene editing components are delivered by a LNP encapsulating spCas9 mRNA and the guide RNA dosed at least 17 days after the AAV-donor cassette was dosed, but not when the LNP was dosed 4 days after the AAV-donor cassette.

### Example 12: Impact of Different Polyadenylation Signal on FVIII Expression

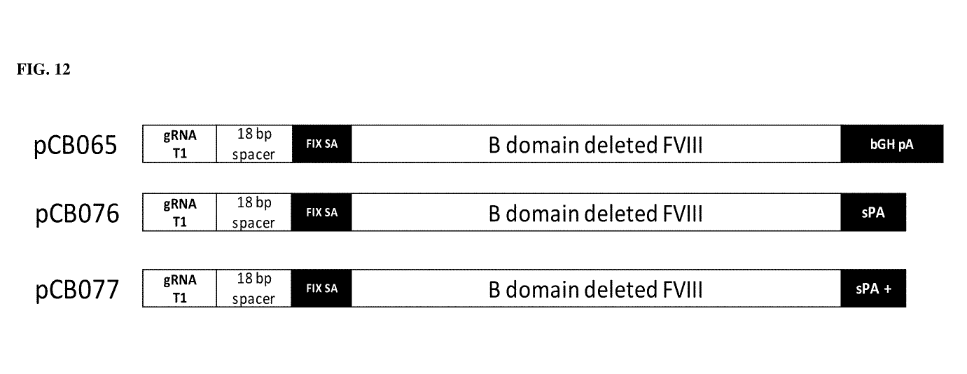

To evaluate the impact of different polyadenylation signal sequences upon the expression of a FVIII gene after targeted integration in to mouse albumin intron 1 we constructed a series of plasmids shown in FIG. 12. These plasmids were designed with a single target site for the mALbT1 gRNA at the 5′ end that will result in linearization of the circular plasmid DNA in vivo after delivery to mice using hydrodynamic injection (HDI). HDI is an established technique for delivery of plasmid DNA to the liver of mice (Budker et al, 1996; Gene Ther., 3, 593-598) in which naked plasmid DNA in saline solution is injected rapidly in to the tail vein of mice (2 to 3 ml volume in 5 to 7 seconds).

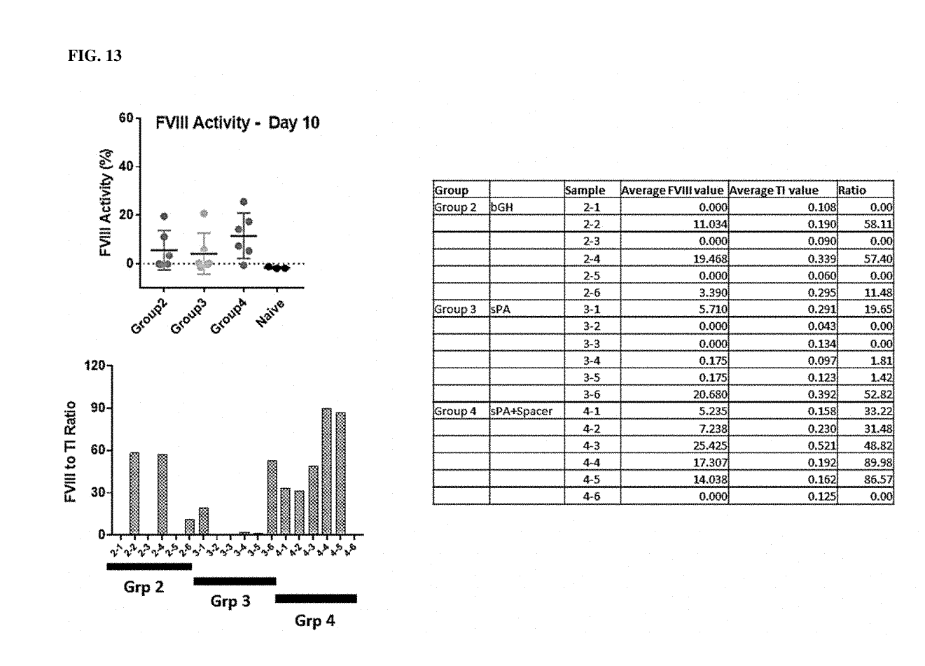

Cohorts of 6 hemophilia A mice were injected hydrodynamically with 25 μg per mouse of pCB065, pCB076 or pCB077. Twenty four hours later the mice were dosed by retroorbital injection with a C12-200 LNP encapsulating spCas9 mRNA and mAlbT1 gRNA at a dose of 1 mg/kg of each RNA. FVIII activity in the blood of the mice was measured on day 10 post LNP dosing. At day 10 the mice were sacrificed, the whole liver was homogenized and genomic DNA was extracted from the homogenate. The frequency of targeted integration of the FVIII donor cassette in the forward orientation in to albumin intron 1 was quantified using quantitative real time PCR. In this real time PCR assay one primer was located in the genomic sequence of the mouse albumin gene 5′ of the expected integration site (the cut site for mAlbT1 gRNA) and the second PCR primer was located at the 5′ end of the FVIII coding sequence in the donor plasmid. A fluorescent probe was located between the two primers. This assay will specifically detect the junction between the mouse genome and the donor cassette when integration occurred in the forward orientation (in which the FVIII gene is in the same orientation as the genomic mouse albumin gene). Synthetic DNA fragments composed of the predicted sequence of the junction fragment spiked in to naïve mouse liver genomic DNA were used as copy number standards to calculate the absolute copies of integration events in the liver genomic DNA. The FVIII activity in mice in groups 2 (injected with pCB065), 3 (injected with pCB076) and 4 (injected with pCB077) was 5.5%, 4.2% and 11.4% respectively. Group 4 that was injected with pCB077 had the highest FVIII activity. Because the delivery of DNA to the liver by hydrodynamic injection is highly variable between mice we calculated the FVIII activity divided by the targeted integration frequency as shown in FIG. 13 for each individual mouse. This ratio represents the FVIII expression per integrated copy of the FVIII gene and demonstrated superior expression from pCB077 (group 4) compared to pCB065 and pCB076. When we excluded the mice that did not express any FVIII, the mean FVIII/TI ratios were 42, 8 and 57 for pCB065, pCB076 and pCB077, respectively. These data indicate that the aPA+ polyadenylation signal in pCB077 enables superior expression of FVIII as compared to the sPA polyadenylation signal in pCB076. The expression of FVIII using the sPA+ polyadenylation signal was similar to that using the bovine growth hormone (bGH) polyadenylation signal. There is an advantage to using a short polyadenylation signal sequence such as the sPA (49 bp) or sPA+ (54 bp) as compared to bGH polyA (225 bp) when delivering the donor using AAV virus, especially in the case of the FVIII gene which at 4.3 Kb in size is close to the packaging limit for AAV (4.4 Kb excluding the ITR). The sPA+ polyadenylation signal differs from the sPA polyadenylation signal only by the presence of a 5 bp spacer (tcgcg, SEQ ID NO: 212) between the stop codon of the FVIII gene and the synthetic polyadenylation signal sequence (aataaaagatctttattttcattagatctgtgtgttggttttttgtgtg, SEQ ID NO: 5). While this synthetic polyadenylation signal sequence has been previously described (Levitt et al, 1989; Genes Dev. (7):1019-25) and used by others in AAV based gene therapy vectors (McIntosh et al, 2013; Blood 121:3335-3344), a benefit of including a spacer sequence has not been explicitly demonstrated. Our data demonstrate that including a short spacer of 5 bp improved expression of a FVIII gene integrated in to albumin intron 1 in which transcription was driven off the strong albumin promoter in the genome. It is possible that the advantage of the spacer is unique to the setting of targeted integration in to a highly expressed locus in the genome.

### Example 13: Repeat Dosing of CRISPR/Cas9 Components Using a LNP Results in Incremental Increases in Expression of a AAV Delivered Donor Cassette Targeted to Mouse Albumin Intron 1

In the setting of administering to a patient a gene editing based gene therapy in which a therapeutic gene is integrated in to intron 1 of albumin it would be advantageous to achieve a level of gene expression that provides the optimal therapeutic benefit to the patient. For example, in Hemophilia A the most desirable level of FVIII protein in the blood would be in the range of 20% to 100% or 30% to 100% or 40% to 100% or most preferable 50% to 100%. FVIII levels that exceed 100% increase the risk of thrombotic events (Jenkins et al, 2012; Br J Haematol. 157:653-63) and are thus undesirable. Standard AAV based gene therapies that use a strong promoter to drive expression of the therapeutic gene from episomal copies of the AAV genome do not enable any control of the level of expression that is achieved because the AAV virus can only be dosed once and the levels of expression that are achieved vary significantly between patients (Rangarajan et al, 2017; N Engl J Med 377:2519-2530). After the patient is dosed with a AAV virus they develop high titer antibodies against the virus capsid proteins that based upon pre-clinical models are expected to prevent effective re-administration of the virus (Petry et al, 2008; Gene Ther. 15:54-60). An approach where the therapeutic gene delivered by a AAV virus is integrated in to the genome at a safe harbor locus, such as albumin intron 1, and this targeted integration occurs via the creation of a double stranded break in the genome provides an opportunity to control the level of targeted integration and thus the levels of the therapeutic gene product. After the liver is transduced by a AAV encapsulating a AAV genome containing a donor DNA cassette encoding the therapeutic gene of interest the AAV genome will be maintained episomally within the nucleus of the transduced cells. These episomal AAV genomes are relatively stable over time and therefore provide a pool of donor template for targeted integration at double strand breaks created by CRISPR/Cas9. The potential to use repeated doses of the CRISPR/Cas9 components delivered in a non-immunogenic LNP to induce stepwise increases in expression of a protein encoded on a AAV delivered donor template was evaluated using AAV8-pCB0047 and spCas9 mRNA and mALbT1 gRNA encapsulated in C12-200 LNP. A cohort of 5 mice were injected in the tail vein with AAV8-pCB0047 at 2e12 vg/kg and 4 days later were injected iv with C12-200 based LNP encapsulating spCas9 mRNA at 1 mg/kg and mAlbT1 gRNA at 1 mg/kg. SEAP levels in the blood were measured weekly for the next 4 weeks and averaged 3306 microU/ml (Table 16). Following the last SEAP measurement on week 4 the same mice were re-dosed with C12-200 LNP encapsulated spCas9 mRNA and mALbT1 gRNA at 1 mg/kg each. SEAP levels in the blood were measured weekly for the next 3 weeks and averaged 6900 microU/ml, 2-fold higher than the mean weekly levels after the first LNP dose. The same 5 mice were then given a third injection of C12-200 LNP encapsulated spCas9 mRNA and mALbT1 gRNA at 1 mg/kg each. SEAP levels in the blood were measured weekly for the next 4 weeks and averaged 13117 microU/ml, 2-fold higher than the mean weekly levels after the second LNP dose. These data demonstrate that repeat dosing of CRISPR/Cas9 gene editing components comprising spCas9 mRNA and gRNA encapsulated in a LNP can result in stepwise increases in gene expression from a AAV delivered donor template. The fact that the SEAP gene encoded on the donor template is dependent upon covalent linkage to a promoter and a signal peptide sequence for expression strongly suggests that the increased expression is due to increased targeted integration in to albumin intron 1. At week 12 the mice were sacrificed, the whole liver was homogenized, and genomic DNA was extracted and assayed for targeted integration at albumin intron 1 using DD-PCR with primers flanking the predicted 5′ junction in the forward orientation (the orientation necessary to produce functional SEAP protein). The integration frequency was on average 0.3% (0.3 copies per 100 albumin alleles).

TABLE 16
SEAP activity in the blood of mice injected with AAV8-pCB0047
followed by C12-200 LNP encapsulating spCas9 mRNA and
mAlbT1 gRNA (1 mg/kg each) 4 days, 4 weeks and 7 weeks
after the AAV

| | Mean SEAP |
|---|---|
| | activity in plasma |
| | (microU/ml) in 5 mice |
| Time post AAV | AAV8-SEAP + LNP at |
| dosing | 4 days, 4 weeks and 7 weeks |
| Week 1 | 2264 ± 765.6 |
| Week 2 | 3470 ± 1480 |
| Week 3 | 4575 ± 1737 |
| Week 4 | 2913 ± 1614 |
| Average SEAP activity after 1 LNP dose | 3306 ± 848 |
| Week 5 | 9817 ± 4322 |
| Week 6 | 6042 ± 2858 |
| Week 7 | 4840 ± 2355 |
| Average SEAP activity after 2 LNP doses | 6900 ± 2120 |
| Week 8 | 12066 ± 3460 |
| Week 9 | 12886 ± 9014 |
| Week 10 | 15333 ± 4678 |
| Week 11 | 12181 ± 2986 |
| Average SEAP activity after 3 LNP doses | 13117 ± 1318 |

### Example 14: Targeted Integration of a FVIII or SEAP Donor into Albumin Intron 1 in Primary Human Hepatocytes Mediated by CRISPR/Cas9 Results in Expression of FVIII or SEAP

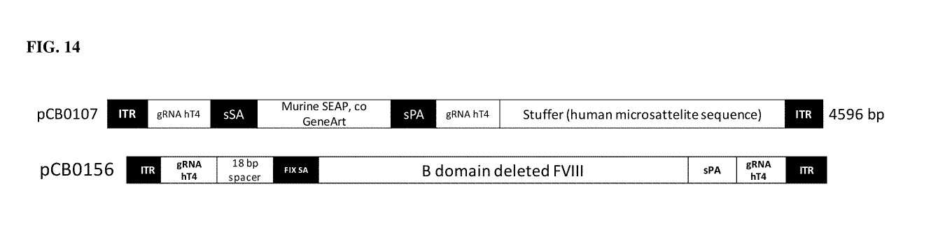

To demonstrate that the concept of targeted integration of a gene cassette in to albumin intron 1 mediated by CRISPR/Cas9 cleavage also works in human cells using a guide RNA specific to the human genome we performed experiments in primary human hepatocytes. Primary human hepatocytes are human hepatocytes collected from the livers of human donors that have undergone minimal in vitro manipulation in order to maintain their normal phenotype. Two donor templates were constructed as shown in FIG. 14 and were packaged in to the AAV-DJ serotype (Grimm et al, 2008; J Virol. 82: 5887-5911) that is particularly effective at transducing hepatocytes in vitro. The AAV-DJ viruses were titered by quantitative PCR using primers and probes located within the coding sequence of the relevant gene (FVIII or mSEAP) resulting in a titer expressed as genome copies (GC) per ml.

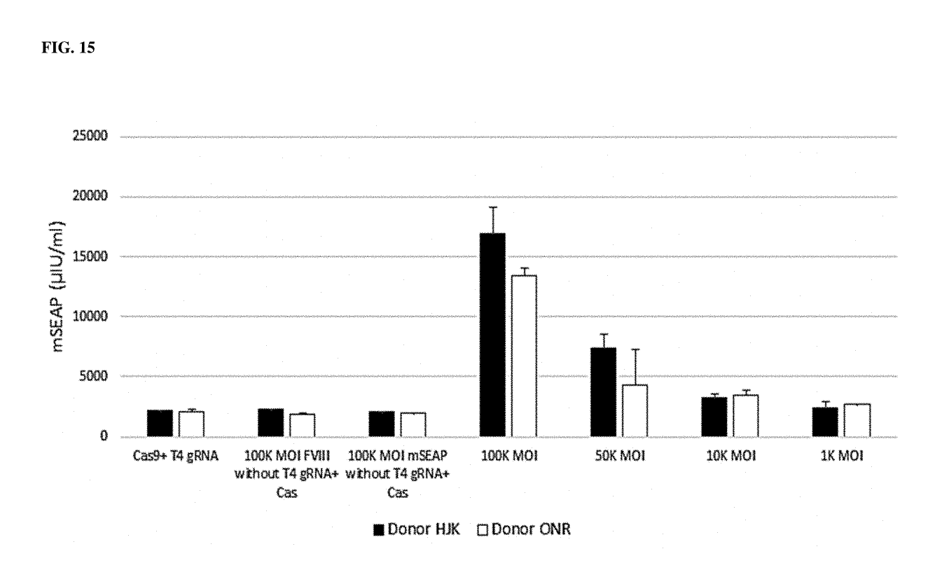

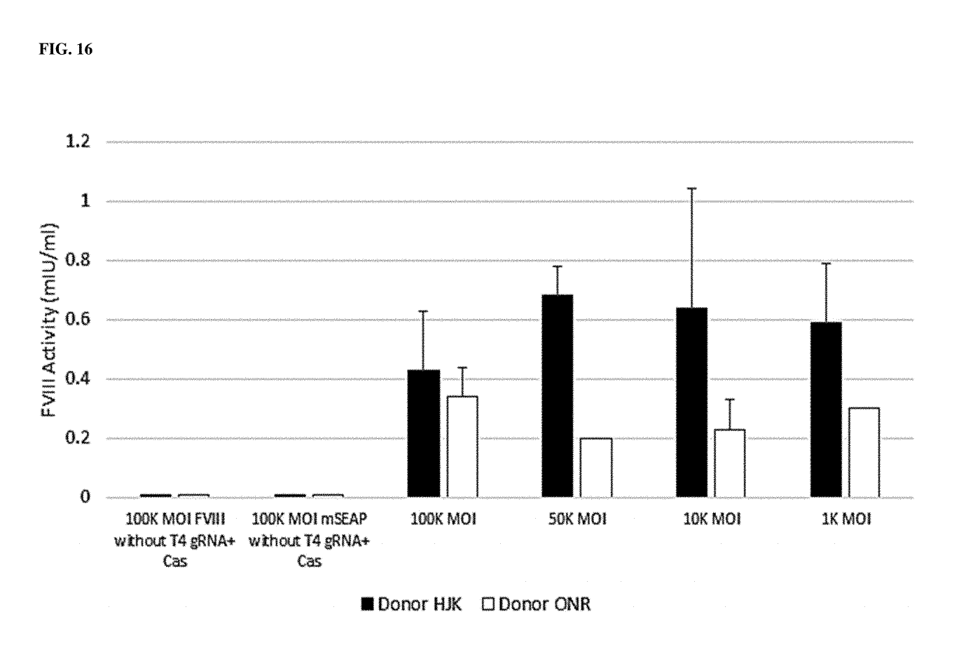

Primary human hepatocytes (obtained from BioIVT, Westbury, N.Y.) were thawed, transferred to Hepatocyte Recovery Medium (CHRM) (Gibco), pelleted at low speed then plated in InVitroGRO™ CP Medium (BioIVT) plus Torpedo™ Antibiotic Mix (BioIVT) at a density of 0.7×10⁶ cells/ml in 24-well plates pre-coated with Collagen IV (Corning). Plates were incubated in 5% CO2 at 37° C. After the cells have adhered (3-4 hours after plating) dead cells that have not adhered to the plate were washed out and fresh warm complete medium was added to the cells. Lipid based transfection mixtures of spCas9 mRNA (made at Trilink) and hAlb T4 guide RNA (made at Synthego Corp, Menlo Park, Calif.) were prepared by adding the RNA to OptiMem media (Gibco) at final concentration of 0.02 ug/ul mRNA and 0.2 uMolar guide. To this was added an equal volume of Lipofectamine diluted 30-fold in Optimem and incubated at room temperature 20 minutes. Either AAV-DJ-pCB0107 or AAV-DJ-pCB0156 was added to relevant wells at various multiplicities of infection ranging from 1,000 GC per cell to 100,000 GC per cell followed immediately (within 5 minutes) with the spCas9 mRNA/gRNA lipid transection mixture. The plates were then incubated in 5% CO₂ at 37° C. for 72 h after which the media was collected and assayed for either FVIII activity using a chromogenic assay (Diapharma, Chromogenix Coatest SP Factor FVIII, cat# K824086kit) or SEAP activity using a commercial kit (InvivoGen). The results are summarized in FIGS. 15 and 16. Controls in which the cells were transfected with the spCas9 mRNA and gRNA alone or the SEAP virus alone or the FVIII virus alone had a low level of SEAP activity representing the background activity in the cells. When both the AAV-DJ-pCB0107 virus and the Cas9 mRNA/hAlbT4 gRNA were transfected the SEAP activity was significantly above the background levels at the higher MOI of 50,000 and 100,000. These data indicate that the combination of CRISPR/Cas9 gene editing components and a AAV delivered donor containing cut sites for the same gRNA can result in the expression of the donor encoded transgene. Because the SEAP gene encoded in the AAV donor lacks a promoter or a signal peptide and because SEAP expression required the gene editing components it is likely that the SEAP was expressed from copies of the donor integrated in to human albumin intron 1. In-out PCR is a method that could be used to confirm integration of the SEAP donor into intron 1 of human albumin.

Controls in which cells were transfected with 100, 000 MOI of either the AAV-DJ-pCB0107 or AAV-DJ-pCB0156 viruses alone (without Cas9 mRNA or gRNA) exhibited low or undetectable levels of FVIII activity in the media at 72 h (FIG. 16). Cells transfected with AAV-DJ-pCB0156 virus at various MOI together with the spCas9 mRNA and hAlbT4 gRNA had measurable levels of FVIII activity in the media at 72 h that ranged from 0.2 to 0.6 mIU/ml. These data indicate that the combination of CRISPR/Cas9 gene editing components and a AAV delivered donor containing cut sites for the same gRNA can result in the expression of the donor encoded FVIII transgene. Because the FVIII gene encoded in the AAV donor lacks a promoter or a signal peptide and because FVIII expression required the gene editing components it is likely that the FVIII was expressed from copies of the donor integrated in to human albumin intron 1. In-out PCR is a method that could be used to confirm integration of the FVIII donor into intron 1 of human albumin.

While the present disclosure has been described at some length and with some particularity with respect to the several described embodiments, it is not intended that it should be limited to any such particulars or embodiments or any particular embodiment, but it is to be construed with references to the appended claims so as to provide the broadest possible interpretation of such claims in view of the prior art and, therefore, to effectively encompass the intended scope of the disclosure.

### SEQUENCE LISTING

In addition to sequences disclosed elsewhere in the present disclosures, the following sequences are provided as they are mentioned or used in various exemplary embodiments of the disclosures, which are provided for the purpose of illustration.

| SEQ | | |
|---|---|---|
| ID | | |
| NO | Sequence | Description |
| 1 | SFSQNPPVLKRHQR | |
| 2 | ctgacctcttctcttcctcccacag | synthetic splice |
| | | acceptor |
| 3 | TTAACAATCCTTTTTTTTCTTCCCTTGCCCAG | native albumin intron |
| | | 1/exon 2 splice |
| | | acceptor, human |
| 4 | ttaaatatgttgtgtggtttttctctccctgtttccacag | native albumin intron |
| | | 1/exon 2 splice |
| | | acceptor, mouse |
| 5 | AATAAAAGATCTTTATTTTCATTAGATCTGTGTGTTGGTTTTTTGTGTG | consensus synthetic |
| | | poly A signal |
| | | sequence |
| 6 | LAGLIDADG | |
| 7 | TGCCTTTACCCCATCGTTAC | target site |
| 8 | TGCCTCCTCCCGATAGTTAC | target site |
| 9 | GGACAGTTCCTGATTGTTAC | target site |
| 10 | TGCCTTTTCCCGATTGTTAA | target site |
| 11 | TTATTACGGTCTCATAGGGC | MALBF3 primer |
| 12 | AGTCTTTCTGTCAATGCACAC | MALBR5 primer |
| 13 | usgscsCAGUUCCCGAUCGUUACGUUUUAGAgcuaGAAAuagcAAGUUAAAAUAA | gRNA |
| | GGCUAGUCCGUUAUCaacuuGAAAaaguggcaccgagucggugcusususU | |
| | ″A, G, U, C″ are native RNA nucleotides | |
| | ″a, g, u, c″ are 2′-O-methyl nucleotides, and | |
| | ″s″ represents a phosphorothioate backbone | |
| 14 | CCCTCCGTTTGTCCTAGCTT | Albumin forward primer |
| 15 | TCTACGAGGCAGCACTGTT | Albumin reverse primer |
| 16 | AACTGCTTCTCCTCTTGGGAAGT | AAVS1 forward primer |
| 17 | CCTCTCCATCCTCTTGCTTTCTTTG | AAVS1 reverse primer |
| 18 | TAATTTTCTTTTGCGCACTAAGG | Human Albumin Intron- |
| | | 1_T1 |
| 19 | TAGTGCAATGGATAGGTCTTTGG | Human Albumin Intron- |
| | | 1_T2 |
| 20 | AGTGCAATGGATAGGTCTTTGGG | Human Albumin Intron- |
| | | 1_T3 |
| 21 | TAAAGCATAGTGCAATGGATAGG | Human Albumin Intron- |
| | | 1_T4 |
| 22 | ATTTATGAGATCAACAGCACAGG | Human Albumin Intron- |
| | | 1_T5 |
| 23 | TGATTCCTACAGAAAAACTCAGG | Human Albumin Intron- |
| | | 1_T6 |
| 24 | TGTATTTGTGAAGTCTTACAAGG | Human Albumin Intron- |
| | | 1_T7 |
| 25 | GACTGAAACTTCACAGAATAGGG | Human Albumin Intron- |
| | | 1_T8 |
| 26 | AATGCATAATCTAAGTCAAATGG | Human Albumin Intron- |
| | | 1_T9 |
| 27 | TGACTGAAACTTCACAGAATAGG | Human Albumin Intron- |
| | | 1_T10 |
| 28 | TTAAATAAAGCATAGTGCAATGG | Human Albumin Intron- |
| | | 1_T11 |
| 29 | GATCAACAGCACAGGTTTTGTGG | Human Albumin Intron- |
| | | 1_T12 |
| 30 | TAATAAAATTCAAACATCCTAGG | Human Albumin Intron- |
| | | 1_T13 |
| 31 | TTCATTTTAGTCTGTCTTCTTGG | Human Albumin Intron- |
| | | 1_T14 |
| 32 | ATTATCTAAGTTTGAATATAAGG | Human Albumin Intron- |
| | | 1_T15 |
| 33 | ATCATCCTGAGTTTTTCTGTAGG | Human Albumin Intron- |
| | | 1_T16 |
| 34 | GCATCTTTAAAGAATTATTTTGG | Human Albumin Intron- |
| | | 1_T17 |
| 35 | TACTAAAACTTTATTTTACTGGG | Human Albumin Intron- |
| | | 1_T18 |
| 36 | TGAATTATTCTTCTGTTTAAAGG | Human Albumin Intron- |
| | | 1_T19 |
| 37 | AATTTTTAAATAGTATTCTTGG | Human Albumin Intron- |
| | | 1_T20 |
| 38 | ATGCATTTGTTTCAAAATATTGG | Human Albumin Intron- |
| | | 1_T21 |
| 39 | TTTGGCATTTATTTCTAAAATGG | Human Albumin Intron- |
| | | 1_T22 |
| 40 | AAAGTTGAACAATAGAAAAATGG | Human Albumin Intron- |
| | | 1_T23 |
| 41 | TTACTAAAACTTTATTTTACTGG | Human Albumin Intron- |
| | | 1_T24 |
| 42 | TGCATTTGTTTCAAAATATTGGG | Human Albumin Intron- |
| | | 1_T26 |
| 43 | TGGGCAAGGGAAGAAAAAAAAGG | Human Albumin Intron- |
| | | 1_T27 |
| 44 | TCCTAGGTAAAAAAAAAAAAAGG | Human Albumin Intron- |
| | | 1_T28 |
| 45 | TAATTTTCTTTTGCCCACTAAGG | |
| 46 | TAGTGCAATGGATAGGTCTTAGG | |
| 47 | AGTGCAATGGATAGGTCTTAGGG | |
| 48 | TAAAGCATAGTGCAATGGATAGG | |
| 49 | ATTTATGAGATCAACAGCACAGG | |
| 50 | TGATTCCTACAGAAAAAGTCAGG | |
| 51 | AATGCATAATCTAAGTCAAATGG | |
| 52 | TTAAATAAAGCATAGTGCAATGG | |
| 53 | ATTTATGAGATCAACAGCACAGG | |
| 54 | TAATAAAATTCAAACATCCTAGG | |
| 55 | ATTATCCTGACTTTTTCTGTAGG | |
| 56 | TACTAAAACTTTATTTTACTTGG | |
| 57 | TGAATTATTCCTCTGTTTAAAGG | |
| 58 | ATGCATTTGTTTCAAAATATTGG | |
| 59 | TTTGGCATTTATTTCTAAAATGG | |
| 60 | AAAGTTGAACAATAGAAAAATGG | |
| 61 | TGCATTTGTTTCAAAATATTGGG | |
| 62 | TAATTTTCTTTTGCCCACTAAGG | |
| 63 | TAGTGCAATGGATAGGTCTTAGG | |
| 64 | AGTGCAATGGATAGGTCTTAGGG | |
| 65 | TAAAGCATAGTGCAATGGATAGG | |
| 66 | ATTTATGAGATCAACAGCACAGG | |
| 67 | TGATTCCTACAGAAAAAGTCAGG | |
| 68 | AATGCATAATCTAAGTCAAATGG | |
| 69 | TTAAATAAAGCATAGTGCAATGG | |
| 70 | ATTTATGAGATCAACAGCACAGG | |
| 71 | TAATAAAATTCAAACATCCTAGG | |
| 72 | ATTATCCTGACTTTTTCTGTAGG | |
| 73 | TACTAAAACTTTATTTTACTTGG | |
| 74 | TGAATTATTCCTCTGTTTAAAGG | |
| 75 | ATGCATTTGTTTCAAAATATTGG | |
| 76 | TTTGGCATTTATTTCTAAAATGG | |
| 77 | AAAGTTGAACAATAGAAAAATGG | |
| 78 | TGCATTTGTTTCAAAATATTGGG | |
| 79 | TGGGGAAGGGGAGAAAAAAAAGG | |
| 80 | tgccagttcccgatcgttacagg | Mouse albumin intron 1 |
| | | gRNA sequence, |
| | | mALbgRNA_T1 |
| 81 | GGAAATAAGAGAGAAAAGAAGAGTAAGAAGAAATATAAGAGCCACCATGGCCCC | spCas9 mRNA |
| | AAAGAAGAAGCGGAAGGTCGGTATCCACGGAGTCCCAGCAGCCGACAAGAAGTA | |
| | CAGCATCGGCCTGGACATCGGCACCAACTCTGTGGGCTGGGCCGTGATCACCGAC | |
| | GAGTACAAGGTGCCCAGCAAGAAATTCAAGGTGCTGGGCAACACCGACCGGCAC | |
| | AGCATCAAGAAGAACCTGATCGGAGCCCTGCTGTTCGACAGCGGCGAAACAGCCG | |
| | AGGCCACCCGGCTGAAGAGAACCGCCAGAAGAAGATACACCAGACGGAAGAACC | |
| | GGATCTGCTATCTGCAAGAGATCTTCAGCAACGAGATGGCCAAGGTGGACGACAG | |
| | CTTCTTCCACAGACTGGAAGAGTCCTTCCTGGTGGAAGAGGACAAGAAGCACGAG | |
| | AGACACCCCATCTTCGGCAACATCGTGGACGAGGTGGCCTACCACGAGAAGTACC | |
| | CCACCATCTACCACCTGAGAAAGAAACTGGTGGACAGCACCGACAAGGCCGACCT | |
| | GAGACTGATCTACCTGGCCCTGGCCCACATGATCAAGTTCAGAGGCCACTTCCTGA | |
| | TCGAGGGCGACCTGAACCCCGACAACAGCGACGTGGACAAGCTGTTCATCCAGCT | |
| | GGTGCAGACCTACAACCAGCTGTTCGAGGAAAACCCCATCAACGCCAGCGGCGTG | |
| | GACGCCAAGGCTATCCTGTCTGCCAGACTGAGCAAGAGCAGAAGGCTGGAAAATC | |
| | TGATCGCCCAGCTGCCCGGCGAGAAGAAGAACGGCCTGTTCGGCAACCTGATTGC | |
| | CCTGAGCCTGGGCCTGACCCCCAACTTCAAGAGCAACTTCGACCTGGCCGAGGAT | |
| | GCCAAACTGCAGCTGAGCAAGGACACCTACGACGACGACCTGGACAACCTGCTGG | |
| | CCCAGATCGGCGACCAGTACGCCGACCTGTTCCTGGCCGCCAAGAACCTGTCTGAC | |
| | GCCATCCTGCTGAGCGACATCCTGAGAGTGAACACCGAGATCACCAAGGCCCCCC | |
| | TGAGCGCCTCTATGATCAAGAGATACGACGAGCACCACCAGGACCTGACCCTGCT | |
| | GAAAGCTCTCGTGCGGCAGCAGCTGCCTGAGAAGTACAAAGAAATCTTCTTCGAC | |
| | CAGAGCAAGAACGGCTACGCCGGCTACATCGATGGCGGCGCTAGCCAGGAAGAG | |
| | TTCTACAAGTTCATCAAGCCCATCCTGGAAAAGATGGACGGCACCGAGGAACTGC | |
| | TCGTGAAGCTGAACAGAGAGGACCTGCTGAGAAAGCAGAGAACCTTCGACAACG | |
| | GCAGCATCCCCCACCAGATCCACCTGGGAGAGCTGCACGCTATCCTGAGAAGGCA | |
| | GGAAGATTTTTACCCATTCCTGAAGGACAACCGGGAAAAGATCGAGAAGATCCTG | |
| | ACCTTCAGGATCCCCTACTACGTGGGCCCCCTGGCCAGAGGCAACAGCAGATTCG | |
| | CCTGGATGACCAGAAAGAGCGAGGAAACCATCACCCCCTGGAACTTCGAGGAAGT | |
| | GGTGGACAAGGGCGCCAGCGCCCAGAGCTTCATCGAGAGAATGACAAACTTCGA | |
| | TAAGAACCTGCCCAACGAGAAGGTGCTGCCCAAGCACAGCCTGCTGTACGAGTAC | |
| | TTCACCGTGTACAACGAGCTGACCAAAGTGAAATACGTGACCGAGGGAATGAGAA | |
| | AGCCCGCCTTCCTGAGCGGCGAGCAGAAAAAGGCCATCGTGGACCTGCTGTTCAA | |
| | GACCAACAGAAAAGTGACCGTGAAGCAGCTGAAAGAGGACTACTTCAAGAAAAT | |
| | CGAGTGCTTCGACTCCGTGGAAATCTCCGGCGTGGAAGATAGATTCAACGCCTCCC | |
| | TGGGCACATACCACGATCTGCTGAAAATTATCAAGGACAAGGACTTCCTGGATAAC | |
| | GAAGAGAACGAGGACATTCTGGAAGATATCGTGCTGACCCTGACACTGTTTGAGG | |
| | ACCGCGAGATGATCGAGGAAAGGCTGAAAACCTACGCTCACCTGTTCGACGACAA | |
| | AGTGATGAAGCAGCTGAAGAGAAGGCGGTACACCGGCTGGGGCAGGCTGAGCA | |
| | GAAAGCTGATCAACGGCATCAGAGACAAGCAGAGCGGCAAGACAATCCTGGATTT | |
| | CCTGAAGTCCGACGGCTTCGCCAACCGGAACTTCATGCAGCTGATCCACGACGAC | |
| | AGCCTGACATTCAAAGAGGACATCCAGAAAGCCCAGGTGTCCGGCCAGGGCGACT | |
| | CTCTGCACGAGCATATCGCTAACCTGGCCGGCAGCCCCGCTATCAAGAAGGGCAT | |
| | CCTGCAGACAGTGAAGGTGGTGGACGAGCTCGTGAAAGTGATGGGCAGACACAA | |
| | GCCCGAGAACATCGTGATCGAGATGGCTAGAGAGAACCAGACCACCCAGAAGGG | |
| | ACAGAAGAACTCCCGCGAGAGGATGAAGAGAATCGAAGAGGGCATCAAAGAGCT | |
| | GGGCAGCCAGATCCTGAAAGAACACCCCGTGGAAAACACCCAGCTGCAGAACGA | |
| | GAAGCTGTACCTGTACTACCTGCAGAATGGCCGGGATATGTACGTGGACCAGGAA | |
| | CTGGACATCAACAGACTGTCCGACTACGATGTGGACCATATCGTGCCTCAGAGCTT | |
| | TCTGAAGGACGACTCCATCGATAACAAAGTGCTGACTCGGAGCGACAAGAACAGA | |
| | GGCAAGAGCGACAACGTGCCCTCCGAAGAGGTCGTGAAGAAGATGAAGAACTAC | |
| | TGGCGACAGCTGCTGAACGCCAAGCTGATTACCCAGAGGAAGTTCGATAACCTGA | |
| | CCAAGGCCGAGAGAGGCGGCCTGAGCGAGCTGGATAAGGCCGGCTTCATCAAGA | |
| | GGCAGCTGGTGGAAACCAGACAGATCACAAAGCACGTGGCACAGATCCTGGACTC | |
| | CCGGATGAACACTAAGTACGACGAAAACGATAAGCTGATCCGGGAAGTGAAAGT | |
| | GATCACCCTGAAGTCCAAGCTGGTGTCCGATTTCCGGAAGGATTTCCAGTTTTACA | |
| | AAGTGCGCGAGATCAACAACTACCACCACGCCCACGACGCCTACCTGAACGCCGT | |
| | CGTGGGAACCGCCCTGATCAAAAAGTACCCTAAGCTGGAAAGCGAGTTCGTGTAC | |
| | GGCGACTACAAGGTGTACGACGTGCGGAAGATGATCGCCAAGAGCGAGCAGGAA | |
| | ATCGGCAAGGCTACCGCCAAGTACTTCTTCTACAGCAACATCATGAACTTTTTCAA | |
| | GACCGAAATCACCCTGGCCAACGGCGAGATCAGAAAGCGCCCTCTGATCGAGACA | |
| | AACGGCGAAACCGGGGAGATCGTGTGGGATAAGGGCAGAGACTTCGCCACAGTG | |
| | CGAAAGGTGCTGAGCATGCCCCAAGTGAATATCGTGAAAAAGACCGAGGTGCAG | |
| | ACAGGCGGCTTCAGCAAAGAGTCTATCCTGCCCAAGAGGAACAGCGACAAGCTGA | |
| | TCGCCAGAAAGAAGGACTGGGACCCCAAGAAGTACGGCGGCTTCGACAGCCCTAC | |
| | CGTGGCCTACTCTGTGCTGGTGGTGGCTAAGGTGGAAAAGGGCAAGTCCAAGAA | |
| | ACTGAAGAGTGTGAAAGAGCTGCTGGGGATCACCATCATGGAAAGAAGCAGCTTT | |
| | GAGAAGAACCCTATCGACTTTCTGGAAGCCAAGGGCTACAAAGAAGTGAAAAAG | |
| | GACCTGATCATCAAGCTGCCTAAGTACTCCCTGTTCGAGCTGGAAAACGGCAGAA | |
| | AGAGAATGCTGGCCTCTGCCGGCGAACTGCAGAAGGGAAACGAGCTGGCCCTGC | |
| | CTAGCAAATATGTGAACTTCCTGTACCTGGCCTCCCACTATGAGAAGCTGAAGGGC | |
| | AGCCCTGAGGACAACGAACAGAAACAGCTGTTTGTGGAACAGCATAAGCACTACC | |
| | TGGACGAGATCATCGAGCAGATCAGCGAGTTCTCCAAGAGAGTGATCCTGGCCGA | |
| | CGCCAATCTGGACAAGGTGCTGTCTGCCTACAACAAGCACAGGGACAAGCCTATC | |
| | AGAGAGCAGGCCGAGAATATCATCCACCTGTTCACCCTGACAAACCTGGGCGCTC | |
| | CTGCCGCCTTCAAGTACTTTGACACCACCATCGACCGGAAGAGGTACACCAGCACC | |
| | AAAGAGGTGCTGGACGCCACCCTGATCCACCAGAGCATCACCGGCCTGTACGAGA | |
| | CAAGAATCGACCTGTCTCAGCTGGGAGGCGACAAGAGACCTGCCGCCACTAAGAA | |
| | GGCCGGACAGGCCAAAAAGAAGAAGTGAGCGGCCGCTTAATTAAGCTGCCTTCTG | |
| | CGGGGCTTGCCTTCTGGCCATGCCCTTCTTCTCTCCCTTGCACCTGTACCTCTTGGTC | |
| | TTTGAATAAAGCCTGAGTAGGAAGAAAAAAAAAAAAAAAAAAAAAAAAAAAAAA | |
| | AAAAAAAAAAAAAAAAAAAAAAAAAAAAAAAAAAAAAAAAAAAAAAAAAA | |
| 82 | ctgacctcttctcttcctcccacag | Synthetic splice |
| | | acceptor |
| 83 | ctttaaatatgttgtgtggtttttctctccctgtttccacag | Mouse albumin Intron 1 |
| | | splice acceptor |
| 84 | cctcatactgaggtttttgtgtctgcttttcag | Mouse albumin Intron 2 |
| | | splice acceptor |
| 85 | ttaacaatccttttttttcttcccttgcccag | Human albumin Intron 1 |
| | | splice acceptor |
| 86 | attatactacatttttctacatcctttgtttcag | Human albumin Intron 2 |
| | | splice acceptor |
| 87 | AATTGCTGACCTCTTCTCTTCCTCCCACAGTGGCCACCAGAAGATACTACCTCGGAG | MAB8A |
| | CCGTCGAATTGAGCTGGGATTACATGCAATCCGACCTGGGAGAACTGCCCGTGGA | |
| | TGCCAGGTTTCCTCCTCGGGTCCCCAAGTCCTTCCCGTTCAACACCTCAGTCGTCTA | |
| | CAAGAAAACCCTCTTCGTGGAGTTCACCGACCATCTGTTCAACATCGCCAAGCCAA | |
| | GACCCCCGTGGATGGGACTCCTCGGTCCGACCATCCAAGCCGAAGTGTACGACAC | |
| | TGTGGTCATTACCCTGAAGAACATGGCCTCCCATCCTGTGTCCCTGCATGCAGTGG | |
| | GCGTGTCCTACTGGAAGGCTTCCGAAGGGGCCGAGTACGACGATCAAACCAGCCA | |
| | GCGGGAAAAGGAGGATGACAAAGTGTTCCCGGGTGGTTCGCACACCTACGTGTG | |
| | GCAAGTGCTCAAGGAGAACGGTCCTATGGCCTCTGATCCCCTGTGTCTGACCTACT | |
| | CCTACCTGTCCCATGTCGACCTCGTGAAGGATCTGAACAGCGGGCTGATTGGCGCC | |
| | CTGCTCGTGTGCCGGGAAGGCTCCCTGGCCAAGGAAAAGACCCAGACACTGCACA | |
| | AGTTCATCTTGCTGTTCGCCGTGTTTGATGAGGGAAAGTCCTGGCATAGCGAGACT | |
| | AAGAACTCCCTTATGCAAGACCGGGATGCTGCCTCCGCTAGGGCTTGGCCTAAGA | |
| | TGCATACTGTGAACGGATACGTGAACAGATCCCTGCCTGGCCTTATCGGTTGCCAC | |
| | CGGAAGTCCGTGTATTGGCATGTGATCGGCATGGGAACCACTCCAGAGGTGCACT | |
| | CCATTTTCTTGGAGGGGCATACCTTCTTGGTGCGCAACCACAGACAGGCCTCCCTG | |
| | GAAATTTCTCCGATCACTTTCCTGACTGCCCAGACCCTCCTTATGGACCTGGGTCAG | |
| | TTCCTGCTGTTCTGCCACATTTCGTCCCACCAACACGATGGCATGGAAGCCTACGT | |
| | GAAAGTGGACTCGTGCCCGGAAGAACCACAGCTGCGGATGAAGAACAACGAAGA | |
| | GGCAGAGGACTACGATGATGATCTTACCGATTCGGAAATGGATGTGGTCCGATTC | |
| | GACGACGATAATAGCCCATCCTTCATCCAAATTAGGAGCGTGGCCAAGAAGCACC | |
| | CCAAAACTTGGGTGCATTACATTGCGGCCGAGGAAGAGGATTGGGACTACGCACC | |
| | CCTCGTGCTTGCACCCGATGATCGGTCCTACAAGTCCCAATACCTGAACAACGGCC | |
| | CGCAGAGGATCGGTCGGAAGTATAAGAAAGTGCGCTTCATGGCCTACACCGACGA | |
| | GACTTTCAAGACCAGAGAGGCCATTCAGCACGAAAGCGGCATTCTGGGGCCGCTG | |
| | TTGTACGGGGAGGTCGGAGATACACTGCTCATCATTTTCAAGAACCAGGCGTCCA | |
| | GACCCTACAACATCTACCCGCACGGAATCACTGACGTCCGCCCCCTGTACTCCCGG | |
| | AGACTCCCGAAGGGAGTCAAGCACTTGAAAGACTTCCCCATCCTGCCTGGGGAAA | |
| | TCTTCAAGTACAAGTGGACCGTGACCGTCGAGGATGGGCCGACCAAGTCCGATCC | |
| | AAGATGCCTCACTAGATACTACTCATCCTTCGTCAACATGGAACGGGACCTGGCCT | |
| | CAGGACTGATTGGCCCCCTGCTCATCTGCTACAAGGAGTCCGTGGATCAGCGCGG | |
| | AAACCAGATCATGTCGGACAAACGCAACGTCATCCTCTTCTCCGTCTTTGACGAGA | |
| | ACCGCTCATGGTACCTTACGGAGAACATCCAGCGGTTCCTCCCCAACCCTGCCGGA | |
| | GTGCAGCTCGAGGACCCGGAATTCCAGGCATCAAACATTATGCACTCCATCAACG | |
| | GTTACGTGTTCGACAGCCTCCAGCTTAGCGTGTGCCTCCATGAAGTCGCATATTGG | |
| | TACATCCTGTCCATTGGAGCACAAACCGACTTTCTCTCCGTGTTCTTCTCCGGATAT | |
| | ACCTTCAAGCACAAGATGGTGTACGAGGATACCCTGACCCTCTTCCCCTTCTCCGG | |
| | AGAGACTGTGTTTATGTCGATGGAAAACCCAGGCCTGTGGATTTTGGGGTGCCAC | |
| | AACTCGGATTTCCGAAACCGGGGCATGACTGCCTTGCTCAAGGTGTCCTCCTGTGA | |
| | CAAGAACACGGGAGACTACTACGAGGACTCCTACGAGGATATTTCCGCCTACCTCC | |
| | TGTCCAAGAACAACGCCATCGAACCCAGGTCCTTCAGCCAGAACCCTCCTGTCCTC | |
| | AAGCGCCATCAGAGAGAAATCACCCGCACGACCCTGCAGTCCGACCAGGAAGAG | |
| | ATCGATTACGACGACACTATCTCCGTCGAAATGAAGAAGGAGGACTTTGACATCTA | |
| | CGACGAAGATGAAAATCAGTCCCCTCGCTCGTTCCAAAAGAAAACGAGACACTAC | |
| | TTCATCGCTGCTGTGGAGCGGCTCTGGGACTACGGCATGTCCTCATCGCCCCACGT | |
| | GCTTAGGAACCGGGCTCAATCCGGGAGCGTCCCTCAGTTCAAGAAAGTGGTGTTT | |
| | CAAGAATTCACCGATGGAAGCTTCACGCAGCCGTTGTACAGGGGCGAACTGAACG | |
| | AGCACCTTGGCCTGCTGGGACCTTACATCAGAGCAGAGGTCGAGGACAACATCAT | |
| | GGTGACCTTCCGGAACCAAGCCTCCCGGCCATATTCATTCTACTCGAGCCTTATCTC | |
| | ATACGAGGAGGATCAGAGACAGGGGGCTGAACCTCGGAAGAACTTCGTCAAGCC | |
| | GAACGAGACAAAGACCTACTTTTGGAAGGTGCAGCACCACATGGCCCCGACCAAG | |
| | GATGAGTTCGACTGCAAGGCCTGGGCGTACTTCTCCGACGTGGATCTCGAAAAGG | |
| | ACGTGCATTCCGGGCTGATCGGACCGCTGCTCGTCTGCCACACTAACACCCTCAAT | |
| | CCTGCTCACGGCAGACAAGTGACCGTGCAGGAGTTCGCCCTGTTCTTCACCATCTT | |
| | CGACGAAACTAAGTCATGGTACTTTACCGAGAACATGGAGCGGAATTGTCGGGCC | |
| | CCATGTAACATCCAGATGGAGGACCCGACATTCAAGGAGAACTACCGGTTCCACG | |
| | CCATTAACGGATACATTATGGACACTCTTCCGGGACTCGTGATGGCACAGGACCAA | |
| | CGCATCAGATGGTATCTTCTGTCGATGGGGAGCAACGAAAACATCCATTCGATCCA | |
| | CTTTAGCGGTCACGTGTTCACAGTGCGCAAGAAGGAAGAGTACAAGATGGCGCTG | |
| | TACAACCTGTACCCTGGGGTGTTCGAGACTGTGGAAATGCTGCCGTCCAAGGCCG | |
| | GAATTTGGCGCGTGGAATGTCTGATCGGTGAACATCTGCATGCCGGAATGTCCAC | |
| | CCTGTTCCTGGTGTACTCCAACAAGTGCCAAACCCCACTGGGAATGGCATCAGGAC | |
| | ACATTAGAGACTTCCAGATTACCGCGAGCGGACAGTACGGACAATGGGCCCCCAA | |
| | GTTGGCCAGGCTGCACTACTCTGGAAGCATTAACGCCTGGAGCACCAAGGAGCCG | |
| | TTCAGCTGGATCAAGGTGGACCTTCTGGCGCCAATGATCATCCACGGAATTAAGAC | |
| | TCAGGGAGCCCGCCAGAAGTTCTCATCGCTCTACATCTCCCAGTTTATCATCATGTA | |
| | CTCACTGGATGGGAAGAAGTGGCAGACTTACCGGGGAAATTCCACCGGTACTCTG | |
| | ATGGTGTTCTTCGGAAACGTGGACAGCTCCGGCATCAAGCACAATATCTTTAACCC | |
| | GCCTATCATCGCCCGATACATCCGGCTCCACCCGACTCACTACTCCATCCGGTCGAC | |
| | TCTGCGGATGGAACTCATGGGTTGCGACCTCAACTCCTGCTCAATGCCACTGGGCA | |
| | TGGAGTCCAAGGCTATCTCGGACGCTCAGATTACTGCATCGTCGTACTTTACCAAC | |
| | ATGTTCGCTACCTGGTCCCCGTCCAAAGCCCGGCTGCATCTCCAAGGCAGATCAAA | |
| | CGCGTGGAGGCCTCAGGTCAACAACCCGAAGGAATGGCTTCAGGTCGACTTCCAA | |
| | AAGACCATGAAAGTCACCGGAGTGACCACCCAGGGCGTGAAATCGCTGCTGACCT | |
| | CTATGTACGTGAAGGAATTCCTGATCTCATCAAGCCAGGACGGCCACCAGTGGAC | |
| | ACTGTTCTTCCAAAATGGAAAGGTCAAGGTCTTTCAGGGAAATCAAGACTCCTTCA | |
| | CCCCCGTGGTGAACTCCCTGGACCCCCCTCTGCTTACCCGCTACTTGCGCATTCATC | |
| | CGCAATCCTGGGTGCACCAGATCGCCCTGCGAATGGAAGTGCTGGGCTGTGAAGC | |
| | GCAGGACCTGTACTAAAATAAAAGATCTTTATTTTCATTAGATCTGTGTGTTGGTTT | |
| | TTTGTGTGCCGC | |
| 88 | AATTGAACTTTGAGTGTAGCAGAGAGGAACCATTGCCACCTTCAGATTTTAATGTC | MAB8B |
| | TGACCTCTTCTCTTCCTCCCACAGTGGCCACCAGAAGATACTACCTCGGAGCCGTC | |
| | GAATTGAGCTGGGATTACATGCAATCCGACCTGGGAGAACTGCCCGTGGATGCCA | |
| | GGTTTCCTCCTCGGGTCCCCAAGTCCTTCCCGTTCAACACCTCAGTCGTCTACAAGA | |
| | AAACCCTCTTCGTGGAGTTCACCGACCATCTGTTCAACATCGCCAAGCCAAGACCC | |
| | CCGTGGATGGGACTCCTCGGTCCGACCATCCAAGCCGAAGTGTACGACACTGTGG | |
| | TCATTACCCTGAAGAACATGGCCTCCCATCCTGTGTCCCTGCATGCAGTGGGCGTG | |
| | TCCTACTGGAAGGCTTCCGAAGGGGCCGAGTACGACGATCAAACCAGCCAGCGG | |
| | GAAAAGGAGGATGACAAAGTGTTCCCGGGTGGTTCGCACACCTACGTGTGGCAA | |
| | GTGCTCAAGGAGAACGGTCCTATGGCCTCTGATCCCCTGTGTCTGACCTACTCCTA | |
| | CCTGTCCCATGTCGACCTCGTGAAGGATCTGAACAGCGGGCTGATTGGCGCCCTG | |
| | CTCGTGTGCCGGGAAGGCTCCCTGGCCAAGGAAAAGACCCAGACACTGCACAAGT | |
| | TCATCTTGCTGTTCGCCGTGTTTGATGAGGGAAAGTCCTGGCATAGCGAGACTAAG | |
| | AACTCCCTTATGCAAGACCGGGATGCTGCCTCCGCTAGGGCTTGGCCTAAGATGCA | |
| | TACTGTGAACGGATACGTGAACAGATCCCTGCCTGGCCTTATCGGTTGCCACCGGA | |
| | AGTCCGTGTATTGGCATGTGATCGGCATGGGAACCACTCCAGAGGTGCACTCCATT | |
| | TTCTTGGAGGGGCATACCTTCTTGGTGCGCAACCACAGACAGGCCTCCCTGGAAAT | |
| | TTCTCCGATCACTTTCCTGACTGCCCAGACCCTCCTTATGGACCTGGGTCAGTTCCT | |
| | GCTGTTCTGCCACATTTCGTCCCACCAACACGATGGCATGGAAGCCTACGTGAAAG | |
| | TGGACTCGTGCCCGGAAGAACCACAGCTGCGGATGAAGAACAACGAAGAGGCAG | |
| | AGGACTACGATGATGATCTTACCGATTCGGAAATGGATGTGGTCCGATTCGACGA | |
| | CGATAATAGCCCATCCTTCATCCAAATTAGGAGCGTGGCCAAGAAGCACCCCAAAA | |
| | CTTGGGTGCATTACATTGCGGCCGAGGAAGAGGATTGGGACTACGCACCCCTCGT | |
| | GCTTGCACCCGATGATCGGTCCTACAAGTCCCAATACCTGAACAACGGCCCGCAGA | |
| | GGATCGGTCGGAAGTATAAGAAAGTGCGCTTCATGGCCTACACCGACGAGACTTT | |
| | CAAGACCAGAGAGGCCATTCAGCACGAAAGCGGCATTCTGGGGCCGCTGTTGTAC | |
| | GGGGAGGTCGGAGATACACTGCTCATCATTTTCAAGAACCAGGCGTCCAGACCCT | |
| | ACAACATCTACCCGCACGGAATCACTGACGTCCGCCCCCTGTACTCCCGGAGACTC | |
| | CCGAAGGGAGTCAAGCACTTGAAAGACTTCCCCATCCTGCCTGGGGAAATCTTCA | |
| | AGTACAAGTGGACCGTGACCGTCGAGGATGGGCCGACCAAGTCCGATCCAAGAT | |
| | GCCTCACTAGATACTACTCATCCTTCGTCAACATGGAACGGGACCTGGCCTCAGGA | |
| | CTGATTGGCCCCCTGCTCATCTGCTACAAGGAGTCCGTGGATCAGCGCGGAAACC | |
| | AGATCATGTCGGACAAACGCAACGTCATCCTCTTCTCCGTCTTTGACGAGAACCGC | |
| | TCATGGTACCTTACGGAGAACATCCAGCGGTTCCTCCCCAACCCTGCCGGAGTGCA | |
| | GCTCGAGGACCCGGAATTCCAGGCATCAAACATTATGCACTCCATCAACGGTTACG | |
| | TGTTCGACAGCCTCCAGCTTAGCGTGTGCCTCCATGAAGTCGCATATTGGTACATC | |
| | CTGTCCATTGGAGCACAAACCGACTTTCTCTCCGTGTTCTTCTCCGGATATACCTTC | |
| | AAGCACAAGATGGTGTACGAGGATACCCTGACCCTCTTCCCCTTCTCCGGAGAGAC | |
| | TGTGTTTATGTCGATGGAAAACCCAGGCCTGTGGATTTTGGGGTGCCACAACTCG | |
| | GATTTCCGAAACCGGGGCATGACTGCCTTGCTCAAGGTGTCCTCCTGTGACAAGAA | |
| | CACGGGAGACTACTACGAGGACTCCTACGAGGATATTTCCGCCTACCTCCTGTCCA | |
| | AGAACAACGCCATCGAACCCAGGTCCTTCAGCCAGAACCCTCCTGTCCTCAAGCGC | |
| | CATCAGAGAGAAATCACCCGCACGACCCTGCAGTCCGACCAGGAAGAGATCGATT | |
| | ACGACGACACTATCTCCGTCGAAATGAAGAAGGAGGACTTTGACATCTACGACGA | |
| | AGATGAAAATCAGTCCCCTCGCTCGTTCCAAAAGAAAACGAGACACTACTTCATCG | |
| | CTGCTGTGGAGCGGCTCTGGGACTACGGCATGTCCTCATCGCCCCACGTGCTTAGG | |
| | AACCGGGCTCAATCCGGGAGCGTCCCTCAGTTCAAGAAAGTGGTGTTTCAAGAAT | |
| | TCACCGATGGAAGCTTCACGCAGCCGTTGTACAGGGGCGAACTGAACGAGCACCT | |
| | TGGCCTGCTGGGACCTTACATCAGAGCAGAGGTCGAGGACAACATCATGGTGACC | |
| | TTCCGGAACCAAGCCTCCCGGCCATATTCATTCTACTCGAGCCTTATCTCATACGAG | |
| | GAGGATCAGAGACAGGGGGCTGAACCTCGGAAGAACTTCGTCAAGCCGAACGAG | |
| | ACAAAGACCTACTTTTGGAAGGTGCAGCACCACATGGCCCCGACCAAGGATGAGT | |
| | TCGACTGCAAGGCCTGGGCGTACTTCTCCGACGTGGATCTCGAAAAGGACGTGCA | |
| | TTCCGGGCTGATCGGACCGCTGCTCGTCTGCCACACTAACACCCTCAATCCTGCTCA | |
| | CGGCAGACAAGTGACCGTGCAGGAGTTCGCCCTGTTCTTCACCATCTTCGACGAAA | |
| | CTAAGTCATGGTACTTTACCGAGAACATGGAGCGGAATTGTCGGGCCCCATGTAA | |
| | CATCCAGATGGAGGACCCGACATTCAAGGAGAACTACCGGTTCCACGCCATTAAC | |
| | GGATACATTATGGACACTCTTCCGGGACTCGTGATGGCACAGGACCAACGCATCA | |
| | GATGGTATCTTCTGTCGATGGGGAGCAACGAAAACATCCATTCGATCCACTTTAGC | |
| | GGTCACGTGTTCACAGTGCGCAAGAAGGAAGAGTACAAGATGGCGCTGTACAAC | |
| | CTGTACCCTGGGGTGTTCGAGACTGTGGAAATGCTGCCGTCCAAGGCCGGAATTT | |
| | GGCGCGTGGAATGTCTGATCGGTGAACATCTGCATGCCGGAATGTCCACCCTGTTC | |
| | CTGGTGTACTCCAACAAGTGCCAAACCCCACTGGGAATGGCATCAGGACACATTA | |
| | GAGACTTCCAGATTACCGCGAGCGGACAGTACGGACAATGGGCCCCCAAGTTGGC | |
| | CAGGCTGCACTACTCTGGAAGCATTAACGCCTGGAGCACCAAGGAGCCGTTCAGC | |
| | TGGATCAAGGTGGACCTTCTGGCGCCAATGATCATCCACGGAATTAAGACTCAGG | |
| | GAGCCCGCCAGAAGTTCTCATCGCTCTACATCTCCCAGTTTATCATCATGTACTCAC | |
| | TGGATGGGAAGAAGTGGCAGACTTACCGGGGAAATTCCACCGGTACTCTGATGGT | |
| | GTTCTTCGGAAACGTGGACAGCTCCGGCATCAAGCACAATATCTTTAACCCGCCTA | |
| | TCATCGCCCGATACATCCGGCTCCACCCGACTCACTACTCCATCCGGTCGACTCTGC | |
| | GGATGGAACTCATGGGTTGCGACCTCAACTCCTGCTCAATGCCACTGGGCATGGA | |
| | GTCCAAGGCTATCTCGGACGCTCAGATTACTGCATCGTCGTACTTTACCAACATGTT | |
| | CGCTACCTGGTCCCCGTCCAAAGCCCGGCTGCATCTCCAAGGCAGATCAAACGCGT | |
| | GGAGGCCTCAGGTCAACAACCCGAAGGAATGGCTTCAGGTCGACTTCCAAAAGAC | |
| | CATGAAAGTCACCGGAGTGACCACCCAGGGCGTGAAATCGCTGCTGACCTCTATG | |
| | TACGTGAAGGAATTCCTGATCTCATCAAGCCAGGACGGCCACCAGTGGACACTGT | |
| | TCTTCCAAAATGGAAAGGTCAAGGTCTTTCAGGGAAATCAAGACTCCTTCACCCCC | |
| | GTGGTGAACTCCCTGGACCCCCCTCTGCTTACCCGCTACTTGCGCATTCATCCGCAA | |
| | TCCTGGGTGCACCAGATCGCCCTGCGAATGGAAGTGCTGGGCTGTGAAGCGCAG | |
| | GACCTGTACTAAAATAAAAGATCTTTATTTTCATTAGATCTGTGTGTTGGTTTTTTGT | |
| | GTGCGATCGGGAACTGGCATCTTCAGGGAGTAGCTTAGGTCAGTGAAGAGAAGC | |
| | CGC | |
| 89 | gcggcctaaggcAATTGTGCCAGTTCCCGATCGTTACAGGAACTTTGAGTGTAGCAGA | MAB8C |
| | GAGGAACCATTGCCACCTTCAGATTTTAATGTCTGACCTCTTCTCTTCCTCCCACAG | |
| | TGGCCACCAGAAGATACTACCTCGGAGCCGTCGAATTGAGCTGGGATTACATGCA | |
| | ATCCGACCTGGGAGAACTGCCCGTGGATGCCAGGTTTCCTCCTCGGGTCCCCAAGT | |
| | CCTTCCCGTTCAACACCTCAGTCGTCTACAAGAAAACCCTCTTCGTGGAGTTCACCG | |
| | ACCATCTGTTCAACATCGCCAAGCCAAGACCCCCGTGGATGGGACTCCTCGGTCCG | |
| | ACCATCCAAGCCGAAGTGTACGACACTGTGGTCATTACCCTGAAGAACATGGCCTC | |
| | CCATCCTGTGTCCCTGCATGCAGTGGGCGTGTCCTACTGGAAGGCTTCCGAAGGG | |
| | GCCGAGTACGACGATCAAACCAGCCAGCGGGAAAAGGAGGATGACAAAGTGTTC | |
| | CCGGGTGGTTCGCACACCTACGTGTGGCAAGTGCTCAAGGAGAACGGTCCTATGG | |
| | CCTCTGATCCCCTGTGTCTGACCTACTCCTACCTGTCCCATGTCGACCTCGTGAAGG | |
| | ATCTGAACAGCGGGCTGATTGGCGCCCTGCTCGTGTGCCGGGAAGGCTCCCTGGC | |
| | CAAGGAAAAGACCCAGACACTGCACAAGTTCATCTTGCTGTTCGCCGTGTTTGATG | |
| | AGGGAAAGTCCTGGCATAGCGAGACTAAGAACTCCCTTATGCAAGACCGGGATGC | |
| | TGCCTCCGCTAGGGCTTGGCCTAAGATGCATACTGTGAACGGATACGTGAACAGA | |
| | TCCCTGCCTGGCCTTATCGGTTGCCACCGGAAGTCCGTGTATTGGCATGTGATCGG | |
| | CATGGGAACCACTCCAGAGGTGCACTCCATTTTCTTGGAGGGGCATACCTTCTTGG | |
| | TGCGCAACCACAGACAGGCCTCCCTGGAAATTTCTCCGATCACTTTCCTGACTGCCC | |
| | AGACCCTCCTTATGGACCTGGGTCAGTTCCTGCTGTTCTGCCACATTTCGTCCCACC | |
| | AACACGATGGCATGGAAGCCTACGTGAAAGTGGACTCGTGCCCGGAAGAACCAC | |
| | AGCTGCGGATGAAGAACAACGAAGAGGCAGAGGACTACGATGATGATCTTACCG | |
| | ATTCGGAAATGGATGTGGTCCGATTCGACGACGATAATAGCCCATCCTTCATCCAA | |
| | ATTAGGAGCGTGGCCAAGAAGCACCCCAAAACTTGGGTGCATTACATTGCGGCCG | |
| | AGGAAGAGGATTGGGACTACGCACCCCTCGTGCTTGCACCCGATGATCGGTCCTA | |
| | CAAGTCCCAATACCTGAACAACGGCCCGCAGAGGATCGGTCGGAAGTATAAGAAA | |
| | GTGCGCTTCATGGCCTACACCGACGAGACTTTCAAGACCAGAGAGGCCATTCAGC | |
| | ACGAAAGCGGCATTCTGGGGCCGCTGTTGTACGGGGAGGTCGGAGATACACTGC | |
| | TCATCATTTTCAAGAACCAGGCGTCCAGACCCTACAACATCTACCCGCACGGAATC | |
| | ACTGACGTCCGCCCCCTGTACTCCCGGAGACTCCCGAAGGGAGTCAAGCACTTGA | |
| | AAGACTTCCCCATCCTGCCTGGGGAAATCTTCAAGTACAAGTGGACCGTGACCGTC | |
| | GAGGATGGGCCGACCAAGTCCGATCCAAGATGCCTCACTAGATACTACTCATCCTT | |
| | CGTCAACATGGAACGGGACCTGGCCTCAGGACTGATTGGCCCCCTGCTCATCTGCT | |
| | ACAAGGAGTCCGTGGATCAGCGCGGAAACCAGATCATGTCGGACAAACGCAACG | |
| | TCATCCTCTTCTCCGTCTTTGACGAGAACCGCTCATGGTACCTTACGGAGAACATCC | |
| | AGCGGTTCCTCCCCAACCCTGCCGGAGTGCAGCTCGAGGACCCGGAATTCCAGGC | |
| | ATCAAACATTATGCACTCCATCAACGGTTACGTGTTCGACAGCCTCCAGCTTAGCG | |
| | TGTGCCTCCATGAAGTCGCATATTGGTACATCCTGTCCATTGGAGCACAAACCGAC | |
| | TTTCTCTCCGTGTTCTTCTCCGGATATACCTTCAAGCACAAGATGGTGTACGAGGAT | |
| | ACCCTGACCCTCTTCCCCTTCTCCGGAGAGACTGTGTTTATGTCGATGGAAAACCCA | |
| | GGCCTGTGGATTTTGGGGTGCCACAACTCGGATTTCCGAAACCGGGGCATGACTG | |
| | CCTTGCTCAAGGTGTCCTCCTGTGACAAGAACACGGGAGACTACTACGAGGACTC | |
| | CTACGAGGATATTTCCGCCTACCTCCTGTCCAAGAACAACGCCATCGAACCCAGGT | |
| | CCTTCAGCCAGAACCCTCCTGTCCTCAAGCGCCATCAGAGAGAAATCACCCGCACG | |
| | ACCCTGCAGTCCGACCAGGAAGAGATCGATTACGACGACACTATCTCCGTCGAAAT | |
| | GAAGAAGGAGGACTTTGACATCTACGACGAAGATGAAAATCAGTCCCCTCGCTCG | |
| | TTCCAAAAGAAAACGAGACACTACTTCATCGCTGCTGTGGAGCGGCTCTGGGACT | |
| | ACGGCATGTCCTCATCGCCCCACGTGCTTAGGAACCGGGCTCAATCCGGGAGCGT | |
| | CCCTCAGTTCAAGAAAGTGGTGTTTCAAGAATTCACCGATGGAAGCTTCACGCAGC | |
| | CGTTGTACAGGGGCGAACTGAACGAGCACCTTGGCCTGCTGGGACCTTACATCAG | |
| | AGCAGAGGTCGAGGACAACATCATGGTGACCTTCCGGAACCAAGCCTCCCGGCCA | |
| | TATTCATTCTACTCGAGCCTTATCTCATACGAGGAGGATCAGAGACAGGGGGCTG | |
| | AACCTCGGAAGAACTTCGTCAAGCCGAACGAGACAAAGACCTACTTTTGGAAGGT | |
| | GCAGCACCACATGGCCCCGACCAAGGATGAGTTCGACTGCAAGGCCTGGGCGTAC | |
| | TTCTCCGACGTGGATCTCGAAAAGGACGTGCATTCCGGGCTGATCGGACCGCTGC | |
| | TCGTCTGCCACACTAACACCCTCAATCCTGCTCACGGCAGACAAGTGACCGTGCAG | |
| | GAGTTCGCCCTGTTCTTCACCATCTTCGACGAAACTAAGTCATGGTACTTTACCGAG | |
| | AACATGGAGCGGAATTGTCGGGCCCCATGTAACATCCAGATGGAGGACCCGACAT | |
| | TCAAGGAGAACTACCGGTTCCACGCCATTAACGGATACATTATGGACACTCTTCCG | |
| | GGACTCGTGATGGCACAGGACCAACGCATCAGATGGTATCTTCTGTCGATGGGGA | |
| | GCAACGAAAACATCCATTCGATCCACTTTAGCGGTCACGTGTTCACAGTGCGCAAG | |
| | AAGGAAGAGTACAAGATGGCGCTGTACAACCTGTACCCTGGGGTGTTCGAGACTG | |
| | TGGAAATGCTGCCGTCCAAGGCCGGAATTTGGCGCGTGGAATGTCTGATCGGTGA | |
| | ACATCTGCATGCCGGAATGTCCACCCTGTTCCTGGTGTACTCCAACAAGTGCCAAA | |
| | CCCCACTGGGAATGGCATCAGGACACATTAGAGACTTCCAGATTACCGCGAGCGG | |
| | ACAGTACGGACAATGGGCCCCCAAGTTGGCCAGGCTGCACTACTCTGGAAGCATT | |
| | AACGCCTGGAGCACCAAGGAGCCGTTCAGCTGGATCAAGGTGGACCTTCTGGCGC | |
| | CAATGATCATCCACGGAATTAAGACTCAGGGAGCCCGCCAGAAGTTCTCATCGCTC | |
| | TACATCTCCCAGTTTATCATCATGTACTCACTGGATGGGAAGAAGTGGCAGACTTA | |
| | CCGGGGAAATTCCACCGGTACTCTGATGGTGTTCTTCGGAAACGTGGACAGCTCC | |
| | GGCATCAAGCACAATATCTTTAACCCGCCTATCATCGCCCGATACATCCGGCTCCAC | |
| | CCGACTCACTACTCCATCCGGTCGACTCTGCGGATGGAACTCATGGGTTGCGACCT | |
| | CAACTCCTGCTCAATGCCACTGGGCATGGAGTCCAAGGCTATCTCGGACGCTCAGA | |
| | TTACTGCATCGTCGTACTTTACCAACATGTTCGCTACCTGGTCCCCGTCCAAAGCCC | |
| | GGCTGCATCTCCAAGGCAGATCAAACGCGTGGAGGCCTCAGGTCAACAACCCGAA | |
| | GGAATGGCTTCAGGTCGACTTCCAAAAGACCATGAAAGTCACCGGAGTGACCACC | |
| | CAGGGCGTGAAATCGCTGCTGACCTCTATGTACGTGAAGGAATTCCTGATCTCATC | |
| | AAGCCAGGACGGCCACCAGTGGACACTGTTCTTCCAAAATGGAAAGGTCAAGGTC | |
| | TTTCAGGGAAATCAAGACTCCTTCACCCCCGTGGTGAACTCCCTGGACCCCCCTCT | |
| | GCTTACCCGCTACTTGCGCATTCATCCGCAATCCTGGGTGCACCAGATCGCCCTGC | |
| | GAATGGAAGTGCTGGGCTGTGAAGCGCAGGACCTGTACTAAAATAAAAGATCTTT | |
| | ATTTTCATTAGATCTGTGTGTTGGTTTTTTGTGTGCGATCGGGAACTGGCATCTTCA | |
| | GGGAGTAGCTTAGGTCAGTGAAGAGAAGTGCCAGTTCCCGATCGTTACAGGCCGC | |
| | gggccgc | |
| 90 | gcggcctaaggcAATTGTGCCAGTTCCCGATCGTTACAGGAACTTTGAGTGTAGCAGA | MAB8D |
| | GAGGAACCATTGCCACCTTCAGATTTTAATGTCTGACCTCTTCTCTTCCTCCCACAG | |
| | TGGCCACCAGAAGGTACTACCTAGGAGCCGTGGAACTGAGCTGGGACTACATGCA | |
| | GTCTGACCTGGGAGAGCTGCCCGTGGACGCTAGATTTCCTCCAAGAGTGCCCAAG | |
| | AGCTTCCCCTTCAACACCTCCGTGGTGTACAAGAAAACCCTGTTCGTGGAATTCAC | |
| | CGACCACCTGTTCAATATCGCCAAGCCTAGACCTCCTTGGATGGGCCTGCTGGGCC | |
| | CTACAATTCAGGCCGAGGTGTACGACACCGTGGTCATCACCCTGAAGAACATGGC | |
| | CAGCCATCCTGTGTCTCTGCACGCCGTGGGAGTGTCTTACTGGAAGGCTTCTGAGG | |
| | GCGCCGAGTACGACGACCAGACAAGCCAGAGAGAGAAAGAGGACGACAAGGTTT | |
| | TCCCTGGCGGCAGCCACACCTATGTCTGGCAGGTCCTGAAAGAAAACGGCCCTAT | |
| | GGCCTCCGATCCTCTGTGCCTGACATACAGCTACCTGAGCCATGTGGACCTGGTCA | |
| | AGGACCTGAACTCTGGCCTGATCGGCGCTCTGCTCGTGTGTAGAGAAGGCAGCCT | |
| | GGCCAAAGAAAAGACCCAGACACTGCACAAGTTCATCCTGCTGTTCGCCGTGTTCG | |
| | ACGAGGGCAAGAGCTGGCACAGCGAGACAAAGAACAGCCTGATGCAGGACAGA | |
| | GATGCCGCCTCTGCTAGAGCTTGGCCCAAGATGCACACCGTGAACGGCTACGTGA | |
| | ACAGAAGCCTGCCTGGACTGATCGGATGCCACAGAAAGTCCGTGTACTGGCATGT | |
| | GATCGGCATGGGCACCACACCTGAGGTGCACAGCATCTTTCTGGAAGGACACACC | |
| | TTCCTCGTGCGGAACCACAGACAGGCCAGCCTGGAAATCAGCCCTATCACCTTCCT | |
| | GACCGCTCAGACCCTGCTGATGGATCTGGGCCAGTTTCTGCTGTTCTGCCACATCA | |
| | GCAGCCACCAGCACGATGGCATGGAAGCCTACGTGAAGGTGGACAGCTGCCCCG | |
| | AAGAACCCCAGCTGAGAATGAAGAACAACGAGGAAGCCGAGGACTACGACGACG | |
| | ACCTGACCGACTCTGAGATGGACGTCGTCAGATTCGACGACGATAACAGCCCCAG | |
| | CTTCATCCAGATCAGAAGCGTGGCCAAGAAGCACCCCAAGACCTGGGTGCACTAT | |
| | ATCGCCGCCGAGGAAGAGGACTGGGATTACGCTCCTCTGGTGCTGGCCCCTGACG | |
| | ACAGAAGCTACAAGAGCCAGTACCTGAACAACGGCCCTCAGAGAATCGGCCGGA | |
| | AGTATAAGAAAGTGCGGTTCATGGCCTACACCGACGAGACATTCAAGACCAGAGA | |
| | GGCTATCCAGCACGAGAGCGGCATTCTGGGACCTCTGCTGTATGGCGAAGTGGGC | |
| | GACACACTGCTGATCATCTTCAAGAACCAGGCCAGCAGACCCTACAACATCTACCC | |
| | TCACGGCATCACCGATGTGCGGCCTCTGTACTCTAGAAGGCTGCCCAAGGGCGTG | |
| | AAGCACCTGAAGGACTTCCCTATCCTGCCTGGCGAGATCTTCAAGTACAAGTGGAC | |
| | CGTGACCGTCGAGGACGGCCCTACCAAGAGCGATCCTAGATGCCTGACACGGTAC | |
| | TACAGCAGCTTCGTGAACATGGAACGCGACCTGGCCAGCGGCCTGATTGGTCCTC | |
| | TGCTGATCTGCTACAAAGAAAGCGTGGACCAGAGGGGCAACCAGATCATGAGCG | |
| | ACAAGAGAAACGTGATCCTGTTCTCCGTCTTTGACGAGAACAGGTCCTGGTATCTG | |
| | ACCGAGAACATCCAGCGGTTTCTGCCCAATCCTGCTGGCGTGCAGCTGGAAGATC | |
| | CTGAGTTCCAGGCCTCCAACATCATGCACTCCATCAACGGCTATGTGTTCGACAGC | |
| | CTGCAGCTGAGCGTGTGCCTGCACGAAGTGGCCTACTGGTACATCCTGTCTATCGG | |
| | CGCCCAGACCGACTTCCTGTCCGTGTTCTTTAGCGGCTACACCTTCAAGCACAAGA | |
| | TGGTGTACGAGGATACCCTGACACTGTTCCCATTCAGCGGCGAGACAGTGTTCATG | |
| | AGCATGGAAAACCCCGGCCTGTGGATCCTGGGCTGTCACAACAGCGACTTCAGAA | |
| | ACAGAGGCATGACAGCCCTGCTGAAGGTGTCCAGCTGCGACAAGAACACCGGCG | |
| | ACTACTACGAGGACTCTTACGAGGACATCAGCGCCTACCTGCTGAGCAAGAACAA | |
| | TGCCATCGAGCCTCGGAGCTTCTCTCAGAACCCTCCTGTGCTGAAGAGACACCAGC | |
| | GCGAGATCACCAGAACCACACTGCAGAGCGACCAAGAGGAAATCGATTACGACG | |
| | ACACCATCAGCGTCGAGATGAAGAAAGAAGATTTCGACATCTACGACGAGGACGA | |
| | GAATCAGAGCCCCAGATCTTTCCAGAAGAAAACGCGGCACTACTTCATTGCCGCCG | |
| | TGGAAAGACTGTGGGACTACGGCATGAGCAGCAGCCCACATGTGCTGAGAAACA | |
| | GGGCCCAGAGCGGAAGCGTGCCCCAGTTCAAGAAAGTGGTGTTCCAAGAGTTCAC | |
| | CGACGGCAGCTTCACCCAGCCTCTGTATAGAGGCGAGCTGAACGAGCACCTGGGA | |
| | CTGCTGGGACCTTACATCAGAGCTGAGGTCGAGGATAACATCATGGTCACCTTTAG | |
| | AAACCAGGCCTCTAGGCCCTACTCCTTCTACAGCTCCCTGATCAGCTACGAAGAGG | |
| | ACCAGAGACAGGGCGCTGAGCCCAGAAAGAACTTCGTGAAGCCCAACGAGACTA | |
| | AGACCTACTTTTGGAAGGTGCAGCACCACATGGCCCCTACAAAGGACGAGTTCGA | |
| | CTGCAAGGCCTGGGCCTACTTCTCTGACGTGGACCTCGAGAAGGATGTGCACAGC | |
| | GGACTCATCGGACCCCTGCTTGTGTGCCACACCAACACACTGAATCCCGCTCACGG | |
| | CAGGCAAGTGACCGTGCAAGAGTTCGCCCTGTTCTTCACCATCTTCGATGAGACAA | |
| | AGTCCTGGTACTTCACCGAAAACATGGAAAGAAACTGCAGGGCCCCTTGCAACAT | |
| | CCAGATGGAAGATCCCACCTTCAAAGAGAACTACCGGTTCCACGCCATCAATGGCT | |
| | ACATCATGGACACTCTGCCCGGCCTGGTTATGGCACAGGATCAGAGGATCAGATG | |
| | GTATCTGCTGTCCATGGGCTCCAACGAGAATATCCACAGCATCCACTTCAGCGGCC | |
| | ATGTGTTCACCGTGCGGAAAAAAGAAGAGTACAAGATGGCCCTGTACAATCTGTA | |
| | CCCCGGCGTGTTCGAGACTGTGGAAATGCTGCCTAGCAAGGCCGGAATCTGGCGC | |
| | GTGGAATGTCTGATCGGAGAGCATCTGCATGCCGGAATGTCTACCCTGTTCCTGGT | |
| | GTACAGCAACAAGTGTCAGACCCCTCTCGGCATGGCCTCTGGACACATCAGAGAC | |
| | TTCCAGATCACCGCCTCTGGCCAGTACGGACAGTGGGCTCCTAAACTGGCTAGACT | |
| | GCACTACAGCGGCAGCATCAACGCCTGGTCCACCAAAGAGCCCTTCAGCTGGATC | |
| | AAGGTGGACCTGCTGGCTCCCATGATCATCCACGGAATCAAGACCCAGGGCGCCA | |
| | GACAGAAGTTCAGCAGCCTGTACATCAGCCAGTTCATCATCATGTACAGCCTGGAC | |
| | GGCAAGAAGTGGCAGACCTACAGAGGCAACAGCACCGGCACACTCATGGTGTTCT | |
| | TCGGCAACGTGGACTCCAGCGGCATTAAGCACAACATCTTCAACCCTCCAATCATT | |
| | GCCCGGTACATCCGGCTGCACCCCACACACTACAGCATCAGATCTACCCTGAGGAT | |
| | GGAACTGATGGGCTGCGACCTGAACAGCTGCTCTATGCCCCTCGGAATGGAAAGC | |
| | AAGGCCATCAGCGACGCCCAGATCACAGCCAGCAGCTACTTCACCAACATGTTCGC | |
| | CACATGGTCCCCATCTAAGGCCCGGCTGCATCTGCAGGGCAGATCTAACGCTTGG | |
| | AGGCCCCAAGTGAACAACCCCAAAGAGTGGCTGCAGGTCGACTTTCAGAAAACCA | |
| | TGAAAGTGACCGGCGTGACCACACAGGGCGTCAAGTCTCTGCTGACCTCTATGTA | |
| | CGTGAAAGAGTTCCTGATCTCCAGCAGCCAGGACGGCCACCAGTGGACCCTGTTTT | |
| | TCCAGAACGGCAAAGTCAAGGTGTTCCAGGGAAACCAGGACAGCTTCACACCCGT | |
| | GGTCAACTCCCTGGATCCTCCACTGCTGACCAGATACCTGAGAATTCACCCTCAGT | |
| | CTTGGGTGCACCAGATCGCTCTGAGAATGGAAGTGCTGGGATGTGAAGCTCAGGA | |
| | CCTCTACTAAAATAAAAGATCTTTATTTTCATTAGATCTGTGTGTTGGTTTTTTGTGT | |
| | GCGATCGGGAACTGGCATCTTCAGGGAGTAGCTTAGGTCAGTGAAGAGAAGTGC | |
| | CAGTTCCCGATCGTTACAGGCCGCgggccgc | |
| 91 | AATTGCTGACCTCTTCTCTTCCTCCCACAGTGGCCACCAGAAGATACTACCTGGGA | MAB8E |
| | GCTGTGGAATTGAGCTGGGATTACATGCAATCTGACCTGGGAGAACTGCCTGTGG | |
| | ATGCCAGGTTTCCTCCTAGGGTCCCCAAGTCCTTCCCATTCAACACCTCAGTGGTCT | |
| | ACAAGAAAACCCTCTTTGTGGAGTTCACAGACCATCTGTTCAACATTGCCAAGCCA | |
| | AGACCCCCATGGATGGGACTCCTGGGTCCAACCATCCAAGCTGAAGTGTATGACA | |
| | CTGTGGTCATTACCCTGAAGAACATGGCCTCCCATCCTGTGTCCCTGCATGCAGTG | |
| | GGAGTGTCCTACTGGAAGGCTTCTGAAGGGGCTGAGTATGATGATCAAACCAGCC | |
| | AGAGAGAAAAGGAGGATGACAAAGTGTTCCCAGGTGGTAGTCACACCTATGTGT | |
| | GGCAAGTGCTCAAGGAGAATGGTCCTATGGCCTCTGATCCCCTGTGTCTGACCTAC | |
| | TCCTACCTGTCCCATGTGGACCTGGTGAAGGATCTGAACTCTGGGCTGATTGGAGC | |
| | CCTGCTGGTGTGCAGAGAAGGCTCCCTGGCCAAGGAAAAGACCCAGACACTGCAC | |
| | AAGTTCATCTTGCTGTTTGCTGTGTTTGATGAGGGAAAGTCCTGGCATTCTGAGAC | |
| | TAAGAACTCCCTTATGCAAGACAGAGATGCTGCCTCAGCTAGGGCTTGGCCTAAG | |
| | ATGCATACTGTGAATGGATATGTGAACAGATCCCTGCCTGGCCTTATTGGTTGCCA | |
| | CAGGAAGTCTGTGTATTGGCATGTGATTGGCATGGGAACCACTCCAGAGGTGCAC | |
| | TCCATTTTCTTGGAGGGGCATACCTTCTTGGTGAGGAACCACAGACAGGCCTCCCT | |
| | GGAAATTTCTCCAATCACTTTCCTGACTGCCCAGACCCTCCTTATGGACCTGGGTCA | |
| | GTTCCTGCTGTTCTGCCACATTTCATCCCACCAACATGATGGCATGGAAGCCTATGT | |
| | GAAAGTGGACTCATGCCCAGAAGAACCACAGCTGAGAATGAAGAACAATGAAGA | |
| | GGCAGAGGACTATGATGATGATCTTACAGATTCAGAAATGGATGTGGTCAGATTT | |
| | GATGATGATAATAGCCCATCCTTCATCCAAATTAGGAGTGTGGCCAAGAAGCACCC | |
| | CAAAACTTGGGTGCATTACATTGCAGCTGAGGAAGAGGATTGGGACTATGCACCC | |
| | TTGGTGCTTGCACCAGATGATAGGTCCTACAAGTCCCAATACCTGAACAATGGCCC | |
| | ACAGAGGATTGGTAGAAAGTATAAGAAAGTGAGATTCATGGCCTACACAGATGA | |
| | GACTTTCAAGACCAGAGAGGCCATTCAGCATGAATCTGGCATTCTGGGGCCACTG | |
| | TTGTATGGGGAGGTTGGAGATACACTGCTCATCATTTTCAAGAACCAGGCCTCCAG | |
| | ACCCTACAACATCTACCCTCATGGAATCACTGATGTCAGACCCCTGTACTCCAGAA | |
| | GACTCCCAAAGGGAGTCAAGCACTTGAAAGACTTCCCCATCCTGCCTGGGGAAAT | |
| | CTTCAAGTACAAGTGGACAGTGACAGTGGAGGATGGGCCAACCAAGTCTGATCCA | |
| | AGATGCCTCACTAGATACTACTCATCCTTTGTCAACATGGAAAGAGACCTGGCCTC | |
| | AGGACTGATTGGCCCCCTGCTCATCTGCTACAAGGAGTCTGTGGATCAGAGAGGA | |
| | AACCAGATCATGTCTGACAAAAGGAATGTCATCCTCTTCTCTGTCTTTGATGAGAA | |
| | CAGATCATGGTACCTTACAGAGAACATCCAGAGGTTCCTCCCCAACCCTGCTGGAG | |
| | TGCAGCTGGAGGACCCAGAATTCCAGGCATCAAACATTATGCACTCCATCAATGGT | |
| | TATGTGTTTGACAGCCTCCAGCTTTCTGTGTGCCTCCATGAAGTGGCATATTGGTAC | |
| | ATCCTGTCCATTGGAGCACAAACAGACTTTCTCTCTGTGTTCTTCTCTGGATATACC | |
| | TTCAAGCACAAGATGGTGTATGAGGATACCCTGACCCTCTTCCCCTTCTCTGGAGA | |
| | GACTGTGTTTATGTCAATGGAAAACCCAGGCCTGTGGATTTTGGGGTGCCACAACT | |
| | CAGATTTCAGAAACAGGGGCATGACTGCCTTGCTCAAGGTGTCCTCCTGTGACAA | |
| | GAACACAGGAGACTACTATGAGGACTCCTATGAGGATATTTCTGCCTACCTCCTGT | |
| | CCAAGAACAATGCCATTGAACCCAGGTCCTTCAGCCAGAACCCTCCTGTCCTCAAG | |
| | AGGCATCAGAGAGAAATCACCAGAACTACCCTGCAGTCTGACCAGGAAGAGATTG | |
| | ATTATGATGACACTATCTCAGTGGAAATGAAGAAGGAGGACTTTGACATCTATGAT | |
| | GAAGATGAAAATCAGTCCCCTAGGTCCTTCCAAAAGAAAACAAGACACTACTTCAT | |
| | TGCTGCTGTGGAGAGACTCTGGGACTATGGCATGTCCTCATCACCCCATGTGCTTA | |
| | GGAACAGGGCTCAATCTGGGTCTGTCCCTCAGTTCAAGAAAGTGGTGTTTCAAGA | |
| | ATTCACAGATGGAAGCTTCACACAGCCATTGTACAGGGGAGAACTGAATGAGCAC | |
| | CTTGGCCTGCTGGGACCTTACATCAGAGCAGAGGTGGAGGACAACATCATGGTGA | |
| | CCTTCAGAAACCAAGCCTCCAGGCCATATTCATTCTACTCCAGCCTTATCTCATATG | |
| | AGGAGGATCAGAGACAGGGGGCTGAACCTAGGAAGAACTTTGTCAAGCCAAATG | |
| | AGACAAAGACCTACTTTTGGAAGGTGCAGCACCACATGGCCCCTACCAAGGATGA | |
| | GTTTGACTGCAAGGCCTGGGCTTACTTCTCTGATGTGGATCTGGAAAAGGATGTGC | |
| | ATTCTGGGCTGATTGGACCTCTGCTGGTCTGCCACACTAACACCCTCAATCCTGCTC | |
| | ATGGCAGACAAGTGACAGTGCAGGAGTTTGCCCTGTTCTTCACCATCTTTGATGAA | |
| | ACTAAGTCATGGTACTTTACAGAGAACATGGAGAGAAATTGTAGGGCCCCATGTA | |
| | ACATCCAGATGGAGGACCCAACATTCAAGGAGAACTACAGATTCCATGCCATTAAT | |
| | GGATACATTATGGACACTCTTCCAGGACTGGTGATGGCACAGGACCAAAGAATCA | |
| | GATGGTATCTTCTGAGCATGGGGAGCAATGAAAACATCCATTCCATCCACTTTTCA | |
| | GGTCATGTGTTCACAGTGAGGAAGAAGGAAGAGTACAAGATGGCTCTGTACAACC | |
| | TGTACCCTGGGGTGTTTGAGACTGTGGAAATGCTGCCATCCAAGGCTGGAATTTG | |
| | GAGGGTGGAATGTCTGATTGGTGAACATCTGCATGCTGGAATGTCCACCCTGTTCC | |
| | TGGTGTACTCCAACAAGTGCCAAACCCCACTGGGAATGGCATCAGGACACATTAG | |
| | AGACTTCCAGATTACAGCATCTGGACAGTATGGACAATGGGCCCCCAAGTTGGCC | |
| | AGGCTGCACTACTCTGGAAGCATTAATGCCTGGAGCACCAAGGAGCCATTCAGCT | |
| | GGATCAAGGTGGACCTTCTGGCTCCAATGATCATCCATGGAATTAAGACTCAGGG | |
| | AGCCAGACAGAAGTTCTCATCCCTCTACATCTCCCAGTTTATCATCATGTACTCACT | |
| | GGATGGGAAGAAGTGGCAGACTTACAGGGGAAATTCCACAGGTACTCTGATGGT | |
| | GTTCTTTGGAAATGTGGACAGCTCTGGCATCAAGCACAATATCTTTAACCCTCCTAT | |
| | CATTGCCAGGTACATCAGACTCCACCCAACTCACTACTCCATCAGGTCCACTCTGAG | |
| | GATGGAACTCATGGGTTGTGACCTCAACTCCTGCTCAATGCCACTGGGCATGGAGT | |
| | CCAAGGCTATCTCAGATGCTCAGATTACTGCATCCTCTTACTTTACCAACATGTTTG | |
| | CTACCTGGTCCCCCTCCAAAGCCAGACTGCATCTCCAAGGCAGATCAAATGCCTGG | |
| | AGGCCTCAGGTCAACAACCCAAAGGAATGGCTTCAGGTGGACTTCCAAAAGACCA | |
| | TGAAAGTCACAGGAGTGACCACCCAGGGAGTGAAATCCCTGCTGACCTCTATGTA | |
| | TGTGAAGGAATTCCTGATCTCATCAAGCCAGGATGGCCACCAGTGGACACTGTTCT | |
| | TCCAAAATGGAAAGGTCAAGGTCTTTCAGGGAAATCAAGACTCCTTCACCCCTGTG | |
| | GTGAACTCCCTGGACCCCCCTCTGCTTACCAGGTACTTGAGAATTCATCCACAATCC | |
| | TGGGTGCACCAGATTGCCCTGAGGATGGAAGTGCTGGGCTGTGAAGCCCAGGAC | |
| | CTGTACTAAAATAAAAGATCTTTATTTTCATTAGATCTGTGTGTTGGTTTTTTGTGT | |
| | GCCGC | |
| 92 | GCTAGCCAATTGctgacctcttctcttcctcccacagtggccaccagaaggtactacctgggagctgtgg | MAB8F |
| | aactgagctgggactacatgcagtctgacctgggagagctgccTgtggatgctagatttcctccaagagtgc | (GA co/CpG free) |
| | ccaagagcttccccttcaacacctctgtggtgtacaagaaaaccctgtttgtggaattcacagaccacctgttc | |
| | aatattgccaagcctagacctccttggatgggcctgctgggccctacaattcaggctgaggtgtatgacacag | |
| | tggtcatcaccctgaagaacatggccagccatcctgtgtctctgcatgctgtgggagtgtcttactggaaggct | |
| | tctgagggggctgagtatgatgaccagacaagccagagagagaaagaggatgacaaggttttccctgggg | |
| | gcagccacacctatgtctggcaggtcctgaaagaaaatggccctatggcctctgatcctctgtgcctgacata | |
| | cagctacctgagccatgtggacctggtcaaggacctgaactctggcctgattggggctctgctggtgtgtaga | |
| | gaaggcagcctggccaaagaaaagacccagacactgcacaagttcatcctgctgtttgctgtgtttgatgag | |
| | ggcaagagctggcactctgagacaaagaacagcctgatgcaggacagagatgctgcctctgctagagcttg | |
| | gcccaagatgcacacagtgaatggctatgtgaacagaagcctgcctggactgattggatgccacagaaagt | |
| | ctgtgtactggcatgtgattggcatgggcaccacacctgaggtgcacagcatctttctggaaggacacacctt | |
| | cctggtgaggaaccacagacaggccagcctggaaatcagccctatcaccttcctgacagctcagaccctgct | |
| | gatggatctgggccagtttctgctgttctgccacatcagcagccaccagcatgatggcatggaagcctatgtg | |
| | aaggtggacagctgccctgaagaaccccagctgagaatgaagaacaatgaggaagctgaggactatgatg | |
| | atgacctgacagactctgagatggatgtggtcagatttgatgatgataacagccccagcttcatccagatcag | |
| | atctgtggccaagaagcaccccaagacctgggtgcactatattgctgctgaggaagaggactgggattatgc | |
| | tcctctggtgctggcccctgatgacagaagctacaagagccagtacctgaacaatggccctcagagaattgg | |
| | caggaagtataagaaagtgaggttcatggcctacacagatgagacattcaagaccagagaggctatccagc | |
| | atgagtctggcattctgggacctctgctgtatggggaagtgggggacacactgctgatcatcttcaagaacca | |
| | ggccagcagaccctacaacatctaccctcatggcatcacagatgtgaggcctctgtactctagaaggctgcc | |
| | caagggggtgaagcacctgaaggacttccctatcctgcctggggagatcttcaagtacaagtggacagtgac | |
| | agtggaggatggccctaccaagtctgatcctagatgcctgacaaggtactacagcagctttgtgaacatgga | |
| | aagggacctggcctctggcctgattggtcctctgctgatctgctacaaagaatctgtggaccagaggggcaa | |
| | ccagatcatgagtgacaagagaaatgtgatcctgttctctgtctttgatgagaacaggtcctggtatctgaca | |
| | gagaacatccagaggtttctgcccaatcctgctggggtgcagctggaagatcctgagttccaggcctccaac | |
| | atcatgcactccatcaatggctatgtgtttgacagcctgcagctgtctgtgtgcctgcatgaagtggcctactg | |
| | gtacatcctgtctattggggcccagacagacttcctgtctgtgttcttttctggctacaccttcaagcacaagat | |
| | ggtgtatgaggataccctgacactgttcccattctctggggagacagtgttcatgagcatggaaaaccctggc | |
| | ctgtggatcctgggctgtcacaacagtgacttcagaaacagaggcatgacagccctgctgaaggtgtccagc | |
| | tgtgacaagaacactggggactactatgaggactcttatgaggacatctctgcctacctgctgagcaagaac | |
| | aatgccattgagcctaggagcttctctcagaaccctcctgtgctgaagagacaccagagggagatcaccaga | |
| | accacactgcagtctgaccaagaggaaattgattatgatgacaccatctctgtggagatgaagaaagaaga | |
| | ttttgacatctatgatgaggatgagaatcagagccccagatctttccagaagaaaacaaggcactacttcatt | |
| | gctgctgtggaaagactgtgggactatggcatgagcagcagcccccatgtgctgagaaacagggcccagtc | |
| | tggaagtgtgccccagttcaagaaagtggtgttccaagagttcacagatggcagcttcacccagcctctgtat | |
| | agaggggagctgaatgagcacctgggactgctgggaccttacatcagagctgaggtggaggataacatcat | |
| | ggtcacctttagaaaccaggcctctaggccctactccttctacagctccctgatcagctatgaagaggaccag | |
| | agacagggggctgagcccagaaagaactttgtgaagcccaatgagactaagacctacttttggaaggtgca | |
| | gcaccacatggcccctacaaaggatgagtttgactgcaaggcctgggcctacttctctgatgtggacctgga | |
| | gaaggatgtgcactctggactcattggacccctgcttgtgtgccacaccaacacactgaatcctgctcatggc | |
| | aggcaagtgacagtgcaagagtttgccctgttcttcaccatctttgatgagacaaagtcctggtacttcacag | |
| | aaaacatggaaagaaactgcagggccccttgcaacatccagatggaagatcccaccttcaaagagaacta | |
| | caggttccatgccatcaatggctacatcatggacactctgcctggcctggttatggcacaggatcagaggatc | |
| | agatggtatctgctgtccatgggctccaatgagaatatccacagcatccacttctctggccatgtgttcacagt | |
| | gaggaaaaaagaagagtacaagatggccctgtacaatctgtaccctggggtgtttgagactgtggaaatgc | |
| | tgcctagcaaggctggaatctggagggtggaatgtctgattggagagcatctgcatgctggaatgtctaccct | |
| | gttcctggtgtacagcaacaagtgtcagacccctctgggcatggcctctggacacatcagagacttccagatc | |
| | acagcctctggccagtatggacagtgggctcctaaactggctagactgcactactctggcagcatcaatgcct | |
| | ggtccaccaaagagcccttcagctggatcaaggtggacctgctggctcccatgatcatccatggaatcaaga | |
| | cccagggggccagacagaagttcagcagcctgtacatcagccagttcatcatcatgtacagcctggatggca | |
| | agaagtggcagacctacagaggcaacagcacaggcacactcatggtgttctttggcaatgtggactcttctg | |
| | gcattaagcacaacatcttcaaccctccaatcattgccaggtacatcaggctgcaccccacacactacagca | |
| | tcagatctaccctgaggatggaactgatgggctgtgacctgaacagctgctctatgcccctgggaatggaaa | |
| | gcaaggccatctctgatgcccagatcacagccagcagctacttcaccaacatgtttgccacatggtccccatc | |
| | taaggccaggctgcatctgcagggcagatctaatgcttggaggccccaagtgaacaaccccaaagagtggc | |
| | tgcaggtggactttcagaaaaccatgaaagtgacaggagtgaccacacagggggtcaagtctctgctgacc | |
| | tctatgtatgtgaaagagttcctgatctccagcagccaggatggccaccagtggaccctgtttttccagaatg | |
| | gcaaagtcaaggtgttccagggaaaccaggacagcttcacacctgtggtcaactccctggatcctccactgc | |
| | tgaccagatacctgagaattcaccctcagtcttgggtgcaccagattgctctgagaatggaagtgctgggatg | |
| | tgaagctcaggacctctacTAAAATAAAAGATCTTTATTTTCATTAGATCTGTGTGTTGGT | |
| | TTTTTGTGTGCCGCGGGTTTAAAC | |
| 93 | GCCACCAGAAGATACTACCTCGGAGCCGTCGAATTGAGCTGGGATTACATGCAAT | F8-BDD1, codon |
| | CCGACCTGGGAGAACTGCCCGTGGATGCCAGGTTTCCTCCTCGGGTCCCCAAGTCC | optimized |
| | TTCCCGTTCAACACCTCAGTCGTCTACAAGAAAACCCTCTTCGTGGAGTTCACCGAC | |
| | CATCTGTTCAACATCGCCAAGCCAAGACCCCCGTGGATGGGACTCCTCGGTCCGAC | |
| | CATCCAAGCCGAAGTGTACGACACTGTGGTCATTACCCTGAAGAACATGGCCTCCC | |
| | ATCCTGTGTCCCTGCATGCAGTGGGCGTGTCCTACTGGAAGGCTTCCGAAGGGGC | |
| | CGAGTACGACGATCAAACCAGCCAGCGGGAAAAGGAGGATGACAAAGTGTTCCC | |
| | GGGTGGTTCGCACACCTACGTGTGGCAAGTGCTCAAGGAGAACGGTCCTATGGCC | |
| | TCTGATCCCCTGTGTCTGACCTACTCCTACCTGTCCCATGTCGACCTCGTGAAGGAT | |
| | CTGAACAGCGGGCTGATTGGCGCCCTGCTCGTGTGCCGGGAAGGCTCCCTGGCCA | |
| | AGGAAAAGACCCAGACACTGCACAAGTTCATCTTGCTGTTCGCCGTGTTTGATGAG | |
| | GGAAAGTCCTGGCATAGCGAGACTAAGAACTCCCTTATGCAAGACCGGGATGCTG | |
| | CCTCCGCTAGGGCTTGGCCTAAGATGCATACTGTGAACGGATACGTGAACAGATC | |
| | CCTGCCTGGCCTTATCGGTTGCCACCGGAAGTCCGTGTATTGGCATGTGATCGGCA | |
| | TGGGAACCACTCCAGAGGTGCACTCCATTTTCTTGGAGGGGCATACCTTCTTGGTG | |
| | CGCAACCACAGACAGGCCTCCCTGGAAATTTCTCCGATCACTTTCCTGACTGCCCA | |
| | GACCCTCCTTATGGACCTGGGTCAGTTCCTGCTGTTCTGCCACATTTCGTCCCACCA | |
| | ACACGATGGCATGGAAGCCTACGTGAAAGTGGACTCGTGCCCGGAAGAACCACA | |
| | GCTGCGGATGAAGAACAACGAAGAGGCAGAGGACTACGATGATGATCTTACCGA | |
| | TTCGGAAATGGATGTGGTCCGATTCGACGACGATAATAGCCCATCCTTCATCCAAA | |
| | TTAGGAGCGTGGCCAAGAAGCACCCCAAAACTTGGGTGCATTACATTGCGGCCGA | |
| | GGAAGAGGATTGGGACTACGCACCCCTCGTGCTTGCACCCGATGATCGGTCCTAC | |
| | AAGTCCCAATACCTGAACAACGGCCCGCAGAGGATCGGTCGGAAGTATAAGAAA | |
| | GTGCGCTTCATGGCCTACACCGACGAGACTTTCAAGACCAGAGAGGCCATTCAGC | |
| | ACGAAAGCGGCATTCTGGGGCCGCTGTTGTACGGGGAGGTCGGAGATACACTGC | |
| | TCATCATTTTCAAGAACCAGGCGTCCAGACCCTACAACATCTACCCGCACGGAATC | |
| | ACTGACGTCCGCCCCCTGTACTCCCGGAGACTCCCGAAGGGAGTCAAGCACTTGA | |
| | AAGACTTCCCCATCCTGCCTGGGGAAATCTTCAAGTACAAGTGGACCGTGACCGTC | |
| | GAGGATGGGCCGACCAAGTCCGATCCAAGATGCCTCACTAGATACTACTCATCCTT | |
| | CGTCAACATGGAACGGGACCTGGCCTCAGGACTGATTGGCCCCCTGCTCATCTGCT | |
| | ACAAGGAGTCCGTGGATCAGCGCGGAAACCAGATCATGTCGGACAAACGCAACG | |
| | TCATCCTCTTCTCCGTCTTTGACGAGAACCGCTCATGGTACCTTACGGAGAACATCC | |
| | AGCGGTTCCTCCCCAACCCTGCCGGAGTGCAGCTCGAGGACCCGGAATTCCAGGC | |
| | ATCAAACATTATGCACTCCATCAACGGTTACGTGTTCGACAGCCTCCAGCTTAGCG | |
| | TGTGCCTCCATGAAGTCGCATATTGGTACATCCTGTCCATTGGAGCACAAACCGAC | |
| | TTTCTCTCCGTGTTCTTCTCCGGATATACCTTCAAGCACAAGATGGTGTACGAGGAT | |
| | ACCCTGACCCTCTTCCCCTTCTCCGGAGAGACTGTGTTTATGTCGATGGAAAACCCA | |
| | GGCCTGTGGATTTTGGGGTGCCACAACTCGGATTTCCGAAACCGGGGCATGACTG | |
| | CCTTGCTCAAGGTGTCCTCCTGTGACAAGAACACGGGAGACTACTACGAGGACTC | |
| | CTACGAGGATATTTCCGCCTACCTCCTGTCCAAGAACAACGCCATCGAACCCAGGT | |
| | CCTTCAGCCAGAACCCTCCTGTCCTCAAGCGCCATCAGAGAGAAATCACCCGCACG | |
| | ACCCTGCAGTCCGACCAGGAAGAGATCGATTACGACGACACTATCTCCGTCGAAAT | |
| | GAAGAAGGAGGACTTTGACATCTACGACGAAGATGAAAATCAGTCCCCTCGCTCG | |
| | TTCCAAAAGAAAACGAGACACTACTTCATCGCTGCTGTGGAGCGGCTCTGGGACT | |
| | ACGGCATGTCCTCATCGCCCCACGTGCTTAGGAACCGGGCTCAATCCGGGAGCGT | |
| | CCCTCAGTTCAAGAAAGTGGTGTTTCAAGAATTCACCGATGGAAGCTTCACGCAGC | |
| | CGTTGTACAGGGGCGAACTGAACGAGCACCTTGGCCTGCTGGGACCTTACATCAG | |
| | AGCAGAGGTCGAGGACAACATCATGGTGACCTTCCGGAACCAAGCCTCCCGGCCA | |
| | TATTCATTCTACTCGAGCCTTATCTCATACGAGGAGGATCAGAGACAGGGGGCTG | |
| | AACCTCGGAAGAACTTCGTCAAGCCGAACGAGACAAAGACCTACTTTTGGAAGGT | |
| | GCAGCACCACATGGCCCCGACCAAGGATGAGTTCGACTGCAAGGCCTGGGCGTAC | |
| | TTCTCCGACGTGGATCTCGAAAAGGACGTGCATTCCGGGCTGATCGGACCGCTGC | |
| | TCGTCTGCCACACTAACACCCTCAATCCTGCTCACGGCAGACAAGTGACCGTGCAG | |
| | GAGTTCGCCCTGTTCTTCACCATCTTCGACGAAACTAAGTCATGGTACTTTACCGAG | |
| | AACATGGAGCGGAATTGTCGGGCCCCATGTAACATCCAGATGGAGGACCCGACAT | |
| | TCAAGGAGAACTACCGGTTCCACGCCATTAACGGATACATTATGGACACTCTTCCG | |
| | GGACTCGTGATGGCACAGGACCAACGCATCAGATGGTATCTTCTGTCGATGGGGA | |
| | GCAACGAAAACATCCATTCGATCCACTTTAGCGGTCACGTGTTCACAGTGCGCAAG | |
| | AAGGAAGAGTACAAGATGGCGCTGTACAACCTGTACCCTGGGGTGTTCGAGACTG | |
| | TGGAAATGCTGCCGTCCAAGGCCGGAATTTGGCGCGTGGAATGTCTGATCGGTGA | |
| | ACATCTGCATGCCGGAATGTCCACCCTGTTCCTGGTGTACTCCAACAAGTGCCAAA | |
| | CCCCACTGGGAATGGCATCAGGACACATTAGAGACTTCCAGATTACCGCGAGCGG | |
| | ACAGTACGGACAATGGGCCCCCAAGTTGGCCAGGCTGCACTACTCTGGAAGCATT | |
| | AACGCCTGGAGCACCAAGGAGCCGTTCAGCTGGATCAAGGTGGACCTTCTGGCGC | |
| | CAATGATCATCCACGGAATTAAGACTCAGGGAGCCCGCCAGAAGTTCTCATCGCTC | |
| | TACATCTCCCAGTTTATCATCATGTACTCACTGGATGGGAAGAAGTGGCAGACTTA | |
| | CCGGGGAAATTCCACCGGTACTCTGATGGTGTTCTTCGGAAACGTGGACAGCTCC | |
| | GGCATCAAGCACAATATCTTTAACCCGCCTATCATCGCCCGATACATCCGGCTCCAC | |
| | CCGACTCACTACTCCATCCGGTCGACTCTGCGGATGGAACTCATGGGTTGCGACCT | |
| | CAACTCCTGCTCAATGCCACTGGGCATGGAGTCCAAGGCTATCTCGGACGCTCAGA | |
| | TTACTGCATCGTCGTACTTTACCAACATGTTCGCTACCTGGTCCCCGTCCAAAGCCC | |
| | GGCTGCATCTCCAAGGCAGATCAAACGCGTGGAGGCCTCAGGTCAACAACCCGAA | |
| | GGAATGGCTTCAGGTCGACTTCCAAAAGACCATGAAAGTCACCGGAGTGACCACC | |
| | CAGGGCGTGAAATCGCTGCTGACCTCTATGTACGTGAAGGAATTCCTGATCTCATC | |
| | AAGCCAGGACGGCCACCAGTGGACACTGTTCTTCCAAAATGGAAAGGTCAAGGTC | |
| | TTTCAGGGAAATCAAGACTCCTTCACCCCCGTGGTGAACTCCCTGGACCCCCCTCT | |
| | GCTTACCCGCTACTTGCGCATTCATCCGCAATCCTGGGTGCACCAGATCGCCCTGC | |
| | GAATGGAAGTGCTGGGCTGTGAAGCGCAGGACCTGTAC | |
| 94 | GCCACCAGAAGGTACTACCTAGGAGCCGTGGAACTGAGCTGGGACTACATGCAGT | F8-BDD2, codon |
| | CTGACCTGGGAGAGCTGCCCGTGGACGCTAGATTTCCTCCAAGAGTGCCCAAGAG | optimized |
| | CTTCCCCTTCAACACCTCCGTGGTGTACAAGAAAACCCTGTTCGTGGAATTCACCG | |
| | ACCACCTGTTCAATATCGCCAAGCCTAGACCTCCTTGGATGGGCCTGCTGGGCCCT | |
| | ACAATTCAGGCCGAGGTGTACGACACCGTGGTCATCACCCTGAAGAACATGGCCA | |
| | GCCATCCTGTGTCTCTGCACGCCGTGGGAGTGTCTTACTGGAAGGCTTCTGAGGGC | |
| | GCCGAGTACGACGACCAGACAAGCCAGAGAGAGAAAGAGGACGACAAGGTTTTC | |
| | CCTGGCGGCAGCCACACCTATGTCTGGCAGGTCCTGAAAGAAAACGGCCCTATGG | |
| | CCTCCGATCCTCTGTGCCTGACATACAGCTACCTGAGCCATGTGGACCTGGTCAAG | |
| | GACCTGAACTCTGGCCTGATCGGCGCTCTGCTCGTGTGTAGAGAAGGCAGCCTGG | |
| | CCAAAGAAAAGACCCAGACACTGCACAAGTTCATCCTGCTGTTCGCCGTGTTCGAC | |
| | GAGGGCAAGAGCTGGCACAGCGAGACAAAGAACAGCCTGATGCAGGACAGAGA | |
| | TGCCGCCTCTGCTAGAGCTTGGCCCAAGATGCACACCGTGAACGGCTACGTGAAC | |
| | AGAAGCCTGCCTGGACTGATCGGATGCCACAGAAAGTCCGTGTACTGGCATGTGA | |
| | TCGGCATGGGCACCACACCTGAGGTGCACAGCATCTTTCTGGAAGGACACACCTTC | |
| | CTCGTGCGGAACCACAGACAGGCCAGCCTGGAAATCAGCCCTATCACCTTCCTGAC | |
| | CGCTCAGACCCTGCTGATGGATCTGGGCCAGTTTCTGCTGTTCTGCCACATCAGCA | |
| | GCCACCAGCACGATGGCATGGAAGCCTACGTGAAGGTGGACAGCTGCCCCGAAG | |
| | AACCCCAGCTGAGAATGAAGAACAACGAGGAAGCCGAGGACTACGACGACGACC | |
| | TGACCGACTCTGAGATGGACGTCGTCAGATTCGACGACGATAACAGCCCCAGCTT | |
| | CATCCAGATCAGAAGCGTGGCCAAGAAGCACCCCAAGACCTGGGTGCACTATATC | |
| | GCCGCCGAGGAAGAGGACTGGGATTACGCTCCTCTGGTGCTGGCCCCTGACGACA | |
| | GAAGCTACAAGAGCCAGTACCTGAACAACGGCCCTCAGAGAATCGGCCGGAAGT | |
| | ATAAGAAAGTGCGGTTCATGGCCTACACCGACGAGACATTCAAGACCAGAGAGGC | |
| | TATCCAGCACGAGAGCGGCATTCTGGGACCTCTGCTGTATGGCGAAGTGGGCGAC | |
| | ACACTGCTGATCATCTTCAAGAACCAGGCCAGCAGACCCTACAACATCTACCCTCA | |
| | CGGCATCACCGATGTGCGGCCTCTGTACTCTAGAAGGCTGCCCAAGGGCGTGAAG | |
| | CACCTGAAGGACTTCCCTATCCTGCCTGGCGAGATCTTCAAGTACAAGTGGACCGT | |
| | GACCGTCGAGGACGGCCCTACCAAGAGCGATCCTAGATGCCTGACACGGTACTAC | |
| | AGCAGCTTCGTGAACATGGAACGCGACCTGGCCAGCGGCCTGATTGGTCCTCTGC | |
| | TGATCTGCTACAAAGAAAGCGTGGACCAGAGGGGCAACCAGATCATGAGCGACA | |
| | AGAGAAACGTGATCCTGTTCTCCGTCTTTGACGAGAACAGGTCCTGGTATCTGACC | |
| | GAGAACATCCAGCGGTTTCTGCCCAATCCTGCTGGCGTGCAGCTGGAAGATCCTG | |
| | AGTTCCAGGCCTCCAACATCATGCACTCCATCAACGGCTATGTGTTCGACAGCCTG | |
| | CAGCTGAGCGTGTGCCTGCACGAAGTGGCCTACTGGTACATCCTGTCTATCGGCGC | |
| | CCAGACCGACTTCCTGTCCGTGTTCTTTAGCGGCTACACCTTCAAGCACAAGATGG | |
| | TGTACGAGGATACCCTGACACTGTTCCCATTCAGCGGCGAGACAGTGTTCATGAGC | |
| | ATGGAAAACCCCGGCCTGTGGATCCTGGGCTGTCACAACAGCGACTTCAGAAACA | |
| | GAGGCATGACAGCCCTGCTGAAGGTGTCCAGCTGCGACAAGAACACCGGCGACT | |
| | ACTACGAGGACTCTTACGAGGACATCAGCGCCTACCTGCTGAGCAAGAACAATGC | |
| | CATCGAGCCTCGGAGCTTCTCTCAGAACCCTCCTGTGCTGAAGAGACACCAGCGCG | |
| | AGATCACCAGAACCACACTGCAGAGCGACCAAGAGGAAATCGATTACGACGACAC | |
| | CATCAGCGTCGAGATGAAGAAAGAAGATTTCGACATCTACGACGAGGACGAGAAT | |
| | CAGAGCCCCAGATCTTTCCAGAAGAAAACGCGGCACTACTTCATTGCCGCCGTGG | |
| | AAAGACTGTGGGACTACGGCATGAGCAGCAGCCCACATGTGCTGAGAAACAGGG | |
| | CCCAGAGCGGAAGCGTGCCCCAGTTCAAGAAAGTGGTGTTCCAAGAGTTCACCGA | |
| | CGGCAGCTTCACCCAGCCTCTGTATAGAGGCGAGCTGAACGAGCACCTGGGACTG | |
| | CTGGGACCTTACATCAGAGCTGAGGTCGAGGATAACATCATGGTCACCTTTAGAA | |
| | ACCAGGCCTCTAGGCCCTACTCCTTCTACAGCTCCCTGATCAGCTACGAAGAGGAC | |
| | CAGAGACAGGGCGCTGAGCCCAGAAAGAACTTCGTGAAGCCCAACGAGACTAAG | |
| | ACCTACTTTTGGAAGGTGCAGCACCACATGGCCCCTACAAAGGACGAGTTCGACT | |
| | GCAAGGCCTGGGCCTACTTCTCTGACGTGGACCTCGAGAAGGATGTGCACAGCGG | |
| | ACTCATCGGACCCCTGCTTGTGTGCCACACCAACACACTGAATCCCGCTCACGGCA | |
| | GGCAAGTGACCGTGCAAGAGTTCGCCCTGTTCTTCACCATCTTCGATGAGACAAAG | |
| | TCCTGGTACTTCACCGAAAACATGGAAAGAAACTGCAGGGCCCCTTGCAACATCCA | |
| | GATGGAAGATCCCACCTTCAAAGAGAACTACCGGTTCCACGCCATCAATGGCTACA | |
| | TCATGGACACTCTGCCCGGCCTGGTTATGGCACAGGATCAGAGGATCAGATGGTA | |
| | TCTGCTGTCCATGGGCTCCAACGAGAATATCCACAGCATCCACTTCAGCGGCCATG | |
| | TGTTCACCGTGCGGAAAAAAGAAGAGTACAAGATGGCCCTGTACAATCTGTACCC | |
| | CGGCGTGTTCGAGACTGTGGAAATGCTGCCTAGCAAGGCCGGAATCTGGCGCGTG | |
| | GAATGTCTGATCGGAGAGCATCTGCATGCCGGAATGTCTACCCTGTTCCTGGTGTA | |
| | CAGCAACAAGTGTCAGACCCCTCTCGGCATGGCCTCTGGACACATCAGAGACTTCC | |
| | AGATCACCGCCTCTGGCCAGTACGGACAGTGGGCTCCTAAACTGGCTAGACTGCA | |
| | CTACAGCGGCAGCATCAACGCCTGGTCCACCAAAGAGCCCTTCAGCTGGATCAAG | |
| | GTGGACCTGCTGGCTCCCATGATCATCCACGGAATCAAGACCCAGGGCGCCAGAC | |
| | AGAAGTTCAGCAGCCTGTACATCAGCCAGTTCATCATCATGTACAGCCTGGACGGC | |
| | AAGAAGTGGCAGACCTACAGAGGCAACAGCACCGGCACACTCATGGTGTTCTTCG | |
| | GCAACGTGGACTCCAGCGGCATTAAGCACAACATCTTCAACCCTCCAATCATTGCC | |
| | CGGTACATCCGGCTGCACCCCACACACTACAGCATCAGATCTACCCTGAGGATGGA | |
| | ACTGATGGGCTGCGACCTGAACAGCTGCTCTATGCCCCTCGGAATGGAAAGCAAG | |
| | GCCATCAGCGACGCCCAGATCACAGCCAGCAGCTACTTCACCAACATGTTCGCCAC | |
| | ATGGTCCCCATCTAAGGCCCGGCTGCATCTGCAGGGCAGATCTAACGCTTGGAGG | |
| | CCCCAAGTGAACAACCCCAAAGAGTGGCTGCAGGTCGACTTTCAGAAAACCATGA | |
| | AAGTGACCGGCGTGACCACACAGGGCGTCAAGTCTCTGCTGACCTCTATGTACGT | |
| | GAAAGAGTTCCTGATCTCCAGCAGCCAGGACGGCCACCAGTGGACCCTGTTTTTCC | |
| | AGAACGGCAAAGTCAAGGTGTTCCAGGGAAACCAGGACAGCTTCACACCCGTGGT | |
| | CAACTCCCTGGATCCTCCACTGCTGACCAGATACCTGAGAATTCACCCTCAGTCTTG | |
| | GGTGCACCAGATCGCTCTGAGAATGGAAGTGCTGGGATGTGAAGCTCAGGACCTC | |
| | TACTAAAATAAAAGATCTTTATTTTCATTAGATCTGTGTGTTGGTTTTTTGTGTGCG | |
| | ATCGGGAACTGGCATCTTCAGGGAGTAGCTTAGGTCAGTGAAGAGAAGTGCCAGT | |
| | TCCCGATCGTTACAGGCCGC | |
| 95 | GCCACCAGAAGATACTACCTGGGAGCTGTGGAATTGAGCTGGGATTACATGCAAT | F8-BDD3, codon |
| | CTGACCTGGGAGAACTGCCTGTGGATGCCAGGTTTCCTCCTAGGGTCCCCAAGTCC | optimized |
| | TTCCCATTCAACACCTCAGTGGTCTACAAGAAAACCCTCTTTGTGGAGTTCACAGAC | |
| | CATCTGTTCAACATTGCCAAGCCAAGACCCCCATGGATGGGACTCCTGGGTCCAAC | |
| | CATCCAAGCTGAAGTGTATGACACTGTGGTCATTACCCTGAAGAACATGGCCTCCC | |
| | ATCCTGTGTCCCTGCATGCAGTGGGAGTGTCCTACTGGAAGGCTTCTGAAGGGGC | |
| | TGAGTATGATGATCAAACCAGCCAGAGAGAAAAGGAGGATGACAAAGTGTTCCC | |
| | AGGTGGTAGTCACACCTATGTGTGGCAAGTGCTCAAGGAGAATGGTCCTATGGCC | |
| | TCTGATCCCCTGTGTCTGACCTACTCCTACCTGTCCCATGTGGACCTGGTGAAGGAT | |
| | CTGAACTCTGGGCTGATTGGAGCCCTGCTGGTGTGCAGAGAAGGCTCCCTGGCCA | |
| | AGGAAAAGACCCAGACACTGCACAAGTTCATCTTGCTGTTTGCTGTGTTTGATGAG | |
| | GGAAAGTCCTGGCATTCTGAGACTAAGAACTCCCTTATGCAAGACAGAGATGCTG | |
| | CCTCAGCTAGGGCTTGGCCTAAGATGCATACTGTGAATGGATATGTGAACAGATC | |
| | CCTGCCTGGCCTTATTGGTTGCCACAGGAAGTCTGTGTATTGGCATGTGATTGGCA | |
| | TGGGAACCACTCCAGAGGTGCACTCCATTTTCTTGGAGGGGCATACCTTCTTGGTG | |
| | AGGAACCACAGACAGGCCTCCCTGGAAATTTCTCCAATCACTTTCCTGACTGCCCA | |
| | GACCCTCCTTATGGACCTGGGTCAGTTCCTGCTGTTCTGCCACATTTCATCCCACCA | |
| | ACATGATGGCATGGAAGCCTATGTGAAAGTGGACTCATGCCCAGAAGAACCACAG | |
| | CTGAGAATGAAGAACAATGAAGAGGCAGAGGACTATGATGATGATCTTACAGATT | |
| | CAGAAATGGATGTGGTCAGATTTGATGATGATAATAGCCCATCCTTCATCCAAATT | |
| | AGGAGTGTGGCCAAGAAGCACCCCAAAACTTGGGTGCATTACATTGCAGCTGAGG | |
| | AAGAGGATTGGGACTATGCACCCTTGGTGCTTGCACCAGATGATAGGTCCTACAA | |
| | GTCCCAATACCTGAACAATGGCCCACAGAGGATTGGTAGAAAGTATAAGAAAGTG | |
| | AGATTCATGGCCTACACAGATGAGACTTTCAAGACCAGAGAGGCCATTCAGCATG | |
| | AATCTGGCATTCTGGGGCCACTGTTGTATGGGGAGGTTGGAGATACACTGCTCAT | |
| | CATTTTCAAGAACCAGGCCTCCAGACCCTACAACATCTACCCTCATGGAATCACTGA | |
| | TGTCAGACCCCTGTACTCCAGAAGACTCCCAAAGGGAGTCAAGCACTTGAAAGAC | |
| | TTCCCCATCCTGCCTGGGGAAATCTTCAAGTACAAGTGGACAGTGACAGTGGAGG | |
| | ATGGGCCAACCAAGTCTGATCCAAGATGCCTCACTAGATACTACTCATCCTTTGTCA | |
| | ACATGGAAAGAGACCTGGCCTCAGGACTGATTGGCCCCCTGCTCATCTGCTACAA | |
| | GGAGTCTGTGGATCAGAGAGGAAACCAGATCATGTCTGACAAAAGGAATGTCATC | |
| | CTCTTCTCTGTCTTTGATGAGAACAGATCATGGTACCTTACAGAGAACATCCAGAG | |
| | GTTCCTCCCCAACCCTGCTGGAGTGCAGCTGGAGGACCCAGAATTCCAGGCATCA | |
| | AACATTATGCACTCCATCAATGGTTATGTGTTTGACAGCCTCCAGCTTTCTGTGTGC | |
| | CTCCATGAAGTGGCATATTGGTACATCCTGTCCATTGGAGCACAAACAGACTTTCT | |
| | CTCTGTGTTCTTCTCTGGATATACCTTCAAGCACAAGATGGTGTATGAGGATACCCT | |
| | GACCCTCTTCCCCTTCTCTGGAGAGACTGTGTTTATGTCAATGGAAAACCCAGGCC | |
| | TGTGGATTTTGGGGTGCCACAACTCAGATTTCAGAAACAGGGGCATGACTGCCTT | |
| | GCTCAAGGTGTCCTCCTGTGACAAGAACACAGGAGACTACTATGAGGACTCCTAT | |
| | GAGGATATTTCTGCCTACCTCCTGTCCAAGAACAATGCCATTGAACCCAGGTCCTTC | |
| | AGCCAGAACCCTCCTGTCCTCAAGAGGCATCAGAGAGAAATCACCAGAACTACCCT | |
| | GCAGTCTGACCAGGAAGAGATTGATTATGATGACACTATCTCAGTGGAAATGAAG | |
| | AAGGAGGACTTTGACATCTATGATGAAGATGAAAATCAGTCCCCTAGGTCCTTCCA | |
| | AAAGAAAACAAGACACTACTTCATTGCTGCTGTGGAGAGACTCTGGGACTATGGC | |
| | ATGTCCTCATCACCCCATGTGCTTAGGAACAGGGCTCAATCTGGGTCTGTCCCTCA | |
| | GTTCAAGAAAGTGGTGTTTCAAGAATTCACAGATGGAAGCTTCACACAGCCATTGT | |
| | ACAGGGGAGAACTGAATGAGCACCTTGGCCTGCTGGGACCTTACATCAGAGCAGA | |
| | GGTGGAGGACAACATCATGGTGACCTTCAGAAACCAAGCCTCCAGGCCATATTCA | |
| | TTCTACTCCAGCCTTATCTCATATGAGGAGGATCAGAGACAGGGGGCTGAACCTA | |
| | GGAAGAACTTTGTCAAGCCAAATGAGACAAAGACCTACTTTTGGAAGGTGCAGCA | |
| | CCACATGGCCCCTACCAAGGATGAGTTTGACTGCAAGGCCTGGGCTTACTTCTCTG | |
| | ATGTGGATCTGGAAAAGGATGTGCATTCTGGGCTGATTGGACCTCTGCTGGTCTG | |
| | CCACACTAACACCCTCAATCCTGCTCATGGCAGACAAGTGACAGTGCAGGAGTTTG | |
| | CCCTGTTCTTCACCATCTTTGATGAAACTAAGTCATGGTACTTTACAGAGAACATGG | |
| | AGAGAAATTGTAGGGCCCCATGTAACATCCAGATGGAGGACCCAACATTCAAGGA | |
| | GAACTACAGATTCCATGCCATTAATGGATACATTATGGACACTCTTCCAGGACTGG | |
| | TGATGGCACAGGACCAAAGAATCAGATGGTATCTTCTGAGCATGGGGAGCAATGA | |
| | AAACATCCATTCCATCCACTTTTCAGGTCATGTGTTCACAGTGAGGAAGAAGGAAG | |
| | AGTACAAGATGGCTCTGTACAACCTGTACCCTGGGGTGTTTGAGACTGTGGAAAT | |
| | GCTGCCATCCAAGGCTGGAATTTGGAGGGTGGAATGTCTGATTGGTGAACATCTG | |
| | CATGCTGGAATGTCCACCCTGTTCCTGGTGTACTCCAACAAGTGCCAAACCCCACT | |
| | GGGAATGGCATCAGGACACATTAGAGACTTCCAGATTACAGCATCTGGACAGTAT | |
| | GGACAATGGGCCCCCAAGTTGGCCAGGCTGCACTACTCTGGAAGCATTAATGCCT | |
| | GGAGCACCAAGGAGCCATTCAGCTGGATCAAGGTGGACCTTCTGGCTCCAATGAT | |
| | CATCCATGGAATTAAGACTCAGGGAGCCAGACAGAAGTTCTCATCCCTCTACATCT | |
| | CCCAGTTTATCATCATGTACTCACTGGATGGGAAGAAGTGGCAGACTTACAGGGG | |
| | AAATTCCACAGGTACTCTGATGGTGTTCTTTGGAAATGTGGACAGCTCTGGCATCA | |
| | AGCACAATATCTTTAACCCTCCTATCATTGCCAGGTACATCAGACTCCACCCAACTC | |
| | ACTACTCCATCAGGTCCACTCTGAGGATGGAACTCATGGGTTGTGACCTCAACTCC | |
| | TGCTCAATGCCACTGGGCATGGAGTCCAAGGCTATCTCAGATGCTCAGATTACTGC | |
| | ATCCTCTTACTTTACCAACATGTTTGCTACCTGGTCCCCCTCCAAAGCCAGACTGCA | |
| | TCTCCAAGGCAGATCAAATGCCTGGAGGCCTCAGGTCAACAACCCAAAGGAATGG | |
| | CTTCAGGTGGACTTCCAAAAGACCATGAAAGTCACAGGAGTGACCACCCAGGGAG | |
| | TGAAATCCCTGCTGACCTCTATGTATGTGAAGGAATTCCTGATCTCATCAAGCCAG | |
| | GATGGCCACCAGTGGACACTGTTCTTCCAAAATGGAAAGGTCAAGGTCTTTCAGG | |
| | GAAATCAAGACTCCTTCACCCCTGTGGTGAACTCCCTGGACCCCCCTCTGCTTACCA | |
| | GGTACTTGAGAATTCATCCACAATCCTGGGTGCACCAGATTGCCCTGAGGATGGA | |
| | AGTGCTGGGCTGTGAAGCCCAGGACCTGTACTAA | |
| 96 | gccaccagaaggtactacctgggagctgtggaactgagctgggactacatgcagtctgacctgggagagct | F8-BDD4, codon |
| | gccTgtggatgctagatttcctccaagagtgcccaagagcttccccttcaacacctctgtggtgtacaagaaa | optimized |
| | accctgtttgtggaattcacagaccacctgttcaatattgccaagcctagacctccttggatgggcctgctggg | |
| | ccctacaattcaggctgaggtgtatgacacagtggtcatcaccctgaagaacatggccagccatcctgtgtct | |
| | ctgcatgctgtgggagtgtcttactggaaggcttctgagggggctgagtatgatgaccagacaagccagaga | |
| | gagaaagaggatgacaaggttttccctgggggcagccacacctatgtctggcaggtcctgaaagaaaatgg | |
| | ccctatggcctctgatcctctgtgcctgacatacagctacctgagccatgtggacctggtcaaggacctgaac | |
| | tctggcctgattggggctctgctggtgtgtagagaaggcagcctggccaaagaaaagacccagacactgca | |
| | caagttcatcctgctgtttgctgtgtttgatgagggcaagagctggcactctgagacaaagaacagcctgatg | |
| | caggacagagatgctgcctctgctagagcttggcccaagatgcacacagtgaatggctatgtgaacagaag | |
| | cctgcctggactgattggatgccacagaaagtctgtgtactggcatgtgattggcatgggcaccacacctgag | |
| | gtgcacagcatctttctggaaggacacaccttcctggtgaggaaccacagacaggccagcctggaaatcag | |
| | ccctatcaccttcctgacagctcagaccctgctgatggatctgggccagtttctgctgttctgccacatcagca | |
| | gccaccagcatgatggcatggaagcctatgtgaaggtggacagctgccctgaagaaccccagctgagaatg | |
| | aagaacaatgaggaagctgaggactatgatgatgacctgacagactctgagatggatgtggtcagatttga | |
| | tgatgataacagccccagcttcatccagatcagatctgtggccaagaagcaccccaagacctgggtgcacta | |
| | tattgctgctgaggaagaggactgggattatgctcctctggtgctggcccctgatgacagaagctacaagag | |
| | ccagtacctgaacaatggccctcagagaattggcaggaagtataagaaagtgaggttcatggcctacacag | |
| | atgagacattcaagaccagagaggctatccagcatgagtctggcattctgggacctctgctgtatggggaag | |
| | tgggggacacactgctgatcatcttcaagaaccaggccagcagaccctacaacatctaccctcatggcatca | |
| | cagatgtgaggcctctgtactctagaaggctgcccaagggggtgaagcacctgaaggacttccctatcctgc | |
| | ctggggagatcttcaagtacaagtggacagtgacagtggaggatggccctaccaagtctgatcctagatgcc | |
| | tgacaaggtactacagcagctttgtgaacatggaaagggacctggcctctggcctgattggtcctctgctgat | |
| | ctgctacaaagaatctgtggaccagaggggcaaccagatcatgagtgacaagagaaatgtgatcctgttctc | |
| | tgtctttgatgagaacaggtcctggtatctgacagagaacatccagaggtttctgcccaatcctgctggggtg | |
| | cagctggaagatcctgagttccaggcctccaacatcatgcactccatcaatggctatgtgtttgacagcctgc | |
| | agctgtctgtgtgcctgcatgaagtggcctactggtacatcctgtctattggggcccagacagacttcctgtct | |
| | gtgttcttttctggctacaccttcaagcacaagatggtgtatgaggataccctgacactgttcccattctctggg | |
| | gagacagtgttcatgagcatggaaaaccctggcctgtggatcctgggctgtcacaacagtgacttcagaaac | |
| | agaggcatgacagccctgctgaaggtgtccagctgtgacaagaacactggggactactatgaggactcttat | |
| | gaggacatctctgcctacctgctgagcaagaacaatgccattgagcctaggagcttctctcagaaccctcctg | |
| | tgctgaagagacaccagagggagatcaccagaaccacactgcagtctgaccaagaggaaattgattatgat | |
| | gacaccatctctgtggagatgaagaaagaagattttgacatctatgatgaggatgagaatcagagccccag | |
| | atctttccagaagaaaacaaggcactacttcattgctgctgtggaaagactgtgggactatggcatgagcag | |
| | cagcccccatgtgctgagaaacagggcccagtctggaagtgtgccccagttcaagaaagtggtgttccaag | |
| | agttcacagatggcagcttcacccagcctctgtatagaggggagctgaatgagcacctgggactgctgggac | |
| | cttacatcagagctgaggtggaggataacatcatggtcacctttagaaaccaggcctctaggccctactcctt | |
| | ctacagctccctgatcagctatgaagaggaccagagacagggggctgagcccagaaagaactttgtgaagc | |
| | ccaatgagactaagacctacttttggaaggtgcagcaccacatggcccctacaaaggatgagtttgactgca | |
| | aggcctgggcctacttctctgatgtggacctggagaaggatgtgcactctggactcattggacccctgcttgtg | |
| | tgccacaccaacacactgaatcctgctcatggcaggcaagtgacagtgcaagagtttgccctgttcttcacca | |
| | tctttgatgagacaaagtcctggtacttcacagaaaacatggaaagaaactgcagggccccttgcaacatcc | |
| | agatggaagatcccaccttcaaagagaactacaggttccatgccatcaatggctacatcatggacactctgc | |
| | ctggcctggttatggcacaggatcagaggatcagatggtatctgctgtccatgggctccaatgagaatatcca | |
| | cagcatccacttctctggccatgtgttcacagtgaggaaaaaagaagagtacaagatggccctgtacaatct | |
| | gtaccctggggtgtttgagactgtggaaatgctgcctagcaaggctggaatctggagggtggaatgtctgatt | |
| | ggagagcatctgcatgctggaatgtctaccctgttcctggtgtacagcaacaagtgtcagacccctctgggca | |
| | tggcctctggacacatcagagacttccagatcacagcctctggccagtatggacagtgggctcctaaactgg | |
| | ctagactgcactactctggcagcatcaatgcctggtccaccaaagagcccttcagctggatcaaggtggacc | |
| | tgctggctcccatgatcatccatggaatcaagacccagggggccagacagaagttcagcagcctgtacatca | |
| | gccagttcatcatcatgtacagcctggatggcaagaagtggcagacctacagaggcaacagcacaggcaca | |
| | ctcatggtgttctttggcaatgtggactcttctggcattaagcacaacatcttcaaccctccaatcattgccagg | |
| | tacatcaggctgcaccccacacactacagcatcagatctaccctgaggatggaactgatgggctgtgacctg | |
| | aacagctgctctatgcccctgggaatggaaagcaaggccatctctgatgcccagatcacagccagcagctac | |
| | ttcaccaacatgtttgccacatggtccccatctaaggccaggctgcatctgcagggcagatctaatgcttgga | |
| | ggccccaagtgaacaaccccaaagagtggctgcaggtggactttcagaaaaccatgaaagtgacaggagt | |
| | gaccacacagggggtcaagtctctgctgacctctatgtatgtgaaagagttcctgatctccagcagccaggat | |
| | ggccaccagtggaccctgtttttccagaatggcaaagtcaaggtgttccagggaaaccaggacagcttcaca | |
| | cctgtggtcaactccctggatcctccactgctgaccagatacctgagaattcaccctcagtcttgggtgcacca | |
| | gattgctctgagaatggaagtgctgggatgtgaagctcaggacctctac | |
| 97 | gccaccagaagatactacctgggtgcagtggaactgtcatgggactatatgcaaagtgatctcggtgagctg | Native FVIII-BDD |
| | cctgtggacgcaagatttcctcctagagtgccaaaatcttttccattcaacacctcagtcgtgtacaaaaaga | mature CDS |
| | ctctgtttgtagaattcacggttcaccttttcaacatcgctaagccaaggccaccctggatgggtctgctaggt | |
| | cctaccatccaggctgaggtttatgatacagtggtcattacacttaagaacatggcttcccatcctgtcagtct | |
| | tcatgctgttggtgtatcctactggaaagcttctgagggagctgaatatgatgatcagaccagtcaaaggga | |
| | gaaagaagatgataaagtcttccctggtggaagccatacatatgtctggcaggtcctgaaagagaatggtcc | |
| | aatggcctctgacccactgtgccttacctactcatatctttctcatgtggacctggtaaaagacttgaattcag | |
| | gcctcattggagccctactagtatgtagagaagggagtctggccaaggaaaagacacagaccttgcacaaa | |
| | tttatactactttttgctgtatttgatgaagggaaaagttggcactcagaaacaaagaactccttgatgcagg | |
| | atagggatgctgcatctgctcgggcctggcctaaaatgcacacagtcaatggttatgtaaacaggtctctgcc | |
| | aggtctgattggatgccacaggaaatcagtctattggcatgtgattggaatgggcaccactcctgaagtgca | |
| | ctcaatattcctcgaaggtcacacatttcttgtgaggaaccatcgccaggcgtccttggaaatctcgccaata | |
| | actttccttactgctcaaacactcttgatggaccttggacagtttctactgttttgtcatatctcttcccaccaac | |
| | atgatggcatggaagcttatgtcaaagtagacagctgtccagaggaaccccaactacgaatgaaaaataat | |
| | gaagaagcggaagactatgatgatgatcttactgattctgaaatggatgtggtcaggtttgatgatgacaact | |
| | ctccttcctttatccaaattcgctcagttgccaagaagcatcctaaaacttgggtacattacattgctgctgaa | |
| | gaggaggactgggactatgctcccttagtcctcgcccccgatgacagaagttataaaagtcaatatttgaac | |
| | aatggccctcagcggattggtaggaagtacaaaaaagtccgatttatggcatacacagatgaaacctttaag | |
| | actcgtgaagctattcagcatgaatcaggaatcttgggacctttactttatggggaagttggagacacactgt | |
| | tgattatatttaagaatcaagcaagcagaccatataacatctaccctcacggaatcactgatgtccgtcctttg | |
| | tattcaaggagattaccaaaaggtgtaaaacatttgaaggattttccaattctgccaggagaaatattcaaat | |
| | ataaatggacagtgactgtagaagatgggccaactaaatcagatcctcggtgcctgacccgctattactcta | |
| | gtttcgttaatatggagagagatctagcttcaggactcattggccctctcctcatctgctacaaagaatctgta | |
| | gatcaaagaggaaaccagataatgtcagacaagaggaatgtcatcctgttttctgtatttgatgagaaccga | |
| | agctggtacctcacagagaatatacaacgctttctccccaatccagctggagtgcagcttgaggatccagag | |
| | ttccaagcctccaacatcatgcacagcatcaatggctatgtttttgatagtttgcagttgtcagtttgtttgcat | |
| | gaggtggcatactggtacattctaagcattggagcacagactgacttcctttctgtcttcttctctggatatacc | |
| | ttcaaacacaaaatggtctatgaagacacactcaccctattcccattctcaggagaaactgtcttcatgtcga | |
| | tggaaaacccaggtctatggattctggggtgccacaactcagactttcggaacagaggcatgaccgccttac | |
| | tgaaggtttctagttgtgacaagaacactggtgattattacgaggacagttatgaagatatttcagcatacttg | |
| | ctgagtaaaaacaatgccattgaaccaagaagcttctcccagaatccaccagtcttgaaacgccatcaacgg | |
| | gaaataactcgtactactcttcagtcagatcaagaggaaattgactatgatgataccatatcagttgaaatga | |
| | agaaggaagattttgacatttatgatgaggatgaaaatcagagcccccgcagctttcaaaagaaaacacga | |
| | cactattttattgctgcagtggagaggctctgggattatgggatgagtagctccccacatgttctaagaaaca | |
| | gggctcagagtggcagtgtccctcagttcaagaaagttgttttccaggaatttactgatggctcctttactcag | |
| | cccttataccgtggagaactaaatgaacatttgggactcctggggccatatataagagcagaagttgaagat | |
| | aatatcatggtaactttcagaaatcaggcctctcgtccctattccttctattctagccttatttcttatgaggaag | |
| | atcagaggcaaggagcagaacctagaaaaaactttgtcaagcctaatgaaaccaaaacttacttttggaaa | |
| | gtgcaacatcatatggcacccactaaagatgagtttgactgcaaagcctgggcttatttctctgatgttgacct | |
| | ggaaaaagatgtgcactcaggcctgattggaccccttctggtctgccacactaacacactgaaccctgctcat | |
| | gggagacaagtgacagtacaggaatttgctctgtttttcaccatctttgatgagaccaaaagctggtacttca | |
| | ctgaaaatatggaaagaaactgcagggctccctgcaatatccagatggaagatcccacttttaaagagaatt | |
| | atcgcttccatgcaatcaatggctacataatggatacactacctggcttagtaatggctcaggatcaaaggat | |
| | tcgatggtatctgctcagcatgggcagcaatgaaaacatccattctattcatttcagtggacatgtgttcactg | |
| | tacgaaaaaaagaggagtataaaatggcactgtacaatctctatccaggtgtttttgagacagtggaaatgt | |
| | taccatccaaagctggaatttggcgggtggaatgccttattggcgagcatctacatgctgggatgagcacact | |
| | ttttctggtgtacagcaataagtgtcagactcccctgggaatggcttctggacacattagagattttcagatta | |
| | cagcttcaggacaatatggacagtgggccccaaagctggccagacttcattattccggatcaatcaatgcct | |
| | ggagcaccaaggagcccttttcttggatcaaggtggatctgttggcaccaatgattattcacggcatcaagac | |
| | ccagggtgcccgtcagaagttctccagcctctacatctctcagtttatcatcatgtatagtcttgatgggaaga | |
| | agtggcagacttatcgaggaaattccactggaaccttaatggtcttctttggcaatgtggattcatctgggata | |
| | aaacacaatatttttaaccctccaattattgctcgatacatccgtttgcacccaactcattatagcattcgcagc | |
| | actcttcgcatggagttgatgggctgtgatttaaatagttgcagcatgccattgggaatggagagtaaagcaa | |
| | tatcagatgcacagattactgcttcatcctactttaccaatatgtttgccacctggtctccttcaaaagctcgac | |
| | ttcacctccaagggaggagtaatgcctggagacctcaggtgaataatccaaaagagtggctgcaagtggac | |
| | ttccagaagacaatgaaagtcacaggagtaactactcagggagtaaaatctctgcttaccagcatgtatgtg | |
| | aaggagttcctcatctccagcagtcaagatggccatcagtggactctcttttttcagaatggcaaagtaaagg | |
| | tttttcagggaaatcaagactccttcacacctgtggtgaactctctagacccaccgttactgactcgctaccttc | |
| | gaattcacccccagagttgggtgcaccagattgccctgaggatggaggttctgggctgcgaggcacaggacc | |
| | tctac | |
| 98 | DAHATRRYY | |
| 99 | T/CNC/TT/CA/GAC/T | Branch site consensus |
| | N is any nucleotide | sequence |
| 100 | NNNNNNNNNNNNNNNNNNNNNRG | target nucleic acid |
| | N is any nucleotide | sequence |
| | R is G or A | |
| 101 | NNNNGATT | Neisseria meningitidis |
| | N is any nucleotide | PAM |
| 102 | NNNNNGTTT | Neisseria meningitidis |
| | N is any nucleotide | PAM |
| 103 | NNNNGCTT | Neisseria meningitidis |
| | N is any nucleotide | PAM |
| 104 | ACCTTTTTTTTTTTTTACCTAGG | Human Albumin Intron- |
| | | 1_T25 |
| 105 | UAAUUUUCUUUUGCGCACUA | Exemplary gRNA spacer |
| 106 | TAAAGCATAGTGCAATGGAT | |
| 107 | GAAAGCATGGTGCAATGGAT | |
| 108 | TATTGCACAGTGCAATGGAT | |
| 109 | TGATGCATATTGCAATGGAT | |
| 110 | TAATGAATAGGGCAATGGAT | |
| 111 | TAAGGCACAGTGTAATGGAT | |
| 112 | AAAAGCATAGACCAATGGAT | |
| 113 | TAGAGTATAGTGCAGTGGAT | |
| 114 | CAAAGCAAAGTGCAATTGAT | |
| 115 | GGAAGCATAGTGCAATGGTT | |
| 116 | TAAAGGATAGAGCAATGTAT | |
| 117 | TAGAGTATAGTGCAATGGAG | |
| 118 | TAAAGAATAGTGAAATGGTT | |
| 119 | ATTTATGAGATCAACAGCAC | |
| 120 | ATTTATGATATCATCAGCAC | |
| 121 | AAATATGACATCAACAGCAC | |
| 122 | ATCTTTGAGATCATCAGCAC | |
| 123 | ATGTATCAGATCATCAGCAC | |
| 124 | AATTATGAGATTCACAGCAC | |
| 125 | ATTTATGTGTTCAACCGCAC | |
| 126 | ATATATGACATCAACAGAAC | |
| 127 | ACTTATGATATCAACAGCAT | |
| 128 | TTAAATAAAGCATAGTGCAA | |
| 129 | TAAAATAAAGCATAGTGCAA | |
| 130 | TTAAATAAAGGATATTGCAA | |
| 131 | TTAAATAAAGCATTGAGCAA | |
| 132 | TACTATAAAGCATAGTGCAA | |
| 133 | TACTATAAAGCATAGTGCAA | |
| 134 | TTAAGGAAACCATAGTGCAA | |
| 135 | ATAAATATATCATAGTGCAA | |
| 136 | CTAAATAGAGAATAGTGCAA | |
| 137 | TTAAAGAAATTATAGTGCAA | |
| 138 | TTAAATATATAATAGTGCAA | |
| 139 | TTAAAAACAGCACAGTGCAA | |
| 140 | TTAAAATAAGCATGGTGCAA | |
| 141 | TTTGATAAAGCATAGGGCAA | |
| 142 | TTTTATAAAGCATAGTCCAA | |
| 143 | TTAAATGAAGAATATTGCAA | |
| 144 | ATAAATAAAGAATAGAGCAA | |
| 145 | TTGAATAAAGCAGAGTGGAA | |
| 146 | TTAATTAATGCATAGTGCCA | |
| 147 | TTAGATAAAGCTTAGTGCTA | |
| 148 | TTAGATAAAGCATACTGGAA | |
| 149 | TTAAAGAAAGCATGGTGCAG | |
| 150 | TTACATAAAGCATACTGCAT | |
| 151 | TTATATAAAGCATAGAGCAG | |
| 152 | TTAAATGAAGCATAGTGAAG | |
| 153 | TAATAAAATTCAAACATCCT | |
| 154 | GAATAAAATTCTAACATCCT | |
| 155 | TAATATAATTCCAACATCCT | |
| 156 | AAATAAAATTCAAACTTCCT | |
| 157 | GAGTTAAATTCAAACATCCT | |
| 158 | TTTTAAAAATCAAACATCCT | |
| 159 | AAATGAAAGTCAAACATCCT | |
| 160 | GATTAAAATTTAAACATCCT | |
| 161 | TCTTAAAATTCCAACATCCT | |
| 162 | AAAAAAAATTCCAACATCCT | |
| 163 | TACTGAAATTCTAACATCCT | |
| 164 | TACAAAAATTCACACATCCT | |
| 165 | TATTAGAATTCAGACATCCT | |
| 166 | TAATAAAGCCCAAACATCCT | |
| 167 | TATTTAAATTCAAATATCCT | |
| 168 | AAATAAAGTTCAAAGATCCT | |
| 169 | TACAAAAATTCAAACTTCCT | |
| 170 | GAATAAAATTTAAATATCCT | |
| 171 | TATAAAAATTCAAACAGCCT | |
| 172 | TACTAAAATTTAAACTTCCT | |
| 173 | TAATAACCTTCAAACATTCT | |
| 174 | TAGTAAAATTCAAATGTCCT | |
| 175 | CAATAAAATTCAACCATCAT | |
| 176 | GAATAAAATTCAAACTTCTT | |
| 177 | AAATAAAATTCAAAAATCCC | |
| 178 | CCCTCCGTTTGTCCTAGCTTTTC | AlbF primer |
| 179 | CCAGATACAGAATATCTTCCTCAACGCAGA | AlbR primer |
| 180 | CCTTTGGCACAATGAAGTGG | For primer |
| 181 | GAATCTGAACCCTGATGACAAG | Rev primer |
| 182 | TAAAGCATAGTGCCAATGGAT | |
| 183 | GAAAGCATAATAGCAATGGAT | |
| 184 | CTCACCATGGGGCGCCTGCAACTGGTT | |
| 185 | TAAAGGATCGTTCAACTCTGTGAGT | |
| 186 | CCAAGCATAGGTAATGGAT | |
| 187 | AAAAGCATAGTGAATGAAT | |
| 188 | CTTCGTATAGAAACAAGACAG | |
| 189 | GAGAGAGAGAGAAAGAGACAG | |
| 190 | ATTCTAGAGGCATAGAGAGTTCAACCT | |
| 191 | TTTTCTGCCATTGACCTTAAAGCGC | |
| 192 | ATCTGTGGGATTATGACTGAAC | |
| 193 | CTTCTCATAAAACCTAGACAG | |
| 194 | ATGTGGAGATCATTGAGCA | |
| 195 | ATTAATATGGTATCATGGGAGCAGGAC | |
| 196 | TTAAAATAAAGCATAGTGCAA | |
| 197 | TTCCAACGAAGGCCTCAA | |
| 198 | TTACTATAAAGCATAGTGCAA | |
| 199 | AAAAAAAAAGAAAAGAAAAGAAA | |
| 200 | TTACCTAAAACAATTTCACA | |
| 201 | TTAATACTGGGCCCTGAAGCCAAATACAGTT | |
| 202 | GGCAACAACACATCATCAGTAGGGTAA | |
| 203 | TTCAGAAATAGAAAAGCTGATCCTCAA | |
| 204 | TTAAAGCGCTTGAAATCTACACTTGCAA | |
| 205 | TGATAGGATGTGTGTGTAGAAGACTCC | |
| 206 | CCATAGAAGATACCAGGACTTCTT | |
| 207 | ATGTAGAAGTTGGTCACGGTCCGCATCGGCT | |
| 208 | AAATAGAATACCTCAGCATTTCT | |
| 209 | AGATGAAAATCTATCAATGGCACCAGCGCCT | |
| 210 | TAAAAAAGGGCTGAGCATAGTGGCTCACACCT | |
| 211 | TATTCAACTCACAGAGTTGAACGATCCT | |
| 212 | tcgcg | Spacer |

1. A system comprising:
a deoxyribonucleic acid (DNA) endonuclease or nucleic acid encoding the DNA endonuclease;
a guide RNA (gRNA) comprising a spacer sequence that is complementary to a sequence within intron 1 of an albumin gene in a cell; and
a donor template comprising a nucleic acid sequence encoding a Factor VIII (FVIII) protein or functional derivative thereof.

2. The system of claim 1, wherein the gRNA comprises a spacer sequence from any one of SEQ ID NOs: 22, 21, 28, 30, 18-20, 23-27, 29, 31-44, and 104.

3-7. (canceled)

8. The system of claim 1, wherein the DNA endonuclease is Cas9.

9. The system of claim 1, wherein the nucleic acid encoding the DNA endonuclease is codon optimized for expression in a host cell and/or the nucleic acid sequence encoding a FVIII protein or functional derivative thereof is codon optimized for expression in a host cell.

10. (canceled)

11. The system of claim 1, wherein the nucleic acid encoding the DNA endonuclease is DNA or ribonucleic acid (RNA).

12-13. (canceled)

14. The system of claim 1, wherein the donor template is encoded in an Adeno Associated Virus (AAV) vector.

15. The system of claim 14, wherein the donor template comprises a donor cassette comprising the nucleic acid sequence encoding a FVIII protein or functional derivative thereof, and wherein the donor cassette is flanked on one or both sides by a gRNA target site.

16-17. (canceled)

18. The system of claim 15, wherein the gRNA target site of the donor template is the reverse complement of a genomic gRNA target site for a gRNA in the system.

19. The system of claim 1, wherein the DNA endonuclease or nucleic acid encoding the DNA endonuclease is formulated in a liposome or lipid nanoparticle and/or the DNA endonuclease is precomplexed with the gRNA as a ribonucleoprotein (RNP) complex.

20-21. (canceled)

22. A method of editing a genome in a cell, the method comprising providing the following to the cell:
(a) a gRNA comprising a spacer sequence that is complementary to a sequence within intron 1 of an albumin gene in the cell;
(b) a DNA endonuclease or nucleic acid encoding the DNA endonuclease; and
(c) a donor template comprising a nucleic acid sequence encoding a FVIII protein or functional derivative thereof.

23. The method of claim 22, wherein the gRNA comprises a spacer sequence from any one of SEQ ID NOs: 22, 21, 28, 30, 18-20, 23-27, 29, 31-44, and 104.

24-28. (canceled)

29. The method of claim 22, wherein the DNA endonuclease is Cas9.

30. The method of claim 22, wherein the nucleic acid encoding the DNA endonuclease is codon optimized for expression in the cell and/or the nucleic acid sequence encoding a FVIII protein or functional derivative thereof is codon optimized for expression in the cell.

31. (canceled)

32. The method of claim 22, wherein the nucleic acid encoding the DNA endonuclease is DNA or RNA.

33-34. (canceled)

35. The method of claim 22, wherein the donor template is encoded in an AAV vector.

36. The method of claim 22, wherein the donor template comprises a donor cassette comprising the nucleic acid sequence encoding a FVIII protein or functional derivative thereof, and wherein the donor cassette is flanked on one or both sides by a gRNA target site.

37-38. (canceled)

39. The method of claim 36, wherein the gRNA target site of the donor template is the reverse complement of a gRNA target site in the cell genome for the gRNA of (a).

40. The method of claim 22, wherein the DNA endonuclease or nucleic acid encoding the DNA endonuclease is formulated in a liposome or lipid nanoparticle and/or the DNA endonuclease is precomplexed with the gRNA as an RNP complex.

41-42. (canceled)

43. The method of claim 22, wherein the gRNA of (a) and the DNA endonuclease or nucleic acid encoding the DNA endonuclease of (b) are provided to the cell more than 4 days or at least 14 days after the donor template of (c) is provided to the cell.

44. (canceled)

45. The method of claim 43, wherein one or more additional doses of the gRNA of (a) and the DNA endonuclease or nucleic acid encoding the DNA endonuclease of (b) are provided to the cell following the first dose of the gRNA of (a) and the DNA endonuclease or nucleic acid encoding the DNA endonuclease of (b).

46. (canceled)

47. The method of claim 22, wherein the nucleic acid sequence encoding a FVIII protein or functional derivative thereof is expressed under the control of the endogenous albumin promoter.

48. The method of claim 22, wherein the cell is a hepatocyte.

49. A genetically modified cell in which the genome of the cell is edited by the method of claim 22.

50. The genetically modified cell of claim 49, wherein
(i) the nucleic acid sequence encoding a FVIII protein or functional derivative thereof is expressed under the control of the endogenous albumin promoter;
(ii) the nucleic acid sequence encoding a FVIII protein or functional derivative thereof is codon-optimized for expression in the cell; and/or
(iii) the cell is a hepatocyte.

51-52. (canceled)

53. A method of treating Hemophilia A in a subject, the method comprising providing the following to a cell in the subject:
(a) a gRNA comprising a spacer sequence that is complementary to a sequence within intron 1 of an albumin gene in the cell;
(b) a DNA endonuclease or nucleic acid encoding the DNA endonuclease; and
(c) a donor template comprising a nucleic acid sequence encoding a FVIII protein or functional derivative thereof.

54. The method of claim 53, wherein the gRNA comprises a spacer sequence from any one of SEQ ID NOs: 22, 21, 28, 30, 18-20, 23-27, 29, 31-44, and 104.

55-58. (canceled)

59. The method of claim 53, wherein the subject is a patient having or suspected of having Hemophilia A or the subject is diagnosed with a risk of Hemophilia A.

60-61. (canceled)

62. The method of claim 53, wherein the DNA endonuclease is Cas9.

63. The method of claim 53, wherein the nucleic acid encoding the DNA endonuclease is codon optimized for expression in the cell and/or the nucleic acid sequence encoding a FVIII protein or functional derivative thereof is codon optimized for expression in the cell.

64. (canceled)

65. The method of claim 53, wherein the nucleic acid encoding the DNA endonuclease is DNA or RNA.

66-67. (canceled)

68. The method of claim 53, wherein one or more of the gRNA of (a), the DNA endonuclease or nucleic acid encoding the DNA endonuclease of (b), and the donor template of (c) are formulated in a liposome or lipid nanoparticle.

69. The method of claim 53, wherein the donor template is encoded in an AAV vector.

70. The method of claim 53, wherein the donor template comprises a donor cassette comprising the nucleic acid sequence encoding a FVIII protein or functional derivative thereof, and wherein the donor cassette is flanked on one or both sides by a gRNA target site.

71-72. (canceled)

73. The method of claim 70, wherein the gRNA target site of the donor template is the reverse complement of the gRNA target site in the cell genome for the gRNA of (a).

74-79. (canceled)

80. The method of claim 53, comprising providing to the cell the DNA endonuclease pre-complexed with the gRNA as an RNP complex.

81. The method of claim 53, wherein the gRNA of (a) and the DNA endonuclease or nucleic acid encoding the DNA endonuclease of (b) are provided to the cell more than 4 days or at least 14 days after the donor template of (c) is provided to the cell.

82. (canceled)

83. The method of claim 81, wherein one or more additional doses of the gRNA of (a) and the DNA endonuclease or nucleic acid encoding the DNA endonuclease of (b) are provided to the cell following the first dose of the gRNA of (a) and the DNA endonuclease or nucleic acid encoding the DNA endonuclease of (b).

84. (canceled)

85. The method of claim 81, wherein providing the gRNA of (a) and the DNA endonuclease or nucleic acid encoding the DNA endonuclease of (b) to the cell comprises administering to the subject a lipid nanoparticle comprising the nucleic acid encoding the DNA endonuclease and the gRNA; and/or providing the donor template of (c) to the cell comprises administering to the subject the donor template encoded in an AAV vector.

86. (canceled)

87. The method of claim 53, wherein the nucleic acid sequence encoding a FVIII protein or functional derivative thereof is expressed under the control of the endogenous albumin promoter; the cell is a hepatocyte; and/or the nucleic acid sequence encoding a FVIII protein or functional derivative thereof is expressed in the liver of the subject.

88-89. (canceled)

90. A method of treating Hemophilia A in a subject comprising:
administering the genetically modified cell of claim 49 to the subject.

91. The method of claim 90, wherein the genetically modified cell is autologous to the subject.

92. The method of claim 90, further comprising:
obtaining a biological sample from the subject wherein the biological sample comprises a hepatocyte cell, and wherein the genetically modified cell is prepared from the hepatocyte.

93. A kit comprising one or more elements of the system of claim 1, and further comprising instructions for use.


SEQUENCE LISTING (212 sequences)

SEQ ID NO: 1 - protein, 14 aa, Artificial sequence
  Synthetic polypeptide
  misc_feature: linker peptide
SFSQNPPVLKRHQR

SEQ ID NO: 2 - DNA, 25 bp, Artificial sequence
  Synthetic polynucleotide
  misc_feature: synthetic splice acceptor
ctgacctcttctcttcctcccacag

SEQ ID NO: 3 - DNA, 32 bp, Homo sapiens
  misc_feature: native albumin intron 1/exon 2 splice acceptor, human
ttaacaatccttttttttcttcccttgcccag

SEQ ID NO: 4 - DNA, 40 bp, Mus musculus
  misc_feature: native albumin intron 1/exon 2 splice acceptor, mouse
ttaaatatgttgtgtggtttttctctccctgtttccacag

SEQ ID NO: 5 - DNA, 49 bp, Artificial sequence
  Synthetic polynucleotide
  misc_feature: consensus synthetic poly A signal sequence
aataaaagatctttattttcattagatctgtgtgttggttttttgtgtg

SEQ ID NO: 6 - protein, 9 aa, Artificial sequence
  Synthetic polypeptide
LAGLIDADG

SEQ ID NO: 7 - DNA, 20 bp, Artificial sequence
  Synthetic polynucleotide
  misc_feature: target site
tgcctttaccccatcgttac

SEQ ID NO: 8 - DNA, 20 bp, Artificial sequence
  Synthetic polynucleotide
  misc_feature: target site
tgcctcctcccgatagttac

SEQ ID NO: 9 - DNA, 20 bp, Artificial sequence
  Synthetic polynucleotide
  misc_feature: target site
ggacagttcctgattgttac

SEQ ID NO: 10 - DNA, 20 bp, Artificial sequence
  Synthetic polynucleotide
  misc_feature: target site
tgccttttcccgattgttaa

SEQ ID NO: 11 - DNA, 20 bp, Artificial sequence
  Synthetic polynucleotide
  misc_feature: MALBF3 primer
ttattacggtctcatagggc

SEQ ID NO: 12 - DNA, 21 bp, Artificial sequence
  Synthetic polynucleotide
  misc_feature: MALBR5 primer
agtctttctgtcaatgcacac

SEQ ID NO: 13 - RNA, 106 nt, Artificial sequence
  Synthetic polynucleotide
  misc_feature: gRNA
  modified_base (1)..(1): um
  modified_base (2)..(2): s represents phosphorothioate backbone
  modified_base (3)..(3): gm
  modified_base (4)..(4): s represents phosphorothioate backbone
  modified_base (5)..(5): cm
  modified_base (6)..(6): s represents phosphorothioate backbone
  modified_base (32)..(32): gm
  modified_base (33)..(33): cm
  modified_base (34)..(34): um
  modified_base (35)..(35): 2′-O-methyladenosine
  modified_base (40)..(40): um
  modified_base (41)..(41): 2′-O-methyladenosine
  modified_base (42)..(42): gm
  modified_base (43)..(43): cm
  modified_base (71)..(72): 2′-O-methyladenosine
  modified_base (73)..(73): cm
  modified_base (74)..(75): um
  modified_base (80)..(81): 2′-O-methyladenosine
  modified_base (82)..(82): gm
  modified_base (83)..(83): um
  modified_base (84)..(85): gm
  modified_base (86)..(86): cm
  modified_base (87)..(87): 2′-O-methyladenosine
  modified_base (88)..(89): cm
  modified_base (90)..(90): gm
  modified_base (91)..(91): 2′-O-methyladenosine
  modified_base (92)..(92): gm
  modified_base (93)..(93): um
  modified_base (94)..(94): cm
  modified_base (95)..(96): gm
  modified_base (97)..(97): um
  modified_base (98)..(98): gm
  modified_base (99)..(99): cm
  modified_base (100)..(100): um
  modified_base (101)..(101): s represents phosphorothioate backbone
  modified_base (102)..(102): um
  modified_base (103)..(103): s represents phosphorothioate backbone
  modified_base (104)..(104): um
  modified_base (105)..(105): s represents phosphorothioate backbone
usgscscaguucccgaucguuacguuuuagagcuagaaauagcaaguuaaaauaaggcuaguccguuaucaacuugaaaaaguggcaccgagucggugcusususu

SEQ ID NO: 14 - DNA, 20 bp, Artificial sequence
  Synthetic polynucleotide
  misc_feature: Albumin forward primer
ccctccgtttgtcctagctt

SEQ ID NO: 15 - DNA, 19 bp, Artificial sequence
  Synthetic polynucleotide
  misc_feature: Albumin reverse primer
tctacgaggcagcactgtt

SEQ ID NO: 16 - DNA, 23 bp, Artificial sequence
  Synthetic polynucleotide
  misc_feature: AAVS1 forward primer
aactgcttctcctcttgggaagt

SEQ ID NO: 17 - DNA, 25 bp, Artificial sequence
  Synthetic polynucleotide
  misc_feature: AAVS1 reverse primer
cctctccatcctcttgctttctttg

SEQ ID NO: 18 - DNA, 23 bp, Homo sapiens
  misc_feature: Human Albumin Intron-1_T1
taattttcttttgcgcactaagg

SEQ ID NO: 19 - DNA, 23 bp, Homo sapiens
  misc_feature: Human Albumin Intron-1_T2
tagtgcaatggataggtctttgg

SEQ ID NO: 20 - DNA, 23 bp, Homo sapiens
  misc_feature: Human Albumin Intron-1_T3
agtgcaatggataggtctttggg

SEQ ID NO: 21 - DNA, 23 bp, Homo sapiens
  misc_feature: Human Albumin Intron-1_T4
taaagcatagtgcaatggatagg

SEQ ID NO: 22 - DNA, 23 bp, Homo sapiens
  misc_feature: Human Albumin Intron-1_T5
atttatgagatcaacagcacagg

SEQ ID NO: 23 - DNA, 23 bp, Homo sapiens
  misc_feature: Human Albumin Intron-1_T6
tgattcctacagaaaaactcagg

SEQ ID NO: 24 - DNA, 23 bp, Homo sapiens
  misc_feature: Human Albumin Intron-1_T7
tgtatttgtgaagtcttacaagg

SEQ ID NO: 25 - DNA, 23 bp, Homo sapiens
  misc_feature: Human Albumin Intron-1_T8
gactgaaacttcacagaataggg

SEQ ID NO: 26 - DNA, 23 bp, Homo sapiens
  misc_feature: Human Albumin Intron-1_T9
aatgcataatctaagtcaaatgg

SEQ ID NO: 27 - DNA, 23 bp, Homo sapiens
  misc_feature: Human Albumin Intron-1_T10
tgactgaaacttcacagaatagg

SEQ ID NO: 28 - DNA, 23 bp, Homo sapiens
  misc_feature: Human Albumin Intron-1_T11
ttaaataaagcatagtgcaatgg

SEQ ID NO: 29 - DNA, 23 bp, Homo sapiens
  misc_feature: Human Albumin Intron-1_T12
gatcaacagcacaggttttgtgg

SEQ ID NO: 30 - DNA, 23 bp, Homo sapiens
  misc_feature: Human Albumin Intron-1_T13
taataaaattcaaacatcctagg

SEQ ID NO: 31 - DNA, 23 bp, Homo sapiens
  misc_feature: Human Albumin Intron-1_T14
ttcattttagtctgtcttcttgg

SEQ ID NO: 32 - DNA, 23 bp, Homo sapiens
  misc_feature: Human Albumin Intron-1_T15
attatctaagtttgaatataagg

SEQ ID NO: 33 - DNA, 23 bp, Homo sapiens
  misc_feature: Human Albumin Intron-1_T16
atcatcctgagtttttctgtagg

SEQ ID NO: 34 - DNA, 23 bp, Homo sapiens
  misc_feature: Human Albumin Intron-1_T17
gcatctttaaagaattattttgg

SEQ ID NO: 35 - DNA, 23 bp, Homo sapiens
  misc_feature: Human Albumin Intron-1_T18
tactaaaactttattttactggg

SEQ ID NO: 36 - DNA, 23 bp, Homo sapiens
  misc_feature: Human Albumin Intron-1_T19
tgaattattcttctgtttaaagg

SEQ ID NO: 37 - DNA, 23 bp, Homo sapiens
  misc_feature: Human Albumin Intron-1_T20
aatttttaaaatagtattcttgg

SEQ ID NO: 38 - DNA, 23 bp, Homo sapiens
  misc_feature: Human Albumin Intron-1_T21
atgcatttgtttcaaaatattgg

SEQ ID NO: 39 - DNA, 23 bp, Homo sapiens
  misc_feature: Human Albumin Intron-1_T22
tttggcatttatttctaaaatgg

SEQ ID NO: 40 - DNA, 23 bp, Homo sapiens
  misc_feature: Human Albumin Intron-1_T23
aaagttgaacaatagaaaaatgg

SEQ ID NO: 41 - DNA, 23 bp, Homo sapiens
  misc_feature: Human Albumin Intron-1_T24
ttactaaaactttattttactgg

SEQ ID NO: 42 - DNA, 23 bp, Homo sapiens
  misc_feature: Human Albumin Intron-1_T26
tgcatttgtttcaaaatattggg

SEQ ID NO: 43 - DNA, 23 bp, Homo sapiens
  misc_feature: Human Albumin Intron-1_T27
tgggcaagggaagaaaaaaaagg

SEQ ID NO: 44 - DNA, 23 bp, Homo sapiens
  misc_feature: Human Albumin Intron-1_T28
tcctaggtaaaaaaaaaaaaagg

SEQ ID NO: 45 - DNA, 23 bp, Artificial sequence
  Synthetic polynucleotide
taattttcttttgcccactaagg

SEQ ID NO: 46 - DNA, 23 bp, Artificial sequence
  Synthetic polynucleotide
tagtgcaatggataggtcttagg

SEQ ID NO: 47 - DNA, 23 bp, Artificial sequence
  Synthetic polynucleotide
agtgcaatggataggtcttaggg

SEQ ID NO: 48 - DNA, 23 bp, Artificial sequence
  Synthetic polynucleotide
taaagcatagtgcaatggatagg

SEQ ID NO: 49 - DNA, 23 bp, Artificial sequence
  Synthetic polynucleotide
atttatgagatcaacagcacagg

SEQ ID NO: 50 - DNA, 23 bp, Artificial sequence
  Synthetic polynucleotide
tgattcctacagaaaaagtcagg

SEQ ID NO: 51 - DNA, 23 bp, Artificial sequence
  Synthetic polynucleotide
aatgcataatctaagtcaaatgg

SEQ ID NO: 52 - DNA, 23 bp, Artificial sequence
  Synthetic polynucleotide
ttaaataaagcatagtgcaatgg

SEQ ID NO: 53 - DNA, 23 bp, Artificial sequence
  Synthetic polynucleotide
atttatgagatcaacagcacagg

SEQ ID NO: 54 - DNA, 23 bp, Artificial sequence
  Synthetic polynucleotide
taataaaattcaaacatcctagg

SEQ ID NO: 55 - DNA, 23 bp, Artificial sequence
  Synthetic polynucleotide
attatcctgactttttctgtagg

SEQ ID NO: 56 - DNA, 23 bp, Artificial sequence
  Synthetic polynucleotide
tactaaaactttattttacttgg

SEQ ID NO: 57 - DNA, 23 bp, Artificial sequence
  Synthetic polynucleotide
tgaattattcctctgtttaaagg

SEQ ID NO: 58 - DNA, 23 bp, Artificial sequence
  Synthetic polynucleotide
atgcatttgtttcaaaatattgg

SEQ ID NO: 59 - DNA, 23 bp, Artificial sequence
  Synthetic polynucleotide
tttggcatttatttctaaaatgg

SEQ ID NO: 60 - DNA, 23 bp, Artificial sequence
  Synthetic polynucleotide
aaagttgaacaatagaaaaatgg

SEQ ID NO: 61 - DNA, 23 bp, Artificial sequence
  Synthetic polynucleotide
tgcatttgtttcaaaatattggg

SEQ ID NO: 62 - DNA, 23 bp, Artificial sequence
  Synthetic polynucleotide
taattttcttttgcccactaagg

SEQ ID NO: 63 - DNA, 23 bp, Artificial sequence
  Synthetic polynucleotide
tagtgcaatggataggtcttagg

SEQ ID NO: 64 - DNA, 23 bp, Artificial sequence
  Synthetic polynucleotide
agtgcaatggataggtcttaggg

SEQ ID NO: 65 - DNA, 23 bp, Artificial sequence
  Synthetic polynucleotide
taaagcatagtgcaatggatagg

SEQ ID NO: 66 - DNA, 23 bp, Artificial sequence
  Synthetic polynucleotide
atttatgagatcaacagcacagg

SEQ ID NO: 67 - DNA, 23 bp, Artificial sequence
  Synthetic polynucleotide
tgattcctacagaaaaagtcagg

SEQ ID NO: 68 - DNA, 23 bp, Artificial sequence
  Synthetic polynucleotide
aatgcataatctaagtcaaatgg

SEQ ID NO: 69 - DNA, 23 bp, Artificial sequence
  Synthetic polynucleotide
ttaaataaagcatagtgcaatgg

SEQ ID NO: 70 - DNA, 23 bp, Artificial sequence
  Synthetic polynucleotide
atttatgagatcaacagcacagg

SEQ ID NO: 71 - DNA, 23 bp, Artificial sequence
  Synthetic polynucleotide
taataaaattcaaacatcctagg

SEQ ID NO: 72 - DNA, 23 bp, Artificial sequence
  Synthetic polynucleotide
attatcctgactttttctgtagg

SEQ ID NO: 73 - DNA, 23 bp, Artificial sequence
  Synthetic polynucleotide
tactaaaactttattttacttgg

SEQ ID NO: 74 - DNA, 23 bp, Artificial sequence
  Synthetic polynucleotide
tgaattattcctctgtttaaagg

SEQ ID NO: 75 - DNA, 23 bp, Artificial sequence
  Synthetic polynucleotide
atgcatttgtttcaaaatattgg

SEQ ID NO: 76 - DNA, 23 bp, Artificial sequence
  Synthetic polynucleotide
tttggcatttatttctaaaatgg

SEQ ID NO: 77 - DNA, 23 bp, Artificial sequence
  Synthetic polynucleotide
aaagttgaacaatagaaaaatgg

SEQ ID NO: 78 - DNA, 23 bp, Artificial sequence
  Synthetic polynucleotide
tgcatttgtttcaaaatattggg

SEQ ID NO: 79 - DNA, 23 bp, Artificial sequence
  Synthetic polynucleotide
tggggaaggggagaaaaaaaagg

SEQ ID NO: 80 - DNA, 23 bp, Mus musculus
  misc_feature: Mouse albumin intron 1 gRNA sequence, mALbgRNA_T1
tgccagttcccgatcgttacagg

SEQ ID NO: 81 - DNA, 4438 bp, Artificial sequence
  Synthetic polynucleotide
  misc_feature: spCas9 mRNA
ggaaataagagagaaaagaagagtaagaagaaatataagagccaccatggccccaaagaagaagcggaaggtcggtatccacggagtcccagcagccgacaagaagtacagcatcggcctggacatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggacaagaagcacgagagacaccccatcttcggcaacatcgtggacgaggtggcctaccacgagaagtaccccaccatctaccacctgagaaagaaactggtggacagcaccgacaaggccgacctgagactgatctacctggccctggcccacatgatcaagttcagaggccacttcctgatcgagggcgacctgaaccccgacaacagcgacgtggacaagctgttcatccagctggtgcagacctacaaccagctgttcgaggaaaaccccatcaacgccagcggcgtggacgccaaggctatcctgtctgccagactgagcaagagcagaaggctggaaaatctgatcgcccagctgcccggcgagaagaagaacggcctgttcggcaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgttcctggccgccaagaacctgtctgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagaaatcttcttcgaccagagcaagaacggctacgccggctacatcgatggcggcgctagccaggaagagttctacaagttcatcaagcccatcctggaaaagatggacggcaccgaggaactgctcgtgaagctgaacagagaggacctgctgagaaagcagagaaccttcgacaacggcagcatcccccaccagatccacctgggagagctgcacgctatcctgagaaggcaggaagatttttacccattcctgaaggacaaccgggaaaagatcgagaagatcctgaccttcaggatcccctactacgtgggccccctggccagaggcaacagcagattcgcctggatgaccagaaagagcgaggaaaccatcaccccctggaacttcgaggaagtggtggacaagggcgccagcgcccagagcttcatcgagagaatgacaaacttcgataagaacctgcccaacgagaaggtgctgcccaagcacagcctgctgtacgagtacttcaccgtgtacaacgagctgaccaaagtgaaatacgtgaccgagggaatgagaaagcccgccttcctgagcggcgagcagaaaaaggccatcgtggacctgctgttcaagaccaacagaaaagtgaccgtgaagcagctgaaagaggactacttcaagaaaatcgagtgcttcgactccgtggaaatctccggcgtggaagatagattcaacgcctccctgggcacataccacgatctgctgaaaattatcaaggacaaggacttcctggataacgaagagaacgaggacattctggaagatatcgtgctgaccctgacactgtttgaggaccgcgagatgatcgaggaaaggctgaaaacctacgctcacctgttcgacgacaaagtgatgaagcagctgaagagaaggcggtacaccggctggggcaggctgagcagaaagctgatcaacggcatcagagacaagcagagcggcaagacaatcctggatttcctgaagtccgacggcttcgccaaccggaacttcatgcagctgatccacgacgacagcctgacattcaaagaggacatccagaaagcccaggtgtccggccagggcgactctctgcacgagcatatcgctaacctggccggcagccccgctatcaagaagggcatcctgcagacagtgaaggtggtggacgagctcgtgaaagtgatgggcagacacaagcccgagaacatcgtgatcgagatggctagagagaaccagaccacccagaagggacagaagaactcccgcgagaggatgaagagaatcgaagagggcatcaaagagctgggcagccagatcctgaaagaacaccccgtggaaaacacccagctgcagaacgagaagctgtacctgtactacctgcagaatggccgggatatgtacgtggaccaggaactggacatcaacagactgtccgactacgatgtggaccatatcgtgcctcagagctttctgaaggacgactccatcgataacaaagtgctgactcggagcgacaagaacagaggcaagagcgacaacgtgccctccgaagaggtcgtgaagaagatgaagaactactggcgacagctgctgaacgccaagctgattacccagaggaagttcgataacctgaccaaggccgagagaggcggcctgagcgagctggataaggccggcttcatcaagaggcagctggtggaaaccagacagatcacaaagcacgtggcacagatcctggactcccggatgaacactaagtacgacgaaaacgataagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgaaatcaccctggccaacggcgagatcagaaagcgccctctgatcgagacaaacggcgaaaccggggagatcgtgtgggataagggcagagacttcgccacagtgcgaaaggtgctgagcatgccccaagtgaatatcgtgaaaaagaccgaggtgcagacaggcggcttcagcaaagagtctatcctgcccaagaggaacagcgacaagctgatcgccagaaagaaggactgggaccccaagaagtacggcggcttcgacagccctaccgtggcctactctgtgctggtggtggctaaggtggaaaagggcaagtccaagaaactgaagagtgtgaaagagctgctggggatcaccatcatggaaagaagcagctttgagaagaaccctatcgactttctggaagccaagggctacaaagaagtgaaaaaggacctgatcatcaagctgcctaagtactccctgttcgagctggaaaacggcagaaagagaatgctggcctctgccggcgaactgcagaagggaaacgagctggccctgcctagcaaatatgtgaacttcctgtacctggcctcccactatgagaagctgaagggcagccctgaggacaacgaacagaaacagctgtttgtggaacagcataagcactacctggacgagatcatcgagcagatcagcgagttctccaagagagtgatcctggccgacgccaatctggacaaggtgctgtctgcctacaacaagcacagggacaagcctatcagagagcaggccgagaatatcatccacctgttcaccctgacaaacctgggcgctcctgccgccttcaagtactttgacaccaccatcgaccggaagaggtacaccagcaccaaagaggtgctggacgccaccctgatccaccagagcatcaccggcctgtacgagacaagaatcgacctgtctcagctgggaggcgacaagagacctgccgccactaagaaggccggacaggccaaaaagaagaagtgagcggccgcttaattaagctgccttctgcggggcttgccttctggccatgcccttcttctctcccttgcacctgtacctcttggtctttgaataaagcctgagtaggaagaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 82 - DNA, 25 bp, Artificial sequence
  Synthetic polynucleotide
  misc_feature: Synthetic splice acceptor
ctgacctcttctcttcctcccacag

SEQ ID NO: 83 - DNA, 42 bp, Mus musculus
  misc_feature: Mouse albumin Intron 1 splice acceptor
ctttaaatatgttgtgtggtttttctctccctgtttccacag

SEQ ID NO: 84 - DNA, 33 bp, Mus musculus
  misc_feature: Mouse albumin Intron 2 splice acceptor
cctcatactgaggtttttgtgtctgcttttcag

SEQ ID NO: 85 - DNA, 32 bp, Homo sapiens; identical to SEQ ID NO: 3
  misc_feature: Human albumin Intron 1 splice acceptor

SEQ ID NO: 86 - DNA, 34 bp, Homo sapiens
  misc_feature: Human albumin Intron 2 splice acceptor
attatactacatttttctacatcctttgtttcag

SEQ ID NO: 87 - DNA, 4402 bp, Artificial sequence
  Synthetic polynucleotide
  misc_feature: MAB8A
aattgctgacctcttctcttcctcccacagtggccaccagaagatactacctcggagccgtcgaattgagctgggattacatgcaatccgacctgggagaactgcccgtggatgccaggtttcctcctcgggtccccaagtccttcccgttcaacacctcagtcgtctacaagaaaaccctcttcgtggagttcaccgaccatctgttcaacatcgccaagccaagacccccgtggatgggactcctcggtccgaccatccaagccgaagtgtacgacactgtggtcattaccctgaagaacatggcctcccatcctgtgtccctgcatgcagtgggcgtgtcctactggaaggcttccgaaggggccgagtacgacgatcaaaccagccagcgggaaaaggaggatgacaaagtgttcccgggtggttcgcacacctacgtgtggcaagtgctcaaggagaacggtcctatggcctctgatcccctgtgtctgacctactcctacctgtcccatgtcgacctcgtgaaggatctgaacagcgggctgattggcgccctgctcgtgtgccgggaaggctccctggccaaggaaaagacccagacactgcacaagttcatcttgctgttcgccgtgtttgatgagggaaagtcctggcatagcgagactaagaactcccttatgcaagaccgggatgctgcctccgctagggcttggcctaagatgcatactgtgaacggatacgtgaacagatccctgcctggccttatcggttgccaccggaagtccgtgtattggcatgtgatcggcatgggaaccactccagaggtgcactccattttcttggaggggcataccttcttggtgcgcaaccacagacaggcctccctggaaatttctccgatcactttcctgactgcccagaccctccttatggacctgggtcagttcctgctgttctgccacatttcgtcccaccaacacgatggcatggaagcctacgtgaaagtggactcgtgcccggaagaaccacagctgcggatgaagaacaacgaagaggcagaggactacgatgatgatcttaccgattcggaaatggatgtggtccgattcgacgacgataatagcccatccttcatccaaattaggagcgtggccaagaagcaccccaaaacttgggtgcattacattgcggccgaggaagaggattgggactacgcacccctcgtgcttgcacccgatgatcggtcctacaagtcccaatacctgaacaacggcccgcagaggatcggtcggaagtataagaaagtgcgcttcatggcctacaccgacgagactttcaagaccagagaggccattcagcacgaaagcggcattctggggccgctgttgtacggggaggtcggagatacactgctcatcattttcaagaaccaggcgtccagaccctacaacatctacccgcacggaatcactgacgtccgccccctgtactcccggagactcccgaagggagtcaagcacttgaaagacttccccatcctgcctggggaaatcttcaagtacaagtggaccgtgaccgtcgaggatgggccgaccaagtccgatccaagatgcctcactagatactactcatccttcgtcaacatggaacgggacctggcctcaggactgattggccccctgctcatctgctacaaggagtccgtggatcagcgcggaaaccagatcatgtcggacaaacgcaacgtcatcctcttctccgtctttgacgagaaccgctcatggtaccttacggagaacatccagcggttcctccccaaccctgccggagtgcagctcgaggacccggaattccaggcatcaaacattatgcactccatcaacggttacgtgttcgacagcctccagcttagcgtgtgcctccatgaagtcgcatattggtacatcctgtccattggagcacaaaccgactttctctccgtgttcttctccggatataccttcaagcacaagatggtgtacgaggataccctgaccctcttccccttctccggagagactgtgtttatgtcgatggaaaacccaggcctgtggattttggggtgccacaactcggatttccgaaaccggggcatgactgccttgctcaaggtgtcctcctgtgacaagaacacgggagactactacgaggactcctacgaggatatttccgcctacctcctgtccaagaacaacgccatcgaacccaggtccttcagccagaaccctcctgtcctcaagcgccatcagagagaaatcacccgcacgaccctgcagtccgaccaggaagagatcgattacgacgacactatctccgtcgaaatgaagaaggaggactttgacatctacgacgaagatgaaaatcagtcccctcgctcgttccaaaagaaaacgagacactacttcatcgctgctgtggagcggctctgggactacggcatgtcctcatcgccccacgtgcttaggaaccgggctcaatccgggagcgtccctcagttcaagaaagtggtgtttcaagaattcaccgatggaagcttcacgcagccgttgtacaggggcgaactgaacgagcaccttggcctgctgggaccttacatcagagcagaggtcgaggacaacatcatggtgaccttccggaaccaagcctcccggccatattcattctactcgagccttatctcatacgaggaggatcagagacagggggctgaacctcggaagaacttcgtcaagccgaacgagacaaagacctacttttggaaggtgcagcaccacatggccccgaccaaggatgagttcgactgcaaggcctgggcgtacttctccgacgtggatctcgaaaaggacgtgcattccgggctgatcggaccgctgctcgtctgccacactaacaccctcaatcctgctcacggcagacaagtgaccgtgcaggagttcgccctgttcttcaccatcttcgacgaaactaagtcatggtactttaccgagaacatggagcggaattgtcgggccccatgtaacatccagatggaggacccgacattcaaggagaactaccggttccacgccattaacggatacattatggacactcttccgggactcgtgatggcacaggaccaacgcatcagatggtatcttctgtcgatggggagcaacgaaaacatccattcgatccactttagcggtcacgtgttcacagtgcgcaagaaggaagagtacaagatggcgctgtacaacctgtaccctggggtgttcgagactgtggaaatgctgccgtccaaggccggaatttggcgcgtggaatgtctgatcggtgaacatctgcatgccggaatgtccaccctgttcctggtgtactccaacaagtgccaaaccccactgggaatggcatcaggacacattagagacttccagattaccgcgagcggacagtacggacaatgggcccccaagttggccaggctgcactactctggaagcattaacgcctggagcaccaaggagccgttcagctggatcaaggtggaccttctggcgccaatgatcatccacggaattaagactcagggagcccgccagaagttctcatcgctctacatctcccagtttatcatcatgtactcactggatgggaagaagtggcagacttaccggggaaattccaccggtactctgatggtgttcttcggaaacgtggacagctccggcatcaagcacaatatctttaacccgcctatcatcgcccgatacatccggctccacccgactcactactccatccggtcgactctgcggatggaactcatgggttgcgacctcaactcctgctcaatgccactgggcatggagtccaaggctatctcggacgctcagattactgcatcgtcgtactttaccaacatgttcgctacctggtccccgtccaaagcccggctgcatctccaaggcagatcaaacgcgtggaggcctcaggtcaacaacccgaaggaatggcttcaggtcgacttccaaaagaccatgaaagtcaccggagtgaccacccagggcgtgaaatcgctgctgacctctatgtacgtgaaggaattcctgatctcatcaagccaggacggccaccagtggacactgttcttccaaaatggaaaggtcaaggtctttcagggaaatcaagactccttcacccccgtggtgaactccctggacccccctctgcttacccgctacttgcgcattcatccgcaatcctgggtgcaccagatcgccctgcgaatggaagtgctgggctgtgaagcgcaggacctgtactaaaataaaagatctttattttcattagatctgtgtgttggttttttgtgtgccgc

SEQ ID NO: 88 - DNA, 4502 bp, Artificial sequence
  Synthetic polynucleotide
  misc_feature: MAB8B
aattgaactttgagtgtagcagagaggaaccattgccaccttcagattttaatgtctgacctcttctcttcctcccacagtggccaccagaagatactacctcggagccgtcgaattgagctgggattacatgcaatccgacctgggagaactgcccgtggatgccaggtttcctcctcgggtccccaagtccttcccgttcaacacctcagtcgtctacaagaaaaccctcttcgtggagttcaccgaccatctgttcaacatcgccaagccaagacccccgtggatgggactcctcggtccgaccatccaagccgaagtgtacgacactgtggtcattaccctgaagaacatggcctcccatcctgtgtccctgcatgcagtgggcgtgtcctactggaaggcttccgaaggggccgagtacgacgatcaaaccagccagcgggaaaaggaggatgacaaagtgttcccgggtggttcgcacacctacgtgtggcaagtgctcaaggagaacggtcctatggcctctgatcccctgtgtctgacctactcctacctgtcccatgtcgacctcgtgaaggatctgaacagcgggctgattggcgccctgctcgtgtgccgggaaggctccctggccaaggaaaagacccagacactgcacaagttcatcttgctgttcgccgtgtttgatgagggaaagtcctggcatagcgagactaagaactcccttatgcaagaccgggatgctgcctccgctagggcttggcctaagatgcatactgtgaacggatacgtgaacagatccctgcctggccttatcggttgccaccggaagtccgtgtattggcatgtgatcggcatgggaaccactccagaggtgcactccattttcttggaggggcataccttcttggtgcgcaaccacagacaggcctccctggaaatttctccgatcactttcctgactgcccagaccctccttatggacctgggtcagttcctgctgttctgccacatttcgtcccaccaacacgatggcatggaagcctacgtgaaagtggactcgtgcccggaagaaccacagctgcggatgaagaacaacgaagaggcagaggactacgatgatgatcttaccgattcggaaatggatgtggtccgattcgacgacgataatagcccatccttcatccaaattaggagcgtggccaagaagcaccccaaaacttgggtgcattacattgcggccgaggaagaggattgggactacgcacccctcgtgcttgcacccgatgatcggtcctacaagtcccaatacctgaacaacggcccgcagaggatcggtcggaagtataagaaagtgcgcttcatggcctacaccgacgagactttcaagaccagagaggccattcagcacgaaagcggcattctggggccgctgttgtacggggaggtcggagatacactgctcatcattttcaagaaccaggcgtccagaccctacaacatctacccgcacggaatcactgacgtccgccccctgtactcccggagactcccgaagggagtcaagcacttgaaagacttccccatcctgcctggggaaatcttcaagtacaagtggaccgtgaccgtcgaggatgggccgaccaagtccgatccaagatgcctcactagatactactcatccttcgtcaacatggaacgggacctggcctcaggactgattggccccctgctcatctgctacaaggagtccgtggatcagcgcggaaaccagatcatgtcggacaaacgcaacgtcatcctcttctccgtctttgacgagaaccgctcatggtaccttacggagaacatccagcggttcctccccaaccctgccggagtgcagctcgaggacccggaattccaggcatcaaacattatgcactccatcaacggttacgtgttcgacagcctccagcttagcgtgtgcctccatgaagtcgcatattggtacatcctgtccattggagcacaaaccgactttctctccgtgttcttctccggatataccttcaagcacaagatggtgtacgaggataccctgaccctcttccccttctccggagagactgtgtttatgtcgatggaaaacccaggcctgtggattttggggtgccacaactcggatttccgaaaccggggcatgactgccttgctcaaggtgtcctcctgtgacaagaacacgggagactactacgaggactcctacgaggatatttccgcctacctcctgtccaagaacaacgccatcgaacccaggtccttcagccagaaccctcctgtcctcaagcgccatcagagagaaatcacccgcacgaccctgcagtccgaccaggaagagatcgattacgacgacactatctccgtcgaaatgaagaaggaggactttgacatctacgacgaagatgaaaatcagtcccctcgctcgttccaaaagaaaacgagacactacttcatcgctgctgtggagcggctctgggactacggcatgtcctcatcgccccacgtgcttaggaaccgggctcaatccgggagcgtccctcagttcaagaaagtggtgtttcaagaattcaccgatggaagcttcacgcagccgttgtacaggggcgaactgaacgagcaccttggcctgctgggaccttacatcagagcagaggtcgaggacaacatcatggtgaccttccggaaccaagcctcccggccatattcattctactcgagccttatctcatacgaggaggatcagagacagggggctgaacctcggaagaacttcgtcaagccgaacgagacaaagacctacttttggaaggtgcagcaccacatggccccgaccaaggatgagttcgactgcaaggcctgggcgtacttctccgacgtggatctcgaaaaggacgtgcattccgggctgatcggaccgctgctcgtctgccacactaacaccctcaatcctgctcacggcagacaagtgaccgtgcaggagttcgccctgttcttcaccatcttcgacgaaactaagtcatggtactttaccgagaacatggagcggaattgtcgggccccatgtaacatccagatggaggacccgacattcaaggagaactaccggttccacgccattaacggatacattatggacactcttccgggactcgtgatggcacaggaccaacgcatcagatggtatcttctgtcgatggggagcaacgaaaacatccattcgatccactttagcggtcacgtgttcacagtgcgcaagaaggaagagtacaagatggcgctgtacaacctgtaccctggggtgttcgagactgtggaaatgctgccgtccaaggccggaatttggcgcgtggaatgtctgatcggtgaacatctgcatgccggaatgtccaccctgttcctggtgtactccaacaagtgccaaaccccactgggaatggcatcaggacacattagagacttccagattaccgcgagcggacagtacggacaatgggcccccaagttggccaggctgcactactctggaagcattaacgcctggagcaccaaggagccgttcagctggatcaaggtggaccttctggcgccaatgatcatccacggaattaagactcagggagcccgccagaagttctcatcgctctacatctcccagtttatcatcatgtactcactggatgggaagaagtggcagacttaccggggaaattccaccggtactctgatggtgttcttcggaaacgtggacagctccggcatcaagcacaatatctttaacccgcctatcatcgcccgatacatccggctccacccgactcactactccatccggtcgactctgcggatggaactcatgggttgcgacctcaactcctgctcaatgccactgggcatggagtccaaggctatctcggacgctcagattactgcatcgtcgtactttaccaacatgttcgctacctggtccccgtccaaagcccggctgcatctccaaggcagatcaaacgcgtggaggcctcaggtcaacaacccgaaggaatggcttcaggtcgacttccaaaagaccatgaaagtcaccggagtgaccacccagggcgtgaaatcgctgctgacctctatgtacgtgaaggaattcctgatctcatcaagccaggacggccaccagtggacactgttcttccaaaatggaaaggtcaaggtctttcagggaaatcaagactccttcacccccgtggtgaactccctggacccccctctgcttacccgctacttgcgcattcatccgcaatcctgggtgcaccagatcgccctgcgaatggaagtgctgggctgtgaagcgcaggacctgtactaaaataaaagatctttattttcattagatctgtgtgttggttttttgtgtgcgatcgggaactggcatcttcagggagtagcttaggtcagtgaagagaagccgc

SEQ ID NO: 89 - DNA, 4567 bp, Artificial sequence
  Synthetic polynucleotide
  misc_feature: MAB8C
gcggcctaaggcaattgtgccagttcccgatcgttacaggaactttgagtgtagcagagaggaaccattgccaccttcagattttaatgtctgacctcttctcttcctcccacagtggccaccagaagatactacctcggagccgtcgaattgagctgggattacatgcaatccgacctgggagaactgcccgtggatgccaggtttcctcctcgggtccccaagtccttcccgttcaacacctcagtcgtctacaagaaaaccctcttcgtggagttcaccgaccatctgttcaacatcgccaagccaagacccccgtggatgggactcctcggtccgaccatccaagccgaagtgtacgacactgtggtcattaccctgaagaacatggcctcccatcctgtgtccctgcatgcagtgggcgtgtcctactggaaggcttccgaaggggccgagtacgacgatcaaaccagccagcgggaaaaggaggatgacaaagtgttcccgggtggttcgcacacctacgtgtggcaagtgctcaaggagaacggtcctatggcctctgatcccctgtgtctgacctactcctacctgtcccatgtcgacctcgtgaaggatctgaacagcgggctgattggcgccctgctcgtgtgccgggaaggctccctggccaaggaaaagacccagacactgcacaagttcatcttgctgttcgccgtgtttgatgagggaaagtcctggcatagcgagactaagaactcccttatgcaagaccgggatgctgcctccgctagggcttggcctaagatgcatactgtgaacggatacgtgaacagatccctgcctggccttatcggttgccaccggaagtccgtgtattggcatgtgatcggcatgggaaccactccagaggtgcactccattttcttggaggggcataccttcttggtgcgcaaccacagacaggcctccctggaaatttctccgatcactttcctgactgcccagaccctccttatggacctgggtcagttcctgctgttctgccacatttcgtcccaccaacacgatggcatggaagcctacgtgaaagtggactcgtgcccggaagaaccacagctgcggatgaagaacaacgaagaggcagaggactacgatgatgatcttaccgattcggaaatggatgtggtccgattcgacgacgataatagcccatccttcatccaaattaggagcgtggccaagaagcaccccaaaacttgggtgcattacattgcggccgaggaagaggattgggactacgcacccctcgtgcttgcacccgatgatcggtcctacaagtcccaatacctgaacaacggcccgcagaggatcggtcggaagtataagaaagtgcgcttcatggcctacaccgacgagactttcaagaccagagaggccattcagcacgaaagcggcattctggggccgctgttgtacggggaggtcggagatacactgctcatcattttcaagaaccaggcgtccagaccctacaacatctacccgcacggaatcactgacgtccgccccctgtactcccggagactcccgaagggagtcaagcacttgaaagacttccccatcctgcctggggaaatcttcaagtacaagtggaccgtgaccgtcgaggatgggccgaccaagtccgatccaagatgcctcactagatactactcatccttcgtcaacatggaacgggacctggcctcaggactgattggccccctgctcatctgctacaaggagtccgtggatcagcgcggaaaccagatcatgtcggacaaacgcaacgtcatcctcttctccgtctttgacgagaaccgctcatggtaccttacggagaacatccagcggttcctccccaaccctgccggagtgcagctcgaggacccggaattccaggcatcaaacattatgcactccatcaacggttacgtgttcgacagcctccagcttagcgtgtgcctccatgaagtcgcatattggtacatcctgtccattggagcacaaaccgactttctctccgtgttcttctccggatataccttcaagcacaagatggtgtacgaggataccctgaccctcttccccttctccggagagactgtgtttatgtcgatggaaaacccaggcctgtggattttggggtgccacaactcggatttccgaaaccggggcatgactgccttgctcaaggtgtcctcctgtgacaagaacacgggagactactacgaggactcctacgaggatatttccgcctacctcctgtccaagaacaacgccatcgaacccaggtccttcagccagaaccctcctgtcctcaagcgccatcagagagaaatcacccgcacgaccctgcagtccgaccaggaagagatcgattacgacgacactatctccgtcgaaatgaagaaggaggactttgacatctacgacgaagatgaaaatcagtcccctcgctcgttccaaaagaaaacgagacactacttcatcgctgctgtggagcggctctgggactacggcatgtcctcatcgccccacgtgcttaggaaccgggctcaatccgggagcgtccctcagttcaagaaagtggtgtttcaagaattcaccgatggaagcttcacgcagccgttgtacaggggcgaactgaacgagcaccttggcctgctgggaccttacatcagagcagaggtcgaggacaacatcatggtgaccttccggaaccaagcctcccggccatattcattctactcgagccttatctcatacgaggaggatcagagacagggggctgaacctcggaagaacttcgtcaagccgaacgagacaaagacctacttttggaaggtgcagcaccacatggccccgaccaaggatgagttcgactgcaaggcctgggcgtacttctccgacgtggatctcgaaaaggacgtgcattccgggctgatcggaccgctgctcgtctgccacactaacaccctcaatcctgctcacggcagacaagtgaccgtgcaggagttcgccctgttcttcaccatcttcgacgaaactaagtcatggtactttaccgagaacatggagcggaattgtcgggccccatgtaacatccagatggaggacccgacattcaaggagaactaccggttccacgccattaacggatacattatggacactcttccgggactcgtgatggcacaggaccaacgcatcagatggtatcttctgtcgatggggagcaacgaaaacatccattcgatccactttagcggtcacgtgttcacagtgcgcaagaaggaagagtacaagatggcgctgtacaacctgtaccctggggtgttcgagactgtggaaatgctgccgtccaaggccggaatttggcgcgtggaatgtctgatcggtgaacatctgcatgccggaatgtccaccctgttcctggtgtactccaacaagtgccaaaccccactgggaatggcatcaggacacattagagacttccagattaccgcgagcggacagtacggacaatgggcccccaagttggccaggctgcactactctggaagcattaacgcctggagcaccaaggagccgttcagctggatcaaggtggaccttctggcgccaatgatcatccacggaattaagactcagggagcccgccagaagttctcatcgctctacatctcccagtttatcatcatgtactcactggatgggaagaagtggcagacttaccggggaaattccaccggtactctgatggtgttcttcggaaacgtggacagctccggcatcaagcacaatatctttaacccgcctatcatcgcccgatacatccggctccacccgactcactactccatccggtcgactctgcggatggaactcatgggttgcgacctcaactcctgctcaatgccactgggcatggagtccaaggctatctcggacgctcagattactgcatcgtcgtactttaccaacatgttcgctacctggtccccgtccaaagcccggctgcatctccaaggcagatcaaacgcgtggaggcctcaggtcaacaacccgaaggaatggcttcaggtcgacttccaaaagaccatgaaagtcaccggagtgaccacccagggcgtgaaatcgctgctgacctctatgtacgtgaaggaattcctgatctcatcaagccaggacggccaccagtggacactgttcttccaaaatggaaaggtcaaggtctttcagggaaatcaagactccttcacccccgtggtgaactccctggacccccctctgcttacccgctacttgcgcattcatccgcaatcctgggtgcaccagatcgccctgcgaatggaagtgctgggctgtgaagcgcaggacctgtactaaaataaaagatctttattttcattagatctgtgtgttggttttttgtgtgcgatcgggaactggcatcttcagggagtagcttaggtcagtgaagagaagtgccagttcccgatcgttacaggccgcgggccgc

SEQ ID NO: 90 - DNA, 4567 bp, Artificial sequence
  Synthetic polynucleotide
  misc_feature: MAB8D
gcggcctaaggcaattgtgccagttcccgatcgttacaggaactttgagtgtagcagagaggaaccattgccaccttcagattttaatgtctgacctcttctcttcctcccacagtggccaccagaaggtactacctaggagccgtggaactgagctgggactacatgcagtctgacctgggagagctgcccgtggacgctagatttcctccaagagtgcccaagagcttccccttcaacacctccgtggtgtacaagaaaaccctgttcgtggaattcaccgaccacctgttcaatatcgccaagcctagacctccttggatgggcctgctgggccctacaattcaggccgaggtgtacgacaccgtggtcatcaccctgaagaacatggccagccatcctgtgtctctgcacgccgtgggagtgtcttactggaaggcttctgagggcgccgagtacgacgaccagacaagccagagagagaaagaggacgacaaggttttccctggcggcagccacacctatgtctggcaggtcctgaaagaaaacggccctatggcctccgatcctctgtgcctgacatacagctacctgagccatgtggacctggtcaaggacctgaactctggcctgatcggcgctctgctcgtgtgtagagaaggcagcctggccaaagaaaagacccagacactgcacaagttcatcctgctgttcgccgtgttcgacgagggcaagagctggcacagcgagacaaagaacagcctgatgcaggacagagatgccgcctctgctagagcttggcccaagatgcacaccgtgaacggctacgtgaacagaagcctgcctggactgatcggatgccacagaaagtccgtgtactggcatgtgatcggcatgggcaccacacctgaggtgcacagcatctttctggaaggacacaccttcctcgtgcggaaccacagacaggccagcctggaaatcagccctatcaccttcctgaccgctcagaccctgctgatggatctgggccagtttctgctgttctgccacatcagcagccaccagcacgatggcatggaagcctacgtgaaggtggacagctgccccgaagaaccccagctgagaatgaagaacaacgaggaagccgaggactacgacgacgacctgaccgactctgagatggacgtcgtcagattcgacgacgataacagccccagcttcatccagatcagaagcgtggccaagaagcaccccaagacctgggtgcactatatcgccgccgaggaagaggactgggattacgctcctctggtgctggcccctgacgacagaagctacaagagccagtacctgaacaacggccctcagagaatcggccggaagtataagaaagtgcggttcatggcctacaccgacgagacattcaagaccagagaggctatccagcacgagagcggcattctgggacctctgctgtatggcgaagtgggcgacacactgctgatcatcttcaagaaccaggccagcagaccctacaacatctaccctcacggcatcaccgatgtgcggcctctgtactctagaaggctgcccaagggcgtgaagcacctgaaggacttccctatcctgcctggcgagatcttcaagtacaagtggaccgtgaccgtcgaggacggccctaccaagagcgatcctagatgcctgacacggtactacagcagcttcgtgaacatggaacgcgacctggccagcggcctgattggtcctctgctgatctgctacaaagaaagcgtggaccagaggggcaaccagatcatgagcgacaagagaaacgtgatcctgttctccgtctttgacgagaacaggtcctggtatctgaccgagaacatccagcggtttctgcccaatcctgctggcgtgcagctggaagatcctgagttccaggcctccaacatcatgcactccatcaacggctatgtgttcgacagcctgcagctgagcgtgtgcctgcacgaagtggcctactggtacatcctgtctatcggcgcccagaccgacttcctgtccgtgttctttagcggctacaccttcaagcacaagatggtgtacgaggataccctgacactgttcccattcagcggcgagacagtgttcatgagcatggaaaaccccggcctgtggatcctgggctgtcacaacagcgacttcagaaacagaggcatgacagccctgctgaaggtgtccagctgcgacaagaacaccggcgactactacgaggactcttacgaggacatcagcgcctacctgctgagcaagaacaatgccatcgagcctcggagcttctctcagaaccctcctgtgctgaagagacaccagcgcgagatcaccagaaccacactgcagagcgaccaagaggaaatcgattacgacgacaccatcagcgtcgagatgaagaaagaagatttcgacatctacgacgaggacgagaatcagagccccagatctttccagaagaaaacgcggcactacttcattgccgccgtggaaagactgtgggactacggcatgagcagcagcccacatgtgctgagaaacagggcccagagcggaagcgtgccccagttcaagaaagtggtgttccaagagttcaccgacggcagcttcacccagcctctgtatagaggcgagctgaacgagcacctgggactgctgggaccttacatcagagctgaggtcgaggataacatcatggtcacctttagaaaccaggcctctaggccctactccttctacagctccctgatcagctacgaagaggaccagagacagggcgctgagcccagaaagaacttcgtgaagcccaacgagactaagacctacttttggaaggtgcagcaccacatggcccctacaaaggacgagttcgactgcaaggcctgggcctacttctctgacgtggacctcgagaaggatgtgcacagcggactcatcggacccctgcttgtgtgccacaccaacacactgaatcccgctcacggcaggcaagtgaccgtgcaagagttcgccctgttcttcaccatcttcgatgagacaaagtcctggtacttcaccgaaaacatggaaagaaactgcagggccccttgcaacatccagatggaagatcccaccttcaaagagaactaccggttccacgccatcaatggctacatcatggacactctgcccggcctggttatggcacaggatcagaggatcagatggtatctgctgtccatgggctccaacgagaatatccacagcatccacttcagcggccatgtgttcaccgtgcggaaaaaagaagagtacaagatggccctgtacaatctgtaccccggcgtgttcgagactgtggaaatgctgcctagcaaggccggaatctggcgcgtggaatgtctgatcggagagcatctgcatgccggaatgtctaccctgttcctggtgtacagcaacaagtgtcagacccctctcggcatggcctctggacacatcagagacttccagatcaccgcctctggccagtacggacagtgggctcctaaactggctagactgcactacagcggcagcatcaacgcctggtccaccaaagagcccttcagctggatcaaggtggacctgctggctcccatgatcatccacggaatcaagacccagggcgccagacagaagttcagcagcctgtacatcagccagttcatcatcatgtacagcctggacggcaagaagtggcagacctacagaggcaacagcaccggcacactcatggtgttcttcggcaacgtggactccagcggcattaagcacaacatcttcaaccctccaatcattgcccggtacatccggctgcaccccacacactacagcatcagatctaccctgaggatggaactgatgggctgcgacctgaacagctgctctatgcccctcggaatggaaagcaaggccatcagcgacgcccagatcacagccagcagctacttcaccaacatgttcgccacatggtccccatctaaggcccggctgcatctgcagggcagatctaacgcttggaggccccaagtgaacaaccccaaagagtggctgcaggtcgactttcagaaaaccatgaaagtgaccggcgtgaccacacagggcgtcaagtctctgctgacctctatgtacgtgaaagagttcctgatctccagcagccaggacggccaccagtggaccctgtttttccagaacggcaaagtcaaggtgttccagggaaaccaggacagcttcacacccgtggtcaactccctggatcctccactgctgaccagatacctgagaattcaccctcagtcttgggtgcaccagatcgctctgagaatggaagtgctgggatgtgaagctcaggacctctactaaaataaaagatctttattttcattagatctgtgtgttggttttttgtgtgcgatcgggaactggcatcttcagggagtagcttaggtcagtgaagagaagtgccagttcccgatcgttacaggccgcgggccgc

SEQ ID NO: 91 - DNA, 4402 bp, Artificial sequence
  Synthetic polynucleotide
  misc_feature: MAB8E
aattgctgacctcttctcttcctcccacagtggccaccagaagatactacctgggagctgtggaattgagctgggattacatgcaatctgacctgggagaactgcctgtggatgccaggtttcctcctagggtccccaagtccttcccattcaacacctcagtggtctacaagaaaaccctctttgtggagttcacagaccatctgttcaacattgccaagccaagacccccatggatgggactcctgggtccaaccatccaagctgaagtgtatgacactgtggtcattaccctgaagaacatggcctcccatcctgtgtccctgcatgcagtgggagtgtcctactggaaggcttctgaaggggctgagtatgatgatcaaaccagccagagagaaaaggaggatgacaaagtgttcccaggtggtagtcacacctatgtgtggcaagtgctcaaggagaatggtcctatggcctctgatcccctgtgtctgacctactcctacctgtcccatgtggacctggtgaaggatctgaactctgggctgattggagccctgctggtgtgcagagaaggctccctggccaaggaaaagacccagacactgcacaagttcatcttgctgtttgctgtgtttgatgagggaaagtcctggcattctgagactaagaactcccttatgcaagacagagatgctgcctcagctagggcttggcctaagatgcatactgtgaatggatatgtgaacagatccctgcctggccttattggttgccacaggaagtctgtgtattggcatgtgattggcatgggaaccactccagaggtgcactccattttcttggaggggcataccttcttggtgaggaaccacagacaggcctccctggaaatttctccaatcactttcctgactgcccagaccctccttatggacctgggtcagttcctgctgttctgccacatttcatcccaccaacatgatggcatggaagcctatgtgaaagtggactcatgcccagaagaaccacagctgagaatgaagaacaatgaagaggcagaggactatgatgatgatcttacagattcagaaatggatgtggtcagatttgatgatgataatagcccatccttcatccaaattaggagtgtggccaagaagcaccccaaaacttgggtgcattacattgcagctgaggaagaggattgggactatgcacccttggtgcttgcaccagatgataggtcctacaagtcccaatacctgaacaatggcccacagaggattggtagaaagtataagaaagtgagattcatggcctacacagatgagactttcaagaccagagaggccattcagcatgaatctggcattctggggccactgttgtatggggaggttggagatacactgctcatcattttcaagaaccaggcctccagaccctacaacatctaccctcatggaatcactgatgtcagacccctgtactccagaagactcccaaagggagtcaagcacttgaaagacttccccatcctgcctggggaaatcttcaagtacaagtggacagtgacagtggaggatgggccaaccaagtctgatccaagatgcctcactagatactactcatcctttgtcaacatggaaagagacctggcctcaggactgattggccccctgctcatctgctacaaggagtctgtggatcagagaggaaaccagatcatgtctgacaaaaggaatgtcatcctcttctctgtctttgatgagaacagatcatggtaccttacagagaacatccagaggttcctccccaaccctgctggagtgcagctggaggacccagaattccaggcatcaaacattatgcactccatcaatggttatgtgtttgacagcctccagctttctgtgtgcctccatgaagtggcatattggtacatcctgtccattggagcacaaacagactttctctctgtgttcttctctggatataccttcaagcacaagatggtgtatgaggataccctgaccctcttccccttctctggagagactgtgtttatgtcaatggaaaacccaggcctgtggattttggggtgccacaactcagatttcagaaacaggggcatgactgccttgctcaaggtgtcctcctgtgacaagaacacaggagactactatgaggactcctatgaggatatttctgcctacctcctgtccaagaacaatgccattgaacccaggtccttcagccagaaccctcctgtcctcaagaggcatcagagagaaatcaccagaactaccctgcagtctgaccaggaagagattgattatgatgacactatctcagtggaaatgaagaaggaggactttgacatctatgatgaagatgaaaatcagtcccctaggtccttccaaaagaaaacaagacactacttcattgctgctgtggagagactctgggactatggcatgtcctcatcaccccatgtgcttaggaacagggctcaatctgggtctgtccctcagttcaagaaagtggtgtttcaagaattcacagatggaagcttcacacagccattgtacaggggagaactgaatgagcaccttggcctgctgggaccttacatcagagcagaggtggaggacaacatcatggtgaccttcagaaaccaagcctccaggccatattcattctactccagccttatctcatatgaggaggatcagagacagggggctgaacctaggaagaactttgtcaagccaaatgagacaaagacctacttttggaaggtgcagcaccacatggcccctaccaaggatgagtttgactgcaaggcctgggcttacttctctgatgtggatctggaaaaggatgtgcattctgggctgattggacctctgctggtctgccacactaacaccctcaatcctgctcatggcagacaagtgacagtgcaggagtttgccctgttcttcaccatctttgatgaaactaagtcatggtactttacagagaacatggagagaaattgtagggccccatgtaacatccagatggaggacccaacattcaaggagaactacagattccatgccattaatggatacattatggacactcttccaggactggtgatggcacaggaccaaagaatcagatggtatcttctgagcatggggagcaatgaaaacatccattccatccacttttcaggtcatgtgttcacagtgaggaagaaggaagagtacaagatggctctgtacaacctgtaccctggggtgtttgagactgtggaaatgctgccatccaaggctggaatttggagggtggaatgtctgattggtgaacatctgcatgctggaatgtccaccctgttcctggtgtactccaacaagtgccaaaccccactgggaatggcatcaggacacattagagacttccagattacagcatctggacagtatggacaatgggcccccaagttggccaggctgcactactctggaagcattaatgcctggagcaccaaggagccattcagctggatcaaggtggaccttctggctccaatgatcatccatggaattaagactcagggagccagacagaagttctcatccctctacatctcccagtttatcatcatgtactcactggatgggaagaagtggcagacttacaggggaaattccacaggtactctgatggtgttctttggaaatgtggacagctctggcatcaagcacaatatctttaaccctcctatcattgccaggtacatcagactccacccaactcactactccatcaggtccactctgaggatggaactcatgggttgtgacctcaactcctgctcaatgccactgggcatggagtccaaggctatctcagatgctcagattactgcatcctcttactttaccaacatgtttgctacctggtccccctccaaagccagactgcatctccaaggcagatcaaatgcctggaggcctcaggtcaacaacccaaaggaatggcttcaggtggacttccaaaagaccatgaaagtcacaggagtgaccacccagggagtgaaatccctgctgacctctatgtatgtgaaggaattcctgatctcatcaagccaggatggccaccagtggacactgttcttccaaaatggaaaggtcaaggtctttcagggaaatcaagactccttcacccctgtggtgaactccctggacccccctctgcttaccaggtacttgagaattcatccacaatcctgggtgcaccagattgccctgaggatggaagtgctgggctgtgaagcccaggacctgtactaaaataaaagatctttattttcattagatctgtgtgttggttttttgtgtgccgc

SEQ ID NO: 92 - DNA, 4419 bp, Artificial sequence
  Synthetic polynucleotide
  misc_feature: MAB8F (GA co/CpG free)
gctagccaattgctgacctcttctcttcctcccacagtggccaccagaaggtactacctgggagctgtggaactgagctgggactacatgcagtctgacctgggagagctgcctgtggatgctagatttcctccaagagtgcccaagagcttccccttcaacacctctgtggtgtacaagaaaaccctgtttgtggaattcacagaccacctgttcaatattgccaagcctagacctccttggatgggcctgctgggccctacaattcaggctgaggtgtatgacacagtggtcatcaccctgaagaacatggccagccatcctgtgtctctgcatgctgtgggagtgtcttactggaaggcttctgagggggctgagtatgatgaccagacaagccagagagagaaagaggatgacaaggttttccctgggggcagccacacctatgtctggcaggtcctgaaagaaaatggccctatggcctctgatcctctgtgcctgacatacagctacctgagccatgtggacctggtcaaggacctgaactctggcctgattggggctctgctggtgtgtagagaaggcagcctggccaaagaaaagacccagacactgcacaagttcatcctgctgtttgctgtgtttgatgagggcaagagctggcactctgagacaaagaacagcctgatgcaggacagagatgctgcctctgctagagcttggcccaagatgcacacagtgaatggctatgtgaacagaagcctgcctggactgattggatgccacagaaagtctgtgtactggcatgtgattggcatgggcaccacacctgaggtgcacagcatctttctggaaggacacaccttcctggtgaggaaccacagacaggccagcctggaaatcagccctatcaccttcctgacagctcagaccctgctgatggatctgggccagtttctgctgttctgccacatcagcagccaccagcatgatggcatggaagcctatgtgaaggtggacagctgccctgaagaaccccagctgagaatgaagaacaatgaggaagctgaggactatgatgatgacctgacagactctgagatggatgtggtcagatttgatgatgataacagccccagcttcatccagatcagatctgtggccaagaagcaccccaagacctgggtgcactatattgctgctgaggaagaggactgggattatgctcctctggtgctggcccctgatgacagaagctacaagagccagtacctgaacaatggccctcagagaattggcaggaagtataagaaagtgaggttcatggcctacacagatgagacattcaagaccagagaggctatccagcatgagtctggcattctgggacctctgctgtatggggaagtgggggacacactgctgatcatcttcaagaaccaggccagcagaccctacaacatctaccctcatggcatcacagatgtgaggcctctgtactctagaaggctgcccaagggggtgaagcacctgaaggacttccctatcctgcctggggagatcttcaagtacaagtggacagtgacagtggaggatggccctaccaagtctgatcctagatgcctgacaaggtactacagcagctttgtgaacatggaaagggacctggcctctggcctgattggtcctctgctgatctgctacaaagaatctgtggaccagaggggcaaccagatcatgagtgacaagagaaatgtgatcctgttctctgtctttgatgagaacaggtcctggtatctgacagagaacatccagaggtttctgcccaatcctgctggggtgcagctggaagatcctgagttccaggcctccaacatcatgcactccatcaatggctatgtgtttgacagcctgcagctgtctgtgtgcctgcatgaagtggcctactggtacatcctgtctattggggcccagacagacttcctgtctgtgttcttttctggctacaccttcaagcacaagatggtgtatgaggataccctgacactgttcccattctctggggagacagtgttcatgagcatggaaaaccctggcctgtggatcctgggctgtcacaacagtgacttcagaaacagaggcatgacagccctgctgaaggtgtccagctgtgacaagaacactggggactactatgaggactcttatgaggacatctctgcctacctgctgagcaagaacaatgccattgagcctaggagcttctctcagaaccctcctgtgctgaagagacaccagagggagatcaccagaaccacactgcagtctgaccaagaggaaattgattatgatgacaccatctctgtggagatgaagaaagaagattttgacatctatgatgaggatgagaatcagagccccagatctttccagaagaaaacaaggcactacttcattgctgctgtggaaagactgtgggactatggcatgagcagcagcccccatgtgctgagaaacagggcccagtctggaagtgtgccccagttcaagaaagtggtgttccaagagttcacagatggcagcttcacccagcctctgtatagaggggagctgaatgagcacctgggactgctgggaccttacatcagagctgaggtggaggataacatcatggtcacctttagaaaccaggcctctaggccctactccttctacagctccctgatcagctatgaagaggaccagagacagggggctgagcccagaaagaactttgtgaagcccaatgagactaagacctacttttggaaggtgcagcaccacatggcccctacaaaggatgagtttgactgcaaggcctgggcctacttctctgatgtggacctggagaaggatgtgcactctggactcattggacccctgcttgtgtgccacaccaacacactgaatcctgctcatggcaggcaagtgacagtgcaagagtttgccctgttcttcaccatctttgatgagacaaagtcctggtacttcacagaaaacatggaaagaaactgcagggccccttgcaacatccagatggaagatcccaccttcaaagagaactacaggttccatgccatcaatggctacatcatggacactctgcctggcctggttatggcacaggatcagaggatcagatggtatctgctgtccatgggctccaatgagaatatccacagcatccacttctctggccatgtgttcacagtgaggaaaaaagaagagtacaagatggccctgtacaatctgtaccctggggtgtttgagactgtggaaatgctgcctagcaaggctggaatctggagggtggaatgtctgattggagagcatctgcatgctggaatgtctaccctgttcctggtgtacagcaacaagtgtcagacccctctgggcatggcctctggacacatcagagacttccagatcacagcctctggccagtatggacagtgggctcctaaactggctagactgcactactctggcagcatcaatgcctggtccaccaaagagcccttcagctggatcaaggtggacctgctggctcccatgatcatccatggaatcaagacccagggggccagacagaagttcagcagcctgtacatcagccagttcatcatcatgtacagcctggatggcaagaagtggcagacctacagaggcaacagcacaggcacactcatggtgttctttggcaatgtggactcttctggcattaagcacaacatcttcaaccctccaatcattgccaggtacatcaggctgcaccccacacactacagcatcagatctaccctgaggatggaactgatgggctgtgacctgaacagctgctctatgcccctgggaatggaaagcaaggccatctctgatgcccagatcacagccagcagctacttcaccaacatgtttgccacatggtccccatctaaggccaggctgcatctgcagggcagatctaatgcttggaggccccaagtgaacaaccccaaagagtggctgcaggtggactttcagaaaaccatgaaagtgacaggagtgaccacacagggggtcaagtctctgctgacctctatgtatgtgaaagagttcctgatctccagcagccaggatggccaccagtggaccctgtttttccagaatggcaaagtcaaggtgttccagggaaaccaggacagcttcacacctgtggtcaactccctggatcctccactgctgaccagatacctgagaattcaccctcagtcttgggtgcaccagattgctctgagaatggaagtgctgggatgtgaagctcaggacctctactaaaataaaagatctttattttcattagatctgtgtgttggttttttgtgtgccgcgggtttaaac

SEQ ID NO: 93 - DNA, 4314 bp, Artificial sequence
  Synthetic polynucleotide
  misc_feature: F8-BDD1, codon optimized
gccaccagaagatactacctcggagccgtcgaattgagctgggattacatgcaatccgacctgggagaactgcccgtggatgccaggtttcctcctcgggtccccaagtccttcccgttcaacacctcagtcgtctacaagaaaaccctcttcgtggagttcaccgaccatctgttcaacatcgccaagccaagacccccgtggatgggactcctcggtccgaccatccaagccgaagtgtacgacactgtggtcattaccctgaagaacatggcctcccatcctgtgtccctgcatgcagtgggcgtgtcctactggaaggcttccgaaggggccgagtacgacgatcaaaccagccagcgggaaaaggaggatgacaaagtgttcccgggtggttcgcacacctacgtgtggcaagtgctcaaggagaacggtcctatggcctctgatcccctgtgtctgacctactcctacctgtcccatgtcgacctcgtgaaggatctgaacagcgggctgattggcgccctgctcgtgtgccgggaaggctccctggccaaggaaaagacccagacactgcacaagttcatcttgctgttcgccgtgtttgatgagggaaagtcctggcatagcgagactaagaactcccttatgcaagaccgggatgctgcctccgctagggcttggcctaagatgcatactgtgaacggatacgtgaacagatccctgcctggccttatcggttgccaccggaagtccgtgtattggcatgtgatcggcatgggaaccactccagaggtgcactccattttcttggaggggcataccttcttggtgcgcaaccacagacaggcctccctggaaatttctccgatcactttcctgactgcccagaccctccttatggacctgggtcagttcctgctgttctgccacatttcgtcccaccaacacgatggcatggaagcctacgtgaaagtggactcgtgcccggaagaaccacagctgcggatgaagaacaacgaagaggcagaggactacgatgatgatcttaccgattcggaaatggatgtggtccgattcgacgacgataatagcccatccttcatccaaattaggagcgtggccaagaagcaccccaaaacttgggtgcattacattgcggccgaggaagaggattgggactacgcacccctcgtgcttgcacccgatgatcggtcctacaagtcccaatacctgaacaacggcccgcagaggatcggtcggaagtataagaaagtgcgcttcatggcctacaccgacgagactttcaagaccagagaggccattcagcacgaaagcggcattctggggccgctgttgtacggggaggtcggagatacactgctcatcattttcaagaaccaggcgtccagaccctacaacatctacccgcacggaatcactgacgtccgccccctgtactcccggagactcccgaagggagtcaagcacttgaaagacttccccatcctgcctggggaaatcttcaagtacaagtggaccgtgaccgtcgaggatgggccgaccaagtccgatccaagatgcctcactagatactactcatccttcgtcaacatggaacgggacctggcctcaggactgattggccccctgctcatctgctacaaggagtccgtggatcagcgcggaaaccagatcatgtcggacaaacgcaacgtcatcctcttctccgtctttgacgagaaccgctcatggtaccttacggagaacatccagcggttcctccccaaccctgccggagtgcagctcgaggacccggaattccaggcatcaaacattatgcactccatcaacggttacgtgttcgacagcctccagcttagcgtgtgcctccatgaagtcgcatattggtacatcctgtccattggagcacaaaccgactttctctccgtgttcttctccggatataccttcaagcacaagatggtgtacgaggataccctgaccctcttccccttctccggagagactgtgtttatgtcgatggaaaacccaggcctgtggattttggggtgccacaactcggatttccgaaaccggggcatgactgccttgctcaaggtgtcctcctgtgacaagaacacgggagactactacgaggactcctacgaggatatttccgcctacctcctgtccaagaacaacgccatcgaacccaggtccttcagccagaaccctcctgtcctcaagcgccatcagagagaaatcacccgcacgaccctgcagtccgaccaggaagagatcgattacgacgacactatctccgtcgaaatgaagaaggaggactttgacatctacgacgaagatgaaaatcagtcccctcgctcgttccaaaagaaaacgagacactacttcatcgctgctgtggagcggctctgggactacggcatgtcctcatcgccccacgtgcttaggaaccgggctcaatccgggagcgtccctcagttcaagaaagtggtgtttcaagaattcaccgatggaagcttcacgcagccgttgtacaggggcgaactgaacgagcaccttggcctgctgggaccttacatcagagcagaggtcgaggacaacatcatggtgaccttccggaaccaagcctcccggccatattcattctactcgagccttatctcatacgaggaggatcagagacagggggctgaacctcggaagaacttcgtcaagccgaacgagacaaagacctacttttggaaggtgcagcaccacatggccccgaccaaggatgagttcgactgcaaggcctgggcgtacttctccgacgtggatctcgaaaaggacgtgcattccgggctgatcggaccgctgctcgtctgccacactaacaccctcaatcctgctcacggcagacaagtgaccgtgcaggagttcgccctgttcttcaccatcttcgacgaaactaagtcatggtactttaccgagaacatggagcggaattgtcgggccccatgtaacatccagatggaggacccgacattcaaggagaactaccggttccacgccattaacggatacattatggacactcttccgggactcgtgatggcacaggaccaacgcatcagatggtatcttctgtcgatggggagcaacgaaaacatccattcgatccactttagcggtcacgtgttcacagtgcgcaagaaggaagagtacaagatggcgctgtacaacctgtaccctggggtgttcgagactgtggaaatgctgccgtccaaggccggaatttggcgcgtggaatgtctgatcggtgaacatctgcatgccggaatgtccaccctgttcctggtgtactccaacaagtgccaaaccccactgggaatggcatcaggacacattagagacttccagattaccgcgagcggacagtacggacaatgggcccccaagttggccaggctgcactactctggaagcattaacgcctggagcaccaaggagccgttcagctggatcaaggtggaccttctggcgccaatgatcatccacggaattaagactcagggagcccgccagaagttctcatcgctctacatctcccagtttatcatcatgtactcactggatgggaagaagtggcagacttaccggggaaattccaccggtactctgatggtgttcttcggaaacgtggacagctccggcatcaagcacaatatctttaacccgcctatcatcgcccgatacatccggctccacccgactcactactccatccggtcgactctgcggatggaactcatgggttgcgacctcaactcctgctcaatgccactgggcatggagtccaaggctatctcggacgctcagattactgcatcgtcgtactttaccaacatgttcgctacctggtccccgtccaaagcccggctgcatctccaaggcagatcaaacgcgtggaggcctcaggtcaacaacccgaaggaatggcttcaggtcgacttccaaaagaccatgaaagtcaccggagtgaccacccagggcgtgaaatcgctgctgacctctatgtacgtgaaggaattcctgatctcatcaagccaggacggccaccagtggacactgttcttccaaaatggaaaggtcaaggtctttcagggaaatcaagactccttcacccccgtggtgaactccctggacccccctctgcttacccgctacttgcgcattcatccgcaatcctgggtgcaccagatcgccctgcgaatggaagtgctgggctgtgaagcgcaggacctgtac

SEQ ID NO: 94 - DNA, 4443 bp, Artificial sequence
  Synthetic polynucleotide
  misc_feature: F8-BDD2, codon optimized
gccaccagaaggtactacctaggagccgtggaactgagctgggactacatgcagtctgacctgggagagctgcccgtggacgctagatttcctccaagagtgcccaagagcttccccttcaacacctccgtggtgtacaagaaaaccctgttcgtggaattcaccgaccacctgttcaatatcgccaagcctagacctccttggatgggcctgctgggccctacaattcaggccgaggtgtacgacaccgtggtcatcaccctgaagaacatggccagccatcctgtgtctctgcacgccgtgggagtgtcttactggaaggcttctgagggcgccgagtacgacgaccagacaagccagagagagaaagaggacgacaaggttttccctggcggcagccacacctatgtctggcaggtcctgaaagaaaacggccctatggcctccgatcctctgtgcctgacatacagctacctgagccatgtggacctggtcaaggacctgaactctggcctgatcggcgctctgctcgtgtgtagagaaggcagcctggccaaagaaaagacccagacactgcacaagttcatcctgctgttcgccgtgttcgacgagggcaagagctggcacagcgagacaaagaacagcctgatgcaggacagagatgccgcctctgctagagcttggcccaagatgcacaccgtgaacggctacgtgaacagaagcctgcctggactgatcggatgccacagaaagtccgtgtactggcatgtgatcggcatgggcaccacacctgaggtgcacagcatctttctggaaggacacaccttcctcgtgcggaaccacagacaggccagcctggaaatcagccctatcaccttcctgaccgctcagaccctgctgatggatctgggccagtttctgctgttctgccacatcagcagccaccagcacgatggcatggaagcctacgtgaaggtggacagctgccccgaagaaccccagctgagaatgaagaacaacgaggaagccgaggactacgacgacgacctgaccgactctgagatggacgtcgtcagattcgacgacgataacagccccagcttcatccagatcagaagcgtggccaagaagcaccccaagacctgggtgcactatatcgccgccgaggaagaggactgggattacgctcctctggtgctggcccctgacgacagaagctacaagagccagtacctgaacaacggccctcagagaatcggccggaagtataagaaagtgcggttcatggcctacaccgacgagacattcaagaccagagaggctatccagcacgagagcggcattctgggacctctgctgtatggcgaagtgggcgacacactgctgatcatcttcaagaaccaggccagcagaccctacaacatctaccctcacggcatcaccgatgtgcggcctctgtactctagaaggctgcccaagggcgtgaagcacctgaaggacttccctatcctgcctggcgagatcttcaagtacaagtggaccgtgaccgtcgaggacggccctaccaagagcgatcctagatgcctgacacggtactacagcagcttcgtgaacatggaacgcgacctggccagcggcctgattggtcctctgctgatctgctacaaagaaagcgtggaccagaggggcaaccagatcatgagcgacaagagaaacgtgatcctgttctccgtctttgacgagaacaggtcctggtatctgaccgagaacatccagcggtttctgcccaatcctgctggcgtgcagctggaagatcctgagttccaggcctccaacatcatgcactccatcaacggctatgtgttcgacagcctgcagctgagcgtgtgcctgcacgaagtggcctactggtacatcctgtctatcggcgcccagaccgacttcctgtccgtgttctttagcggctacaccttcaagcacaagatggtgtacgaggataccctgacactgttcccattcagcggcgagacagtgttcatgagcatggaaaaccccggcctgtggatcctgggctgtcacaacagcgacttcagaaacagaggcatgacagccctgctgaaggtgtccagctgcgacaagaacaccggcgactactacgaggactcttacgaggacatcagcgcctacctgctgagcaagaacaatgccatcgagcctcggagcttctctcagaaccctcctgtgctgaagagacaccagcgcgagatcaccagaaccacactgcagagcgaccaagaggaaatcgattacgacgacaccatcagcgtcgagatgaagaaagaagatttcgacatctacgacgaggacgagaatcagagccccagatctttccagaagaaaacgcggcactacttcattgccgccgtggaaagactgtgggactacggcatgagcagcagcccacatgtgctgagaaacagggcccagagcggaagcgtgccccagttcaagaaagtggtgttccaagagttcaccgacggcagcttcacccagcctctgtatagaggcgagctgaacgagcacctgggactgctgggaccttacatcagagctgaggtcgaggataacatcatggtcacctttagaaaccaggcctctaggccctactccttctacagctccctgatcagctacgaagaggaccagagacagggcgctgagcccagaaagaacttcgtgaagcccaacgagactaagacctacttttggaaggtgcagcaccacatggcccctacaaaggacgagttcgactgcaaggcctgggcctacttctctgacgtggacctcgagaaggatgtgcacagcggactcatcggacccctgcttgtgtgccacaccaacacactgaatcccgctcacggcaggcaagtgaccgtgcaagagttcgccctgttcttcaccatcttcgatgagacaaagtcctggtacttcaccgaaaacatggaaagaaactgcagggccccttgcaacatccagatggaagatcccaccttcaaagagaactaccggttccacgccatcaatggctacatcatggacactctgcccggcctggttatggcacaggatcagaggatcagatggtatctgctgtccatgggctccaacgagaatatccacagcatccacttcagcggccatgtgttcaccgtgcggaaaaaagaagagtacaagatggccctgtacaatctgtaccccggcgtgttcgagactgtggaaatgctgcctagcaaggccggaatctggcgcgtggaatgtctgatcggagagcatctgcatgccggaatgtctaccctgttcctggtgtacagcaacaagtgtcagacccctctcggcatggcctctggacacatcagagacttccagatcaccgcctctggccagtacggacagtgggctcctaaactggctagactgcactacagcggcagcatcaacgcctggtccaccaaagagcccttcagctggatcaaggtggacctgctggctcccatgatcatccacggaatcaagacccagggcgccagacagaagttcagcagcctgtacatcagccagttcatcatcatgtacagcctggacggcaagaagtggcagacctacagaggcaacagcaccggcacactcatggtgttcttcggcaacgtggactccagcggcattaagcacaacatcttcaaccctccaatcattgcccggtacatccggctgcaccccacacactacagcatcagatctaccctgaggatggaactgatgggctgcgacctgaacagctgctctatgcccctcggaatggaaagcaaggccatcagcgacgcccagatcacagccagcagctacttcaccaacatgttcgccacatggtccccatctaaggcccggctgcatctgcagggcagatctaacgcttggaggccccaagtgaacaaccccaaagagtggctgcaggtcgactttcagaaaaccatgaaagtgaccggcgtgaccacacagggcgtcaagtctctgctgacctctatgtacgtgaaagagttcctgatctccagcagccaggacggccaccagtggaccctgtttttccagaacggcaaagtcaaggtgttccagggaaaccaggacagcttcacacccgtggtcaactccctggatcctccactgctgaccagatacctgagaattcaccctcagtcttgggtgcaccagatcgctctgagaatggaagtgctgggatgtgaagctcaggacctctactaaaataaaagatctttattttcattagatctgtgtgttggttttttgtgtgcgatcgggaactggcatcttcagggagtagcttaggtcagtgaagagaagtgccagttcccgatcgttacaggccgc

SEQ ID NO: 95 - DNA, 4317 bp, Artificial sequence
  Synthetic polynucleotide
  misc_feature: F8-BDD3, codon optimized
gccaccagaagatactacctgggagctgtggaattgagctgggattacatgcaatctgacctgggagaactgcctgtggatgccaggtttcctcctagggtccccaagtccttcccattcaacacctcagtggtctacaagaaaaccctctttgtggagttcacagaccatctgttcaacattgccaagccaagacccccatggatgggactcctgggtccaaccatccaagctgaagtgtatgacactgtggtcattaccctgaagaacatggcctcccatcctgtgtccctgcatgcagtgggagtgtcctactggaaggcttctgaaggggctgagtatgatgatcaaaccagccagagagaaaaggaggatgacaaagtgttcccaggtggtagtcacacctatgtgtggcaagtgctcaaggagaatggtcctatggcctctgatcccctgtgtctgacctactcctacctgtcccatgtggacctggtgaaggatctgaactctgggctgattggagccctgctggtgtgcagagaaggctccctggccaaggaaaagacccagacactgcacaagttcatcttgctgtttgctgtgtttgatgagggaaagtcctggcattctgagactaagaactcccttatgcaagacagagatgctgcctcagctagggcttggcctaagatgcatactgtgaatggatatgtgaacagatccctgcctggccttattggttgccacaggaagtctgtgtattggcatgtgattggcatgggaaccactccagaggtgcactccattttcttggaggggcataccttcttggtgaggaaccacagacaggcctccctggaaatttctccaatcactttcctgactgcccagaccctccttatggacctgggtcagttcctgctgttctgccacatttcatcccaccaacatgatggcatggaagcctatgtgaaagtggactcatgcccagaagaaccacagctgagaatgaagaacaatgaagaggcagaggactatgatgatgatcttacagattcagaaatggatgtggtcagatttgatgatgataatagcccatccttcatccaaattaggagtgtggccaagaagcaccccaaaacttgggtgcattacattgcagctgaggaagaggattgggactatgcacccttggtgcttgcaccagatgataggtcctacaagtcccaatacctgaacaatggcccacagaggattggtagaaagtataagaaagtgagattcatggcctacacagatgagactttcaagaccagagaggccattcagcatgaatctggcattctggggccactgttgtatggggaggttggagatacactgctcatcattttcaagaaccaggcctccagaccctacaacatctaccctcatggaatcactgatgtcagacccctgtactccagaagactcccaaagggagtcaagcacttgaaagacttccccatcctgcctggggaaatcttcaagtacaagtggacagtgacagtggaggatgggccaaccaagtctgatccaagatgcctcactagatactactcatcctttgtcaacatggaaagagacctggcctcaggactgattggccccctgctcatctgctacaaggagtctgtggatcagagaggaaaccagatcatgtctgacaaaaggaatgtcatcctcttctctgtctttgatgagaacagatcatggtaccttacagagaacatccagaggttcctccccaaccctgctggagtgcagctggaggacccagaattccaggcatcaaacattatgcactccatcaatggttatgtgtttgacagcctccagctttctgtgtgcctccatgaagtggcatattggtacatcctgtccattggagcacaaacagactttctctctgtgttcttctctggatataccttcaagcacaagatggtgtatgaggataccctgaccctcttccccttctctggagagactgtgtttatgtcaatggaaaacccaggcctgtggattttggggtgccacaactcagatttcagaaacaggggcatgactgccttgctcaaggtgtcctcctgtgacaagaacacaggagactactatgaggactcctatgaggatatttctgcctacctcctgtccaagaacaatgccattgaacccaggtccttcagccagaaccctcctgtcctcaagaggcatcagagagaaatcaccagaactaccctgcagtctgaccaggaagagattgattatgatgacactatctcagtggaaatgaagaaggaggactttgacatctatgatgaagatgaaaatcagtcccctaggtccttccaaaagaaaacaagacactacttcattgctgctgtggagagactctgggactatggcatgtcctcatcaccccatgtgcttaggaacagggctcaatctgggtctgtccctcagttcaagaaagtggtgtttcaagaattcacagatggaagcttcacacagccattgtacaggggagaactgaatgagcaccttggcctgctgggaccttacatcagagcagaggtggaggacaacatcatggtgaccttcagaaaccaagcctccaggccatattcattctactccagccttatctcatatgaggaggatcagagacagggggctgaacctaggaagaactttgtcaagccaaatgagacaaagacctacttttggaaggtgcagcaccacatggcccctaccaaggatgagtttgactgcaaggcctgggcttacttctctgatgtggatctggaaaaggatgtgcattctgggctgattggacctctgctggtctgccacactaacaccctcaatcctgctcatggcagacaagtgacagtgcaggagtttgccctgttcttcaccatctttgatgaaactaagtcatggtactttacagagaacatggagagaaattgtagggccccatgtaacatccagatggaggacccaacattcaaggagaactacagattccatgccattaatggatacattatggacactcttccaggactggtgatggcacaggaccaaagaatcagatggtatcttctgagcatggggagcaatgaaaacatccattccatccacttttcaggtcatgtgttcacagtgaggaagaaggaagagtacaagatggctctgtacaacctgtaccctggggtgtttgagactgtggaaatgctgccatccaaggctggaatttggagggtggaatgtctgattggtgaacatctgcatgctggaatgtccaccctgttcctggtgtactccaacaagtgccaaaccccactgggaatggcatcaggacacattagagacttccagattacagcatctggacagtatggacaatgggcccccaagttggccaggctgcactactctggaagcattaatgcctggagcaccaaggagccattcagctggatcaaggtggaccttctggctccaatgatcatccatggaattaagactcagggagccagacagaagttctcatccctctacatctcccagtttatcatcatgtactcactggatgggaagaagtggcagacttacaggggaaattccacaggtactctgatggtgttctttggaaatgtggacagctctggcatcaagcacaatatctttaaccctcctatcattgccaggtacatcagactccacccaactcactactccatcaggtccactctgaggatggaactcatgggttgtgacctcaactcctgctcaatgccactgggcatggagtccaaggctatctcagatgctcagattactgcatcctcttactttaccaacatgtttgctacctggtccccctccaaagccagactgcatctccaaggcagatcaaatgcctggaggcctcaggtcaacaacccaaaggaatggcttcaggtggacttccaaaagaccatgaaagtcacaggagtgaccacccagggagtgaaatccctgctgacctctatgtatgtgaaggaattcctgatctcatcaagccaggatggccaccagtggacactgttcttccaaaatggaaaggtcaaggtctttcagggaaatcaagactccttcacccctgtggtgaactccctggacccccctctgcttaccaggtacttgagaattcatccacaatcctgggtgcaccagattgccctgaggatggaagtgctgggctgtgaagcccaggacctgtactaa

SEQ ID NO: 96 - DNA, 4314 bp, Artificial sequence
  Synthetic polynucleotide
  misc_feature: F8-BDD4, codon optimized
gccaccagaaggtactacctgggagctgtggaactgagctgggactacatgcagtctgacctgggagagctgcctgtggatgctagatttcctccaagagtgcccaagagcttccccttcaacacctctgtggtgtacaagaaaaccctgtttgtggaattcacagaccacctgttcaatattgccaagcctagacctccttggatgggcctgctgggccctacaattcaggctgaggtgtatgacacagtggtcatcaccctgaagaacatggccagccatcctgtgtctctgcatgctgtgggagtgtcttactggaaggcttctgagggggctgagtatgatgaccagacaagccagagagagaaagaggatgacaaggttttccctgggggcagccacacctatgtctggcaggtcctgaaagaaaatggccctatggcctctgatcctctgtgcctgacatacagctacctgagccatgtggacctggtcaaggacctgaactctggcctgattggggctctgctggtgtgtagagaaggcagcctggccaaagaaaagacccagacactgcacaagttcatcctgctgtttgctgtgtttgatgagggcaagagctggcactctgagacaaagaacagcctgatgcaggacagagatgctgcctctgctagagcttggcccaagatgcacacagtgaatggctatgtgaacagaagcctgcctggactgattggatgccacagaaagtctgtgtactggcatgtgattggcatgggcaccacacctgaggtgcacagcatctttctggaaggacacaccttcctggtgaggaaccacagacaggccagcctggaaatcagccctatcaccttcctgacagctcagaccctgctgatggatctgggccagtttctgctgttctgccacatcagcagccaccagcatgatggcatggaagcctatgtgaaggtggacagctgccctgaagaaccccagctgagaatgaagaacaatgaggaagctgaggactatgatgatgacctgacagactctgagatggatgtggtcagatttgatgatgataacagccccagcttcatccagatcagatctgtggccaagaagcaccccaagacctgggtgcactatattgctgctgaggaagaggactgggattatgctcctctggtgctggcccctgatgacagaagctacaagagccagtacctgaacaatggccctcagagaattggcaggaagtataagaaagtgaggttcatggcctacacagatgagacattcaagaccagagaggctatccagcatgagtctggcattctgggacctctgctgtatggggaagtgggggacacactgctgatcatcttcaagaaccaggccagcagaccctacaacatctaccctcatggcatcacagatgtgaggcctctgtactctagaaggctgcccaagggggtgaagcacctgaaggacttccctatcctgcctggggagatcttcaagtacaagtggacagtgacagtggaggatggccctaccaagtctgatcctagatgcctgacaaggtactacagcagctttgtgaacatggaaagggacctggcctctggcctgattggtcctctgctgatctgctacaaagaatctgtggaccagaggggcaaccagatcatgagtgacaagagaaatgtgatcctgttctctgtctttgatgagaacaggtcctggtatctgacagagaacatccagaggtttctgcccaatcctgctggggtgcagctggaagatcctgagttccaggcctccaacatcatgcactccatcaatggctatgtgtttgacagcctgcagctgtctgtgtgcctgcatgaagtggcctactggtacatcctgtctattggggcccagacagacttcctgtctgtgttcttttctggctacaccttcaagcacaagatggtgtatgaggataccctgacactgttcccattctctggggagacagtgttcatgagcatggaaaaccctggcctgtggatcctgggctgtcacaacagtgacttcagaaacagaggcatgacagccctgctgaaggtgtccagctgtgacaagaacactggggactactatgaggactcttatgaggacatctctgcctacctgctgagcaagaacaatgccattgagcctaggagcttctctcagaaccctcctgtgctgaagagacaccagagggagatcaccagaaccacactgcagtctgaccaagaggaaattgattatgatgacaccatctctgtggagatgaagaaagaagattttgacatctatgatgaggatgagaatcagagccccagatctttccagaagaaaacaaggcactacttcattgctgctgtggaaagactgtgggactatggcatgagcagcagcccccatgtgctgagaaacagggcccagtctggaagtgtgccccagttcaagaaagtggtgttccaagagttcacagatggcagcttcacccagcctctgtatagaggggagctgaatgagcacctgggactgctgggaccttacatcagagctgaggtggaggataacatcatggtcacctttagaaaccaggcctctaggccctactccttctacagctccctgatcagctatgaagaggaccagagacagggggctgagcccagaaagaactttgtgaagcccaatgagactaagacctacttttggaaggtgcagcaccacatggcccctacaaaggatgagtttgactgcaaggcctgggcctacttctctgatgtggacctggagaaggatgtgcactctggactcattggacccctgcttgtgtgccacaccaacacactgaatcctgctcatggcaggcaagtgacagtgcaagagtttgccctgttcttcaccatctttgatgagacaaagtcctggtacttcacagaaaacatggaaagaaactgcagggccccttgcaacatccagatggaagatcccaccttcaaagagaactacaggttccatgccatcaatggctacatcatggacactctgcctggcctggttatggcacaggatcagaggatcagatggtatctgctgtccatgggctccaatgagaatatccacagcatccacttctctggccatgtgttcacagtgaggaaaaaagaagagtacaagatggccctgtacaatctgtaccctggggtgtttgagactgtggaaatgctgcctagcaaggctggaatctggagggtggaatgtctgattggagagcatctgcatgctggaatgtctaccctgttcctggtgtacagcaacaagtgtcagacccctctgggcatggcctctggacacatcagagacttccagatcacagcctctggccagtatggacagtgggctcctaaactggctagactgcactactctggcagcatcaatgcctggtccaccaaagagcccttcagctggatcaaggtggacctgctggctcccatgatcatccatggaatcaagacccagggggccagacagaagttcagcagcctgtacatcagccagttcatcatcatgtacagcctggatggcaagaagtggcagacctacagaggcaacagcacaggcacactcatggtgttctttggcaatgtggactcttctggcattaagcacaacatcttcaaccctccaatcattgccaggtacatcaggctgcaccccacacactacagcatcagatctaccctgaggatggaactgatgggctgtgacctgaacagctgctctatgcccctgggaatggaaagcaaggccatctctgatgcccagatcacagccagcagctacttcaccaacatgtttgccacatggtccccatctaaggccaggctgcatctgcagggcagatctaatgcttggaggccccaagtgaacaaccccaaagagtggctgcaggtggactttcagaaaaccatgaaagtgacaggagtgaccacacagggggtcaagtctctgctgacctctatgtatgtgaaagagttcctgatctccagcagccaggatggccaccagtggaccctgtttttccagaatggcaaagtcaaggtgttccagggaaaccaggacagcttcacacctgtggtcaactccctggatcctccactgctgaccagatacctgagaattcaccctcagtcttgggtgcaccagattgctctgagaatggaagtgctgggatgtgaagctcaggacctctac

SEQ ID NO: 97 - DNA, 4314 bp, Artificial sequence
  Synthetic polynucleotide
  misc_feature: Native FVIII-BDD mature CDS
gccaccagaagatactacctgggtgcagtggaactgtcatgggactatatgcaaagtgatctcggtgagctgcctgtggacgcaagatttcctcctagagtgccaaaatcttttccattcaacacctcagtcgtgtacaaaaagactctgtttgtagaattcacggttcaccttttcaacatcgctaagccaaggccaccctggatgggtctgctaggtcctaccatccaggctgaggtttatgatacagtggtcattacacttaagaacatggcttcccatcctgtcagtcttcatgctgttggtgtatcctactggaaagcttctgagggagctgaatatgatgatcagaccagtcaaagggagaaagaagatgataaagtcttccctggtggaagccatacatatgtctggcaggtcctgaaagagaatggtccaatggcctctgacccactgtgccttacctactcatatctttctcatgtggacctggtaaaagacttgaattcaggcctcattggagccctactagtatgtagagaagggagtctggccaaggaaaagacacagaccttgcacaaatttatactactttttgctgtatttgatgaagggaaaagttggcactcagaaacaaagaactccttgatgcaggatagggatgctgcatctgctcgggcctggcctaaaatgcacacagtcaatggttatgtaaacaggtctctgccaggtctgattggatgccacaggaaatcagtctattggcatgtgattggaatgggcaccactcctgaagtgcactcaatattcctcgaaggtcacacatttcttgtgaggaaccatcgccaggcgtccttggaaatctcgccaataactttccttactgctcaaacactcttgatggaccttggacagtttctactgttttgtcatatctcttcccaccaacatgatggcatggaagcttatgtcaaagtagacagctgtccagaggaaccccaactacgaatgaaaaataatgaagaagcggaagactatgatgatgatcttactgattctgaaatggatgtggtcaggtttgatgatgacaactctccttcctttatccaaattcgctcagttgccaagaagcatcctaaaacttgggtacattacattgctgctgaagaggaggactgggactatgctcccttagtcctcgcccccgatgacagaagttataaaagtcaatatttgaacaatggccctcagcggattggtaggaagtacaaaaaagtccgatttatggcatacacagatgaaacctttaagactcgtgaagctattcagcatgaatcaggaatcttgggacctttactttatggggaagttggagacacactgttgattatatttaagaatcaagcaagcagaccatataacatctaccctcacggaatcactgatgtccgtcctttgtattcaaggagattaccaaaaggtgtaaaacatttgaaggattttccaattctgccaggagaaatattcaaatataaatggacagtgactgtagaagatgggccaactaaatcagatcctcggtgcctgacccgctattactctagtttcgttaatatggagagagatctagcttcaggactcattggccctctcctcatctgctacaaagaatctgtagatcaaagaggaaaccagataatgtcagacaagaggaatgtcatcctgttttctgtatttgatgagaaccgaagctggtacctcacagagaatatacaacgctttctccccaatccagctggagtgcagcttgaggatccagagttccaagcctccaacatcatgcacagcatcaatggctatgtttttgatagtttgcagttgtcagtttgtttgcatgaggtggcatactggtacattctaagcattggagcacagactgacttcctttctgtcttcttctctggatataccttcaaacacaaaatggtctatgaagacacactcaccctattcccattctcaggagaaactgtcttcatgtcgatggaaaacccaggtctatggattctggggtgccacaactcagactttcggaacagaggcatgaccgccttactgaaggtttctagttgtgacaagaacactggtgattattacgaggacagttatgaagatatttcagcatacttgctgagtaaaaacaatgccattgaaccaagaagcttctcccagaatccaccagtcttgaaacgccatcaacgggaaataactcgtactactcttcagtcagatcaagaggaaattgactatgatgataccatatcagttgaaatgaagaaggaagattttgacatttatgatgaggatgaaaatcagagcccccgcagctttcaaaagaaaacacgacactattttattgctgcagtggagaggctctgggattatgggatgagtagctccccacatgttctaagaaacagggctcagagtggcagtgtccctcagttcaagaaagttgttttccaggaatttactgatggctcctttactcagcccttataccgtggagaactaaatgaacatttgggactcctggggccatatataagagcagaagttgaagataatatcatggtaactttcagaaatcaggcctctcgtccctattccttctattctagccttatttcttatgaggaagatcagaggcaaggagcagaacctagaaaaaactttgtcaagcctaatgaaaccaaaacttacttttggaaagtgcaacatcatatggcacccactaaagatgagtttgactgcaaagcctgggcttatttctctgatgttgacctggaaaaagatgtgcactcaggcctgattggaccccttctggtctgccacactaacacactgaaccctgctcatgggagacaagtgacagtacaggaatttgctctgtttttcaccatctttgatgagaccaaaagctggtacttcactgaaaatatggaaagaaactgcagggctccctgcaatatccagatggaagatcccacttttaaagagaattatcgcttccatgcaatcaatggctacataatggatacactacctggcttagtaatggctcaggatcaaaggattcgatggtatctgctcagcatgggcagcaatgaaaacatccattctattcatttcagtggacatgtgttcactgtacgaaaaaaagaggagtataaaatggcactgtacaatctctatccaggtgtttttgagacagtggaaatgttaccatccaaagctggaatttggcgggtggaatgccttattggcgagcatctacatgctgggatgagcacactttttctggtgtacagcaataagtgtcagactcccctgggaatggcttctggacacattagagattttcagattacagcttcaggacaatatggacagtgggccccaaagctggccagacttcattattccggatcaatcaatgcctggagcaccaaggagcccttttcttggatcaaggtggatctgttggcaccaatgattattcacggcatcaagacccagggtgcccgtcagaagttctccagcctctacatctctcagtttatcatcatgtatagtcttgatgggaagaagtggcagacttatcgaggaaattccactggaaccttaatggtcttctttggcaatgtggattcatctgggataaaacacaatatttttaaccctccaattattgctcgatacatccgtttgcacccaactcattatagcattcgcagcactcttcgcatggagttgatgggctgtgatttaaatagttgcagcatgccattgggaatggagagtaaagcaatatcagatgcacagattactgcttcatcctactttaccaatatgtttgccacctggtctccttcaaaagctcgacttcacctccaagggaggagtaatgcctggagacctcaggtgaataatccaaaagagtggctgcaagtggacttccagaagacaatgaaagtcacaggagtaactactcagggagtaaaatctctgcttaccagcatgtatgtgaaggagttcctcatctccagcagtcaagatggccatcagtggactctcttttttcagaatggcaaagtaaaggtttttcagggaaatcaagactccttcacacctgtggtgaactctctagacccaccgttactgactcgctaccttcgaattcacccccagagttgggtgcaccagattgccctgaggatggaggttctgggctgcgaggcacaggacctctac

SEQ ID NO: 98 - protein, 9 aa, Artificial sequence
  Synthetic polypeptide
DAHATRRYY

SEQ ID NO: 99 - DNA, 12 bp, Artificial sequence
  Synthetic polynucleotide
  misc_feature: Branch site consensus sequence
  misc_feature (3)..(3): n is a, c, g, or t
tcncttcagact

SEQ ID NO: 100 - DNA, 22 bp, Artificial sequence
  Synthetic polynucleotide
  misc_feature: target nucleic acid sequence
  misc_feature (1)..(20): N is any nucleotide
  misc_feature (21)..(21): R can be G or A
nnnnnnnnnnnnnnnnnnnnrg

SEQ ID NO: 101 - DNA, 8 bp, Neisseria meningitidis
  misc_feature: Neisseria meningitidis PAM
  misc_feature (1)..(4): N is any nucleotide
nnnngatt

SEQ ID NO: 102 - DNA, 9 bp, Neisseria meningitidis
  misc_feature: Neisseria meningitidis PAM
  misc_feature (1)..(5): N is any nucleotide
nnnnngttt

SEQ ID NO: 103 - DNA, 8 bp, Neisseria meningitidis
  misc_feature: Neisseria meningitidis PAM
  misc_feature (1)..(4): N is any nucleotide
nnnngctt

SEQ ID NO: 104 - DNA, 23 bp, Homo sapiens
  misc_feature: Human Albumin Intron-1_T25
acctttttttttttttacctagg

SEQ ID NO: 105 - RNA, 20 nt, Artificial sequence
  Synthetic polynucleotide
  misc_feature: Exemplary gRNA spacer
uaauuuucuuuugcgcacua

SEQ ID NO: 106 - DNA, 20 bp, Artificial sequence
  Synthetic polynucleotide
taaagcatagtgcaatggat

SEQ ID NO: 107 - DNA, 20 bp, Artificial sequence
  Synthetic polynucleotide
gaaagcatggtgcaatggat

SEQ ID NO: 108 - DNA, 20 bp, Artificial sequence
  Synthetic polynucleotide
tattgcacagtgcaatggat

SEQ ID NO: 109 - DNA, 20 bp, Artificial sequence
  Synthetic polynucleotide
tgatgcatattgcaatggat

SEQ ID NO: 110 - DNA, 20 bp, Artificial sequence
  Synthetic polynucleotide
taatgaatagggcaatggat

SEQ ID NO: 111 - DNA, 20 bp, Artificial sequence
  Synthetic polynucleotide
taaggcacagtgtaatggat

SEQ ID NO: 112 - DNA, 20 bp, Artificial sequence
  Synthetic polynucleotide
aaaagcatagaccaatggat

SEQ ID NO: 113 - DNA, 20 bp, Artificial sequence
  Synthetic polynucleotide
tagagtatagtgcagtggat

SEQ ID NO: 114 - DNA, 20 bp, Artificial sequence
  Synthetic polynucleotide
caaagcaaagtgcaattgat

SEQ ID NO: 115 - DNA, 20 bp, Artificial sequence
  Synthetic polynucleotide
ggaagcatagtgcaatggtt

SEQ ID NO: 116 - DNA, 20 bp, Artificial sequence
  Synthetic polynucleotide
taaaggatagagcaatgtat

SEQ ID NO: 117 - DNA, 20 bp, Artificial sequence
  Synthetic polynucleotide
tagagtatagtgcaatggag

SEQ ID NO: 118 - DNA, 20 bp, Artificial sequence
  Synthetic polynucleotide
taaagaatagtgaaatggtt

SEQ ID NO: 119 - DNA, 20 bp, Artificial sequence
  Synthetic polynucleotide
atttatgagatcaacagcac

SEQ ID NO: 120 - DNA, 20 bp, Artificial sequence
  Synthetic polynucleotide
atttatgatatcatcagcac

SEQ ID NO: 121 - DNA, 20 bp, Artificial sequence
  Synthetic polynucleotide
aaatatgacatcaacagcac

SEQ ID NO: 122 - DNA, 20 bp, Artificial sequence
  Synthetic polynucleotide
atctttgagatcatcagcac

SEQ ID NO: 123 - DNA, 20 bp, Artificial sequence
  Synthetic polynucleotide
atgtatcagatcatcagcac

SEQ ID NO: 124 - DNA, 20 bp, Artificial sequence
  Synthetic polynucleotide
aattatgagattcacagcac

SEQ ID NO: 125 - DNA, 20 bp, Artificial sequence
  Synthetic polynucleotide
atttatgtgttcaaccgcac

SEQ ID NO: 126 - DNA, 20 bp, Artificial sequence
  Synthetic polynucleotide
atatatgacatcaacagaac

SEQ ID NO: 127 - DNA, 20 bp, Artificial sequence
  Synthetic polynucleotide
acttatgatatcaacagcat

SEQ ID NO: 128 - DNA, 20 bp, Artificial sequence
  Synthetic polynucleotide
ttaaataaagcatagtgcaa

SEQ ID NO: 129 - DNA, 20 bp, Artificial sequence
  Synthetic polynucleotide
taaaataaagcatagtgcaa

SEQ ID NO: 130 - DNA, 20 bp, Artificial sequence
  Synthetic polynucleotide
ttaaataaaggatattgcaa

SEQ ID NO: 131 - DNA, 20 bp, Artificial sequence
  Synthetic polynucleotide
ttaaataaagcattgagcaa

SEQ ID NO: 132 - DNA, 20 bp, Artificial sequence
  Synthetic polynucleotide
tactataaagcatagtgcaa

SEQ ID NO: 133 - DNA, 20 bp, Artificial sequence
  Synthetic polynucleotide
tactataaagcatagtgcaa

SEQ ID NO: 134 - DNA, 20 bp, Artificial sequence
  Synthetic polynucleotide
ttaaggaaaccatagtgcaa

SEQ ID NO: 135 - DNA, 20 bp, Artificial sequence
  Synthetic polynucleotide
ataaatatatcatagtgcaa

SEQ ID NO: 136 - DNA, 20 bp, Artificial sequence
  Synthetic polynucleotide
ctaaatagagaatagtgcaa

SEQ ID NO: 137 - DNA, 20 bp, Artificial sequence
  Synthetic polynucleotide
ttaaagaaattatagtgcaa

SEQ ID NO: 138 - DNA, 20 bp, Artificial sequence
  Synthetic polynucleotide
ttaaatatataatagtgcaa

SEQ ID NO: 139 - DNA, 20 bp, Artificial sequence
  Synthetic polynucleotide
ttaaaaacagcacagtgcaa

SEQ ID NO: 140 - DNA, 20 bp, Artificial sequence
  Synthetic polynucleotide
ttaaaataagcatggtgcaa

SEQ ID NO: 141 - DNA, 20 bp, Artificial sequence
  Synthetic polynucleotide
tttgataaagcatagggcaa

SEQ ID NO: 142 - DNA, 20 bp, Artificial sequence
  Synthetic polynucleotide
ttttataaagcatagtccaa

SEQ ID NO: 143 - DNA, 20 bp, Artificial sequence
  Synthetic polynucleotide
ttaaatgaagaatattgcaa

SEQ ID NO: 144 - DNA, 20 bp, Artificial sequence
  Synthetic polynucleotide
ataaataaagaatagagcaa

SEQ ID NO: 145 - DNA, 20 bp, Artificial sequence
  Synthetic polynucleotide
ttgaataaagcagagtggaa

SEQ ID NO: 146 - DNA, 20 bp, Artificial sequence
  Synthetic polynucleotide
ttaattaatgcatagtgcca

SEQ ID NO: 147 - DNA, 20 bp, Artificial sequence
  Synthetic polynucleotide
ttagataaagcttagtgcta

SEQ ID NO: 148 - DNA, 20 bp, Artificial sequence
  Synthetic polynucleotide
ttagataaagcatactggaa

SEQ ID NO: 149 - DNA, 20 bp, Artificial sequence
  Synthetic polynucleotide
ttaaagaaagcatggtgcag

SEQ ID NO: 150 - DNA, 20 bp, Artificial sequence
  Synthetic polynucleotide
ttacataaagcatactgcat

SEQ ID NO: 151 - DNA, 20 bp, Artificial sequence
  Synthetic polynucleotide
ttatataaagcatagagcag

SEQ ID NO: 152 - DNA, 20 bp, Artificial sequence
  Synthetic polynucleotide
ttaaatgaagcatagtgaag

SEQ ID NO: 153 - DNA, 20 bp, Artificial sequence
  Synthetic polynucleotide
taataaaattcaaacatcct

SEQ ID NO: 154 - DNA, 20 bp, Artificial sequence
  Synthetic polynucleotide
gaataaaattctaacatcct

SEQ ID NO: 155 - DNA, 20 bp, Artificial sequence
  Synthetic polynucleotide
taatataattccaacatcct

SEQ ID NO: 156 - DNA, 20 bp, Artificial sequence
  Synthetic polynucleotide
aaataaaattcaaacttcct

SEQ ID NO: 157 - DNA, 20 bp, Artificial sequence
  Synthetic polynucleotide
gagttaaattcaaacatcct

SEQ ID NO: 158 - DNA, 20 bp, Artificial sequence
  Synthetic polynucleotide
ttttaaaaatcaaacatcct

SEQ ID NO: 159 - DNA, 20 bp, Artificial sequence
  Synthetic polynucleotide
aaatgaaagtcaaacatcct

SEQ ID NO: 160 - DNA, 20 bp, Artificial sequence
  Synthetic polynucleotide
gattaaaatttaaacatcct

SEQ ID NO: 161 - DNA, 20 bp, Artificial sequence
  Synthetic polynucleotide
tcttaaaattccaacatcct

SEQ ID NO: 162 - DNA, 20 bp, Artificial sequence
  Synthetic polynucleotide
aaaaaaaattccaacatcct

SEQ ID NO: 163 - DNA, 20 bp, Artificial sequence
  Synthetic polynucleotide
tactgaaattctaacatcct

SEQ ID NO: 164 - DNA, 20 bp, Artificial sequence
  Synthetic polynucleotide
tacaaaaattcacacatcct

SEQ ID NO: 165 - DNA, 20 bp, Artificial sequence
  Synthetic polynucleotide
tattagaattcagacatcct

SEQ ID NO: 166 - DNA, 20 bp, Artificial sequence
  Synthetic polynucleotide
taataaagcccaaacatcct

SEQ ID NO: 167 - DNA, 20 bp, Artificial sequence
  Synthetic polynucleotide
tatttaaattcaaatatcct

SEQ ID NO: 168 - DNA, 20 bp, Artificial sequence
  Synthetic polynucleotide
aaataaagttcaaagatcct

SEQ ID NO: 169 - DNA, 20 bp, Artificial sequence
  Synthetic polynucleotide
tacaaaaattcaaacttcct

SEQ ID NO: 170 - DNA, 20 bp, Artificial sequence
  Synthetic polynucleotide
gaataaaatttaaatatcct

SEQ ID NO: 171 - DNA, 20 bp, Artificial sequence
  Synthetic polynucleotide
tataaaaattcaaacagcct

SEQ ID NO: 172 - DNA, 20 bp, Artificial sequence
  Synthetic polynucleotide
tactaaaatttaaacttcct

SEQ ID NO: 173 - DNA, 20 bp, Artificial sequence
  Synthetic polynucleotide
taataaccttcaaacattct

SEQ ID NO: 174 - DNA, 20 bp, Artificial sequence
  Synthetic polynucleotide
tagtaaaattcaaatgtcct

SEQ ID NO: 175 - DNA, 20 bp, Artificial sequence
  Synthetic polynucleotide
caataaaattcaaccatcat

SEQ ID NO: 176 - DNA, 20 bp, Artificial sequence
  Synthetic polynucleotide
gaataaaattcaaacttctt

SEQ ID NO: 177 - DNA, 20 bp, Artificial sequence
  Synthetic polynucleotide
aaataaaattcaaaaatccc

SEQ ID NO: 178 - DNA, 23 bp, Artificial sequence
  Synthetic polynucleotide
  misc_feature: AlbF primer
ccctccgtttgtcctagcttttc

SEQ ID NO: 179 - DNA, 30 bp, Artificial sequence
  Synthetic polynucleotide
  misc_feature: AlbR primer
ccagatacagaatatcttcctcaacgcaga

SEQ ID NO: 180 - DNA, 20 bp, Artificial sequence
  Synthetic polynucleotide
  misc_feature: For primer
cctttggcacaatgaagtgg

SEQ ID NO: 181 - DNA, 22 bp, Artificial sequence
  Synthetic polynucleotide
  misc_feature: Rev primer
gaatctgaaccctgatgacaag

SEQ ID NO: 182 - DNA, 21 bp, Artificial sequence
  Synthetic polynucleotide
taaagcatagtgccaatggat

SEQ ID NO: 183 - DNA, 21 bp, Artificial sequence
  Synthetic polynucleotide
gaaagcataatagcaatggat

SEQ ID NO: 184 - DNA, 27 bp, Artificial sequence
  Synthetic polynucleotide
ctcaccatggggcgcctgcaactggtt

SEQ ID NO: 185 - DNA, 25 bp, Artificial sequence
  Synthetic polynucleotide
taaaggatcgttcaactctgtgagt

SEQ ID NO: 186 - DNA, 19 bp, Artificial sequence
  Synthetic polynucleotide
ccaagcataggtaatggat

SEQ ID NO: 187 - DNA, 19 bp, Artificial sequence
  Synthetic polynucleotide
aaaagcatagtgaatgaat

SEQ ID NO: 188 - DNA, 21 bp, Artificial sequence
  Synthetic polynucleotide
cttcgtatagaaacaagacag

SEQ ID NO: 189 - DNA, 21 bp, Artificial sequence
  Synthetic polynucleotide
gagagagagagaaagagacag

SEQ ID NO: 190 - DNA, 27 bp, Artificial sequence
  Synthetic polynucleotide
attctagaggcatagagagttcaacct

SEQ ID NO: 191 - DNA, 25 bp, Artificial sequence
  Synthetic polynucleotide
ttttctgccattgaccttaaagcgc

SEQ ID NO: 192 - DNA, 22 bp, Artificial sequence
  Synthetic polynucleotide
atctgtgggattatgactgaac

SEQ ID NO: 193 - DNA, 21 bp, Artificial sequence
  Synthetic polynucleotide
cttctcataaaacctagacag

SEQ ID NO: 194 - DNA, 19 bp, Artificial sequence
  Synthetic polynucleotide
atgtggagatcattgagca

SEQ ID NO: 195 - DNA, 27 bp, Artificial sequence
  Synthetic polynucleotide
attaatatggtatcatgggagcaggac

SEQ ID NO: 196 - DNA, 21 bp, Artificial sequence
  Synthetic polynucleotide
ttaaaataaagcatagtgcaa

SEQ ID NO: 197 - DNA, 18 bp, Artificial sequence
  Synthetic polynucleotide
ttccaacgaaggcctcaa

SEQ ID NO: 198 - DNA, 21 bp, Artificial sequence
  Synthetic polynucleotide
ttactataaagcatagtgcaa

SEQ ID NO: 199 - DNA, 23 bp, Artificial sequence
  Synthetic polynucleotide
aaaaaaaaagaaaagaaaagaaa

SEQ ID NO: 200 - DNA, 20 bp, Artificial sequence
  Synthetic polynucleotide
ttacctaaaacaatttcaca

SEQ ID NO: 201 - DNA, 31 bp, Artificial sequence
  Synthetic polynucleotide
ttaatactgggccctgaagccaaatacagtt

SEQ ID NO: 202 - DNA, 27 bp, Artificial sequence
  Synthetic polynucleotide
ggcaacaacacatcatcagtagggtaa

SEQ ID NO: 203 - DNA, 27 bp, Artificial sequence
  Synthetic polynucleotide
ttcagaaatagaaaagctgatcctcaa

SEQ ID NO: 204 - DNA, 28 bp, Artificial sequence
  Synthetic polynucleotide
ttaaagcgcttgaaatctacacttgcaa

SEQ ID NO: 205 - DNA, 27 bp, Artificial sequence
  Synthetic polynucleotide
tgataggatgtgtgtgtagaagactcc

SEQ ID NO: 206 - DNA, 24 bp, Artificial sequence
  Synthetic polynucleotide
ccatagaagataccaggacttctt

SEQ ID NO: 207 - DNA, 31 bp, Artificial sequence
  Synthetic polynucleotide
atgtagaagttggtcacggtccgcatcggct

SEQ ID NO: 208 - DNA, 23 bp, Artificial sequence
  Synthetic polynucleotide
aaatagaatacctcagcatttct

SEQ ID NO: 209 - DNA, 31 bp, Artificial sequence
  Synthetic polynucleotide
agatgaaaatctatcaatggcaccagcgcct

SEQ ID NO: 210 - DNA, 32 bp, Artificial sequence
  Synthetic polynucleotide
taaaaaagggctgagcatagtggctcacacct

SEQ ID NO: 211 - DNA, 28 bp, Artificial sequence
  Synthetic polynucleotide
tattcaactcacagagttgaacgatcct

SEQ ID NO: 212 - DNA, 5 bp, Artificial sequence
  Synthetic polynucleotide
  misc_feature: Spacer
tcgcg